(19) United States
(12) Patent Application Publication
(10) Pub. No.: US 2014/0140959 A1
(43) Pub. Date: May 22, 2014

(54) Energy Absorbing-Based Diagnostic and Therapeutic Methods Employing Nucleic Acid Molecules Encoding Chromophore-Producing Enzymes

(71) Applicants: Aladar A. Szalay, Highland, CA (US); Jochen Stritzker, La Jolla, CA (US)

(72) Inventors: Aladar A. Szalay, Highland, CA (US); Jochen Stritzker, La Jolla, CA (US)

(21) Appl. No.: 13/998,130

(22) Filed: Oct. 4, 2013

Related U.S. Application Data
(60) Provisional application No. 61/795,025, filed on Oct. 5, 2012.
(60) Provisional application No. 61/850,256, filed on Feb. 11, 2013.

(51) Int. Cl. A61K 41/00

(52) U.S. Cl. CPC A61K 41/0057
USPC 424/93.2; 514/44 R; 435/235.1

(57) ABSTRACT

Provided are diagnostic and therapeutic methods using a composition containing a nucleic acid encoding a chromophore-producing enzyme(s), such as a melanin-producing enzyme or enzymes. The nucleic acid molecules can be used for directed or localized delivery to tumors or other cells associated with disease resulting in production of a chromophore product that can be used for diagnosis and therapy, including of tumors and metastases.

### RELATED APPLICATIONS

Benefit of priority is claimed to U.S. Provisional Application No. 61/795,025, filed Oct. 5, 2012, entitled “Energy Absorbing-Based Diagnostic and Therapeutic Methods Employing Nucleic Acid Molecules Encoding Chromophore-Producing Enzymes” and U.S. Provisional Application No. 61/850,256, entitled “Energy Absorbing-Based Diagnostic And Therapeutic Methods Employing Nucleic Acid Molecules Encoding Chromophore-Producing Enzymes,” filed Feb. 11, 2013.

This application is related to corresponding International Application No. [Attorney Docket No. 33316.04836.WO01/4836PC], filed the same day herewith, entitled “Energy Absorbing-Based Diagnostic and Therapeutic Methods Employing Nucleic Acid Molecules Encoding Chromophore-Producing Enzymes,” which also claims priority to U.S. Provisional Application Nos. 61/795,025 and 61/850,256.

The subject matter of each of the above-referenced applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Oct. 4, 2013, is identical, 10.1 megabytes in size, and titled 4836seq.001.txt.

### FIELD OF THE INVENTION

Provided are diagnostic and therapeutic methods using a composition containing a nucleic acid encoding a chromophore-producing enzyme(s), such as a melanin-producing enzyme or enzymes. The nucleic acid molecules can be used for directed or localized delivery to tumors or other cells associated with disease resulting in production of a chromophore product that can be used for diagnosis and therapy, including of tumors and metastases.

### BACKGROUND

Each year over ten million people worldwide are diagnosed with cancer, and there are over six and half million deaths from the disease. Treatment with various chemotherapeutic agents, including chemotherapeutic compounds and radiation, are an important part of modern clinical cancer treatment. Biological therapies, such as gene therapies, cell therapies and oncolytic viral therapies, also are viable treatment modalities. Early and accurate diagnosis of tumors and directed therapy of tumor cells remains a limitation for many diagnostic and treatment methods. Hence, there exists a need for improved diagnostic and/or therapeutic methods. These and other needs are addressed herein.

### SUMMARY

Provided are methods for killing or inhibiting growth or proliferation of cells, which are involved in a disease, by an energy-absorbing therapy. The method can be practiced by: a) administering a nucleic acid molecule encoding a chromophore-producing enzyme(s) to a subject, where the nucleic acid molecule is directed or localized to cells in the subject that are involved in the disease process and expression of the encoded chromophore-producing enzyme(s) produces a chromophore product in the cells in the subject into which the nucleic acid molecule is delivered; and then, b) exposing the subject to an energy source that is absorbed by the chromophore product to effect local production of heat and/or toxic chemicals in the cell and/or electromagnetic radiation, thereby killing the cells or inhibiting the proliferation of the cells that express the enzyme(s), thereby effecting treatment of the disease. The encoded nucleic acid can encode one enzyme that catalyzes production of a chromophore (including electromagnetic radiation or other radiation absorbing molecules) or a plurality of enzymes in a metabolic pathway for the same or different chromophore(s). Absorption of the energy by the chromophore(s) can produce localized heat in the cells or lead to production of a toxin by the cell, to thereby kill or inhibit or prevent replication of the cell. Step b) typically occurs after a time sufficient for the nucleic acid molecule to be expressed to produce an enzyme, and the chromophore product whose production is catalyzed by the enzyme produced.

Also provided are methods of use of nucleic acid molecules encoding a chromophore-producing enzyme for formulation of a medicament for killing or inhibiting growth or proliferation of cells, which are involved in a disease, by energy absorbing therapy, where the chromophore-producing enzyme catalyzes production of a chromophore product when introduced into a cell; and the chromosome product absorbs energy from an energy source.

Also provided are pharmaceutical compositions used for killing or inhibiting growth or proliferation of cells, which are involved in a disease, by energy absorbing therapy, where: the compositions contain a nucleic acid molecule encoding a chromophore-producing enzyme(s); the chromophore-producing enzyme catalyzes production of a chromophore product when introduced into a cell; and the chromosome product absorbs energy from an energy source.

The following discussion of particular embodiments and details applies to the methods, methods of use and compositions. The cells, targeted by the methods, methods of use and compositions provided, are those that are involved in disease whose killing or inhibition effects treatment of the disease(s). Diseases include, but are not limited to, one or more of a proliferative disease, an inflammatory disease or an immune-mediated disease, such as a myeloproliferative disease, a lymphoproliferative disease, or a solid tumor disease. Proliferative diseases include, but are not limited to, cancer, atherosclerosis, rheumatoid arthritis, psoriasis, idiopathic pulmonary fibrosis, scleroderma and cirrhosis. Cancers include, but are not limited to, carcinoma, sarcoma, lymphoma and leukemia, such as cancer of the tongue, mouth, throat, stomach, cecum, colon, rectum, breast, ovary, uterus, thyroid, adrenal cortex, lung, kidney, prostate or pancreas. Proliferative disorders also include tumors and metastases. The cells involved include, but are not limited to, an inflammatory cell, an immune cell, a tumor cell. Tumors and tumor cells include solid tumors, circulating tumor cells and metastatic cells. In particular, the cells are non-melanoma cells and/or the tumors treated are not melanomas.

The nucleic acid molecule encoding the chromophore-producing enzyme(s) can be delivered by any suitable vehicle. For example, the molecule can be operatively inserted into a vector for expression in a cell. Vectors include, but are not limited to, viral vectors and non-viral vectors. Viral vectors include oncolytic viruses. such as, but not limited to, a Newcastle Disease virus, parvovirus, vaccinia virus, measles virus, reovirus, vesicular stomatitis virus (VSV), oncolytic adenoviruses, poliovirus and herpes viruses, or a derivative of any of these viruses that is modified to contain nucleic acid encoding a heterologous gene product. When the virus a poxvirus, it can be a vaccinia virus, such as, but not limited to, strains, such as Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC16m8, LC16M0, LIVP, ACAM2000, WR 65-16, Connaught, New York City Board of Health (NYCBH), EM-63 and the NYVAC strain. Lister viruses include LIVP. The viruses can be clonal strains of an oncolytic virus. The sequence of nucleotides encoding a chromophore-producing enzyme can be inserted into or in place of a non-essential gene or region in the genome of an unmodified oncolytic virus or is inserted into or in place of nucleic acid encoding a heterologous gene product in the genome of an unmodified oncolytic virus.

Exemplary unmodified (not including the chromophore-producing enzyme) viruses include, an LIVP or derivative thereof comprising a sequence of nucleotides set forth in SEQ ID NO:1 or 188, or a sequence of nucleotides that has at least 85% 86%, 87%, 88%, 89%, 90%, 91%, 92%, 93%, 94%, 95%, 96%, 97%, 98%, 99% or more sequence identity to SEQ ID NO:1 or 188. LIVP and derivatives thereof can contain, for example, a sequence of nucleotides selected from: a) nucleotides 2,256-181,114 of SEQ ID NO:55, nucleotides 11,243-182,721 of SEQ ID NO:56, nucleotides 6,264-181,390 of SEQ ID NO:57, nucleotides 7,044-181,820 of SEQ ID NO:58, nucleotides 6,674-181,409 of SEQ ID NO:59, nucleotides 6,716-181,367 of SEQ ID NO:60 or nucleotides 6,899-181,870 of SEQ ID NO:61; or b) a sequence of nucleotides that has at least 85% sequence identity to a sequence of nucleotides 2,256-181,114 of SEQ ID NO:55, nucleotides 11,243-182,721 of SEQ ID NO:56, nucleotides 6,264-181,390 of SEQ ID NO:57, nucleotides 7,044-181,820 of SEQ ID NO:58, nucleotides 6,674-181,409 of SEQ ID NO:59, nucleotides 6,716-181,367 of SEQ ID NO:60 or nucleotides 6,899-181,870 of SEQ ID NO:61. The unmodified (virus not encoding the chromophore producing enzyme) oncolytic virus can be a LIVP clonal strain or derivative thereof, such as a strain comprising the sequence of nucleotides set forth in one of SEQ ID NOS: 55-61, or a sequence of nucleotides that has at least 86%, 87%, 88%, 89%, 90%, 91%, 92%, 93%, 94%, 95%, 96%, 97%, 98%, 99% or more sequence identity to one of SEQ ID NOS: 55-61. The unmodified oncolytic virus can contain nucleic acid encoding a heterologous gene product. The heterologous nucleic acid can be inserted into or in place of a non-essential gene or region in the genome of the virus. Heterologous gene products include, but are not limited to, therapeutic products and reporter gene products. A plurality of such products can be included in the virus. Exemplary products, include, but are not limited to, an anticancer agent, an antimetastatic agent, an antiangiogenic agent, and an immunomodulatory molecule, such as, for example, a therapeutic agent selected from among a hormone, a growth factor, a cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein and a tissue factor. These include, for example, a therapeutic agent selected from among a granulocyte macrophage colony stimulating factor (GM-CSF), monocyte chemotactic protein-1 (MCP-1), interleukin-6 (IL-6), interleukin-24 (IL-24), interferon gamma-induced protein 10 (IP-10), lymphotoxin inducible expression competes with HSV glycoprotein D for HVEM a receptor expressed on T-lymphocytes (LIGHT), p60 superantigen, OspF, OspG, signal transducer and activator of transcription protein (STAT1alpha), STAT1beta, plasminogen k5 domain (hK5), pigment epithelium-differentiation factor (PEDF), single chain anti-VEGF antibody, single chain anti-DLL4 antibody, single chain anti-fibroblast activation protein (FAP), NM23, cadherin 1 (ECAD or cdh1), relaxin 1 (RLN1), matrix metallopeptidase 9 (MMP9), erythropoietin (EPO), microRNA126 (miR-126), microRNA 181, microRNA 335, manganese superoxide dismutase (MnSOD), E3 ubiquitin protein ligase 1 (HACE1), natriuretic peptide precursor A (nppal), carboxypeptidase G2 (CPG2), alcohol dehydrogenase (ADH), CDC6, and bone morphogenetic protein 4 (BMP4). Reporter genes and reporter gene products include any that produce a signal or induce a product the produces a signal. Signals can be detected by any suitable method, including light and imaging, such as MRI and PET imaging. Products include a reporter gene product selected from among a fluorescent protein, a bioluminescent protein, an enzyme, or a cell surface protein that is capable of detection.

Other unmodified oncolytic viruses include any known to those of skill in the art, including those selected from among viruses designated GLV-1h68, JX-594, JX-954, ColoAd1, MV-CEA, MV-NIS, ONYX-015, B18R, H101, OncoVEX GM-CSF, Reolysin, NTX-010, CCTG-102, Cavatak, Oncorine, TNFerade, GLV-1h64, GLV-1i69, GLV-1h70, GLV-1h71, GLV-1h72, GLV-1h73, GLV-1h74, GLV-1h76, GLV-1h77, GLV-1h78, GLV-1h79, GLV-1h80, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1h92, GLV-1h93, GLV-1h94, GLV-1h95, GLV-1h96, GLV-1h97, GLV-1h98, GLV-1h99, GLV-1h100, GLV-1h101, GLV-1h102, GLV-1h103, GLV-1h104, GLV-1h105, GLV-1h106, GLV-1h107, GLV-1h108, GLV-1h109, GLV-1h110, GLV-1h111, GLV-1h112, GLV-1h113, GLV-1h114, GLV-1h115, GLV-1h116, GLV-1h117, GLV-1h118, GLV-1h119, GLV-1h120, GLV-1h121, GLV-1h122, GLV-1h123, GLV-1h124, GLV-1h125, GLV-1h126, GLV-1h127, GLV-1h128, GLV-1h129, GLV-1h130, GLV-1h131, GLV-1h132, GLV-1h133, GLV-1h134, GLV-1e135, GLV-1h136, GLV-1h137, GLV-1h138, GLV-1h139, GLV-1h140, GLV-1h141, GLV-1h142, GLV-1h143, GLV-1h144, GLV-1h145, GLV-1h146, GLV-1h147, GLV-1h148, GLV-1h149, GLV-1h150, GLV-1h151, GLV-1h153, GLV-1h154, GLV-1h155, GLV-1h156, GLV-1h157, GLV-1h158, GLV-1h159, GLV-1h160, GLV-1h161, GLV-1h162, GLV-1h163, GLV-1h164, GLV-1h165, GLV-1h166, GLV-1h167, GLV-1h168, GLV-1h169, GLV-1h170, GLV-1h171, GLV-1h172, GLV-1h173, GLV-1h174, GLV-1h175, GLV-1h176, GLV-1h177, GLV-1h178, GLV-1h179, GLV-1h180, GLV-1h181, GLV-1h182, GLV-1h183, GLV-1h184, GLV-1h185, GLV-1h186, GLV-1h187, GLV-1h188, GLV-1h189, GLV-1h190, GLV-1h191, GLV-1h192, GLV-1h193, GLV-1h194, GLV-1h195, GLV-1h196, GLV-1h197, GLV-1h198, GLV-1h199, GLV-1h201, GLV-1h202, GLV-1h203, GLV-1h204, GLV-1h205, GLV-1h208, GLV-1h210, GLV-1h211, GLV-1h212, GLV-1h213, GLV-1h214, GLV-1h215, GLV-1h216, GLV-1h217, GLV-1h218, GLV-1h219, GLV-1h220, GLV-1h221, GLV-1h222, GLV-1h223, GLV-1h224, GLV-1h225, GLV-1h226, GLV-1h227, GLV-1h228, GLV-1h229, GLV-1h230, GLV-1h231, GLV-1h232, GLV-1h233, GLV-1h234, GLV-1h235, GLV-1h236, GLV-1h237, GLV-1h238, GLV-1h239, GLV-1h240, GLV-1h241, GLV-1h242, GLV-1h243, GLV-1h244, GLV-1h245, GLV-1h246, GLV-1h247, GLV-1h248, GLV-1h249, GLV-1h250, GLV-1h251, GLV-1h252, GLV-1h253, GLV-1h254, GLV-1h255, GLV-1h256, GLV-1h257, GLV-1h258, GLV-1h259, GLV-1h260, GLV-1h261, GLV-1h262, GLV-1h263, GLV-1h264, GLV-1h265, GLV-1h266, GLV-1h267, GLV-1h268, GLV-1h269, GLV-1h270, GLV-1h271, GLV-1h272, GLV-1h273, GLV-1h274, GLV-1h275, GLV-1h276, GLV-1h277, GLV-1h284, GLV-1h285, GLV-1b372, GLV-1h286, GLV-1h311, GLV-1h312, GLV-1h330, GLV-1h354, GLV-2b372, GLV-0b348, GLV-0b358 and GLV-0e365. Viruses among these have a sequence of nucleotides selected from among any of SEQ ID NOS: 2, 3, 65, 66, 189-194, 201-203, 210, or 211, or a sequence of nucleotides that exhibits at least 86%, 87%, 88%, 89%, 90%, 91%, 92%, 93%, 94%, 95%, 96%, 97%, 98%, 99% or more s sequence identity to any of SEQ ID NOS: 2, 3, 65, 66, 189-194, 201-203, 210, or 211.

The nucleic acid encoding the chromophore-producing enzyme can be provided to cells involved in a disease process in any manner in which they will be expressed primarily in such cells. This includes targeted delivery, such as delivery in vehicles that specifically interact with the cells, such as by virtue of a specific ligand, or vehicles in which the nucleic acid is expressed in such cells, such as control of expression by tissue/cell specific regulatory elements. Thus, the nucleic acid molecules encoding the enzymes can be inserted into any suitable vehicle and can be operatively inserted for expression in a cell. Such vehicles include, but are not limited to, non-viral vectors and viral vectors. Non-viral vectors include, but are not limited to, plasmids, cosmids, minicircles and artificial chromosomes.

The vehicles and vectors can be formulated for delivery, such as in a liposome, micelle, or nanoparticle. The vector, liposome or nanoparticle can be conjugated to a protein that targets the vector, liposome or nanoparticle to the cell involved in the disease. Conjugation can be direct or indirect. It can be via covalent or other interactions. Targeting to the cell, includes conjugation to a protein that targets to a tumor cell. Such a protein, that targets a tumor, can be selected from among transferrin, an arginine-glycine-aspartate (RGD) peptide, an αvβ3 binding targeting peptide, folate and an antibody or fragment thereof that specifically binds to a protein expressed or overexpressed on the surface of a tumor cell.

The chromophores produced upon expression of the encoded chromophore-producing enzyme include any that can be produced in a cell via a biochemical processes in the cell in which the enzyme participates. Any chromophore (energy-absorbing moiety) is contemplated, including those that heat up and/or produce a toxic product, including free radicals when exposed to energy. Energy includes any form of electromagnetic energy. Exemplary chromophores are melanins and/or precursors of melanins. Melanins include eumelanin and/or pheomelanin. The enzymes selected can produce a variety of ratios of eumelanin to total melanin.

Ratios include at least 0.2, 0.3, 0.4, 0.5, 0.6, 0.7, 0.8, 0.9 or more.

The chromophore-producing enzyme, encoded by the nucleic acid molecule, can be a tyrosinase, enzymatically active portion thereof or an enzymatically active variant thereof. Tyrosinases can be human or non-human tyrosinase. Exemplary tyrosinase enzymes include those having the sequence of amino acids set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence or a sequence of amino acids that exhibits at least 60%, 65%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 7, 81, 82, 84, 85, 87, 89, 91, 93, 95, 97, 99, 101; 103, 105, 107, 109, 111, 113, 115, or 117, or a mature form thereof lacking the signal sequence.

Exemplary nucleic acid molecules are those that include the sequence of nucleotides set forth in any of SEQ ID NOS: 6, 80, 83, 86, 88, 90, 92, 94, 96, 98, 100, 102, 104, 106, 108, 110, 112, 114, 116 or 118 or a sequence of nucleotides that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 6, 80, 83, 86, 88, 90, 92, 94, 96, 98, 100, 102, 104, 106, 108, 110, 112, 114, 116 or 118. Exemplary nucleic acid molecules are those that encode the sequence of amino acids set forth in any of SEQ ID NOS: 7, 81, 82 and 84, or a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 7, 81, 82 and 84. Exemplary nucleic acid molecules are those that include the sequence of nucleotides set forth in any of SEQ ID NOS: 6, 80 or 83, or a sequence of nucleotides that exhibits at least 70% 75%, 80%, 85%, 86%, 87%, 88%, 89%, 90%, 91%, 92%, 93%, 94%, 95%, 96%, 97%, 98%, 99% or more sequence identity to any of SEQ ID NOS: 6, 80 or 83.

A tyrosinase, enzymatically active portion thereof or an enzymatically active variant thereof, can be the only chromophore-producing enzyme encoded by the nucleic acid molecule, or the nucleic acid can further encode other chromophore-producing enzyme(s), such as a tyrosinase-related protein 1 (TRP-1) and/or a dopachrome tautomerase (DCT), or an enzymatically active portion thereof or an enzymatically active variant thereof, that are part of a melanin-producing pathway.

Any human or non-human TRP-1 enzyme is contemplated. Exemplary TRP-1 enzymes are those having the sequence of amino acids set forth in any of SEQ ID NOS: 20, 120, 121, 123, 124, 126, 127, 129, 131, 133, 135, 137, 139, 141, 142 or 144, or a mature form thereof lacking the signal sequence, or a sequence of amino acids that exhibits at least 75%, 80%, 85%, 86%, 87%, 88%, 89%, 90%, 91%, 92%, 93%, 94%, 95%, 96%, 97%, 98%, 99% or more sequence identity to any of SEQ ID NOS: 20, 120, 121, 123, 124, 126, 127, 129, 131, 133, 135, 137, 139, 141, 142 or 144 or a mature form thereof lacking the signal sequence. TRP-1-encoding nucleic acid molecules include those that contain the sequence of nucleotides set forth in any of SEQ ID NOS: 19, 119, 122, 125, 128, 130, 132, 134, 136, 138, 140, 143 or 145 or a sequence of nucleotides that exhibits at least 70%, 80%, 85%, 86%, 87%, 88%, 89%, 90%, 91%, 92%, 93%, 94%, 95%, 96%, 97%, 98%, 99% or more sequence identity to any of SEQ ID NOS: 19, 119, 122, 125, 128, 130, 132, 134, 136, 138, 140, 143 or 145.

The encoded DCT, or an enzymatically active portion thereof or an enzymatically active variant thereof, includes any such enzyme, including human and non-human enzymes. DCT enzymes include those that have the sequence of amino acids set forth in any of SEQ ID NOS: 30, 146, 147, 148, 150, 152, 154, 156, 158, 160, 162, 164 or 166, or a mature form thereof lacking the signal sequence, or a sequence of amino acids that exhibits at least 75%, 80%, 85%, 86%, 87%, 88%, 89%, 90%, 91%, 92%, 93%, 94%, 95%, 96%, 97%, 98%, 99% or more sequence identity to any of SEQ ID NOS: 30, 146, 147, 148, 150, 152, 154, 156, 158, 160, 162, 164 or 166, or the mature form thereof lacking the signal sequence. Included among the nucleic acid molecules are those where the nucleic acid molecule contains the sequence of nucleotides set forth in any of SEQ ID NOS: 29, 149, 151, 153, 155, 157, 159, 161, 163, 165 or 167 or a sequence of nucleotides that exhibits at least 70%, 80%, 85%, 86%, 87%, 88%, 89%, 90%, 91%, 92%, 93%, 94%, 95%, 96%, 97%, 98%, 99% or more sequence identity to any of SEQ ID NOS: 29, 149, 151, 153, 155, 157, 159, 161, 163, 165 or 167.

The nucleic acid molecule can encode a tyrosinase (Tyr) alone or in combination with a Trp 1 and/or DCT enzyme. Hence, exemplary nucleic acids are those that encode these enzymes as set forth above, including combinations of any of the sequences of nucleotides or encoded polypeptides set forth above. For example, the nucleic acid molecule can encode a chromophore-producing enzyme that is a tyrosinase or enzymatically active portion thereof or enzymatically active variant thereof and a tyrosinase-related protein 1 or enzymatically active portion thereof or enzymatically active variant thereof, where:

the nucleic acid molecule encodes a tyrosinase enzyme having a sequence of nucleotides selected from among: i) a sequence of nucleotides that encodes a tyrosinase enzyme having the sequence of amino acids set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence or a sequence of amino acids that exhibits at least 75% sequence identity to any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence; and ii) a sequence of nucleotides containing the sequence of nucleotides set forth in any of SEQ ID NOS: 6, 80, 83, 86, 88, 90, 92, 94, 96, 98, 100, 102, 104, 106, 108, 110, 112, 114, 116 or 118 or a sequence of nucleotides that exhibits at least 70% sequence identity to any of SEQ ID NOS: 6, 80, 83, 86, 88, 90, 92, 94, 96, 98, 100, 102, 104, 106, 108, 110, 112, 114, 116 or 118; and

the nucleic acid molecule encodes a TRP-1 enzyme having a sequence of nucleotides that is i) a sequence of nucleotides encoding a TRP-1 enzyme having the sequence of amino acids set forth in any of SEQ ID NOS: 20, 120, 121, 123, 124, 126, 127, 129, 131, 133, 135, 137, 139, 141, 142 or 144, or a mature form thereof lacking the signal sequence, or a sequence of amino acids that exhibits at least 75% or more sequence identity to any of SEQ ID NOS: 20, 120, 121, 123, 124, 126, 127, 129, 131, 133, 135, 137, 139, 141, 142 or 144 or the mature form thereof lacking the signal sequence; or ii) a sequence of nucleotides that contains the sequence of nucleotides set forth in any of SEQ ID NOS: 19, 119, 122, 125, 128, 130, 132, 134, 136, 138, 140, 143 or 145 or a sequence of nucleotides that exhibits at least 70% sequence identity to any of SEQ ID NOS: 19, 119, 122, 125, 128, 130, 132, 134, 136, 138, 140, 143 or 145.

The nucleic acid molecule can be operatively linked to regulatory regions for expression of the encoded enzymes to achieve constitutive or regulated expression of the encoded enzymes. Hence, the nucleic acid molecule can include a promoter operatively linked to the open reading frame encoding the chromophore-producing enzyme(s). Promoters include eukaryotic promoters, including eukaryotic virus promoters. The promoter can be a strong promoter and/or a promoter that is expressed at a particular time. Exemplary promoters included viral promoters, such as an adenoviral major late promoter, vaccinia synthetic early-late promoter (P_{SEL}), vaccinia synthetic late promoter (P_{SL}), simian virus 40 (SV40) promoter, cytomegalovirus (CMV) promoter, respiratory syncytial virus (RSV) promoter, a retroviral LTR promoter, human elongation factor 1α-subunit (EF1-1α) promoter, a ubiquitin C promoter (Ubc), a phosphoglycerate kinase-1 (PGK) promoter, small nuclear RNA U1b promoter and glucose 6-phosphate dehydrogenase promoter. Exemplary promoters include vaccinia virus promoters, such as, but not limited to, promoters selected from among P_{7.5k}, P₁₁ₖ, P_{SE}, P_{SEL}, P_{SL}, H5R, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b and K1 promoters.

The promoters include those that are expressed in specific tissues or cells. Exemplary promoters include tumor-specific promoters, such as, but not limited to, a promoter selected from among c-erbB-2 oncogene, carcinoembryonic antigen (CEA), mucin (MUC1), prostate specific antigen (PSA), alpha-fetoprotein (AFP), L-plastin (LP-P), α-lactalbumin (ALA), midkine (MK), cyclooxygenase-2 (COX-2), probasin (ARR2PB), hypoxic response elements (HRE), hTERT, flt-1, flk1/KDR, E-selectin, endoglin, ICAM-2, preproendothelin 1 (PPE-1), prolactin (PRL), osteocalcin 2, CXCR4 tumor-specific promoters, E2F-1, antigen 33, cyclin A (CycA), cell division cycle 2 (Cdc2), cell division cycle 25 (Cdc25), B-myb, p107, tyrosine kinase (TK), DNA polymerase alpha, histone 2A (H2A), c-myc and a synthetic cell cycle-dependent promoter.

Particular embodiments of the nucleic acid molecules include, but are not limited to, nucleic acid molecules encoding a melanin-producing enzyme(s), operatively inserted into an oncolytic virus, whereby the oncolytic virus contains the sequence of nucleotides set forth in any of SEQ ID NOS: 64, 67-70, 198-200, 204-206 or 212-216 or a sequence of nucleotides that exhibits at least 85%, 86%, 87%, 88%, 89%, 90%, 91%, 92%, 93%, 94%, 95%, 96%, 97%, 98%, 99% or more sequence identity to any of SEQ ID NOS: 64, 67-70, 198-200, 204-206 or 212-216.

After administration of the nucleic acid to a subject, energy is administered. The energy should be administered after a sufficient time for the chromophore to be expressed. Timing depends upon the delivery vehicle, the targeted cell, the enzyme expressed and other such parameters known to the skilled artisan. Such times can be empirically determined, if necessary, or predetermined. Hence, the methods (and compositions) are employed such that the subject is exposed to an energy source, the energy of which is absorbed by the produced chromophore after administration of the nucleic acid molecule encoding the chromophore-producing enzyme(s). The time can be predetermined, and includes time sufficient for the nucleic acid molecule to express the chromophore-producing enzyme(s) in a cell in the subject and the production of the chromophore product in the cell. Examples of such timing is at least 8 hours, 10 hours, 12 hours to 1 week, 8 days, 1 month or 24 hours to 2 weeks after delivery of the nucleic acid molecule. As noted above, various regimens will include cyclical administration of the nucleic acid, such that the energy can be applied at regular intervals after the first administration of the nucleic acid. The energy can be administered/applied at least, 8 hours, 12 hours, 24 hours, 48 hours, 72 hours, 4 days, 5 days, 6 days, 7 days, 8 days, 9 days, 10 days, 11 days, 12 days, 13 days and 14 days post-nucleic acid delivery or at such regular intervals after the first administration of the nucleic acid molecule(s).

Energy includes electromagnetic energy and chemical energy. Energy includes any that is absorbed by the resulting chromophore. These include, but are not limited to, chemical energy, electric energy, radiant energy, microwave energy, nuclear energy, magnetic energy, elastic energy, sound energy, mechanical energy and luminous energy. The energy can be applied externally or internally. External energy can be applied where the target cells are on or near the surface of the skin, and/or where the energy penetrates into the body. Thus, for example, energy can be applied externally to an area of the subject to be treated, including where there is a targeted tissue/cell, such as a tumor located no more than 30 mm below the skin.

The energy can be applied internally, for example, by employing fiber optics to administer directly to a tissue or cell or organ in the subject. The energy source includes electromagnetic (EM) energy applied to effect photothermal therapy. EM energy includes that where the wavelength of electromagnetic energy applied is 500 to 1500 nm, 600 to 1200 nm or 700 to 900 nm, such as but are not limited to, less than 1500 nm, 1400 nm, 1300 nm, 1200 nm, 1100 nm, 1000 nm, 900 nm and/or is at least or at least about 500 nm, 600 nm, 700 nm, 800 nm and 900 nm. The energy can be electromagnetic energy applied to effect photodynamic therapy. For such therapy, the wavelength of electromagnetic energy applied can be 100 to 400 nm, 100 to 280 nm, 280 nm to 320 nm, 280 to 315 nm, 315 to 400 nm or 320 to 420 nm or other suitable range, such as less than 500 nm and/or is at least 50 nm, 100 nm, 200 nm, 300 nm, or 400 nm. Energy sources include, but are not limited to a light source selected from among a laser, light-emitting diodes, fluorescent lamps, dichroic lamps, and a light box. Energy can be applied internally, such as with an endoscope or fiber optic catheter.

The energy is applied for a sufficient time to effect treatment. The time should be sufficient to release sufficient heat for hyperthermia and/or to produce a product, such as free radical or toxic product. The time depends upon the energy administered as well as the source and target. Exemplary time periods include, but are not limited to, 30 seconds to 30 minutes, 1 minute to 20 minutes, 2 minutes to 15 minutes or 1 minute to 10 minutes, including at least 60 or 90 seconds, 5 minutes, 10 minutes, 15 minutes or longer. The energy can be applied one time, repeatedly or intermittently after administration of the nucleic acid molecule.

The amount of nucleic acid molecule to administer to the subject depends upon the form of the nucleic acid molecule and other parameters known to those of skill in the art. For example, where the nucleic acid molecule is a non-viral vector or DNA, the nucleic acid molecule can be administered in exemplary amounts in the range of from or from about 0.005 mg/kg body weight to 50 mg/kg body weight, such as administered in an amount in the range of from or from about 0.005 mg/kg to 20 mg/kg or 0.05 mg/kg to 5 mg/kg. Where the nucleic acid molecule is a viral vector, exemplary dosages includes 1×10⁴ to 1×10¹⁴ pfu, such as 1×10⁵ to 1×10⁹, including for example 1×10⁴ to 1×10⁸ pfu, 1×10⁵ to 1×10⁷ pfu, 1×10⁷ to 1×10¹⁴ pfu, 1×10⁷ to 1×10¹⁰ pfu or 1×10⁹ to 1×10¹⁰ pfu, such as at least or about 1×10⁶, 1×10⁷, 1×10⁸, 1×10⁹, 2×10⁹, 3×10⁹, 4×10⁹, or 5×10⁹ pfu.

Where the nucleic acid molecule is administered in a composition, the composition is administered in a suitable volume, such as, but not limited to, from or from about 0.01 mL to 100 mL, such as, but not limited to, from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL or 0.5 mL to 5 mL or at least or 0.05 mL, 0.5 mL, 1 mL, 2 mL, 3 mL, 4 mL, 5 mL, 6 mL, 7 mL, 8 mL, 9 mL or 10 mL.

The nucleic acid molecule can be administered, topically, locally or systemically, such as administered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, topically, ocularly, vaginally, or rectally. Local administration includes locally inside a body cavity. The nucleic acid molecule can be delivered with a method that facilitates or enhances delivery, such, for example delivery in the presence of a physical method to facilitate entry into cells selected from among electroporation, sonoporation, hydrodynamic, pressure, ultrasound and gene gun.

Subjects include any living organism, including animals and plants. Animals, include human and non-human mammals and other animals such as a gorilla, bonobo, ape, monkey, mouse, rat, amphibian, rabbit, ferret, chicken, goat, cow, deer, sheep, horse, pig, dog, cat and other zoo animals, farm animals and pets. The subject is one who has a disease or is suspected of having a disease, such as cancer. Cancers include solid tumors and tumors of the blood and lymph system, such as cancer of the lung, breast, colon, brain, prostate, liver, pancreas, esophagus, kidney, stomach, thyroid, bladder, uterus, cervix, ovary and leukemias and lymphomas. The tumors can be localized or metastatic.

The methods and methods of use herein include, not only treatment, but also diagnosis prior to treatment and monitoring during and after treatment, including to confirm that the nucleic acid is delivered and the chromophore produced. Hence the methods and methods of use include those where the nucleic acid molecule encoding a chromophore-producing enzyme is administered to the subject and the method of diagnosing the subject involves detecting the chromophore-product in the subject. Detection of expression of the chromophore in the subject can employ any method and/or product suitable for detection of the particular chromophore, including, for example, optoacoustic imaging, multispectral optoacoustic tomographic (MSOT) imaging and magnetic resonance imaging (MRI).

As noted above, the nucleic acid can be administered one or a plurality of times, including cyclically. The nucleic acid molecule can be administered, for example, two times, three times, four times, five times, six times or seven times. The nucleic acid administration can be for detection, detection and treatment, and/or treatment and monitoring. In addition, the nucleic acid administration and energy treatment can be employed with other therapies for a particular disorder. For example, the energy treatment can be employed in combination with a second (or several) therapeutic agent or treatment for the particular disease, such as a proliferative disorder or disease, such as cancer. The different treatments can be administered together, sequentially, simultaneously, intermittently and/or other combinations thereof.

Other treatments include, but are not limited to, surgery, radiation therapy, immunosuppressive therapy, administration of an anticancer agent or administration of an oncolytic virus (such as a virus different from the virus encoding the chromophore-producing enzyme, where the nucleic acid administered is a virus). Other treatments include, for example, administration of an anticancer agent selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent and a combination of any of the preceding. These include, for example, cisplatin, carboplatin, gemcitabine, irinotecan, an anti-EGFR antibody and an anti-VEGF antibody.

Where the energy therapy employs an oncolytic virus and/or another therapy is oncolytic virus therapy, the oncolytic viruses employed include, but are not limited to, newcastle Disease virus, parvovirus, reovirus, measles virus, vaccinia virus, vesicular stomatitis virus (VSV), oncolytic adenoviruses and herpes viruses, including such viruses modified to contain nucleic acid encoding a heterologous gene product, such as a therapeutic product. The heterologous gene product includes a therapeutic and/or reporter gene product, such as, but not limited to, an anticancer agent, an antimetastatic agent, an antiangiogenic agent, and/or an immunomodulatory molecule.

Oncolytic viruses include, but are not limited to, those designated GLV-1h68, JX-594, JX-954, ColoAd1, MV-CEA, MV-NIS, ONYX-015, B18R, H101, OncoVEX GM-CSF, Reolysin, NTX-010, CCTG-102, Cavatak, Oncorine, TNFerade, GLV-1h64, GLV-1i69, GLV-1h70, GLV-1h71, GLV-1h72, GLV-1h73, GLV-1h74, GLV-1h76, GLV-1h77, GLV-1h78, GLV-1h79, GLV-1h80, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1h92, GLV-1h93, GLV-1h94, GLV-1h95, GLV-1h96, GLV-1h97, GLV-1h98, GLV-1h99, GLV-1h100, GLV-1h101, GLV-1h102, GLV-1h103, GLV-1h104, GLV-1h105, GLV-1h106, GLV-1h107, GLV-1h108, GLV-1h109, GLV-1h110, GLV-1h111, GLV-1h112, GLV-1h113, GLV-1h114, GLV-1h115, GLV-1h116, GLV-1h117, GLV-1h118, GLV-1h119, GLV-1h120, GLV-1h121, GLV-1h122, GLV-1h123, GLV-1h124, GLV-1h125, GLV-1h126, GLV-1h127, GLV-1h128, GLV-1h129, GLV-1h130, GLV-1h131, GLV-1h132, GLV-1h133, GLV-1h134, GLV-1e135, GLV-1h136, GLV-1h137, GLV-1h138, GLV-1h139, GLV-1h140, GLV-1h141, GLV-1h142, GLV-1h143, GLV-1h144, GLV-1h145, GLV-1h146, GLV-1h147, GLV-1h148, GLV-1h149, GLV-1h150, GLV-1h151, GLV-1h153, GLV-1h154, GLV-1h155, GLV-1h156, GLV-1h157, GLV-1h158, GLV-1h159, GLV-1h160, GLV-1h161, GLV-1h162, GLV-1h163, GLV-1h164, GLV-1h165, GLV-1h166, GLV-1h167, GLV-1h168, GLV-1h169, GLV-1h170, GLV-1h171, GLV-1h172, GLV-1h173, GLV-1h174, GLV-1h175, GLV-1h176, GLV-1h177, GLV-1h178, GLV-1h179, GLV-1h180, GLV-1h181, GLV-1h182, GLV-1h183, GLV-1h184, GLV-1h185, GLV-1h186, GLV-1h187, GLV-1h188, GLV-1h189, GLV-1h190, GLV-1h191, GLV-1h192, GLV-1h193, GLV-1h194, GLV-1h195, GLV-1h196, GLV-1h197, GLV-1h198, GLV-1h199, GLV-1h201, GLV-1h202, GLV-1h203, GLV-1h204, GLV-1h205, GLV-1h208, GLV-1h210, GLV-1h211, GLV-1h212, GLV-1h213, GLV-1h214, GLV-1h215, GLV-1h216, GLV-1h217, GLV-1h218, GLV-1h219, GLV-1h220, GLV-1h221, GLV-1h222, GLV-1h223, GLV-1h224, GLV-1h225, GLV-1h226, GLV-1h227, GLV-1h228, GLV-1h229, GLV-1h230, GLV-1h231, GLV-1h232, GLV-1h233, GLV-1h234, GLV-1h235, GLV-1h236, GLV-1h237, GLV-1h238, GLV-1h239, GLV-1h240, GLV-1h241, GLV-1h242, GLV-1h243, GLV-1h244, GLV-1h245, GLV-1h246, GLV-1h247, GLV-1h248, GLV-1h249, GLV-1h250, GLV-1h251, GLV-1h252, GLV-1h253, GLV-1h254, GLV-1h255, GLV-1h256, GLV-1h257, GLV-1h258, GLV-1h259, GLV-1h260, GLV-1h261, GLV-1h262, GLV-1h263, GLV-1h264, GLV-1h265, GLV-1h266, GLV-1h267, GLV-1h268, GLV-1h269, GLV-1h270, GLV-1h271, GLV-1h272, GLV-1h273, GLV-1h274, GLV-1h275, GLV-1h276, GLV-1h277, GLV-1h284, GLV-1h285, GLV-1b372, GLV-1h286, GLV-1h311, GLV-1h312, GLV-1h330, GLV-1h354, GLV-2b372, GLV-0b348, GLV-0b358 and GLV-0e365.

Also provided are compositions that contain any Lister strain virus or vaccinia virus that encodes one or more chromophore-encoding enzyme(s). These include any of the above-noted viruses modified to encode the enzyme(s), including the enzymes described above. The compositions include pharmaceutical compositions, which can include a pharmaceutically acceptable carrier. They can be formulated for local or systemic administration.

Also provided are the viruses. For example, also provided are Lister strain viruses or vaccinia viruses, and compositions containing them, that contain a sequence of nucleotides encoding a tyrosinase or an enzymatically active portion or an enzymatically active variant of the tyrosinase enzyme. The viruses include those discussed above that encode the tyrosinases. Vaccinia viruses include those of strains, such as Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC 16m8, LC 16M0, LIVP, ACAM2000, WR 65-16, NYCBH, EM-63 and NYVAC strain. These include LIVP or WR viruses, including all of those described herein. Hence, the Lister strain viruses or vaccinia viruses provided contain a sequence of nucleotides encoding the tyrosinase that is operatively linked to a promoter, such as a eukaryotic promoter or a viral promoter, which can be native or heterologous, such as vaccinia viral promoters selected from among P_{7.5k}, P₁₁ₖ, P_{SE}, P_{SEL}, P_{SL}, H5R, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b and K1 promoters.

Vaccinia viruses or Lister strain viruses provided include those that contain a sequence of nucleotides encoding a tyrosinase, or an enzymatically active portion thereof, or an enzymatically active variant of the enzyme operatively linked to a strong promoter, or one selected for high expression of the tyrosinase. In particular, the promoters include those that are strong promoters for high expression or for expression timed after viral replication to increase the amount of enzyme produced. Exemplary promoters include a virus late promoter, a retroviral LTR, and other strong eukaryotic promoters, such as, but not limited to, adenoviral major late promoter, vaccinia synthetic early-late promoter (P_{SEL}), vaccinia synthetic late promoter (P_{SL}), simian virus 40 (SV40) promoter, cytomegalovirus (CMV) promoter, respiratory syncytial virus (RSV) promoter, human elongation factor 1α-subunit (EF1-1α) promoter, a ubiquitin C promoter (Ubc), a phosphoglycerate kinase-1 (PGK) promoter, small nuclear RNA U1b promoter and glucose 6-phosphate dehydrogenase promoter.

Among vaccinia viruses and Lister strain viruses provided herein are those containing a sequence of nucleotides encoding a tyrosinase enzyme, and also contain a sequence of nucleotides encoding an accessory melanin-producing enzyme. For example, provided herein are vaccinia viruses, including WR or Lister strain, such as LIVP viruses, including any of those described herein, that contain: a first sequence of nucleotides encoding a tyrosinase, or an enzymatically active portion or an enzymatically active variant of the enzyme, operatively linked to a promoter; and a second sequence of nucleotides encoding an accessory melanin-producing enzyme operatively linked to a promoter, wherein the accessory melanin-producing enzyme is a tyrosinase-related protein 1 (TRP-1) and/or a dopachrome tautomerase (DCT), or an enzymatically active portion or an enzymatically active variant thereof, or combinations thereof. The first and second sequence can be expressed under control of the same promoter or under control of a different promoter. The first and second sequence can be expressed under the control of the same promoter separated by an internal ribosome entry site (IRES). The two sequences can be linked to different promoters of different strengths, such as embodiments where the promoter operatively linked to the second sequence of nucleotides is a strong promoter, such as a virus late promoter, a retroviral LTR, such as the adenoviral major late promoter, vaccinia synthetic early-late promoter (P_{SEL}), vaccinia synthetic late promoter (P_{SL}), simian virus 40 (SV40) promoter, cytomegalovirus (CMV) promoter, respiratory syncytial virus (RSV) promoter, human elongation factor 1α-subunit (EF1-1α) promoter, a ubiquitin C promoter (Ubc), a phosphoglycerate kinase-1 (PGK) promoter, small nuclear RNA U1b promoter or glucose 6-phosphate dehydrogenase promoter. The sequence of nucleotides encoding a tyrosinase and/or sequence of nucleotides encoding an accessory melanin-producing enzyme is inserted into or in place of a non-essential gene or region in the genome of an unmodified oncolytic virus or is inserted into or in place of nucleic acid encoding a heterologous gene product in the genome of an unmodified oncolytic virus.

The unmodified Lister strain virus can be an LIVP or derivative thereof comprising a sequence of nucleotides set forth in SEQ ID NO:1 or 188, or a sequence of nucleotides that has at least 85%, 86%, 87%, 88%, 89%, 90%, 91%, 92%, 93%, 94%, 95%, 96%, 97%, 98%, 99% or more sequence identity to SEQ ID NO:1 or 188. Examples of the unmodified Lister strain virus or vaccinia viruses include clonal strains of LIVP or a derivatives thereof that contain a sequence of nucleotides selected from: a) nucleotides 2,256-181,114 of SEQ ID NO:55, nucleotides 11,243-182,721 of SEQ ID NO:56, nucleotides 6,264-181,390 of SEQ ID NO:57, nucleotides 7,044-181,820 of SEQ ID NO:58, nucleotides 6,674-181,409 of SEQ ID NO:59, nucleotides 6,716-181,367 of SEQ ID NO:60 or nucleotides 6,899-181,870 of SEQ ID NO:61; or b) a sequence of nucleotides that has at least 85% sequence identity to a sequence of nucleotides 2,256-181,114 of SEQ ID NO:55, nucleotides 11,243-182,721 of SEQ ID NO:56, nucleotides 6,264-181,390 of SEQ ID NO:57, nucleotides 7,044-181,820 of SEQ ID NO:58, nucleotides 6,674-181,409 of SEQ ID NO:59, nucleotides 6,716-181,367 of SEQ ID NO:60 or nucleotides 6,899-181,870 of SEQ ID NO:61, and viruses that have at least 86%, 87%, 88%, 89%, 90%, 91%, 92%, 93%, 94%, 95%, 96%, 97%, 98%, 99% or more sequence identity to any of SEQ ID NOS: 55-61.

As described herein, the viruses can encode another heterologous gene product, such as a therapeutic product and/or reporter product, as described above and herein, and inserted into or in place of a non-essential gene or region in the genome of the virus as described above.

All of the vaccinia viruses, particularly Lister strain, such as LIVP viruses, described above for use in the methods and compositions, also are provided herein, as are compositions containing such viruses. Examples of such Lister strain virus or vaccinia viruses are any that contain the sequence of nucleotides set forth in any of SEQ ID NOS: 64, 67-70, 198-200, 204-206 or 212-216, or a sequence of nucleotides that exhibits at least 85% sequence identity to any of SEQ ID NOS: 64, 67-70, 198-200, 204-206 or 212-216, such as at least 86%, 87%, 88%, 89%, 90%, 91%, 92%, 93%, 94%, 95%, 96%, 97%, 98%, 99% or more sequence identity to any of SEQ ID NOS: 64, 67-70, 198-200, 204-206 or 212-216. For example, provided herein are viruses selected from among those designated GLV-1h326, GLV-1h327, GLV-1h459, GLV-1h460, GLV-1h461, GLV-2b482, GLV-0e407, GLV-1h310, GLV-1h322, GLV-1h323, GLV-1h324, GLV-1h325, GLV-1h458, GLV-2b452, GLV-2b453 and GLV-0e406.

### BRIEF DESCRIPTION OF THE FIGURES

FIG. 1 depicts the biosynthetic pathways of eumelanin and pheomelanin forms of melanin. The figure is adapted from Kobayashi et al. (1995) J Cell Science, 108:2301-2309, and depicts the order of molecules involved in the biosynthesis of eumelanin and pheomelanin, starting with the amino acid, tyrosine. In addition, the figure depicts the known enzymes (i.e., tyrosinase (Tyr), tyrosinase related protein-1 (TRP-1), and dopachrome tautomerase (Dct/TRP2)), which catalyze the reactions to generate the various melanin precursor molecules.

FIG. 2A-C depicts a lineage map of modified Vaccinia viruses derived from (A) GLV-1h68, (B) LIVP 1.1.1, and (C) Western Reserve (WR) viruses.

### DETAILED DESCRIPTION

### Outline

A. Definitions
B. Gene-Evoked Chromophore (e.g., Melanin) Production as Theranostic Mediator
C. Melanin Biosynthesis and Properties
   1. Biosynthesis of Melanin
   2. Properties of Melanin
      a. Optical Properties
      b. Metal Binding and reduction-oxidation (redox)
D. Nucleic Acid Molecules Encoding Chromophore-Producing Enzymes
   1. Chromophore-Producing Enzymes
      a. Tyrosinase
      b. Tyrosinase-Related Protein-1
      c. Dopachrome Tautomerase
   2. Nucleic Acid Molecules
      a. Virus and Viral Vectors
      b. Non-Viral Vectors
   3. Localized Delivery or Expression of Nucleic Acid Molecules
      a. Targeted Delivery
      b. Cell-Specific (e.g., Tumor-specific) expression
      c. Oncolytic Viruses
E. Vaccinia Viruses and LIVP
   1. Lister and LIVP Strains
   2. Heterologous Nucleic Acid and Modified Viruses
      a. Exemplary Modifications
         i. Diagnostic or Reporter Gene Products
         ii. Therapeutic Gene Products
         iii. Modifications to alter attenuation of the viruses
      b. Control of Heterologous Gene Expression
      c. Exemplary Modified or Recombinant Viruses (Parental Strains)
      d. Exemplary Vaccinia Viruses Encoding a Chromophore-Producing Enzyme or Enzymes
      e. Methods of Generating Modified Viruses
   3. Methods of Producing Viruses
      a. Host cells for Propagation
      b. Concentration Determination
      c. Storage Methods
      d. Preparation of Virus
   4. Anti-Tumorigenicity and Efficacy
      a. Tumor-Associated Replication Initiator
      b. Cytotoxicity
      c. Tumor Growth
   5. Toxicity/Safety
F. Pharmaceutical Compositions, Combinations and Kits
   1. Pharmaceutical Compositions
   2. Host Cells
   3. Combinations
   4. Kits
G. Therapeutic, Diagnostic and Monitoring Methods
   1. Delivery of Nucleic Acid to Subjects for Treatment, Diagnosis, or Theragnosis
      a. Methods of Nucleic Acid Delivery
      b. Dosages and Dosage Regimes
   2. Energy Absorbing Therapeutic Methods
      a. Heat Therapies: Hyperthermia and Thermotherapies
         i. Photothermal Therapy Methods
         a) Laser Light Therapy
         i) Parameters
         ii) Laser Positioning
         b) Microwave-based Heat Therapy
         c) Infrared light therapy
         ii. Ultrasound Heat Therapy
         iii. Magnetic Conduction Methods
      b. Photodynamic Therapy
   3. Methods of Detection
      a. Photoacoustic Tomography
      b. Multispectral Optoacoustic Tomography (MSOT)
      c. Magnetic Resonance Imaging (MRI)
   4. Monitoring
      a. Monitoring viral gene expression
      b. Monitoring tumor size
      c. Monitoring antibody titer
      d. Monitoring general health diagnostics
      e. Monitoring coordinated with treatment
H. Combination Therapy
   1. Surgical Procedures
   2. Radiotherapy
   3. Therapeutic Compounds
I. Examples

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, energy-absorbing therapy refers to any therapy that effects treatment of a subject to ameliorate or reduce symptoms of a condition, disorder or disease by virtue of absorption of energy by a moiety in a cell or tissue and conversion of absorbed energy into a form that can be used for therapeutic treatment. For example, absorbed energy is converted to generate heat and/or other chemical change or changes. The converted energy can occur in a cell, tissue or organ of a subject following exposure of the cell, tissue or organ to an energy source. In some cases, the converted energy can result in toxicity to the cell, tissue or organ leading to cell death, which can effect treatment where the cell, tissue or organ are involved in a disease. Exemplary forms of energy that can effect therapy include chemical energy, electric energy, radiant energy, electromagnetic energy, nuclear energy, magnetic energy, elastic energy, sound energy, mechanical energy or luminous energy.

As used herein, an energy source refers to any source from which energy can be obtained to provide heat, light, sound or power. For example, a desired wavelength of electromagnetic energy can be obtained from a laser, light-emitting diode, fluorescent lamp, dichroic lamp, a light box or other similar device.

As used herein, electromagnetic energy refers to a form of energy that is reflected or emitted from objects in the form of electrical and magnetic waves that can travel through space. Electromagnetic energy includes, for example, gamma rays, x-rays, ultraviolet radiation, visible light, infrared radiation, microwaves and radiowaves. For purposes of methods herein, and depending on the particular application, wavelengths of electromagnetic energy can be applied at 100 nm to 1500 nm, such as 100 to 400 nm, 100 to 280 nm, 280 nm to 320 nm, 280 to 315 nm, 315 to 400 nm, 320 to 420 nm, 500 to 1500 nm, 600 to 1200 nm or 700 to 900 nm.

As used herein, sound or acoustic energy is a form mechanical energy produced from sound vibrations or acoustic waves.

As used herein, ultrasound energy is a cyclic sound pressure wave with a frequency greater than the upper limit of the human hearing range, generally greater than 20,000 Hz. Ultrasound energy can be applied using a device capable of applying sonic energy, such as a sonicator or other ultrasound device. Ultrasound waves can be applied by focused ultrasound with bursts of ultrasound energy of 1-4 mHz repeated at a repetition frequency. The applied energy can be converted to heat by a cell, tissue or organ. For example, chromophore products, such as melanin, can absorb ultrasound acoustic waves to produce heat.

As used herein, a chromophore-producing enzyme refers to any enzyme that participates in a biochemical pathway that produces a chromophore or chromophore product. The enzymes catalyzes one or more reactions that result in the production of a chromophore or chromophore product in a cell. A chromophore-producing enzyme includes enzymes that are sufficient to achieve catalysis in the absence of other enzymes in the cell. Chromophore-producing enzymes also include enzymes that alone are not sufficient to produce a chromophore product but that act as an accessory enzyme to alter production of a chromophore product in the cell, stabilize the formation of another enzyme or chromophore product, or otherwise contribute to the formation of a chromophore product in a cell.

As used herein, a melanin-producing enzyme refers to any enzyme that participates in a biochemical pathway that produces melanin or a melanin product. The enzyme catalyzes one or more reactions that result in the production of melanin or a melanin product in a cell. Hence, a melanin-producing enzyme includes a tyrosinase enzyme, which is an enzyme that is sufficient to achieve catalysis of melanin in the absence of other enzymes in the cell. Melanin-producing enzymes also include accessory chromophore-producing enzymes that alone are not sufficient to produce melanin but contribute to the production of melanin in a cell. Such accessory melanin-producing enzymes alter production of a melanin product in the cell, stabilize the formation of another enzyme (e.g., tyrosinase) or a melanin product, or otherwise contribute to the formation of a melanin product in a cell. For example, tyrosinase-related protein 1 (TRP-1) and dopachrome tautomerase (DCT) are accessory melanin-producing enzymes that, among other roles, catalyze reactions to alter the ratio of eumelanin to pheomelanin in a cell.

As used herein, a chromophore refers to a compound or chemical group that is capable of selective energy absorption (e.g., light absorption) resulting in the coloration of a molecule.

As used herein, a chromophore product (e.g., melanin product) is a chromophore catalyzed de novo in a cell by an exogenously introduced chromophore-producing enzyme. Hence, the chromophore product is one whose presence is achieved, increased or overproduced in a cell by externally applied or administered source providing a chromophore-producing enzyme, such as a nucleic acid molecule encoding a chromophore-producing enzyme. It is understood that the exogenously introduced chromophore-producing enzyme can be an enzyme that is present or exists in the cell, but whose level or activity is increased compared to the level or activity of the chromophore-producing enzyme naturally present in the cell.

As used herein, melanin refers to a polymer built from compounds produced by the oxidation of the amino acid tyrosine. Melanin includes eumelanin and/or pheomelanin.

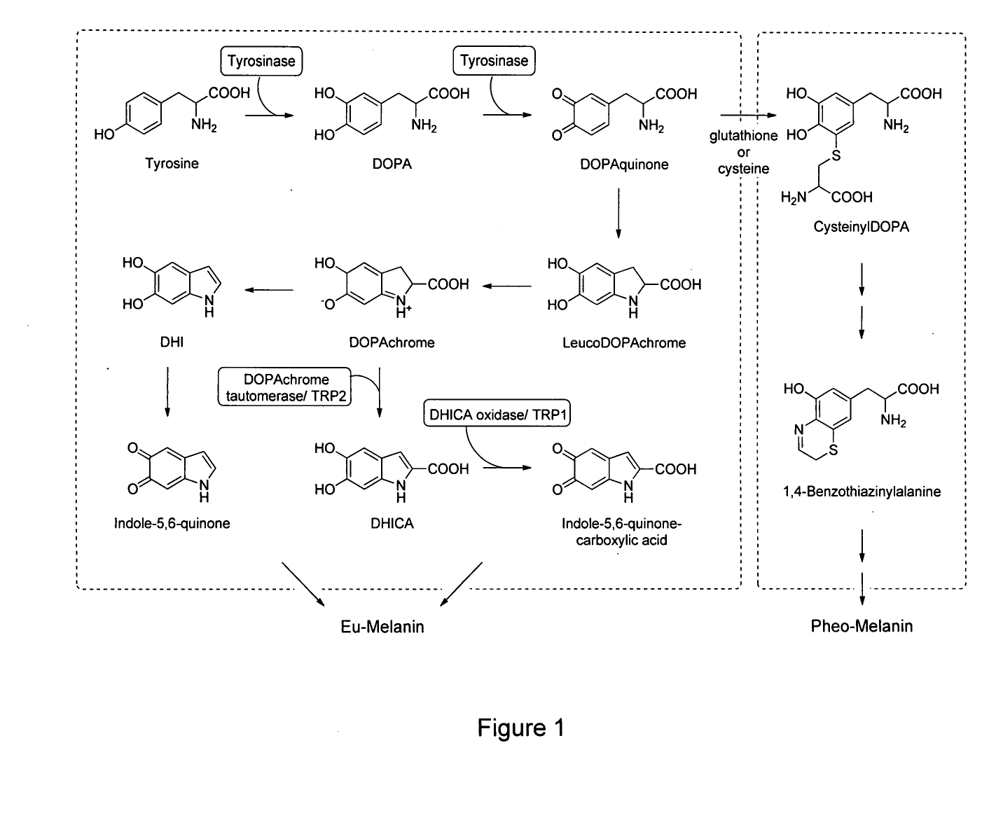

As used herein, eumelanin refers to brown-black polymers that are insoluble, nitrogenous pigments produced by the oxidative polymerization of 5,6-dihydroxyindoles derived enzymatically from tyrosine via DOPA. Eumelanin is made up of 5,6-dihydroxyindolequinone (DHI, or hydroquinone), 5,6-dihydroxyindole 2-carboxylic acid (denoted as DHICA), and their derived forms. Eumelanin is found in hair, areolae and skin and is abundant in people with dark skin. As shown in FIG. 1, the production of eumelanin is catalyzed by tyrosinase to produce DOPA and then dopaquinone, which is then converted to leucodopachrome (L-DOPA) to produce DHI or DHICA and the oxidized forms.

As used herein, pheomelanin refers to sulfur-containing, alkali-soluble, pigments produced by oxidative polymerization of cysteinyldopas via 1,4-benzothiazine intermediates. Thus, pheomelanin is a cysteine-containing red-brown polymer of benzothiazine units. Pheomelanin differs from eumelanin in that its oligomer structure incorporates benzothiazine and benzothiazole units that are produced instead of DHI and DHICA, when the amino acid L-cysteine is present. As shown in FIG. 1, the production of pheomelanin is catalyzed by tyrosinase to produce DOPA and then dopaquinone, which is then combined with cysteine to ultimately produce pheomelanin.

As used herein, a tyrosinase refers to a copper-containing melanin-producing enzyme that exhibits tyrosine hydroxylase and dopa oxidase catalytic activities that requires copper binding for activity. Tyrosinase is present in eukaryotic and prokaryotic cells, including in plants, insects, amphibians and mammals. Tyrosinase enzymes are produced as precursor molecules containing a signal sequence (generally amino acids 1-18), which is processed to generate a mature protein. Tyrosinase is a membrane protein containing three domains, an N-terminal lumenal ectodomain that contains the copper binding site and is responsible for catalytic activity, a transmembrane domain and a C-terminal cytoplasmic domain. The amino acid and nucleotide sequence of exemplary tyrosinase enzymes are known in the art or provided herein. An example of a tyrosinase is human tyrosinase set forth in SEQ ID NO:81 as a precursor or in SEQ ID NO:82 as the mature form lacking the signal sequence, and which is encoded by a sequence of amino acids set forth in SEQ ID NO:80. Another exemplary tyrosinase is mouse tyrosinase set forth in SEQ ID NO:7 as the precursor or in SEQ ID NO:84 as the mature enzyme, and which is encoded by the sequence of nucleotides set forth in SEQ ID NO:6 or 83. Tyrosinases include variants of any known or naturally occurring tyrosinase, including allelic and species variants. For example, examples of tyrosinases known in the art and described herein are tyrosinases from gorilla, chimpanzee, orangutan, gibbon, cynomolgus monkey, Rhesus macaque, elephant, rabbit, mole rat, pig, cat, sheep, dog, goat, rat, guinea pig, human or mouse. Tyrosinases include enzymatically active portions of any tyrosinase enzyme or an enzymatically active variant of a tyrosinase enzyme. Generally, tyrosinases provided herein exhibit at least 70%, 75%, 80%, 85%, 86%, 87%, 88%, 89%, 90%, 91%, 92%, 93%, 94%, 95%, 96%, 97%, 98%, 99% or more amino acid sequence identity to SEQ ID NOS: 7, 81, 82 or 84 or other known species variants described herein.

As used herein, a tyrosinase-related protein 1 (TRP-1) refers to a metal-binding enzyme (e.g., copper-binding enzyme) that exhibits 5,6-dihydroxy-indole-2-carboxylic acid (DHICA) oxidase that requires metal (e.g., copper) binding for activity. TRP-1 enzymes are produced as precursor molecules containing a signal sequence (generally amino acids 1-24), which is processed to generate a mature protein. TRP-1 is a membrane protein containing three domains: an N-terminal lumenal ectodomain that contains the metal (e.g., copper) binding site and is responsible for catalytic activity, a transmembrane domain and a C-terminal cytoplasmic domain. The amino acid and nucleotide sequence of exemplary TRP-1 enzymes are known in the art or provided herein. An example of TRP-1 is human TRP-1 with the amino acid sequence set forth in SEQ ID NO:20 as a precursor or in SEQ ID NO:120 as the mature form lacking the signal sequence, and which is encoded by a sequence of nucleotides set forth in SEQ ID NO:19 or 119. TRP-1 includes variants of any known or naturally occurring TRP-1 enzymes, including allelic and species variants. Exemplary TRP-1 enzymes known in the art and described herein are TRP-1 enzymes from chimpanzee, gorilla, gibbon, orangutan, cynomolgus monkey, rhesus macaque, rabbit, pig, elephant, dog, goat, bovine, sheep or mouse among many other known species variants. Reference to TRP-1 includes enzymatically active portions of any TRP-1 enzyme or an enzymatically active variant of a TRP-1 enzyme. Generally, TRP-1 enzymes provided herein exhibit at least 70%, 75%, 80%, 85%, 86%, 87%, 88%, 89%, 90%, 91%, 92%, 93%, 94%, 95%, 96%, 97%, 98%, 99% or more amino acid sequence identity to SEQ ID NOS: 20 or 120 or other known species variants such as any described herein.

As used herein, dopachrome tautomerase (DCT) refers to a metal-binding enzyme (e.g., copper-binding enzyme) that exhibits dopachrome tautomerase activity that requires zinc binding for activity. DCT enzymes are produced as precursor molecules containing a signal sequence (generally amino acids 1-23), which is processed to generate a mature protein. DCT is a membrane protein containing three domains: an N-terminal lumenal ectodomain that contains the zinc binding site and is responsible for catalytic activity, a transmembrane domain and a C-terminal cytoplasmic domain. The amino acid and nucleotide sequence of exemplary DCT enzymes are known in the art or are provided herein. For example, human DCT set forth in SEQ ID NO:30 as a precursor or in SEQ ID NO:146 as the mature form lacking the signal sequence, and which is encoded by a sequence of amino acids set forth in SEQ ID NO:29, is an exemplary DCT. DCT enzymes include variants of any known or naturally occurring DCT enzymes, including allelic and species variants. For example, examples of DCT enzymes known in the art and described herein are DCT enzymes from gorilla, orangutan, cynomolgus monkey, rhesus macaque, white-tufted-ear marmoset, horse, pig, panda, rabbit and sheep among many other known species variants. Reference to DCT includes enzymatically active portions of any DCT enzyme or an enzymatically active variant of a DCT enzyme. Generally, DCT enzymes, provided herein exhibit at least 70%, 75%, 80%, 85%, 86%, 87%, 88%, 89%, 90%, 91%, 92%, 93%, 94%, 95%, 96%, 97%, 98%, 99% or more sequence identity to SEQ ID NOS: 30 or 146 or other known species variants such as any described herein.

As used herein, enzymatically active portion of a melanin-producing enzyme (e.g., tyrosinase) refers to a polypeptide that contains at least the minimal amino acid residues to catalyze one or more reactions that result in the production of melanin. For example, an enzymatically active portion of a tyrosinase exhibits hydroxylase and dopa oxidase catalytic activities, an enzymatically active portion of a TRP-1 enzyme exhibits 5,6-dihydroxy-indole-2-carboxylic acid (DHICA) oxidase activity. An enzymatically active portion of a DCT enzyme exhibits dopachrome tautomerase activity. Typically, an active portion contains contiguous amino acids from a melanin-producing polypeptide (e.g., tyrosinase) that at least includes an enzymatically active portion of the N-terminal lumenal ectodomain containing the metal binding sites. Active fragments and the minimal amino acid residues can be empirically determined by expressing the melanin-producing enzyme in a cell and assessing production of melanin or precursors. It is understood that an accessory melanin-producing enzyme (e.g., TRP-1 or DCT) requires the cell to also be co-transformed with tyrosinase for formation of melanin. Activity can be any percentage of activity (more or less) of the full-length polypeptide, including but not limited to, 1% of the activity, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more activity compared to the full polypeptide. An enzymatically active fragment or portion is a truncated fragment in which about or at least 1, 2, 3, 4, 5, 6, 7, 8, 9, 10, 11, 12, 13, 14, 15, 16, 17, 18, 19, 20, 30, 40, 50, 60, 70, 80, 90 or 100 amino acids at the N- or C-terminus of the polypeptide are missing.

As used herein, an enzymatically active variant of a melanin-producing enzyme (e.g., tyrosinase) refers to an enzyme that contains a modification, such as an amino acid insertion, deletion or replacement (substitution) of one or more amino acids, compared to a reference or wildtype enzyme, and exhibits one or more activities of the reference or wildtype enzyme to participate in a biochemical pathway that produces melanin or a melanin product such as to catalyze one or more reactions that results in the production of melanin. For example, an enzymatically active variant of a tyrosinase enzyme is a tyrosinase that contains a modification, such as an amino acid insertion, deletion or replacement (substitution) of one or more amino acids, compared to a reference or wildtype tyrosinase, and exhibits tyrosine hydroxylase and dopa oxidase catalytic activities. An enzymatically active variant of a TRP-1 enzyme is a TRP-1 that contains a modification, such as an amino acid insertion, deletion or replacement (substitution) of one or more amino acids, compared to a reference or wildtype TRP-1, and exhibits 5,6-dihydroxy-indole-2-carboxylic acid (DHICA) oxidase activity. An enzymatically active variant of a DCT enzyme is a DCT that contains a modification, such as an amino acid insertion, deletion or replacement (substitution) of one or more amino acids, compared to a reference or wildtype TRP-1, and exhibits 5 dopachrome tautomerase activity. The modification, such as amino acid replacement, can be a conservative amino acid change or a non-conservative amino acid change. The activity can be reduced or increased compared to the activity of a native or wildtype melanin-producing enzyme (e.g., tyrosinase). For example, a melanin-producing enzyme (e.g., tyrosinase) includes polypeptides that exhibit at least 20%, 30%, 40%, 50%, 60%, 70%, 80%, 90%, 100%, 110%, 120%, 130%, 140%, 150%, 160%, 170%, 180%, 190%, 200% or more activity of a native or wildtype form of the polypeptide.

As used herein, nucleic acid molecule refers to single-stranded and/or double-stranded polynucleotides, such as deoxyribonucleic acid (DNA) and ribonucleic acid (RNA), as well as analogs or derivatives of either RNA or DNA. Also included in the term “nucleic acid” are analogs of nucleic acids such as peptide nucleic acid (PNA), phosphorothioate DNA, and other such analogs and derivatives. Nucleic acids can encode gene products, such as, for example, polypeptides, regulatory RNAs, microRNAs, siRNAs and functional RNAs. Hence, nucleic acid molecule is meant to include all types and sizes of nucleic acid molecules including siRNA, aptamers, ribozymes, cDNA, plasmids and DNA, including modified nucleotides and nucleotide analogs. Reference to a nucleic acid molecule herein refers to any agent that is composed of or contains the nucleic acid molecule. Hence, reference to a nucleic acid molecule includes the agent or conduit, such as vehicle, vector, or construct, that contains a nucleic acid molecule packaged therein or associated therewith. This includes viral and non-viral vectors or naked DNA. For example, nucleic acid molecules include a virus, virus-like particles, mini-circles, a plasmid or vector, a liposome and/or a nanoparticle.

As used herein, a construct refers to a piece of circular double-stranded DNA, such as a vector or plasmid.

As used herein, “plasmid” or “vector” is used interchangeably and is meant a circular DNA vector. Plasmids contain an origin of replication that allows many copies of the plasmid to be produced in a bacterial or eukaryotic cells without integration of the plasmid into the host cell DNA.

As used herein, a non-viral vector refers to a nucleic acid molecule that contains an origin of replication and other elements for replication of the nucleic acid, but does not include all of the requisite elements that result in a viral particle, such as elements for viral replication, packaging and/or expression. Such elements include, but are not limited to, one or more of the nucleic acid molecules encoding a capsid protein or coat protein, a packaging signal, an early promoter and regulators of late viral gene expression. Hence, for example, a non-viral nucleic acid vector is not packaged as a viral vector particle.

As used herein, a “virus” or virus vector refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells. Viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus.

As used herein, oncolytic viruses refer to viruses that replicate selectively in tumor cells in tumorous subjects. Some oncolytic viruses can kill a tumor cell following infection of the tumor cell. For example, an oncolytic virus can cause death of the tumor cell by lysing the tumor cell or inducing cell death of the tumor cell.

As used herein the term “vaccinia virus” or “VACV” or “VV” denotes a large, complex, enveloped virus belonging to the poxvirus family. It has a linear, double-stranded DNA genome approximately 190 kbp in length, which encodes approximately 200 proteins. Vaccinia virus strains include, but are not limited to, strains of, derived from, or modified forms of Western Reserve (WR), Copenhagen, Tashkent, Tian Tan, Lister, Wyeth, IHD-J, and IHD-W, Brighton, Ankara, MVA, Dairen I, LIPV, LC16M8, LC 16MO, LIVP, WR 65-16, NYCBH vaccinia virus strains.

As used herein, Lister Strain of the Institute of Viral Preparations (LIVP) or LIVP virus strain refers to a virus strain that is the attenuated Lister strain (ATCC Catalog No. VR-1549) that was produced by adaption to calf skin at the Institute of Viral Preparations, Moscow, Russia (Al'tshtein et al. (1985) Dokl. Akad. Nauk USSR 285:696-699). The LIVP strain can be obtained, for example, from the Institute of Viral Preparations, Moscow, Russia (see. e.g., Kutinova et al. (1995) Vaccine 13:487-493); the Microorganism Collection of FSRI SRC VB Vector (Kozlova et al. (2010) Environ. Sci. Technol. 44:5121-5126); or can be obtained from the Moscow Ivanovsky Institute of Virology (C0355 K0602; Agranovski et al. (2006) Atmospheric Environment 40:3924-3929). It also is well-known to those of skill in the art; as it was the vaccine strain used for vaccination in the USSR and throughout Asia and India. The strain now is used by researchers and is well-known (see e.g., Altshteyn et al. (1985) Dokl. Akad. Nauk USSR 285:696-699; Kutinova et al. (1994) Arch. Virol. 134:1-9; Kutinova et al. (1995) Vaccine 13:487-493; Shchelkunov et al. (1993) Virus Research 28:273-283; Sroller et al. (1998) Archives Virology 143:1311-1320; Zinoviev et al., (1994) Gene 147:209-214; and Chkheidze et al. (1993) FEBS 336:340-342). Among the LIVP strains is one that contains a genome having a sequence of nucleotides set forth in SEQ ID NO: 1 or 188, or a sequence that is at least or at least about 97%, 98% or 99% identical to the sequence of nucleotides set forth in SEQ ID NO: 1 or 188. An LIVP virus strain encompasses any virus strain or virus preparation that is obtained by propagation of LIVP through repeat passage in cell lines.

As used herein, an LIVP clonal strain or LIVP clonal isolate refers to a virus that is derived from the LIVP virus strain by plaque isolation, or other method in which a single clone is propagated, and that has a genome that is homogenous in sequence. Hence, an LIVP clonal strain includes a virus whose genome is present in a virus preparation propagated from LIVP. An LIVP clonal strain does not include a recombinant LIVP virus that is genetically engineered by recombinant means using recombinant DNA methods to introduce heterologous nucleic acid. In particular, an LIVP clonal strain has a genome that does not contain heterologous nucleic acid that contains an open reading frame encoding a heterologous protein. For example, an LIVP clonal strain has a genome that does not contain non-viral heterologous nucleic acid that contains an open reading frame encoding a non-viral heterologous protein. As described herein, however, it is understood that any of the LIVP clonal strains provided herein can be modified in its genome by recombinant means to generate a recombinant virus. For example, an LIVP clonal strain can be modified to generate a recombinant LIVP virus that contains insertion of nucleotides that contain an open reading frame encoding a heterologous protein.

As used herein, LIVP 1.1.1 is an LIVP clonal strain that has a genome having a sequence of nucleotides set forth in SEQ ID NO: 55, or a genome having a sequence of nucleotides that has at least 97%, 98%, or 99% sequence identity to the sequence of nucleotides set forth in SEQ ID NO: 55.

As used herein, LIVP 2.1.1 is an LIVP clonal strain that has a genome having a sequence of nucleotides set forth in SEQ ID NO: 56, or a genome having a sequence of nucleotides that has at least 97%, 98%, or 99% sequence identity to the sequence of nucleotides set forth in SEQ ID NO: 56.

As used herein, LIVP 4.1.1 is an LIVP clonal strain that has a genome having a sequence of nucleotides set forth in SEQ ID NO: 57, or a genome having a sequence of nucleotides that has at least 97%, 98% or 99% sequence identity to the sequence of nucleotides set forth in SEQ ID NO: 57.

As used herein, LIVP 5.1.1 is an LIVP clonal strain that has a genome having a sequence of nucleotides set forth in SEQ ID NO: 58, or a genome having a sequence of nucleotides that has at least 97%, 98% or 99% sequence identity to the sequence of nucleotides set forth in SEQ ID NO: 58.

As used herein, LIVP 6.1.1 is an LIVP clonal strain that has a genome having a sequence of nucleotides set forth in SEQ ID NO: 59, or a genome having a sequence of nucleotides that has at least 97%, 98% or 99% sequence identity to the sequence of nucleotides set forth in SEQ ID NO: 59.

As used herein, LIVP 7.1.1 is an LIVP clonal strain that has a genome having a sequence of nucleotides set forth in SEQ ID NO: 60, or a genome having a sequence of nucleotides that has at least 97%, 98% or 99% sequence identity to the sequence of nucleotides set forth in SEQ ID NO: 60.

As used herein, LIVP 8.1.1 is an LIVP clonal strain that has a genome having a sequence of nucleotides set forth in SEQ ID NO: 61, or a genome having a sequence of nucleotides that has at least 97%, 98% or 99% sequence identity to the sequence of nucleotides set forth in SEQ ID NO: 61.

As used herein, the term “modified virus” refers to a virus that is altered compared to a parental strain of the virus. Typically modified viruses have one or more truncations, mutations, insertions or deletions in the genome of virus. A modified virus can have one or more endogenous viral genes modified and/or one or more intergenic regions modified. Exemplary modified viruses can have one or more heterologous nucleic acid sequences inserted into the genome of the virus. Modified viruses can contain one or more heterologous nucleic acid sequences in the form of a gene expression cassette for the expression of a heterologous gene.

As used herein, a modified LIVP virus strain refers to an LIVP virus that has a genome that is not contained in LIVP, but is a virus that is produced by modification of a genome of a strain derived from LIVP. Typically, the genome of the virus is modified by substitution (replacement), insertion (addition) or deletion (truncation) of nucleotides. Modifications can be made using any method known to one of skill in the art such as genetic engineering and recombinant DNA methods. Hence, a modified virus is a virus that is altered in its genome compared to the genome of a parental virus. Exemplary modified viruses have one or more heterologous nucleic acid sequences inserted into the genome of the virus. Typically, the heterologous nucleic acid contains an open reading frame encoding a heterologous protein. For example, modified viruses herein can contain one or more heterologous nucleic acid sequences in the form of a gene expression cassette for the expression of a heterologous gene.

As used herein, synthetic, with reference to, for example, a synthetic nucleic acid molecule or a synthetic gene or a synthetic peptide, refers to a nucleic acid molecule or polypeptide molecule that is produced by recombinant methods and/or by chemical synthesis methods.

As used herein, “production by recombinant methods” or “methods using recombinant DNA methods” or variations thereof refers to the use of the well-known methods of molecular biology for expressing proteins encoded by cloned DNA.

As used herein a “gene expression cassette” or “expression cassette” is a nucleic acid construct, containing nucleic acid elements that are capable of effecting expression of a gene in hosts that are compatible with such sequences. Expression cassettes include at least promoters and optionally, transcription termination signals. Typically, the expression cassette includes a nucleic acid to be transcribed operably linked to a promoter. Expression cassettes can contain genes that encode, for example, a therapeutic gene product, or a detectable protein or a selectable marker gene.

As used herein, a heterologous nucleic acid (also referred to as exogenous nucleic acid or foreign nucleic acid) refers to a nucleic acid that is not normally produced in vivo by an organism or virus from which it is expressed or that is produced by an organism or a virus but is at a different locus, or that mediates or encodes mediators that alter expression of endogenous nucleic acid, such as DNA, by affecting transcription, translation, or other regulatable biochemical processes. Hence, heterologous nucleic acid is often not normally endogenous to a virus into which it is introduced. Heterologous nucleic acid can refer to a nucleic acid molecule from another virus in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the virus or in the same way in the virus in which it is expressed. Any nucleic acid, such as DNA, that one of skill in the art recognizes or considers as heterologous, exogenous or foreign to the virus in which the nucleic acid is expressed is herein encompassed by heterologous nucleic acid. Examples of heterologous nucleic acids include, but are not limited to, nucleic acid that encodes exogenous peptides/proteins, including diagnostic and/or therapeutic agents. Proteins that are encoded by heterologous nucleic acid can be expressed within the virus, secreted, or expressed on the surface of the virus in which the heterologous nucleic acid has been introduced.

As used herein, a heterologous protein or heterologous polypeptide (also referred to as exogenous protein, exogenous polypeptide, foreign protein or foreign polypeptide) refers to a protein that is not normally produced by a virus.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon, which can enhance expression (see, e.g., Kozak J. Biol. Chem. 266: 19867-19870 (1991) and Shine and Delgarno, Nature 254(5495):34-38 (1975)). The desirability of (or need for) such modification can be empirically determined.

As used herein, the phrases “operatively positioned,” “operatively linked,” “under control,” and “under transcriptional control” mean that a promoter is in a correct functional location and orientation in relation to a nucleic acid sequence to control transcriptional initiation and expression of that sequence.

As used herein, an “internal ribosome entry site” (IRES) refers to a nucleotide sequence that allows for translation initiation in the middle of a messenger RNA (mRNA) sequence as part of protein synthesis.

As used herein, a heterologous promoter refers to a promoter that is not normally found in the wild-type organism or virus or that is at a different locus as compared to a wild-type organism or virus. A heterologous promoter is often not endogenous to a virus into which it is introduced, but has been obtained from another virus or prepared synthetically. A heterologous promoter can refer to a promoter from another virus in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome.

A synthetic promoter is a heterologous promoter that has a nucleotide sequence that is not found in nature. A synthetic promoter can be a nucleic acid molecule that has a synthetic sequence or a sequence derived from a native promoter or portion thereof. A synthetic promoter also can be a hybrid promoter composed of different elements derived from different native promoters.

As used herein, a strong promoter refers to any promoter that allows the gene under its control to be expressed at a high level. The strong promoter can be naturally occurring, or it can be a modified promoter or synthetic promoter, e.g., a derivative of a naturally occurring promoter. It can thus be native or non-native. Hence, such a promoter can produce large amounts of transcript and final protein product from the gene of interest. For example, strong promoters can express proteins at a level of at least 1% of the total cellular protein. Typically, a strong promoter can express proteins at a level of 2, 5, 10, 15, 20, 25, 30, 35, 40, 45 or 50% of the total cellular protein. The term “strong promoter” is a well-known term in the art and strong promoters are widely described in the literature. Strong promoters include viral promoters and eukaryotic promoters. Exemplary strong promoters include, but are not limited to, adenoviral major late promoter, vaccinia synthetic early-late promoter (P_{SEL}), vaccinia synthetic late promoter (P_{SL}), simian virus 40 (SV40) promoter, cytomegalovirus (CMV) promoter, respiratory syncytial virus (RSV) promoter, a retroviral LTR promoter, human elongation factor 1α-subunit (EF1-1α) promoter, a ubiquitin C promoter (Ubc), a phosphoglycerate kinase-1 (PGK) promoter, small nuclear RNA U1b promoter and glucose 6-phosphate dehydrogenase promoter and others as described herein or known to one of skill in the art.

As used herein, tissue-specific or cell-specific promoter refers to a promoter that is capable of driving transcription of a gene in a particular tissue (e.g., lung, liver, breast, or others) or cell (e.g., leukocyte, myocyte, tumor cell, or others) while remaining largely “silent” or expressed at relatively low levels in other tissue or cell types. A tissue-specific or cell-specific promoter can be selective for any tissue or cell-type in a subject. Such promoters are known to one of skill in the art and are described herein. Exemplary tissue-specific or cell-specific promoters are tumor-specific promoters. It is understood, however, that tissue-specific or cell-specific promoters can have a detectable amount of “background” or “base” activity in those tissues or cells where they are silent. Generally, the promoter is active to a greater degree in a predetermined target cell or tissue as compared to other cells or tissues. For example, the promoter has about or 2-fold, 3, 4, 5, 6, 7, 8, 9, 10, 20, 30, 40, 50, 60, 70, 80, 90, 100, 200, 300, 400, 500, 600, 700, 800, 900 or more fold activity (i.e. ability to express a nucleic acid sequence operatively linked thereto) in a predetermined tissue or cell compared to in other tissue or cell types. Thus, a tissue-specific or cell-specific promoter that exhibits some low level activity, such as at or about 10% or less in another cell type is still considered to be a tissue-specific or cell-specific promoter if its activity is greater in the predetermined target tissue or cell than the activity in the other cell type.

As used herein, a tumor-specific promoter is a promoter that is capable of driving transcription of a gene in a tumor cell, while remaining largely “silent” or expressed at relatively low levels in other tissue or cell types, such as for example, in normal cells. For example, a tumor-specific promoter has about 2-fold or 3, 4, 5, 6, 7, 8, 9, 10, 20, 30, 40, 50, 60,70, 80, 90, 100, 200, 300, 400, 500, 600, 700, 800, 900 or more fold activity (i.e. ability to express a nucleic acid sequence operatively linked thereto) in a tumor cell compared to in a normal cell.

As used herein, an “inducible expression system” refers to a transcription unit whose transcription is reversible turned on or off in the presence of an inducer. An example of an inducible expression system is a tetracycline expression system, whereby transcription is reversible turned on or off in the presence of tetracycline or a derivative of tetracycline (e.g., doxycycline). For example, an inducible expression system includes a nucleotide sequence encoding tetracycline repressor protein (TetR) and a nucleotide sequence of interest (e.g., encoding a tyrosinase enzyme or other chromophore-producing enzyme) operably linked to an inducible promoter composed of a minimal promoter operably linked to at least one tetracycline operon (tetO) sequence.

As used herein, the term, “therapeutic gene product” or “therapeutic polypeptide” or “therapeutic agent” refers to any heterologous protein expressed by the therapeutic virus that ameliorates the symptoms of a disease or disorder or ameliorates the disease or disorder. Therapeutic agents include, but are not limited to, moieties that inhibit cell growth or promote cell death, that can be activated to inhibit cell growth or promote cell death, or that activate another agent to inhibit cell growth or promote cell death. Optionally, the therapeutic agent can exhibit or manifest additional properties, such as, properties that permit its use as an imaging agent, as described elsewhere herein. Exemplary therapeutic agents include, for example, cytokines, growth factors, photosensitizing agents, radionuclides, toxins, anti-metabolites, signaling modulators, anti-cancer antibiotics, anti-cancer antibodies, angiogenesis inhibitors, chemotherapeutic compounds or a combination thereof.

As used herein, a “reporter gene” is a gene that encodes a reporter molecule that can be detected when expressed by a virus provided herein or encodes a molecule that modulates expression of a detectable molecule, such as nucleic acid molecule or a protein, or modulates an activity or event that is detectable. Hence reporter molecules include, nucleic acid molecules, such as expressed RNA molecules, and proteins.

As used herein, a detectable label or detectable moiety or diagnostic moiety (also imaging label, imaging agent, or imaging moiety) refers to an atom, molecule or composition, wherein the presence of the atom, molecule or composition can be directly or indirectly measured. Detectable labels can be used to image one or more of any of the viruses provided herein. Detectable labels include, for example, chemiluminescent moieties, bioluminescent moieties, fluorescent moieties, radionuclides, and metals. Methods for detecting labels are well-known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Direct detection of a detectable label refers to, for example, measurement of a physical phenomenon of the detectable label itself, such as energy or particle emission or absorption of the label itself, such as by X-ray or MRI. Indirect detection refers to measurement of a physical phenomenon of an atom, molecule or composition that binds directly or indirectly to the detectable label, such as energy or particle emission or absorption, of an atom, molecule or composition that binds directly or indirectly to the detectable label. In a non-limiting example of indirect detection, a detectable label can be biotin, which can be detected by binding to avidin. Non-labeled avidin can be administered systemically to block non-specific binding, followed by systemic administration of labeled avidin. Thus, included within the scope of a detectable label or detectable moiety is a bindable label or bindable moiety, which refers to an atom, molecule or composition, wherein the presence of the atom, molecule or composition can be detected as a result of the label or moiety binding to another atom, molecule or composition. Exemplary detectable labels include, for example, metals such as colloidal gold, iron, gadolinium, and gallium-67, fluorescent moieties, and radionuclides. Exemplary fluorescent moieties and radionuclides are provided elsewhere herein.

As used herein, a tumor cell or cancer cell refers to a cell that divides and reproduces abnormally because growth and division are not regulated or controlled, i.e. cells that are susceptible to uncontrolled growth. A tumor cell can be a benign or malignant cell. Typically, the tumor cell is a malignant cell that can spread to other parts of the body, a process known as metastasis.

As used herein, LIVP GLV-1h68 (also designated GLV-ONC1) is an LIVP virus that contains ruc-GFP (a luciferase and green fluorescent protein fusion gene (see e.g., U.S. Pat. No. 5,976,796), beta-galactosidase (LacZ) and beta-glucuronidase (gusA) reporter genes inserted into the F14.5L, J2R (thymidine kinase) and A56R (hemagglutinin) loci, respectively. The genome of GLV-1h68 has a sequence of nucleotides set forth in SEQ ID NO: 2 or a sequence of nucleotides that has at least 97%, 98% or 99% sequence identity to the sequence of nucleotides set forth in SEQ ID NO: 2.

As used herein, a virus preparation or virus composition, for example an LIVP virus preparation, refers to a virus composition obtained by propagation of a virus strain, for example an LIVP virus strain, an LIVP clonal strain or a modified or recombinant virus strain, in vivo or in vitro in a culture system. For example, an LIVP virus preparation refers to a viral composition obtained by propagation of a virus strain in host cells, typically upon purification from the culture system using standard methods known in the art. A virus preparation generally is made up of a number of virus particles or virions. If desired, the number of virus particles in the sample or preparation can be determined using a plaque assay to calculate the number of plaque forming units per sample unit volume (pfu/mL), assuming that each plaque formed is representative of one infective virus particle. Each virus particle or virion in a preparation can have the same genomic sequence compared to other virus particles (i.e., the preparation is homogenous in sequence) or can have different genomic sequences (i.e., the preparation is heterogenous in sequence). It is understood to those of skill in the art that, in the absence of clonal isolation, heterogeneity or diversity in the genome of a virus can occur as the virus reproduces, such as by homologous recombination events that occur in the natural selection processes of virus strains (Plotkin & Orenstein (eds) “Recombinant Vaccinia Virus Vaccines” in Vaccines, 3^{rd} edition (1999)).

As used herein, plaque forming unit (pfu) or infectious unit (IU) refers to the number of infectious or live viruses. It thus reflects the amount of active virus in the preparation. The pfu can be determined using a plaque formation assay or an end-point dilution assay, which are standard assays known to one of skill in the art.

As used herein, a nanoparticle refers to a colloidal particle for delivery of a molecule or agent that is microscopic in size of between or about between 1 and 1000 nanometers (nm), such as between 1 and 100 nm and behave as a whole unit in terms of transport and properties. Nanoparticles include those that are uniform in size. Nanoparticles include those that contain a targeting molecule attached to the outside.

As used herein, “targeting molecule” or “targeting ligand” refers to any molecular signal directing localization to specific cells, tissues or organs. Examples of targeting ligands include, but are not limited to, proteins, polypeptides or portions thereof that bind to cell surface molecules, including, but not limited to, proteins, carbohydrates, lipids or other such moieties. For example, targeting ligands include proteins or portions thereof that bind to cell surface receptors or antibodies directed to antigens expressed selectively on a target cell. Targeting ligands include, but are not limited to growth factors, cytokines, adhesion molecules, neuropeptides, protein hormones and single-chain antibodies (scFv).

As used herein, a delivery vehicle for administration refers to a lipid-based or other polymer-based composition, such as liposome, micelle or reverse micelle, that associates with an agent, such as a virus provided herein, for delivery into a host subject.

As used herein, accumulation of a virus in a particular tissue refers to the distribution or colonization of the virus in particular tissues of a host organism after a time period following administration of the virus to the host, long enough for the virus to infect the host's organs or tissues. As one skilled in the art will recognize, the time period for infection of a virus will vary depending on the virus, the organ(s) or tissue(s) to be infected, the immunocompetence of the host, and the dosage of the virus. Generally, accumulation can be determined at time points from about less than 1 day, about 1 day to about 2, 3, 4, 5, 6 or 7 days, about 1 week to about 2, 3 or 4 weeks, about 1 month to about 2, 3, 4, 5, 6 months or longer after infection with the virus. For purposes herein, the viruses preferentially accumulate in immunoprivileged tissue, such as inflamed tissue or tumor tissue, but are cleared from other tissues and organs, such as non-tumor tissues, in the host to the extent that toxicity of the virus is mild or tolerable and at most, not fatal.

As used herein, “directed to cells” or “localized to cells” with reference to a nucleic acid molecule provided herein means that the nucleic acid molecule, when administered to a subject, preferentially replicates or accumulates for expression of a chromophore-producing enzyme in a pre-determined target cell or cells, such as a cell (e.g., a tumor cell) involved in a disease process, compared to other cells. For example, included among nucleic acid molecules provided herein are nucleic acid molecules that, when administered to a subject, are directed or localized to tumor cells. A nucleic acid molecule can be made to be directed or localized to a cell by virtue of targeting the nucleic acid molecule to the cell or by conferring selective or specific expression of the nucleic acid molecule in a cell (e.g., cell-specific or tumor-specific expression) such as by using a cell-specific promoter or tumor-specific promoter.

As used herein, to target a nucleic acid molecule means to direct it to a cell that expresses a selected receptor or cell surface molecule by linking the agent to a targeting moiety. Upon binding to the receptor the nucleic acid molecule linked directly or indirectly to the targeting moiety is internalized or otherwise taken up by the cell.

As used herein, “predetermined” with reference to a time sufficient for the nucleic acid molecule to express the chromophore-producing enzyme refers to a limited time that is known or has been previously determined. Typically, the predetermined time is a time in which subsequent to administration or delivery of a nucleic acid molecule to a subject, the nucleic acid molecule is expressed in a subject and the chromophore product produced. For example, the predetermined time can be 3 hours to 3 months, such as generally at least 24 hours, 48 hrs, 72 hours, 96 hours, 5 days, 6 days, 7 days, or 8 days after delivery of the nucleic acid.

As used herein, the term assessing or determining is intended to include quantitative and qualitative determination in the sense of obtaining an absolute value for the activity of a product, and also of obtaining an index, ratio, percentage, visual or other value indicative of the level of the activity. Assessment can be direct or indirect.

As used herein, activity refers to the in vitro or in vivo activities of a compound or virus provided herein. For example, in vivo activities refer to physiological responses that result following in vivo administration of a compound or virus provided herein (or of a composition or other mixture thereof). Activity, thus, encompasses resulting therapeutic effects and pharmaceutical activity of such compounds, compositions and mixtures. Activities can be observed in in vitro and/or in vivo systems designed to test or use such activities.

As used herein, “anti-tumor activity” or “anti-tumorigenic” refers to virus strains that prevent or inhibit the formation or growth of tumors in vitro or in vivo in a subject. Anti-tumor activity can be determined by assessing a parameter or parameters indicative of anti-tumor activity.

As used herein, a “parameter indicative of anti-tumor activity or anti-tumorigenic activity” refers to a property mediated by a virus that is associated with anti-tumor activity. Parameters indicative of anti-tumor activity can be assessed in vitro or in vivo upon administration to a subject. Exemplary parameters indicative of anti-tumor activity include, but are not limited to, infectivity of tumor cells, accumulation of virus in tumor tissues, viral nucleic acid replication in tumor cells, virus production in tumor cells, viral gene expression in tumor cells, cytotoxicity of tumor cells, tumor cell selectivity, tumor cell type selectivity, decreased tumor size, decreased tumor volume, decreased tumor weight, and initiation of specific and nonspecific anti-tumor immune responses. Assays that assess any of the above parameters or other anti-tumorigenic properties are known to one of skill in the art. Exemplary assays are described herein. Hence, a virus that exhibits any one or more of the above activities or properties exhibits anti-tumor activity.

As used herein, “toxicity” (also referred to as virulence or pathogenicity herein) with reference to a virus refers to the deleterious or toxic effects to a host upon administration of the virus. For an oncolytic virus, such as LIVP, the toxicity of a virus is associated with its accumulation in non-tumorous organs or tissues, which can impact the survival of the host or result in deleterious or toxic effects. Toxicity can be measured by assessing one or more parameters indicative of toxicity. These include accumulation in non-tumorous tissues and effects on viability or health of the subject to whom it has been administered, such as effects on body weight.

As used herein, a “parameter indicative of toxicity” refers to a property mediated by a virus that is associated with its toxicity, virulence or pathogenicity. Parameters indicative of toxicity generally are assessed in vivo upon administration to a subject. Exemplary parameters indicative of toxicity include, but are not limited to, decreased survival of the subject, decreased body weight, fever, rash, allergy, fatigue, abdominal pain, induction of an immune response in the subject and pock formation. Assays or measures that assess any of the above parameters or other toxic properties known to one of skill in the art are described herein or are known to one of skill in the art. Hence, a virus that mediates any one or more of the above activities or properties in a host exhibits some degree of toxicity.

As used herein, nucleic acids include DNA, RNA and analogs thereof, including peptide nucleic acids (PNA) and mixtures thereof. Nucleic acids can be single or double-stranded. Nucleic acids can encode gene products, such as, for example, polypeptides, regulatory RNAs, microRNAs, siRNAs and functional RNAs.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem. 243:3557-3559 (1968), and adopted 37 C.F.R. §§1.821-1.822, abbreviations for amino acid residues are shown in Table 1:

TABLE 1
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-stereoisomers of amino acids. Exemplary non-natural amino acids are described herein and are known to those of skill in the art.

As used herein, suitable conservative substitutions of amino acids are known to those of skill in the art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in TABLE 2 as follows:

TABLE 2

| | Exemplary conservative |
|---|---|
| Original residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, recitation that nucleotides or amino acids “correspond to” nucleotides or amino acids in a disclosed sequence, such as set forth in the Sequence Listing, refers to nucleotides or amino acids identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073).

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity-over most of their length. Exemplary algorithms for performing global alignment include the Needleman-Wunsch algorithm (Needleman et al. J. Mol. Biol. 48: 443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or the Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non-identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences (or any two polypeptides have amino acid sequences) that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Generally, when comparing nucleotide sequences herein, an alignment with no penalty for end gaps (e.g., terminal gaps are not penalized) is used.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differ from those of the reference polypeptides. Similar comparisons can be made between test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, the terms immunoprivileged cells and immunoprivileged tissues refer to cells and tissues, such as solid tumors, which are sequestered from the immune system. Generally, administration of a virus to a subject elicits an immune response that clears the virus from the subject. Immunoprivileged sites, however, are shielded or sequestered from the immune response, permitting the virus to survive and generally to replicate. Immunoprivileged tissues include proliferating tissues, such as tumor tissues and other tissues and cells involved in other proliferative disorders, wounds and other tissues involved in inflammatory responses.

As used herein, a wound or lesion refers to any damage to any tissue in a living organism. The tissue can be an internal tissue, such as the stomach lining or a bone, or an external tissue, such as the skin. As such, a wound or lesion can include, but is not limited to, a gastrointestinal tract ulcer, a broken bone, a neoplasia, and cut or abraded skin. A wound or lesion can be in a soft tissue, such as the spleen, or in a hard tissue, such as bone. The wound or lesion can have been caused by any agent, including traumatic injury, infection or surgical intervention.

As used herein, a skin lesion refers to a lesion on the surface of the skin. The skin lesion can be have been caused by a traumatic injury, infection, surgical intervention or an environmental factor. Exemplary skin lesions include, but are not limited to, precancerous lesion (e.g., actinic keratosis of the skin), a cancerous lesion (e.g., skin cancer), a traumatic wound (e.g., burn or scar) or a post-surgical wound (e.g., surgically resected tumor). In particular, the lesion is a skin cancer lesion such as basal cell carcinoma or squamous cell carcinoma.

As used herein, a tumor, also known as a neoplasm, is an abnormal mass of tissue that results when cells proliferate at an abnormally high rate. Tumors can show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g., breast, prostate, lung, colon, pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells), including components of the immune system. Other malignant tumors include, but are not limited to, tumors of the nervous system (e.g., neurofibromatomas), germ cell tumors, and blastic tumors.

As used herein, a resected tumor refers to a tumor in which a significant portion of the tumor has been excised. The excision can be effected by surgery (i.e., a surgically resected tumor). The resection can be partial or complete.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, a cell involved in a disease or disease process refers to cells whose presence contributes to, exacerbates, causes or otherwise is involved in the etiology of a disease or disease process. Inhibition or killing of such cells can ameliorate the symptoms of the disease or can ameliorate the disease. Examples of such cells are tumor cells. Killing or inhibiting the growth or proliferation of tumor cells effects treatment of tumors. Other examples are immune effector cells, which participate in inflammatory responses that contribute to the pathology of a variety of diseases. Inhibiting or killing immune effector cells can treat diseases that have an inflammatory component.

As used herein, “killing or inhibiting growth or proliferation of cells” means that the cells die or are eliminated. Inhibiting growth or proliferation means that the number of such cells does not increase, and can decrease.

As used herein, proliferative disorders or hyperproliferative disorders include any disorders involving abnormal proliferation of cells. Such disorders include, but are not limited to, neoplastic diseases, inflammatory responses and disorders, e.g., including wound healing responses, psoriasis, restenosis, macular degeneration, diabetic retinopathies, endometriosis, benign prostatic hypertrophy, hypertrophic scarring, cirrhosis, proliferative vitreoretinopathy, retinopathy of prematurity, and immunoproliferative diseases or disorders, e.g., inflammatory bowel disease, rheumatoid arthritis, systemic lupus erythematosus (SLE) and vascular hyperproliferation secondary to retinal hypoxia or vasculitis.

As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma. epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia or Wilm's tumor. Exemplary cancers commonly diagnosed in humans include, but are not limited to, cancers of the bladder, brain, breast, bone marrow, cervix, colon/rectum, kidney, liver, lung/bronchus, ovary, pancreas, prostate, skin, stomach, thyroid, or uterus. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, a “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, an anticancer agent or compound (used interchangeably with “antitumor or antineoplastic agent”) refers to any agent or compound used in anticancer treatment. These include any agents, when used alone or in combination with other compounds or treatments, that can alleviate, reduce, ameliorate, prevent, or place or maintain in a state of remission of clinical symptoms or diagnostic markers associated with neoplastic disease, tumors and cancer, and can be used in methods, combinations and compositions provided herein. Anticancer agents include antimetastatic agents. Exemplary anticancer agents include, but are not limited to, chemotherapeutic compounds (e.g., toxins, alkylating agents, nitrosoureas, anticancer antibiotics, antimetabolites, antimitotics, topoisomerase inhibitors), cytokines, growth factors, hormones, photosensitizing agents, radionuclides, signaling modulators, anticancer antibodies, anticancer oligopeptides, anticancer oligonucleotides (e.g., antisense RNA and siRNA), angiogenesis inhibitors, radiation therapy, or a combination thereof. Exemplary chemotherapeutic compounds include, but are not limited to, Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. As used herein, reference to an anticancer or chemotherapeutic agent includes combinations or a plurality of anticancer or chemotherapeutic agents unless otherwise indicated.

As used herein, a “chemosensitizing agent” is an agent which modulates, attenuates, reverses, or affects a cell's or organism's resistance to a given chemotherapeutic drug or compound. The terms “modulator”, “modulating agent”, “attenuator”, “attenuating agent”, or “chemosensitizer” can be used interchangeably to mean “chemosensitizing agent.” In some examples, a chemosensitizing agent can also be a chemotherapeutic agent. Examples of chemosensitizing agents include, but are not limited to, radiation, calcium channel blockers (e.g., verapamil), calmodulin inhibitors (e.g., trifluoperazine), indole alkaloids (e.g., reserpine), quinolines (e.g., quinine), lysosomotropic agents (e.g., chloroquine), steroids (e.g., progesterone), triparanol analogs (e.g., tamoxifen), detergents (e.g., Cremophor EL), texaphyrins, and cyclic antibiotics (e.g., cyclosporine).

As used herein, a subject includes any organism, including an animal for whom diagnosis, screening, monitoring or treatment is contemplated. Animals include mammals such as primates and domesticated animals. An exemplary primate is a human. A patient refers to a subject, such as a mammal, primate, human, or livestock subject afflicted with a disease condition or for which a disease condition is to be determined or risk of a disease condition is to be determined.

As used herein, a patient refers to a human subject exhibiting symptoms of a disease or disorder.

As used herein, treatment of a subject that has a condition, disorder or disease means any manner of treatment in which the symptoms of the condition, disorder or disease are ameliorated or otherwise beneficially altered. Treatment encompasses any pharmaceutical use of the viruses described and provided herein.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, treatment of a wound refers to any manner of treatment in which the signs or symptoms of having a wound are ameliorated or otherwise beneficially altered. Typically, treatment encompasses alleviation of the wound, shrinkage of the wound, reduction in the size of the wound or other similar result that is associated with wound healing.

As used herein, treatment of a subject that has a neoplastic disease, including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, efficacy means that upon administration of a virus or virus composition, the virus will colonize proliferating or immunoprivileged cells, such as tumor cells, and replicate. Colonization and replication in tumor cells is indicative that the treatment is or will be an effective treatment.

As used herein, effective treatment with a virus is one that can increase survival compared to the absence of treatment therewith. For example, a virus is an effective treatment if it stabilizes disease, causes tumor regression, decreases severity of disease or slows down or reduces metastasizing of the tumor.

As used herein, a composition refers to any mixture. It can be a solution, suspension, liquid, gel, powder, paste, aqueous, non-aqueous or any combination thereof.

As used herein, a formulation refers to a composition containing at least one active pharmaceutical or therapeutic agent and one or more excipients.

As used herein, a co-formulation refers to a composition containing two or more active or pharmaceutical or therapeutic agents and one or more excipients.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, direct administration refers to administration of a composition without dilution.

As used herein, a kit is a packaged combination, optionally, including instructions for use of the combination and/or other reactions and components for such use.

As used herein, an “article of manufacture” is a product that is made and sold. As used throughout this application, the term is intended to encompass articles containing a vaccinia virus alone or in combination with a second therapy or a therapeutic energy source contained in the same or separate articles of packaging.

As used herein, a device refers to a thing made or adapted for a particular task. Exemplary devices herein are devices that cover or coat or are capable of contacting the epidermis or surface of the skin. Examples of such devices include, but are not limited to, a wrap, bandage, bind, dress, suture, patch, gauze or dressing.

As used herein, the singular forms “a,” “an” and “the” include plural referents unless the context clearly dictates otherwise.

As used herein, ranges and amounts can be expressed as “about” or “approximately” a particular value or range. “About” or “approximately” also includes the exact amount. Hence, “about 5 milliliters” means “about 5 milliliters” and also “5 milliliters.” Generally “about” includes an amount that would be expected to be within experimental error.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amounts can vary depending upon the tolerance for variation in the particular composition by subjects.

As used herein, “optional” or “optionally” means that the subsequently described event or circumstance does or does not occur, and that the description includes instances where said event or circumstance occurs and instances where it does not.

As used herein, the abbreviations for any protective groups, amino acids and other compounds are, unless indicated otherwise, in accord with their common usage, recognized abbreviations, or the IUPAC-IUB Commission on Biochemical Nomenclature (see, (1972) Biochem. 11:1726).

For clarity of disclosure, and not by way of limitation, the detailed description is divided into the subsections that follow.

### B. GENE-EVOKED CHROMOPHORE (e.g., MELANIN) PRODUCTION AS THERANOSTIC MEDIATOR

Provided are methods of theranostics, whereby nucleic acid molecule(s), encoding one or more proteins (e.g., chromophore-producing enzyme, such as melanin-producing enzymes) are introduced into a cell, for example a tumor cell, resulting in production or increased production of a compound (e.g., a chromophore or pigment such as melanin) or protein, that induces toxicity of the host cell following absorbance of electromagnetic radiation. Thus, the nucleic acid compositions provided herein can be used in therapeutic methods to eliminate cells that are associated with, cause or otherwise exacerbate a disease or condition, such as tumor cells. In addition, the properties of the accumulated compound (e.g., melanin) can be further exploited for diagnostic purposes, for example to facilitate diagnostic imaging of the host cell, in particular a host cell that is a tumor cell.

For purposes of the methods herein, the chromophore-producing enzyme or enzymes are any that are sufficient for the production of a chromophore in the host cell that is capable of inducing toxicity of the host cell following absorbance of electromagnetic radiation. In particular, the absorbance spectrum of the introduced chromophore product and/or precursors thereof differ from those of biological tissue, for example human tissue, and components thereof, such as blood, which contains known chromophores hemoglobin, oxyhemoglobin, water and lipid. For example, melanin exhibits enhanced absorption of light in the near-infrared optical window, which is characterized by low absorption and maximum light penetration in tissues. The transferred energy is then converted to thermal energy eventually heating the chromophore (e.g., melanin) producing cells and cells in their vicinity to temperatures that cause protein denaturation and cell death. For example, chromophore (e.g., melanin) containing cells exposed to electromagnetic radiation in the near-infrared optical range of from or from about 700-900 nm, such as about 808 nm, can exhibit a temperature of greater than 20° C., 30° C., 40° C., 50° C., 60° C., 70° C., 80° C. or higher. The temperature can increase greater than 5° C., 10° C., 15° C., 20° C., 30° C., 40° C., 50° C. or more compared to cells that are not made to overproduce the chromophore.

Thus, in applications of thermotherapy methods provided herein, cells producing the chromophore (e.g., melanin), or precursors thereof, can be treated with electromagnetic radiation at one or more wavelengths that are less absorbed by biological tissue than the chromophore product, for example wavelengths in the near infrared spectrum, that are well-absorbed by the chromophore resulting in thermal radiation, which results in selective hyperthermia or thermotherapy of the chromophore-containing tissue. For example, a near-infrared laser beam can be used to induce thermal transduction of a chromophore (e.g., melanin), produced as a result of transgenic expression of biosynthetic enzymes, thereby increasing the temperature of host cells, particularly host tumor cells, and effect host cell toxicity. Upon absorbance of electromagnetic radiation, at wavelengths that are greater absorbed by the chromophore than the components of biological tissue (e.g., hemoglobin, oxyhemoglobin), the chromophore product and/or precursors thereof absorb the electromagnetic radiation and effect selective cell toxicity of the host cell, for example by emitting thermal radiation and/or producing reactive oxygen species in sufficient quantities to induce cytotoxicity. Melanin is an example of such a chromophore product, which can be synthesized or produced in a cell carrying a tyrosinase enzyme in the presence or absence of other melanin-producing enzymes (e.g., tyrosinase related protein 1).

While chromophores such as melanin are present in cells and tissues of the body, such as pigment cells and tissues, it is found herein that their synthesis and production can be directed by delivery of exogenous genes to particular host cells. This can result in the production or overproduction of a desired chromophore product in any desired host cell or tissue that is desired to be eliminated for the treatment of a disease or condition. In particular aspects of the methods herein, nucleic acid molecules encoding a chromophore-producing enzyme or enzymes are delivered to a subject for the directed delivery to tumor cells. Subsequent exposure to electromagnetic radiation at one or more wavelengths that are less absorbed by the biological tissue than the chromophore product results in the killing and cell death of the tumor cells, thereby reducing tumor volume and size. Accordingly, the methods provided herein can be practiced in non-melanoma tumor cells to which exogenous enzymes are delivered for the de novo synthesis of melanin in the cell.

In addition to photothermal and photodynamic therapeutic applications, the chromophore product (e.g., melanin) introduced into a host cell using the methods provided herein, can be used for diagnostic purposes, for example, diagnostic imaging. For example, metal chelating chromophores, such as melanin, can enhance the contrast in optical and acoustic imaging, as well as magnetic resonance imagining, and thereby improve detection of tissues containing the chromophore. Such imaging techniques permit the visualization and presence of the chromophore deep within the host body, thereby permitting not only the diagnosis of surface tumors but also tumors deep within the body, such as metastases. In addition, since the chromophore is produced enzymatically it is expressed relatively quickly upon delivery of a nucleic acid molecule encoding a chromophore-producing enzyme. Thus, nucleic acids encoding chromophore-producing enzymes, such as melanin-producing enzymes, can be used in diagnostic methods to facilitate fast detection within 18 hours post nucleic acid delivery, such as post viral infection, with increasing intensity over time. This is an advantage over other detection methods using fluorescent proteins that are not produced in detectable amounts as quickly. The use of fluorescent proteins as a detectable moiety also are hampered by the fact that many tissues are autofluorescent, which can interfere with the signal. This is not a problem for melanin, and in fact, the production of melanin can reduce tissue autofluorescence. In addition, other advantages over fluorescent proteins include, for example, the stability of melanin, the fact that no special equipment is needed to visualize the dark pigments, and that the introduction of melanin synthesis should be adaptable to most species because of the ubiquitous presence of melanin in all kingdoms of life.

Diagnostic imaging can be used alone or in combination with photothermal and/or photodynamic applications. For example, diagnostic imaging, enhanced by production of the chromophore product (e.g., melanin) can be used prior to photothermal therapy to aid in identification of the tissue(s) to be treated by photothermal therapy and also to assist with more accurate direction of the laser light. Diagnostic imaging also can be used to monitor the progress and effectiveness of the photothermal/photodynamic treatments.

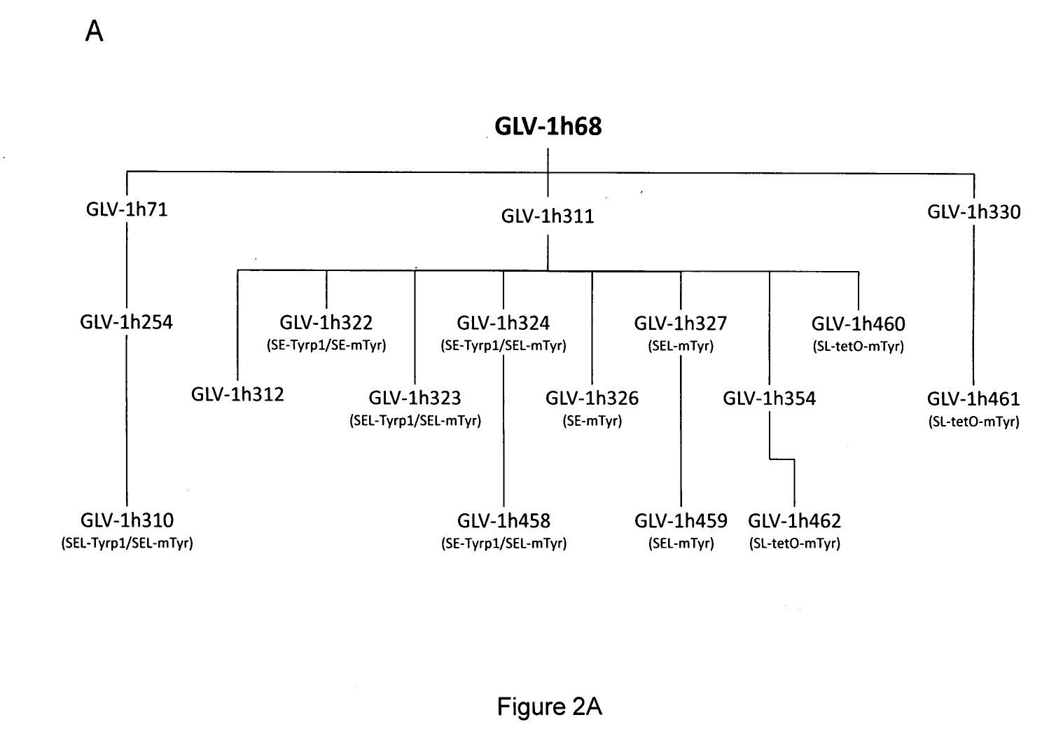

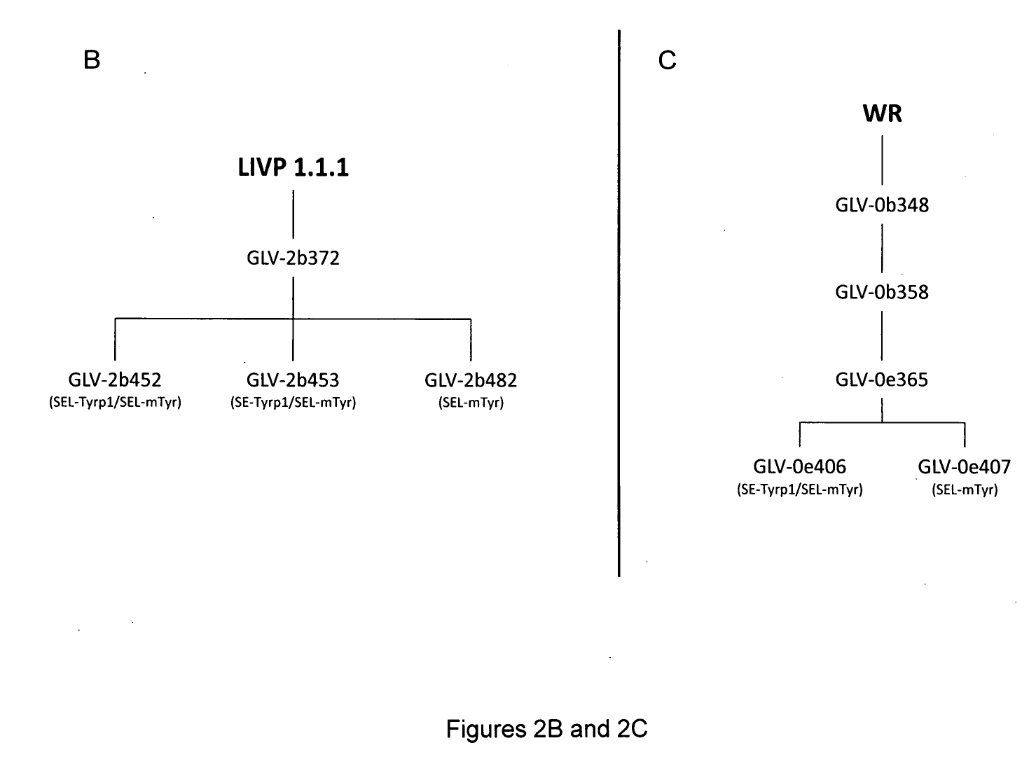

The nucleic acid can be a viral vector or non-viral vector. In the methods herein, the nucleic acid is delivered to host cells by any method that effects delivery to cells, including but not limited to, electroporation, direct injection, systemic injection or infusion or other methods as described herein or known in the art. Typically, the nucleic acid that is delivered is one that is capable of directed or localized expression in immunoprivileged cells or tissues, such as tumor cells or inflamed cells or tissues (e.g., atherosclerotic plaques). In particular, viral vectors (e.g., oncolytic viruses) are used to infect cells, in particular cancer cells, including tumor cells. For example, the viral vectors used in the methods described herein preferentially infect tumor cells and contain exogenous nucleic acid molecule(s) that encode one or more chromophore-producing enzymes that are capable of synthesis of proteins or compounds that induce toxicity as a result of exposure to electromagnetic radiation. In particular, oncolytic viral vectors are used to deliver nucleic acid molecules encoding enzymes (e.g., tyrosinase and/or tyrosinase-related protein-1) that are directly or indirectly involved in the biosynthesis of a chromophore product (e.g., melanin) and/or the precursors of a chromophore to a tumor cell. Exemplary oncolytic viral vectors are described herein (see also FIG. 2).

The compositions and methods provided herein are exemplified with nucleic acid compositions encoding melanin-producing enzymes, such as tyrosinase or tyrosinase-related protein 1. The following sections describe in further detail the biosynthetic pathways for melanin production, exemplary nucleic acid molecules and compositions encoding chromophore-producing enzymes for use in performing the energy-absorbing therapeutic methods herein. Also described below are methods and applications of the energy-absorbing therapeutic method alone or in combination with other therapy or diagnostic methods. The compositions used in the method can be adapted by one of skill in the art based on the description herein for use in any particular application or therapeutic indication in which a cell or cells is desired to be eliminated (e.g., tumor cell), and depending on the particular subject or host and other similar considerations.

### C. MELANIN BIOSYNTHESIS AND PROPERTIES

Melanin pigments are polymers of oxidized derivatives of the amino acid tyrosine. There are many different types of melanin, with differing properties. Aside from the melanin found in the brain, mammalian melanin is classified into the brown/black pigment eumelanin and the red/yellow pigment pheomelanin. Eumelanin is the most abundant melanin in humans, found in the eyes and black hair, and is also found in the ink sac of some cephalopods (e.g., cuttlefish). Red/yellow pheomelanin is found in the lips, genitals, and red hair.

1. Biosynthesis of Melanin

FIG. 1 depicts the biosynthetic pathway for melanin production. Biosynthesis of eumelanin and pheomelanin begins with the hydroxylation of tyrosine to 3,4-dihydroxphenylalanine (DOPA) and oxidation of DOPA to Dopaquinone by the enzyme tyrosinase (Tyr). At this point, whether eumelanin or pheomelanin is produced depends on the presence of the amino acid cysteine. In the presence of cysteine, Dopaquinone is converted to Cysteinyldopa, the metabolites of which lead to the production of pheomelanin. In the absence of cysteine, Dopaquinone is converted to Dopachrome, which is isomerized to 5,6-dihydroxyindole-2-carboxylic acid (DHICA) in a reaction catalyzed by tyrosinase-related protein-2/dopachrome tautomerase (TRP-2/Dct) or spontaneously decomposed to form 5,6-dihydroxyindole (DHI). Oxidation of DHICA, to yield indole-2-carboxylic acid quinone is catalyzed by tyrosinase-related protein-1 (Trp-1). DHI is oxidized to form Indolequinone in a reaction catalyzed by tyrosinase (Tyr). The enzyme tyrosinase is involved in the rate-limiting step in both pathways leading to eumelanin production and pheomelanin production.

Eumelanin pigments are heterogeneous macromolecules constructed from a combination of reduced and oxidized DHICA and DHI units. As shown in the examples herein, the ratio and extent of eumelanin to pheomelanin produced can be dependent on the amount of tyrosinase that is present. In addition, the secondary enzymes tyrosinase-related protein-2/dopachrome tautomerase (TRP-2/Dct) and tyrosinase-related protein-1 (Trp-1) are only involved in the pathway leading to production of eumelanin. The presence of these secondary enzymes in addition to tyrosinase can promote the production of eumelanin over pheomelanin. The ratios of eumelanin to pheomelanin also can be affected by peroxidase oxidizing enzymes and Zn(II) ions. Eumelanin strongly absorbs ultraviolet radiation, while pheomelanin weakly absorbs ultraviolet radiation.

2. Properties of Melanin

Melanin polymers exhibit several properties, including light absorbing, redox and chelating properties. Theses physical and chemical properties can be leveraged by a variety of methods, as disclosed herein, for therapeutic purposes.

a. Optical Properties

Unlike other biological pigments present in tissue (e.g., hemoglobin, bilirubin and β-carotene), which exhibit distinct absorption spectra with distinct absorbance peaks, melanin exhibits a broadband absorption spectrum. Melanin's broadband absorbance spectrum is likely a summation of the individual peaked spectra of the multiple melanin species that make up the heterogeneous melanin pigment population, although the wide spectral absorbance has also been attributed to the high degree of conjugation within melanin molecules. The absorbance of melanin increases monotonically as the wavelength decreases and the energy of the electromagnetic radiation increases, enabling melanin's ability to absorb high energy ultraviolet radiation and offer photoprotection to melanin-containing cells.

Biological pigments other than melanin typically exhibit peak absorbance between about 400-500 nm. Wavelengths greater than about 500 nm are more absorbed by melanin than the other major biological pigments, including oxyhemoglobin, deoxyhemoglobin, bilirubin and β-carotene. In addition to biological pigments, water and lipids also absorb electromagnetic radiation. The absorption spectrum of water shows that water exhibits strong absorption at the short wavelengths (up to approximately 200 nm), minimal absorbance between 200 and 420 nm and then increasing absorption from 420 to 10,000 nm. Lipids also contribute to electromagnetic absorption of tissue, absorbing increasing amounts of radiation at increasing wavelengths greater than approximately 600 nm. At electromagnetic wavelengths of approximately 500 to 1500 nm, melanin is the most absorbent component of human tissue.

Transmission of electromagnetic radiation through human tissue is a function of the refractive indices of the tissue and distribution of chromophores (e.g., hemoglobin, melanin, water, lipid) within the tissue which will bring about different reflection, transmission, scattering and absorption characteristics depending on the wavelength. The optical window, also called the therapeutic window, defines the range of wavelengths where electromagnetic radiation has its maximum depth of penetrance in tissue. Because there is little correlation between scattering and wavelength, the optical window is primarily limited to absorption by blood at shorter wavelengths and water at longer wavelengths. Due to the reduced absorption by tissue chromophores at red and near infrared (NIR) wavelengths of 600-1200 nm, wavelengths within this region of the spectrum exhibit the deepest tissue penetration. As discussed above, melanin exhibits greater absorption at these wavelengths than other components of the tissue. Therefore, in some embodiments provided herein, wavelengths within the range of 600-1200 nm are used to achieve specific absorption of electromagnetic energy in melanin-containing cells as well as maximum tissue penetrance.

b. Metal Binding and Reduction-Oxidation (Redox)

In addition to the optical properties discussed above, melanins, especially eumelanin exhibit strong redox properties which contribute to melanin's role as a photoprotector. In addition to absorbing ultraviolet irradiation directly, melanin protects cells from oxidative stress by scavenging intracellular reactive oxygen species (ROS) (Tada et al., (2010) J Clin Biochem Nutr. 46(3): 224-228). Melanin pigments exist in equilibria between quinole, semi-quinone and quinone oxidation states. Semiquinone free radicals generated as a result of electron delocalization between orthoquinone and catecholic moieties of melanin enable melanins to take part in one-electron and two-electron redox reactions and thereby neutralize ROS.

Metal chelation is also one of the main biological functions of melanin (see Hong and Simon (2007) J Phys Chem B. 111(28):7938-7947). The DHICA moiety of eumelanin coordinates with many metals, in particular, Ca, Mg, Na and K. The DHI moiety of eumelanin binds Zn under neutral pH or slightly acidic conditions. Both DHICA and DHI moieties form complexes with Fe ions.

Melanin has a high capacity for binding metal ions, which enables it to function as an intracellular reservoir for metal ions (e.g., Ca(II) and Zn(II)) and regulate metal storage, release and exchange. While melanin has a moderate affinity for metals such as Ca(II) and Zn(II), melanin has a very strong affinity for heavier and more reactive metals, such as Fe(III), Cu(II), Pb(II), La(III) and Gd(III). Melanin's strong binding and sequestration of such reactive metals serves to prevent their reduction by cellular components and mitigate their role in inducing oxidative stress.

Despite its role in preventing oxidative stress by sequestering reactive metals, melanin itself, as well as DHICA and DHI precursor moieties, can produce free radicals when irradiated with UV light, making melanin a photosensitizer in addition to a photoprotector. Binding metal ions enhances this effect by making melanin more easily oxidized and susceptible to reaction with oxygen to form superoxide and other cytotoxic reactive oxygen species (Sarna et al., (1976) Science 192:101-106; Farmer et al., (2004) Pigment Cell Research, 17(4):434). UV irradiation contributes to this process. As described in the methods provided herein, this photosensitizing property of melanin can be leveraged to induce cell toxicity to target cells (e.g., tumor cells) which accumulate melanin and are subjected to phototherapy.

The metal chelating ability of melanin also can enhance detection of target cells, containing accumulated melanin, using methods provided herein. Semiquinone free radicals within melanin pigments are detectable by paramagnetic resonance (EPR). The paramagnetic resonance signal is enhanced by binding a paramagnetic or superparamagnetic metal such as gadolinium, iron, nickel, copper, erbium, europium, chromium or manganese. These metals can be incorporated into melanin, and utilized to enhance ultrasound, optoacoustic, and magnetic resonance images (see e.g., U.S. Pat. No. 5,310,539).

### D. NUCLEIC ACID MOLECULES ENCODING CHROMOPHORE-PRODUCING ENZYMES

Provided herein are nucleic acid molecules that encode an enzyme or enzymes that produces a chromophore product, for example, melanin or other chromophore. The encoded enzyme or enzymes can be any enzyme, or enzymatically active portion thereof, that is a part of the biosynthetic pathway for generation of a chromophore and is required for the production of the chromophore in a host cell of a subject. In particular, provided herein are nucleic acid molecules that encode a melanin-producing enzyme or an enzymatically active portion thereof, such as a nucleic acid molecule encoding a tyrosinase and/or tyrosinase-related protein 1 or an enzymatically active portion of a tyrosinase or tyrosinase-related protein 1. The nucleic acid molecules, when delivered to cells, can achieve overproduction of the chromophore in the cell. The overproduction of the chromophore, such as overproduction of melanin, can be used in diagnostic and therapeutic applications as described herein.

The nucleic acid molecules encoding an enzyme or enzymes that produce a chromophore product can be any non-viral or viral nucleic acid molecule that contains a sequence of nucleotides encoding an enzyme or enzymes that produce a chromophore product. Generally, the nucleic acid molecule is a DNA, such as a double stranded circular or linear DNA. The DNA can be a naked DNA. The nucleic acid molecule can be provided as a construct containing a heterologous nucleic acid molecule. There are a number of constructs that are known to one of skill in the art for in vitro or in vivo delivery of nucleic acid to cells. Such constructs include viral based delivery systems and non-viral based deliver systems. For example, the nucleic acid molecule can be a construct containing a nucleic acid molecule that is delivered in a nanoparticle (e.g., a targeted or radiolabeled nanoparticle), a plasmid or a vector (e.g., a viral vector or an expression vector). Such constructs are well-known in the art and readily adaptable for use with the compositions and methods described herein. The nucleic acid molecule can be delivered in a vehicle or as a complex. For example, the nucleic acid molecule can be encapsulated in liposomes. The nucleic acid molecule can be complexed to other agents, such as target ligands or other moieties and delivered as a nanoparticle. The nucleic acid molecule can be delivered by any method known in the art, including, but not limited to, injection, infusion, transfection, microinjection, gene gun and electroporation. For example, the nucleic acid molecules can be delivered systemically or directly to cells of a host.

The nucleic acid molecule can be driven by a promoter to enhance, control or regulate expression. The promoter is operably linked to the coding region of the gene encoding the chromophore-producing enzyme. Any strong promoter known to those skilled in the art can be used for driving the expression of DNA. The promoter can be a constitutive promoter, such as a CMV promoter, a tissue-specific promoter, or an inducible or regulatable promoter. The promoter can be a viral promoter or a eukaryotic promoter. For example, suitable strong promoters include, but are not limited to, adenoviral promoters, such as the adenoviral major late promoter; vaccinia synthetic early-late promoter (P_{SEL}; SEQ ID NO: 169); vaccinia synthetic late promoter (P_{SL}); the cytomegalovirus (CMV) promoter; the respiratory syncytial virus (RSV) promoter; the simian virus 40 (SV40) promoter; respiratory syncytial virus (RSV) promoter; human elongation factor 1α-subunit (EF1-1α) promoter; a ubiquitin C promoter (Ubc); a phosphoglycerate kinase-1 (PGK) promoter; small nuclear RNA U1b promoter; glucose 6-phosphate dehydrogenase promoter; heat shock promoters; the albumin promoter; the ApoAI promoter; human globin promoters; viral thymidine kinase promoters, such as the Herpes Simplex thymidine kinase promoter; retroviral LTRs; the β-actin promoter; human growth hormone promoters; phage promoters, such as the T7, SP6 and T3 RNA polymerase promoters; and the cauliflower mosaic virus 35S (CaMV 35S) promoter. Retroviral LTRs include, for example, Rous sarcoma virus long terminal repeat (RSV-LTR) and Moloney murine leukemia virus (MoMLV) LTR. Suitable strong promoters also can include inducible promoters, such as the metallothionein (e.g., MMT) promoter. The promoter can also be a tissue- or cell-specific promoter, including any described elsewhere herein or known in the art (see e.g., Papadakis et al. (2004) Current Gene Therapy, 4:89-113).

In some examples, the nucleic acid molecule is formulated with an agent or delivery vehicle that binds to or complexes with the nucleic acid and mediates its entry into cells. Exemplary agents include, but are not limited to, cationic liposomes and lipids, lipoproteins, synthetic polymers or polypeptides, mineral compounds or vitamins. Exemplary polymers include polycations or polyanions. For example, a nucleic acid molecule can be formulated with polyamine, calcium phosphate precipitate, histone protein, protamine, polyethyleneimine, polylysine, polyarginine, polyornithine, DEAE dextran, polybrene, polyampholyte complex, spermine, spermidine, putrescine, human serum albumin, DNA binding proteins, non-histone chromosomal proteins, coat proteins from DNA viruses and polymers of N-substituted glycines.

For example, the nucleic acid molecule can be encapsulated in lipids or packaged in liposomes prior to delivery to the subject. Lipid encapsulation is generally accomplished using liposomes that are able to stably bind or entrap and retain nucleic acid. The ratio of condensed nucleic acid delivered agent to lipid preparation can vary but will generally be around 1:1 (mg DNA:micromoles lipid) or more of lipid. Liposomal preparations include cationic (positively charged), anionic (negatively charged) and neutral preparations. Such preparations are well-known to one of skill in the art and readily available. For example, exemplary cationic lipids include, but are not limited to, N[1-2,3-dioleyloxy)propyl]-N,N,N-trimethylammonium (DOTMA; available under the product line Lipofectin®); DDAB/DOPE and DOTAP/DOPE. Anionic and neutral liposomes also are readily available and can be prepared from phosphatidyl choline, cholesterol, phosphatidyl ethanolamine, dioleoylphosphatidyl choline (DOPC), dioleoylphosphatidyl glycerol (DOPG), dioleoylphosphatidyl ethanolamine (DOPE), such as the commercially available preparation Avanti Polar Lipids. The liposomes include multilamellar vesicles (MLVs), small unilamellar vesicles (SUVs), or large unilamellar vesicles (LUCs).

The nucleic acid molecules also can be formulated as nanoparticles, generally of 3-200 nm in size. The generation of nanoparticles for gene therapy is well-known in the art (see e.g., Cho et al. (2008) Clin. Cancer. Res., 14:1310; Jin et al. (2007) Biotechnol. Prog., 23:32-41). The nanoparticle can be made as a polymer, such as by using polymer carriers (e.g., polylactic acid, polysaccharides, poly(cyanoacrylates, poly(lactide-co-glycolide)) or branched polymers to generate dendrimers, such as by growth polymerization steps from poly(L-glutamic acid (PGA), polyamidoamine (PAMAM), poly(ethylene glycol) (PEG) and polyethyleneimine (PEI). Biodegradable polymers can be used which include, for example, polylactic acid, polyglycolic acid, polylactic-glycolic acid (PLGA) or poly(methyl methacrylate (PMMA). Other types of nanoparticles can be generated as a liposome using various lipid mixtures; as a magnetic nanoparticle using iron oxide, as a silica nanoparticle using SiO₂ or as a gold nanoparticle using chlorauric acid or sodium citrate. Nanoparticle systems are well-known to one of skill in the art.

In particular, the nucleic acid compositions provided herein can be used in targeted synthesis or production of the chromophore (e.g., melanin) in a cell or tissue. In aspects of the compositions and methods provided herein, the nucleic acid molecule is capable of targeting or accumulating in immunoprivileged cells or tissues, such as wounds, inflammatory cells or tumor cells. For example, the nucleic acid molecule compositions provided herein are delivered to tumor cells of a subject to encode a chromophore-producing enzyme, such as a melanin-producing enzyme, to produce or synthesize the chromophore (e.g., melanin) in the tumor cells. The nucleic acid molecules can be used in diagnostic, treatment or theranostic methods provided herein for generating or producing a chromophore in a cell (e.g., melanin) for diagnosis or treatment of any immunoprivileged cell or tissue including, but not limited to, a wound, inflamed tissue, atherosclerotic plaque, or tumor. In particular, the nucleic acid molecules are used in energy-absorbing therapeutic methods (e.g., hyperthermia, photodynamic therapy or other applications) to eliminate cells that cause disease, such as cells associated with atherosclerotic plaques or tumors. In particular examples, the nucleic acid molecules are targeted for delivery to non-melanoma cells or tissues.

1. Chromophore-Producing Enzyme

Provided herein are nucleic acid molecules that contain a sequence of nucleotides that encodes a chromophore-producing enzyme or enzymes. The nucleic acid molecule compositions provided herein can be delivered to host cells of a subject to encode a chromophore-producing enzyme, such as a melanin-producing enzyme, to produce or synthesize a chromophore in the host cell. The nucleic acid molecule can encode any chromophore-producing enzyme or enzymes that is sufficient for the production or overproduction of a chromophore in a host in vivo. In particular, the produced chromophore is one that is capable of absorbing energy so that it can be exploited for various therapy applications. Generally, the chromophore is one that exhibits light absorbing, redox and chelating properties. For example, the chromophore is one that is capable of absorbing electromagnetic wavelengths of from or from about 500 nm to 1500 nm, and typically 600 to 1200 nm. In addition, or in other examples, the chromophore is one that is a photosensensitzer capable of producing free radicals when irradiated with ultraviolet light, such as electromagnetic radiation having a wavelength in the range of from or from about 10 nm to 400 nm. For example, the chromophore has a binding affinity for one or more metal ions selected from among Ca(II), Zn(II), Fe(III), Cu(II), Pb(II), La(III) and/or Gd(III). One of skill in the art is familiar with chromophores and enzymes sufficient for the production of the chromophore in a cell of a host. Exemplary chromophore-producing enzymes herein are enzymes that are sufficient for the production of the chromophore melanin (eumelanin and/or pheomelanin), and in particular eumelanin.

In some examples, the encoded chromophore-producing enzyme(s) (e.g., melanin-producing enzyme) is exogenous or heterologous to the host cell. In other examples, the encoded chromophore-producing enzyme(s) (e.g., melanin-producing enzyme) is present in the host cells, but delivery of the nucleic acid molecule achieves increased expression of the enzyme(s) and overproduction of the resulting chromophore. For example, while some cells or tumor cells contain very high levels of the melanin-producing enzyme tyrosinase, not all cells or tumor cells contain a detectable level of tyrosinase (see e.g., Solano et al. (2012) AIP Advances, 2:011102-1). One of skill in the art can empirically choose a nucleic acid molecule encoding a chromophore-producing enzyme so as to achieve overproduction of the chromophore in a desired cell or tissue of interest for use in the methods and applications herein. For example, a nucleic acid molecule encoding the chromophore-producing enzyme tyrosinase can be used for delivery to non-pigment cells that do not normally express tyrosinase or produce melanin, including non-melanoma cancer cells or tissues.

In particular, delivery of the encoded enzyme(s) can achieve production or synthesis of the chromophore in the host cell that exceeds the amounts normally present in the cell. For example, the chromophore is produced in an amount that is or is at least 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 120-fold, 140-fold, 160-fold, 180-fold, 200-fold or more than the amount of the chromophore produced in the cell in the absence of the delivered nucleic acid molecule. It is within the level of one skilled in the art to determine the level or amount of a chromophore in a host cell in the absence of a delivered or introduced nucleic acid encoding a chromophore producing enzyme or enzymes and compare such levels to the level or amount produced in the presence of a nucleic acid molecule encoding a chromophore-producing enzyme or enzymes. For example, optical density measurements of cell pellets or tissues, such as from biopsy samples, can be determined based on the optical absorption of the particular chromophore. In such an example, melanin displays maximal optical absorption at ultraviolet wavelengths, but has a broad spectrum of optical absorption across a broad range of absorbance spectra, for example, 350 nm to 1000 nm. The absorbance can be normalized to the number of cells or tissue or sample size or weight. The absorbance also can be compared to control or reference samples in which a nucleic acid molecule encoding a chromophore producing enzyme or enzymes was not delivered or introduced.

In particular, provided herein are nucleic acid molecules that encode a melanin-producing enzyme or enzymes. Melanin is a pigment that can be subdivided into the brownish/black eumelanin and the reddish brown pheomelanin. Many genes are known to be involved in melanin biosynthesis (see e.g., Simon et al. (2009) Pigment Cell Melanoma Res, 22:563-79). Examples of such genes include, but are not limited to, tyrosinase (TYR), tyrosinase related protein 1 (tyrp1 or TRP-1) and Dopachrome tautomerase/tyrosinase related protein 2 (DCT, Tyrp2 or TRP2). In one example, nucleic acid molecules provided herein encode a tyrosinase enzyme or enzymatically active portion thereof. In another example, nucleic acid molecules provided herein encode a tyrosinase enzyme or enzymatically active portion thereof and a TRP-1 enzyme or enzymatically active portion thereof. In a further example, nucleic acid molecules provided herein encode a tyrosinase enzyme or enzymatically active portion thereof and a TRP2 enzyme or enzymatically active portion thereof. In an additional example, nucleic acid molecules provided herein encode a tyrosinase enzyme or enzymatically active portion thereof, a TRP-1 enzyme or enzymatically active portion thereof and a TRP2 enzyme or enzymatically active portion thereof. A description of exemplary nucleic acid molecules encoding such enzymes are described further below.

The nucleic acid molecules provided herein, when delivered to cells, can result in production of eumelanin or pheomelanin in the cell. In particular examples, the nucleic acid molecules provided herein, when delivered to cells, result in production of greater eumelanin than pheomelanin. Eumelanin is known to have more efficient binding capacity for certain drugs and metal ions, and has more potential in storing iron compared to pheomelanin. Therefore, production of greater eumelanin is likely to give a better MR signal. For example, the ratio of eumelanin to pheomelanin in the cell containing the introduced and encoded melanin-producing enzyme or enzymes is at least or greater than 1.2:1, 1.5:1, 2.0:1, 3.0:1, 4.0:1, 5.0:1, 6.0:1, 7.0:1, 8.0:1, 9.0:1, 10.0:1 or greater. The ratio or fold amount of eumelanin to total melanin produced in a cell into which a nucleic acid molecule encoding a melanin-producing enzyme is delivered can be at least 0.2, 0.3, 0.4, 0.5, 0.6, 0.7, 0.8, 0.9 or more. It is within the level of one skilled in the art to choose a particular nucleic acid molecule (e.g., virus, nanoparticle or other nucleic acid molecule) encoding a melanin-producing enzyme or enzymes that is capable of primarily achieving eumelanin production in cells into which it is delivered. Assays to quantitatively analyze eumelanin and pheomelanin or total melanin in cell or tissue samples are known to one of skill in the art (see e.g., Wakamatsu et al. (2002) Pigment Cell Res., 15:174-183; Ozeki et al. (1996) Pigment Cell Research, 9:265-270; Ito et al. (2003) Pigment Cell Research, 16:523-531). In such examples, the nucleic acid can encode TRP-1, which is known to increase the ratio of eumelanin to pheomelanin when expressed in cells. Generally, the TRP-1 is co-expressed with tyrosinase (Tyr).

a. Tyrosinase

Provided herein are nucleic acid molecules that encode a tyrosinase, a variant tyrosinase, or an enzymatically active portion of a tyrosinase, whereby the encoded enzyme exhibits tyrosinase activity. Tyrosinase (monophenol, L-dopa: oxygen oxidoreductase, EC 1.14.18.1; also known as TYR and TYRL) is a copper-containing enzyme that is present in plant and animal tissues that is a key enzyme in the melanin biosynthetic pathway. The enzyme has both tyrosine hydroxylase and dopa oxidase catalytic activities, and requires copper for function. Tyrosinase is the only enzyme absolutely required for melanin synthesis, and it is involved in the production of both eumelanin and pheomelanin. Pheomelanogenesis occurs in cells having a lower expression and activity of tyrosinase, whereas eumelanogenesis generally occurs in cells producing high levels of tyrosinase.

Tyrosinase catalyzes the first two steps in the pathway for melanin (eumelanin and pheomelanin) synthesis: hydroxylation of the amino acid tyrosine into dihydroxyphenylalanine (DOPA) and then oxidation into dopaquinone. Tyrosinase also catalyzes the subsequent oxidations of further downstream products 5,6-dihydroxyindole (DHI) and 5,6-dihydroxyindole-2-carboxylic acid (DHICA) into the eumelanin precursors indole-5,6-quinone and indole-5,6, quinone carboxylic acid, respectively. Pheomelanin is produced in the presence of thiol compounds (e.g., glutathione and cysteine) resulting in the conversion of the tyrosinase-produced product dopaquinone to cysteinyldopa, followed by cyclization and polymerization of the cysteinyldopa to result in pheomelanin. Mutations in the tyrosinase gene have been associated with oculocutaneous albinism type I (OCA1), an autosomal recessive disorder wherein the phenotype is a complete lack of melanin biosynthesis in the eyes, hair, and skin (Oetting, Pigment Cell Res., 2000, 13, 320-325).

The activity of tyrosinase can be modulated by other melanocyte-specific proteins, such as tyrosinase related protein 1 (Tyrp 1) and 2 (Tyrp 2, DCT), p-protein and membrane-associated transporter protein (MATP) (see e.g., Wang et al. (2006) Pigment Cell Res., 19:3-18). These proteins act to stabilize tyrosinase in cells, assist in the maturation and trafficking of tyrosinase in the cell and/or control processing and intracellular transport of tyrosinase. In particular, Tyrp 1 and Tyrp 2, while also being involved in melanin synthesis, have been shown to stabilize tyrosinase. As shown herein, however, delivery of nucleic acid encoding only the tyrosinase enzyme is sufficient for melanin production.

Tyrosinase is present in eukaryotic and prokaryotic cells, including in plants, insects, amphibians and mammals. It was initially identified in mushroom extracts. Tyrosinase is expressed in a tissue-specific manner in pigment cells, including epidermal melanocytes as well as the pigment epithelia of the retina, iris and ciliary body of the eye. The tissue-specific expression of tyrosinase is driven by its native promoter, which is approximately 2.1 kb in human and mice.

Tyrosinase is synthesized in melanosomal ribosomes found on the rough endoplasmic reticulum. Cleavage of the signal sequence is one of the first events to take place in the ER during tyrosinase maturation. After synthesis, tyrosinase is glycosylated within the ER and Golgi and then delivered to melanosomes via coated vesicles. The mature tyrosinase protein can be divided into three domains: an N-terminal lumenal ectodomain of about 455 amino acids that resides inside of melanosomes, a transmembrane domain, and a C-terminal cytoplasmic domain that extends into the cytoplasm of melanocytes (Oetting, Pigment Cell Res., 2000, 13, 320-325). The major portion of the enzyme is found inside the melanosome with only 10% (approximately 30 amino acids residues) acting as the cytoplasmic domain. Functionally, the ectodomain contains a copper-binding site and is responsible for catalytic activity which is consistent with melanin formation occurring exclusively within melanosomes (Park and Gilchrest, Cell Mol. Biol. (Noisy-le-grand), 1999, 45, 919-930). The ectodomain contains six conserved glycosylation sites that mediate N- and O-glycosidic linkages and 15 conserved, lumenal cysteine residues arranged in three Cys-rich clusters that can form disulfide bonds under the oxidizing conditions of the endoplasmic reticulum. The residues that make up the copper-binding site, cysteine-rich clusters, glycosylation sites, and other residues that are involved in metal binding and interactions to maintain globular folding, are highly conserved between and among species (Olivares et al. (2009) Pigment Cell Melanoma Res., 22:750-760).

In humans, the tyrosinase locus maps to human chromosome 11q14-21 (Barton et al., Genomics, 1988, 3, 17-24). The gene contains five exons spanning more than 65 kb (Giebel et al., Genomics, 1991, 9, 435-445). The size of the introns range from 10 kb for intron 4 to over 30 kb for intron 2. Over 50% of the coding region is found in exon 1 (Oetting, Pigment Cell Res., 2000, 13, 320-325). Human tyrosinase is encoded by a sequence of nucleotides set forth in SEQ ID NO:80 (accession number AAB37227). The encoded precursor human tyrosinase is a type I integral membrane glycoprotein that contains 529 amino acids (SEQ ID NO:81; Accession No. NP_{—}000363). Mature tyrosinase lacks the 18 amino acid signal sequence and has the sequence of amino acids set forth in SEQ ID NO:82. With reference to SEQ ID NO:81, the enzymatically active N-terminal lumenal ectodomain corresponds to amino acids 19-476, the transmembrane domain corresponds to amino acids 477-497 and the C-terminal cytoplasmic domain corresponds to amino acids 498-529 of SEQ ID NO:81. Human tyrosinase contains seven glycosylation sites that correspond to amino acid residues 68, 93, 143, 212, 272, 319 and 353 as set forth in mature tyrosinase set forth in SEQ ID NO:82 (corresponding to residues 86, 111, 161, 230, 290, 337 and 371 of SEQ ID NO:81). Among the cysteine residues are residues that correspond to residues C36, C55, C89, C289 of SEQ ID NO:81. The glycosylation and cysteine residues are required for tyrosinase activity and tyrosinase maturation, and mutations disrupting maturation are associated with deficiencies in tyrosinase that can lead to human Oculocutaneous Albinism type I (OCAI) (e.g., C18Y, C37Y, C71R, C271G/R/Y and T355K with reference to SEQ ID NO:82, corresponding to residues C36Y, C55Y, C89R, C289G/R/Y and T373K with reference to SEQ ID NO:81). An alternative splicing variant of human tyrosinase exists, but it lacks tyrosinase activity.

Copper binding is required for the activity of tyrosinase. Tyrosinase is a type 3 copper-binding protein that contains a binuclear copper active site, which brings together the molecular oxygen and the substrates tyrosine and DOPA. Each copper-binding site is coordinated by three (3) histidine residues. Specifically, in human tyrosinase, the CuA copper-binding site corresponds to residues His 162, 184 and 193 of SEQ ID NO:82 (corresponding to residues 180, 202 and 211 of SEQ ID NO:81) and the CuB copper-binding site corresponds to residues His345, 349 and 371 of SEQ ID NO:82 (corresponding to residues 363, 367 and 390 of SEQ ID NO:81). These residues are required for activity. For example, the mutant H349Y is known variant associated with Oculocutaneous Albinism type I (OCAI).

Defects in human tyrosinase are the cause of OCAI, which is also known as tyrosinase negative oculocutaneous albinism. OCAI is caused due to the production of an inactive enzyme and the lack of tyrosinase activity. Thus, the biosynthesis of melanin cannot be achieved. Defects in human tyrosinase are also the cause of albinism oculocutaneous type 1B (OCA1B) (albinism yellow mutant type) in which the biosynthesis of melanin is reduced due to a partial lack of tyrosinase activity. Exemplary mutations associated with OCAI and/or OCA1B include, but are not limited to, H19Q, P21 S, C36Y, D42G, S44G, S44R, G47D, G47V, R52I, C55I, Q68H, R77Q, R77RRins, R77W, S79L, W80R, P81L, C89R, G97R, G109R, P152S, T155S, F176I, V177F, M179L, H180N, S192Y, D199N, A201S, P205T, A206T, L216M, R217G, R217Q, R217S, R217W, de1217, de1227, W236L, W236S, R239W, D240V, K243T, G253R, H256Y, W272C, L288S, C289G, C289R, E294G, R299H, R299S, L312V, P313R, V318E, T325A, E328Q, S329P, M332T, S339G, F340L, E345G, G346E, A355E, A355P, S361R, H367Y, M370T, N371T, N371Y, T373K, Q378K, S380P, N382K, D383N, H390D, V393F, S395N, S395R, E398A, E398V, W400L, R402Q, R402G, R402L, R403S, H404N, H404P, R405L, P406L, Q408H, E409D, A416S, P417H, G419R, R422Q, S424F, M426K, V427G, P431L, R434I, N435D, F439V, D444G, G446S or D448N corresponding to SEQ ID NO:81. Other mutations are known to those of skill in the art (see e.g., the Albinism database, albinismdb.med.umn.edu/oca1mut.html).

Provided herein, for the methods and methods of use herein, are nucleic acid molecules encoding a human tyrosinase enzyme or a variant thereof or an enzymatically active portion thereof that exhibits tyrosinase enzyme activity. In particular, provided herein, for the methods and methods of use herein, is a nucleic acid molecule encoding the precursor tyrosinase enzyme set forth in SEQ ID NO:81 or the mature tyrosinase enzyme set forth in SEQ ID NO:82, or a variant or enzymatically active portion thereof that encodes a tyrosinase enzyme having tyrosinase activity and that exhibits at least 70% sequence identity to SEQ ID NO:81 or 82. For example, the encoded variant or enzymatically active portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:81 or 82. Included among such nucleic acid molecules is a nucleic acid molecule that contains the sequence of nucleotides set forth in SEQ ID NO:80 or a variant thereof or portion thereof that exhibits at least 70% sequence identity to SEQ ID NO:80 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:80.

One of skill in the art understands that a variant or truncated fragment of a nucleic acid molecule that encodes a tyrosinase enzyme that exhibits tyrosinase activity is one that does not exhibit a mutation or change that results in the loss of tyrosinase activity. Hence, the encoded tyrosinase enzyme typically contains the residues that are associated with copper binding, glycosylation and disulfide bond formation. In addition, the encoded tyrosinase enzyme does not contain a mutation that encodes an inactive tyrosinase, such as any of the mutations described above or known in the art that is associated with OCAI and/or OCA1B. An encoded tyrosinase enzyme, such as any provided herein, can include variants that are known in the art that result in enzymes that exhibit tyrosinase activity, such as allelic variants or polymorphisms that exist between and among various human subjects or other variants known to one of skill in the art, including but not limited to, F134C, K142N, M179I, R308T or L495P corresponding to residues set forth in SEQ ID NO:81. In particular, nucleic acid molecules provided herein can encode a tyrosinase enzyme that contains one or more conservative amino acid replacements compared to the tyrosinase set forth in SEQ ID NO:81 or 82.

Also provided herein are nucleic acid molecules encoding a non-human tyrosinase enzyme or a variant thereof or an enzymatically active portion thereof that exhibits tyrosinase enzyme activity. The sequence identity between and among species variants of tyrosinase is highly conserved. For example, the human and mouse tyrosinase protein possess 85% sequence identity.

In mice, the gene encoding tyrosinase maps to the mouse c-locus, which is the locus associated with the albino phenotype. Several isoforms of tyrosinase are expressed in mice as a result of alternative splicing, but only the major transcript confers tyrosinase enzyme activity (Ruppert et al. (1988) The EMBO Journal, 7:2715-2722; Muller et al. (1988) The EMBO Journal, 7:2723-2730). The major transcript (mTYR1) is encoded by a sequence of nucleotides set forth in SEQ ID NO: 6 or 83 (see e.g., accession number AAA40516.1) and encodes a mouse tyrosinase that contains 533 amino acids as set forth in SEQ ID NO:7 (see e.g., Accession No. NP_{—}035791). Mature mouse tyrosinase lacks the 18 amino acid signal sequence and has the sequence of amino acids set forth in SEQ ID NO:84. With reference to SEQ ID NO:7, the enzymatically active N-terminal lumenal ectodomain corresponds to amino acids 19-476, the transmembrane domain corresponds to amino acids 477-497 and the C-terminal cytoplasmic domain corresponds to amino acids 498-533 of SEQ ID NO:7. Mouse tyrosinase contains seven glycosylation sites that correspond to amino acid residues 86, 111, 161, 230, 337 and 371 of SEQ ID NO:7. The CuA binding site corresponds to residues 180, 202 and 211 and the CuB binding site corresponds to residues 363, 367 and 390 of SEQ ID NO:7. Exemplary mutations associated with loss or partial activity of tyrosinase, as evidenced in mice with an albino phenotype, are C103S, H420R or A482T with reference to SEQ ID NO:7.

Tyrosinases also include, but are not limited to, tyrosinase from gorilla (SEQ ID NO:85, DNA set forth in SEQ ID NO:86), chimpanzee (SEQ ID NO:87, DNA set forth in SEQ ID NO:88), orangutan (SEQ ID NO:89, DNA set forth in SEQ ID NO:90), gibbon (SEQ ID NO:91, DNA set forth in SEQ ID NO:92), cynomolgus monkey (SEQ ID NO:93, DNA set forth in SEQ ID NO:94), Rhesus macaque (SEQ ID NO:95, DNA set forth in SEQ ID NO:96), elephant (SEQ ID NO:97, DNA set forth in SEQ ID NO:98), rabbit (SEQ ID NO:99, DNA set forth in SEQ ID NO:100), naked mole rat (SEQ ID NO:101, DNA set forth in SEQ ID NO:102), pig (SEQ ID NO:103, DNA set forth in SEQ ID NO:104), cat (SEQ ID NO:105, DNA set forth in SEQ ID NO:106), sheep (SEQ ID NO:107, DNA set forth in SEQ ID NO:108), dog (SEQ ID NO:109, DNA set forth in SEQ ID NO:110), goat (SEQ ID NO:111, DNA set forth in SEQ ID NO:112), bovine (SEQ ID NO:113, DNA set forth in SEQ ID NO:114), rat (SEQ ID NO:115, DNA set forth in SEQ ID NO: 116) and guinea pig (SEQ ID NO:117, DNA set forth in SEQ ID NO:118). Each of the above encoded tyrosinase species variants, or mature form thereof lacking the signal sequence (amino acids 1-18 therein), exhibit at least 85% sequence identity to human tyrosinase set forth in SEQ ID NO: 81 or the mature form thereof set forth in SEQ ID NO:82, respectively.

Residues associated with glycosylation, cysteine residues involved in disulfide bond formation, copper-binding site residues and other residues known to be associated or required for tyrosinase activity, such as any described herein for human and mouse tyrosinase, are highly conserved between and among species. For example, with reference to dog tyrosinase set forth in SEQ ID NO:109, the enzymatically active N-terminal lumenal ectodomain corresponds to amino acids 19-473, the transmembrane domain corresponds to amino acids 474-494 and the C-terminal cytoplasmic domain corresponds to amino acids 495-530 of SEQ ID NO:109. Dog tyrosinase contains six glycosylation sites that correspond to amino acid residues 86, 111, 161, 230, 337 and 371 of SEQ ID NO:109. The CuA binding site corresponds to residues 180, 202 and 211 and the CuB binding site corresponds to residues 363, 367 and 390 of SEQ ID NO:109. Based on exemplification herein, one of skill in the art knows or can identify residues required for tyrosinase activity, and thereby generate variants of any of the above tyrosinase enzymes or portions thereof that are enzymatically active.

Hence, also provided herein, for the methods and methods of use herein, are nucleic acid molecules encoding a tyrosinase enzyme set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence (amino acids 1-18) or a variant thereof or an enzymatically active portion thereof that has tyrosinase enzyme activity that exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 7, 81, 82, 84, 85, 87, 89, 91, 93, 95, 97, 99, 101, 103, 105, 107, 109, 111, 113, 115, or 117 or the mature form thereof lacking the signal sequence. For example, provided herein is a nucleic acid molecule encoding a mature mouse tyrosinase (mTyr) set forth in SEQ ID NO:84, or a variant thereof or an enzymatically active portion thereof that has tyrosinase activity and that exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:84. Included among such nucleic acid molecules provided herein are nucleic acid molecules that contain the sequence of nucleotides set forth in any of SEQ ID NOS: 6, 83, 86, 88, 90, 92, 94, 96, 98, 100, 102, 104, 106, 108, 110, 112, 114, 116 or 118 or a variant thereof or portion thereof that exhibits at least 70% sequence identity to any of SEQ ID NOS: 6, 83, 86, 88, 90, 92, 94, 96, 98, 100, 102, 104, 106, 108, 110, 112, 114, 116 or 118 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 6, 83, 86, 88, 90, 92, 94, 96, 98, 100, 102, 104, 106, 108, 110, 112, 114, 116 or 118. One of skill in the art understands that a variant or truncated fragment of a nucleic acid molecule that encodes a tyrosinase enzyme that exhibits tyrosinase activity is one that does not exhibit a mutation or change that results in the loss of tyrosinase activity. Hence, the encoded tyrosinase enzyme typically contains the residues that are associated with copper binding, glycosylation and disulfide bond formation and do not contain amino acid mutations that render the enzyme inactive.

b. Tyrosinase-Related Protein-1

Provided herein are nucleic acid molecules that encode a tyrosinase-related Protein-1 (TRP-1 or Tyrp 1; also known as 5,6-dihydroxyindole-2-carboxylic acid oxidase or gp75), a variant TRP-1 or an enzymatically active portion thereof that exhibits enzyme activity. Typically, nucleic acid molecules provide herein that encode a TRP-1 enzyme or enzymatically active portion thereof also encode a tyrosinase enzyme or enzymatically active portion thereof.

TRP-1 is an enzyme that exhibits structural and functional similarity to tyrosinase. For example, it exhibits about 40% amino acid sequence identity to tyrosinase. TRP-1 originated by duplication of the ancestral tyrosinase gene. Like tyrosinase, TRP-1 is a transmembrane glycoprotein that is present in melanosomes where it spans the melanosomal membrane. TRP-1 contains a cytoplasmic and a transmembrane domain, a glycosylated lumenal domain and two metal-binding regions. The enzyme contains conserved glycosylation sites, cysteine-rich regions and two metal-binding regions involved in the structure of the catalytic site. TRP-1 is processed in the ER to remove the signal sequence, is post-translationally modified in the ER and Golgi and is delivered to melanosomes where it is present as a transmembrane protein. TRP-1 has been shown to bind to copper (Olivares et al. (2009) Pigment Cell Melanoma Res., 22:750-760). The enzyme exhibits various enzymatic activities including, for example, activity as a Dopachrome tautomerase, tyrosine hydroxylase, DOPA oxidase, catalase and/or 5,6-dihydroxy-indole-2-carboxylic acid (DHICA) oxidase. In addition to playing a role in melanin biosynthesis (discussed below), TRP-1 also is involved in melanosomal biogenesis and the structural integrity of melanosomes.

With respect to melanin biosynthesis, TRP-1 is involved in the distal reactions of melanogenesis that control the quality of the melanin polymer (Negroiu et al. (1999) Biochem. J., 344:659-665). As noted above, tyrosinase is required for the generation of dopaquinone, which is the rate-limiting step of melanin synthesis. TRP-1, however, is involved in the regulation of eumelanogenesis by catalyzing the oxidative polymerization of DHICA (Wakamatsu et al. (2002) Pigment Cell Res., 15:174-183). It has been shown that the presence of TRP-1 in cells increases the ratio of eumelanin to pheomelanin (Kobayashi et al. (1995) J. Cell Science, 108:2301-2309). This is exemplified in the Examples herein, which shows that co-transformation of the TRP-1-expressing plasmid resulted in production of a darker (eu-)melanin.

In humans, the tyrp1 (encoding TRP-1) locus is located on chromosome 9 and in mice is located on chromosome 4. Human TRP-1 is encoded by a sequence of nucleotides set forth in SEQ ID NO: 19 or 119 (see e.g., CAA35785.1). The encoded precursor human TRP-1 contains 537 amino acids and is set forth in SEQ ID NO:20. Mature TRP-1 lacks the 24 amino acid signal sequence and has the sequence of amino acids set forth in SEQ ID NO:120. With reference to SEQ ID NO:20, the enzymatically active N-terminal lumenal ectodomain corresponds to amino acids 25-477, the transmembrane domain corresponds to amino acids 478-501 and the C-terminal cytoplasmic domain corresponds to amino acids 502-537. Human TRP-1 contains six glycosylation sites that correspond to amino acid residues 96, 104, 181, 304, 350 and 385 as set forth in mature tyrosinase set forth in SEQ ID NO:20. The metal-binding site is coordinated by three (3) histidine residues and is required for activity. Specifically, in human TRP-1, the Metal A (MeA) binding site corresponds to residues 192, 215 and 224 of SEQ ID NO:20 and the MeB binding site corresponds to residues 377, 381 and 404. An alternative splicing isoform of TRP-1 exists, but it is an enzymatically inactive protein.

Defects in TRP1 are the cause of albinism oculocutaneous type 3 (OCA3) and other pigmentation defects. Specifically, because this enzyme plays a role in normal pigmentation, its loss leads to the changes in skin, hair, and eye coloration that are characteristic of oculocutaneous albinism. Exemplary mutations that result in an inactive enzyme and are associated with albinism phenotypes include, but are not limited to L36X, R93C, nonsense mutation S166X (introduction of a premature stop codon at position 166), H215Y, T253M, R356Q, 368del, R373X, M451V. Other mutations are known to those of skill in the art. (see e.g., the Albinism database, albinismdb.med.umn.edu/oca3mut.html).

Provided herein, for the methods of use and methods herein, are nucleic acid molecules encoding a human TRP-1 enzyme or a variant thereof or an enzymatically active portion thereof that exhibits TRP-1 enzyme activity. In particular, provided herein, for the methods of use and methods herein, is a nucleic acid molecule encoding the precursor tyrosinase enzyme set forth in SEQ ID NO:20 or the mature TRP-1 enzyme set forth in SEQ ID NO:120, or a variant or enzymatically active portion thereof that encodes a TRP-1 enzyme having TRP-1 enzyme activity and that exhibits at least 70% sequence identity to SEQ ID NO:20 or 120. For example, the encoded variant or enzymatically active portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:20 or 120. Included among such nucleic acid molecules provided herein is a nucleic acid molecule that contains the sequence of nucleotides set forth in SEQ ID NO:19 or 119 or a variant thereof or portion thereof that exhibits at least 70% sequence identity to SEQ ID NO:19 or 119 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:19 or 119.

One of skill in the art understands that a variant or truncated fragment of a nucleic acid molecule that encodes a TRP-1 enzyme that exhibits TRP-1 enzyme activity is one that does not exhibit a mutation or change that results in the loss of activity. Hence, the encoded TRP-1 enzyme typically contains the residues that are associated with copper binding, glycosylation and/or make up cysteine clusters. In addition, the encoded TRP-1 enzyme does not contain a mutation that encodes an inactive enzyme, such as any of the mutations described above or known in the art that is associated with OCA3 or other albinism-associated disease or condition. An encoded TRP-1 enzyme, such as any provided herein, can include variants that are known in the art that result in enzymes that exhibit TRP-1 activity, such as allelic variants or polymorphisms that exist between and among various human subjects or other variants known to one of skill in the art, including but not limited to, D308M, R326H, E413K, G458A, R505C, Y519X, Q530R corresponding to residues set forth in SEQ ID NO:20. In particular, nucleic acid molecules provided herein can encode a TRP-1 enzyme that contains one or more conservative amino acid replacements compared to the TRP-1 set forth in SEQ ID NO:20 or 120.

Also provided herein are nucleic acid molecules encoding a non-human TRP-1 enzyme or a variant thereof or an enzymatically active portion thereof that exhibits TRP-1 enzyme activity. The sequence identity between and among species variants of TRP-1 is highly conserved, as are the residues required for activity. Mouse TRP-1 is encoded by a sequence of nucleotides set forth in SEQ ID NO: 145 and encodes a mouse TRP-1 that contains 537 amino acids as set forth in SEQ ID NO:144. Mature mouse tyrosinase lacks the 24 amino acid signal sequence and has the sequence of amino acids 25-537 set forth in SEQ ID NO:144. With reference to SEQ ID NO:144, the enzymatically active N-terminal lumenal ectodomain corresponds to amino acids 25-477, the transmembrane domain corresponds to amino acids 478-501 and the C-terminal cytoplasmic domain corresponds to amino acids 502-537 of SEQ ID NO:144. Mouse TRP-1 contains glycosylation sites that correspond to amino acid residues 96, 104, 181, 304, 350 and 385 of SEQ ID NO:144. The MeA binding site corresponds to residues 192, 215 and 224 and the MeB binding site corresponds to residues 377, 381 and 404 of SEQ ID NO:144.

Also included among the TRP-1 enzymes are enzymes that include, but are not limited to TRP-1 from chimpanzee (SEQ ID NO:121; DNA set forth in SEQ ID NO:122), gorilla (SEQ ID NO:123), gibbon (SEQ ID NO:124; DNA set forth in SEQ ID NO:125), orangutan (SEQ ID NO:126), cynomolgus monkey (SEQ ID NO:127, DNA set forth in SEQ ID NO:128), Rhesus macaque (SEQ ID NO:129, DNA set forth in SEQ ID NO:130), rabbit (SEQ ID NO:131, DNA set forth in SEQ ID NO:132), pig (SEQ ID NO:133, DNA set forth in SEQ ID NO:134), elephant (SEQ ID NO:135, DNA set forth in SEQ ID NO:136), dog (SEQ ID NO:137, DNA set forth in SEQ ID NO:138), goat (SEQ ID NO:139, DNA set forth in SEQ ID NO:140), bovine (SEQ ID NO:141) and sheep (SEQ ID NO:142, DNA set forth in SEQ ID NO:143). Each of the above encoded TRP-1 species variants, or mature form thereof lacking the signal sequence (amino acids 1-24 therein), exhibit at least 80% sequence identity to human TRP-1 set forth in SEQ ID NO: 20 or the mature form thereof set forth in SEQ ID NO:120, respectively.

Hence, also provided herein, for the methods and methods of use herein, are nucleic acid molecules encoding a TRP-1 enzyme set forth in any of SEQ ID NOS: 121, 123, 124, 126, 127, 129, 131, 133, 135, 137, 139, 141, 142 or 144, or a mature form thereof lacking the signal sequence (amino acids 1-24) or a variant thereof or an enzymatically active portion thereof that has TRP-1 enzyme activity that exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 121, 123, 124, 126, 127, 129, 131, 133, 135, 137, 139, 141, 142 or 144 or the mature form thereof lacking the signal sequence. Included among such nucleic acid molecules provided herein are nucleic acid molecules that contains the sequence of nucleotides set forth in any of SEQ ID NOS: 122, 125, 128, 130, 132, 134, 136, 138, 140, 143 or 145 or a variant thereof or portion thereof that exhibits at least 70% sequence identity to any of SEQ ID NOS: 122, 125, 128, 130, 132, 134, 136, 138, 140, 143 or 145 and encodes an enzyme or portion thereof that exhibits TRP-1 enzyme activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 122, 125, 128, 130, 132, 134, 136, 138, 140, 143 or 145. One of skill in the art understands that a variant or truncated fragment of a nucleic acid molecule that encodes a TRP-1 enzyme that exhibits TRP-1 enzyme activity is one that does not exhibit a mutation or change that results in the loss of activity. Hence, the encoded TRP-1 enzyme typically contains the residues that are associated with copper binding, glycosylation and/or cysteine clusters and do not contain amino acid mutations that render the enzyme inactive.

In particular examples, the nucleic acid provided herein, for the methods and methods of use herein, contain a sequence of nucleotides that encode a TRP-1 enzyme or enzymatically active portion thereof and a sequence of nucleotides that encodes a tyrosinase enzyme or enzymatically active portion thereof. For example, provided herein are nucleic acid molecules containing: a sequence of nucleotides encoding a TRP-1 enzyme set forth in any of SEQ ID NOS: 20, 120, 121, 123, 124, 126, 127, 129, 131, 133, 135, 137, 139, 141, 142 or 144, or a mature form thereof lacking the signal sequence (amino acids 1-24) or a variant thereof or an enzymatically active portion thereof that has TRP-1 enzyme activity that exhibits at least 75% or more sequence identity to any of SEQ ID NOS: 20, 120, 121, 123, 124, 126, 127, 129, 131, 133, 135, 137, 139, 141, 142 or 144 or the mature form thereof lacking the signal sequence; and a sequence of nucleotides encoding a tyrosinase enzyme set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence (amino acids 1-18) or a variant thereof or an enzymatically active portion thereof that has tyrosinase enzyme activity that exhibits at least 75% sequence identity to any of SEQ ID NOS: 7, 81, 82, 84, 85, 87, 89, 91, 93, 95, 97, 99, 101, 103, 105, 107, 109, 111, 113, 115, or 117. The variant or enzymatically active portion can exhibit at least 80%, 81%, 82%, 83%, 84%, 85%, 86%, 87%, 88%, 89%, 90%, 91%, 92%, 93%, 94%, 95%, 96%, 97%, 98%, 99% or more sequence identity to any of the above-recited sequence of amino acids. Included among such nucleic acid molecules provided herein-are nucleic acid molecules that contain: the sequence of nucleotides set forth in any of SEQ ID NOS: 19, 119, 122, 125, 128, 130, 132, 134, 136, 138, 140, 143 or 145 or a variant thereof or portion thereof that exhibits at least 70% sequence identity to any of SEQ ID NOS: 19, 119, 122, 125, 128, 130, 132, 134, 136, 138, 140, 143 or 145 and encodes an enzyme or portion thereof that exhibits TRP-1 enzyme activity; and the sequence of nucleotides set forth in any of SEQ ID NOS: 6, 80, 83, 86, 88, 90, 92, 94, 96, 98, 100, 102, 104, 106, 108, 110, 112, 114, 116 or 118 or a variant thereof or portion thereof that exhibits at least 70% sequence identity to any of SEQ ID NOS: 6, 80, 83, 86, 88, 90, 92, 94, 96, 98, 100, 102, 104, 106, 108, 110, 112, 114, 116 or 118 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of the above-recited SEQ ID NOS.

c. Dopachrome Tautomerase

Provided herein, for the methods and methods of use herein, are nucleic acid molecules that encode a dopachrome tautomerase (DCT, also called Tryp2 or TRP-2, L-dopachrome tautomerase), a variant DCT or an enzymatically active portion thereof that exhibits enzyme activity. Typically, nucleic acid molecules provided herein, in the methods and methods of use herein, that encode a DCT enzyme or enzymatically active portion thereof also encode a tyrosinase enzyme or enzymatically active portion thereof.

DCT is an enzyme that exhibits structural and functional similarity to tyrosinase. For example, it exhibits about 40% amino acid sequence identity to tyrosinase. Like tyrosinase and TRP-1, DCT is a transmembrane glycoprotein that is present in melanosomes where it spans the melanosomal membrane. DCT contains a cytoplasmic and a transmembrane domain, a glycosylated lumenal domain and two metal-binding regions. The enzyme contains conserved glycosylation sites, cysteine-rich regions and two metal-binding regions involved in the structure of the catalytic site. The catalytic function of DCT is dependent on binding to a zinc metal cofactor. DCT exhibits dopachrome tautomerase activity DHI-2-carboxylic acid (DHICA) from Dopachrome rather than the spontaneously decarboxylated product DHI. With respect to melanin biosynthesis, DCT is another enzyme that is involved in the regulation of eumelanogenesis. Specifically, DCT has been shown to catalyze the tautomerization of dopachrome to DHICA. Like, TRP-1, DCT can affect the quantity of eumelanin produced in cells (Wakamatsu et al. (2002) Pigment Cell Res., 15:174-183).

In humans, the DCT gene is located on chromosome 13 and in mice is located on chromosome 14. Human DCT is encoded by a sequence of nucleotides set forth in SEQ ID NO:29. The encoded precursor human DCT contains 519 amino acids and is set forth in SEQ ID NO:30. Mature DCT lacks the 23 amino acid signal sequence and has the sequence of amino acids set forth in SEQ ID NO:146. With reference to SEQ ID NO:30, the enzymatically active N-terminal lumenal ectodomain corresponds to amino acids 24-472, the transmembrane domain corresponds to amino acids 473-493 and the C-terminal cytoplasmic domain corresponds to amino acids 494-519 of SEQ ID NO:30. Human DCT contains six glycosylation sites that correspond to amino acid residues 170, 178, 237, 300, 342 and 377 as set forth in mature tyrosinase set forth in SEQ ID NO:30. The metal-binding site is coordinated by three (3) histidine residues and is required for activity. Specifically, in human DCT, the Metal A (MeA) binding site corresponds to residues 189, 211, 220 of SEQ ID NO:30 and the MeB binding site corresponds to residues 369, 373 and 396. Several alternative splicing isoforms of DCT have been identified, but the other isoforms are soluble or enzymatically inactive proteins lacking the transmembrane domain.

Provided herein, for the methods and methods of use herein, are nucleic acid molecules encoding a human DCT enzyme or a variant thereof or an enzymatically active portion thereof that exhibits DCT enzyme activity. In particular, provided herein, for the methods and methods of use herein, is a nucleic acid molecule encoding the precursor tyrosinase enzyme set forth in SEQ ID NO:30 or the mature DCT enzyme set forth in SEQ ID NO:146, or a variant or enzymatically active portion thereof that encodes a DCT enzyme having DCT enzyme activity that exhibits at least 70% sequence identity to SEQ ID NO:30 or 146. For example, the encoded variant or enzymatically active portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30 or 146. Included among such nucleic acid molecules provided herein is a nucleic acid molecule that contains the sequence of nucleotides set forth in SEQ ID NO:29 or a variant thereof or portion thereof that exhibits at least 70% sequence identity to SEQ ID NO:29 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:29.

One of skill in the art understands that a variant or truncated fragment of a nucleic acid molecule that encodes a DCT enzyme that exhibits DCT enzyme activity is one that does not exhibit a mutation or change that results in the loss of activity. Hence, the encoded DCT enzyme typically contains the residues that are associated with zinc binding, glycosylation and/or make up cysteine clusters. In addition, the encoded DCT enzyme does not contain a mutation that encodes an inactive enzyme. An encoded DCT enzyme, such as any provided herein, can include variants that are known in the art that result in enzymes that exhibit DCT activity, such as allelic variants or polymorphisms that exist between and among various human subjects or other variants known to one of skill in the art. In particular, nucleic acid molecules provided herein can encode a DCT enzyme that contains one or more conservative amino acid replacements compared to the DCT set forth in SEQ ID NO:30 or 146.

Also provided herein, for the methods and methods of use herein, are nucleic acid molecules encoding a non-human DCT enzyme or a variant thereof or an enzymatically active portion thereof that exhibits DCT enzyme activity. The sequence identity between and among species variants of DCT is highly conserved, as are the residues required for activity. Mouse DCT is encoded by a sequence of nucleotides set forth in SEQ ID NO: 167 and encodes a mouse DCT that contains 517 amino acids as set forth in SEQ ID NO:166. Mature mouse DCT lacks the 23 amino acid signal sequence and has the sequence of amino acids 24-517 set forth in SEQ ID NO:166. With reference to SEQ ID NO:166, the enzymatically active N-terminal lumenal ectodomain corresponds to amino acids 24-472, the transmembrane domain corresponds to amino acids 473-491 and the C-terminal cytoplasmic domain corresponds to amino acids 492-517 of SEQ ID NO:166. Mouse DCT contains glycosylation sites that correspond to amino acid residues 92, 170, 178, 237, 300, 342 and 377 of SEQ ID NO:166. The zinc MeA binding site corresponds to residues 189, 211 and 220 and the zinc MeB binding site corresponds to residues 369, 373 and 396 of SEQ ID NO:166.

Also included among the DCT enzymes are enzymes that include, but are not limited to DCT from gorilla (SEQ ID NO:147), orangutan (SEQ ID NO:148, DNA set forth in SEQ ID NO:149), cynomolgus monkey (SEQ ID NO:150, DNA set forth in SEQ ID NO:151), Rhesus macaque (SEQ ID NO:152, DNA set forth in SEQ ID NO:153), white-tufted-ear marmoset (SEQ ID NO:154, DNA set forth in SEQ ID NO:155), horse (SEQ ID NO:156, DNA set forth in SEQ ID NO:157), pig (SEQ ID NO:158, DNA set forth in SEQ ID NO:159), panda (SEQ ID NO:160, DNA set forth in SEQ ID NO:161), rabbit (SEQ ID NO:162, DNA set forth in SEQ ID NO:163) and sheep (SEQ ID NO:164, DNA set forth in SEQ ID NO:165). Each of the above encoded DCT species variants, or mature form thereof lacking the signal sequence (amino acids 1-23 therein), exhibit at least 80% sequence identity to human DCT set forth in SEQ ID NO: 30 or the mature form thereof set forth in SEQ ID NO:146, respectively.

Hence, also provided herein, for the methods and methods of use herein, are nucleic acid molecules encoding a DCT enzyme set forth in any of SEQ ID NOS: 147, 148, 150, 152, 154, 156, 158, 160, 162, 164 or 166, or a mature form thereof lacking the signal sequence (amino acids 1-23) or a variant thereof or an enzymatically active portion thereof that has DCT enzyme activity that exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 147, 148, 150, 152, 154, 156, 158, 160, 162, 164 or 166 or the mature form thereof lacking the signal sequence. Included among such nucleic acid molecules provided herein, for the methods and methods of use herein, are nucleic acid molecules that contains the sequence of nucleotides set forth in any of SEQ ID NOS: 149, 151, 153, 155, 157, 159, 161, 163, 165 or 167 or a variant thereof or portion thereof that exhibits at least 70% sequence identity to any of SEQ ID NOS: 149, 151, 153, 155, 157, 159, 161, 163, 165 or 167 and encodes an enzyme or portion thereof that exhibits DCT enzyme activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 149, 151, 153, 155, 157, 159, 161, 163, 165 or 167. One of skill in the art understands that a variant or truncated fragment of a nucleic acid molecule that encodes a DCT enzyme that exhibits DCT enzyme activity is one that does not exhibit a mutation or change that results in the loss of activity. Hence, the encoded DCT enzyme typically contains the residues that are associated with zinc binding, glycosylation and/or cysteine clusters and do not contain amino acid mutations that render the enzyme inactive.

In particular examples, the nucleic acid provided herein, for the methods and methods of use herein, contain a sequence of nucleotides that encode a DCT enzyme or enzymatically active portion thereof and a sequence of nucleotides that encodes a tyrosinase enzyme or enzymatically active portion thereof. For example, provided herein are nucleic acid molecules containing: a sequence of nucleotides encoding a DCT enzyme set forth in any of SEQ ID NOS: 30, 146, 147, 148, 150, 152, 154, 156, 158, 160, 162, 164 or 166, or a mature form thereof lacking the signal sequence (amino acids 1-23) or a variant thereof or an enzymatically active portion thereof that has DCT enzyme activity that exhibits at least 75% or more sequence identity to any of SEQ ID NOS: 30, 146, 147, 148, 150, 152, 154, 156, 158, 160, 162, 164 or 166 or the mature form thereof lacking the signal sequence; and a sequence of nucleotides encoding a tyrosinase enzyme set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence (amino acids 1-18) or a variant thereof or an enzymatically active portion thereof that has tyrosinase enzyme activity that exhibits at least 75% sequence identity to any of SEQ ID NOS: 7, 81, 82, 84, 85, 87, 89, 91, 93, 95, 97, 99, 101, 103, 105, 107, 109, 111, 113, 115, or 117. The variant or enzymatically active portion can exhibit at least 80%, 81%, 82%, 83%, 84%, 85%, 86%, 87%, 88%, 89%, 90%, 91%, 92%, 93%, 94%, 95%, 96%, 97%, 98%, 99% or more sequence identity to any of the above-recited sequence of amino acids. Included among such nucleic acid molecules provided herein are nucleic acid molecules that contain: the sequence of nucleotides set forth in any of SEQ ID NOS: 29, 149, 151, 153, 155, 157, 159, 161, 163, 165 or 167 or a variant thereof or portion thereof that exhibits at least 70% sequence identity to any of SEQ ID NOS: 29, 149, 151, 153, 155, 157, 159, 161, 163, 165 or 167 and encodes an enzyme or portion thereof that exhibits DCT enzyme activity; and the sequence of nucleotides set forth in any of SEQ ID NOS: 6, 80, 83, 86, 88, 90, 92, 94, 96, 98, 100, 102, 104, 106, 108, 110, 112, 114, 116 or 118 or a variant thereof or portion thereof that exhibits at least 70% sequence identity to any of SEQ ID NOS: 6, 80, 83, 86, 88, 90, 92, 94, 96, 98, 100, 102, 104, 106, 108, 110, 112, 114, 116 or 118 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of the above-recited SEQ ID NOS.

In particular examples, the nucleic acid provided herein, for the methods and methods of use herein, contain a sequence of nucleotides that encodes a TRP-1 enzyme or enzymatically active portion thereof, a DCT enzyme or enzymatically active portion thereof, and a sequence of nucleotides that encodes a tyrosinase enzyme or enzymatically active portion thereof. For example, provided herein are nucleic acid molecules containing: a sequence of nucleotides encoding a TRP-1 enzyme set forth in any of SEQ ID NOS: 20, 120, 121, 123, 124, 126, 127, 129, 131, 133, 135, 137, 139, 141, 142 or 144, or a mature form thereof lacking the signal sequence (amino acids 1-24) or a variant thereof or an enzymatically active portion thereof that has TRP-1 enzyme activity that exhibits at least 75% or more sequence identity to any of SEQ ID NOS: 20, 120, 121, 123, 124, 126, 127, 129, 131, 133, 135, 137, 139, 141, 142 or 144 or the mature form thereof lacking the signal sequence; a DCT enzyme set forth in any of SEQ ID NOS: 30, 146, 147, 148, 150, 152, 154, 156, 158, 160, 162, 164 or 166, or a mature form thereof lacking the signal sequence (amino acids 1-23) or a variant thereof or an enzymatically active portion thereof that has DCT enzyme activity that exhibits at least 75% or more sequence identity to any of SEQ ID NOS: 30, 146, 147, 148, 150, 152, 154, 156, 158, 160, 162, 164 or 166 or the mature form thereof lacking the signal sequence; and a sequence of nucleotides encoding a tyrosinase enzyme set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence (amino acids 1-18) or a variant thereof or an enzymatically active portion thereof that has tyrosinase enzyme activity that exhibits at least 75% sequence identity to any of SEQ ID NOS: 7, 81, 82, 84, 85, 87, 89, 91, 93, 95, 97, 99, 101, 103, 105, 107, 109, 111, 113, 115, or 117. The variant or enzymatically active portion can exhibit at least 80%, 81%, 82%, 83%, 84%, 85%, 86%, 87%, 88%, 89%, 90%, 91%, 92%, 93%, 94%, 95%, 96%, 97%, 98%, 99% or more sequence identity to any of the above-recited sequence of amino acids. Included among such nucleic acid molecules provided herein are nucleic acid molecules that contain: the sequence of nucleotides set forth in any of SEQ ID NOS: 19, 119, 122, 125, 128, 130, 132, 134, 136, 138, 140, 143 or 145 or a variant thereof or portion thereof that exhibits at least 70% sequence identity to any of SEQ ID NOS: 19, 119, 122, 125, 128, 130, 132, 134, 136, 138, 140, 143 or 145 and encodes an enzyme or portion thereof that exhibits TRP-1 enzyme activity; the sequence of nucleotides set forth in any of SEQ ID NOS: 29, 149, 151, 153, 155, 157, 159, 161, 163, 165 or 167 or a variant thereof or portion thereof that exhibits at least 70% sequence identity to any of SEQ ID NOS: 29, 149, 151, 153, 155, 157, 159, 161, 163, 165 or 167 and encodes an enzyme or portion thereof that exhibits DCT enzyme activity; and the sequence of nucleotides set forth in any of SEQ ID NOS: 6, 80, 83, 86, 88, 90, 92, 94, 96, 98, 100, 102, 104, 106, 108, 110, 112, 114, 116 or 118 or a variant thereof or portion thereof that exhibits at least 70% sequence identity to any of SEQ ID NOS: 6, 80, 83, 86, 88, 90, 92, 94, 96, 98, 100, 102, 104, 106, 108, 110, 112, 114, 116 or 118 and encodes an enzyme or portion thereof that exhibits tyrosinase activity. For example, the variant or portion thereof exhibits at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any of the above-recited SEQ ID NOS.

2. Nucleic Acid Molecule

a. Virus and Viral Vectors

The nucleic acid molecule containing nucleotides encoding a chromophore-producing enzyme or enzymes can be a virus or a viral vector. The virus or viral vector can contain nucleotides encoding any chromophore-producing enzyme or enzymes described in subsection 1 above, such as a melanin-producing enzyme or enzymes. For example, the virus or viral vector can contain nucleotides encoding any tyrosinase enzyme, enzymatically active portion thereof or variant thereof, such as any described in Section 1.a above. In some examples, the virus also can additionally contain nucleotides encoding a tyrosinase-related protein 1 (TRP-1) and/or a dopachrome tautomerase (DCT), or enzymatically active portions thereof or variants thereof, such as any described in Section 1.b and/or Section 1.c above.

Viruses are useful in delivering nucleic acid molecules in vivo because they are efficient at transferring viral DNA into host cells. They can infect and be taken up by specific target cells depending on the viral attachment proteins (e.g., capsid or glycoproteins), and they can be manipulated to remove non-essential genes and add heterologous nucleic acid molecules. Many viral vectors are known to those skilled in the art. Examples of viruses that can be used in the compositions and methods herein include, but are not limited to, adenoviruses, adeno-associated viruses, alphaviruses, baculoviruses, hepadnaviruses, baculoviruses, poxviruses, herpesviruses, retroviruses, lentiviruses, orthomyxoviruses, papovaviruses, paramyxoviruses, and parvoviruses. The choice of virus is within the level of one of skill in the art and is dependent on a number of factors, such as the desire for replication or integration of viral DNA, the tropism of the virus, and/or the immunogenicity of the virus. Such viruses and derivatives thereof, are well-known and available to one of skill in the art. For example, many are available from the American Type Culture Collection (ATCC, Rockville, Md.). In particular examples, the virus is an oncolytic virus that is capable of accumulating in tumor cells or other immunoprivileged cells or tissues. Exemplary oncolytic viruses for use in the compositions and methods herein are vaccinia viruses described in Section E.

The viral vectors used in the methods herein also can contain expression cassettes that include regulatory elements, such as promoters and enhancers, operably linked to a transgene of choice. As discussed above, any suitable promoter can be used. Suitable promoters and enhancers are widely available in the art for use in the viral vector of choice. Typically the promoter is a constitutive promoter. Exemplary promoters include, but are not limited to, a CMV promoter, a truncated CMV promoter, a human serum albumin promoter or an α-1-antitrypsin promoter. In some examples, the promoter is a truncated CMV promoter in which binding sites for known transcriptional repressors have been deleted. In other examples, the promoter is an inducible promoter. For example, the promoter can be the inducible ecdysone promoter. Other examples of promoters include steroid promoters, such as estrogen and androgen promoters, and metallothionein promoters. The enhancer can be a tissue specific or non-specific enhancer. Other regulatory sequences (e.g., promoters, enhancers or other regulatory sequence) to regulate expression of an open reading frame encoding the chromophore producing enzyme or enzymes can be any described below in Section E.2.b.

b. Non-Viral Vectors

The nucleic acid molecule containing nucleotides encoding a chromophore-producing enzyme or enzymes can be a non-viral vector. The non-viral vector can contain nucleotides encoding any chromophore-producing enzyme or enzymes described in subsection 1 above, such as a melanin-producing enzyme or enzymes. For example, the non-viral vector can contain nucleotides encoding any tyrosinase enzyme, enzymatically active portion thereof or variant thereof, such as any described in Section 1.a above. In some examples, the non-viral vector also can additionally contain nucleotides encoding a tyrosinase-related protein 1 (TRP-1) and/or a dopachrome tautomerase (DCT), or enzymatically active portions thereof or variants thereof, such as any described in Section 1.b and/or Section 1.c above.

Non-viral vectors include any DNA molecule as long as it does not include all of the requisite elements for viral replication, packaging and/or expression. These include non-viral expression vectors. Non-viral expression vectors contain a nucleic acid encoding chromophore-producing enzyme or enzymes, wherein the nucleic acids are operably linked to an expression control sequence (e.g., promoter). Suitable vector backbones include, for example, those routinely used in the art such as plasmids, minicircles, cosmids and artificial chromosomes (e.g., human artificial chromosomes, mammalian artificial chromosomes (MACs), bacterial artificial chromosomes (BACs), yeast artificial chromosomes (YACs), or plant artificial chromosomes (PACs)). Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

Vectors typically contain one or more regulatory regions, which are functionally inked to the encoding region. Regulatory regions include, without limitation, promoter sequences, enhancer sequences, SMARs (scaffold matrix attachment regions), insulators, response elements, protein recognition sites, inducible elements, protein binding sequences, 5′ and 3′ untranslated regions (UTRs), transcriptional start sites, termination sequences, polyadenylation sequences, and introns.

Promoters controlling transcription from vectors in mammalian host cells can be obtained from various sources, for example, the genomes of viruses such as polyoma, Simian Virus 40 (SV40), adenovirus, retroviruses, hepatitis B virus, and most generally cytomegalovirus (CMV), or from heterologous mammalian promoters, e.g., β-actin promoter or EF1α promoter, or from hybrid or chimeric promoters (e.g., CMV promoter fused to the β-actin promoter). Promoters from the host cell or related species are also useful herein. Exemplary promoters that can be used include any known in the art, such as any as described elsewhere herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically, one will use an enhancer from a eukaryotic cell virus for general expression. Examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Examples of promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype on a cell (e.g., antibiotiC resistance) or be otherwise detectable. Examples of detectable markers include the E. coli lacZ gene, green fluorescent protein (GFP), and luciferase. In addition, an expression vector can include a tag sequence designed to facilitate manipulation or detection (e.g., localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion polypeptide, including the encoded polypeptide and the marker. Such tags can be inserted anywhere within the encoded polypeptide including at either the carboxyl or amino terminus.

In particular, a nucleic acid molecule vector, such as a non-viral vector or expression vector, containing a nucleic acid encoding a chromophore-producing enzyme or enzymes, can be delivered as naked DNA. The efficiency of delivery of the naked DNA in the methods herein can be increased by using various methods well-known to one of skill in the art (see e.g., Li and Huang (2006) Gene Therapy, 13:1313-1319). Such methods include, for example, such as electroporation, sonoporation or “gene gun” approaches.

Also, the efficiency of delivery can be increased by encapsulation in liposomes or complexing with polymers as described herein. In a particular example, the nucleic acid can be delivered as a nanoparticle as described herein above. The nanoparticles can be functionalized by conjugating or coating a targeting molecule onto the surface, for example, a targeting molecule that is a ligand for or otherwise binds to receptors expressed in the cells to be targeted.

3. Localized Delivery or Expression of Nucleic Acid Molecules (e.g., Localized Expression in Tumors)

For use in the methods herein any of the nucleic acid molecules provided herein are delivered to cells or tissues in which elimination is desired, for example, because the cells or tissues are associated with, exacerbate or otherwise cause disease. Such cells or tissues include, but are not limited to, artherosclerotic plaques or tumors. Localized delivery and expression of nucleic acid molecules provided herein encoding one or more chromophore-producing enzyme can be achieved, for example, by targeted delivery, cell-specific expression and/or by delivery in an oncolytic virus. One or more of the above methods, which are further described below, can be combined to achieve localized expression of a chromophore-producing enzyme or enzymes.

a. Targeted Delivery

Any of the nucleic acid molecules provided herein can be complexed, conjugated, provided in a vehicle or otherwise modified to achieve targeted gene delivery, whereby the nucleic acid molecule is primarily transported to a target tissue and taken up by target cells wherein transcription of the transgene(s) occur(s). Such nucleic acid molecules exhibit localized expression by virtue of differential uptake into target cells or tissues compared to non-target tissue.

For example, any of the nucleic acid molecules provided herein is encapsulated or conjugated to a particular carrier that contain a targeting molecule for specific targeting to cells of interest. Such targeting molecules include cell-targeting moieties that enhances the association of the agent or complex with a cell including, but not limited to, protein, peptide, lipid, steroid, sugar, carbohydrate, (non-expressing) polynucleic acid or synthetic compound. For example, the targeting moiety can be a ligand or antibody. In particular examples, a dual-ligand approach can be used to increase the selectivity for a cell. The choice of targeting molecule depends on the particular application, including the tissue or organ to be targeted, and can be empirically determined by one of skill in the art. Targeted nanoparticles are known in the art (see, e.g., Franzen (2011) Expert Opin. Drug. Deliv. 8(3):281-98; Faraji and Wipf (2009) Bioorg. Med. Chem. 17(8):2950-62; Salija et al., (2009) Curr. Drug. Discov. Technol. 6(1):43-51). In particular, methods for tissue-specific gene delivery of nanoparticles are known in the art (see e.g., Harris et al. (2010) Biomaterials, 31:998-1006). For example, targeted delivery, can be achieved by incorporating cell-binding ligands that recognize target-specific cellular receptors and/or enhance cellular binding to receptors. Such ligands include, but are not limited to, insulin, growth factor (e.g., EGF of FGF), transferrin, peptides that include the RGD sequence. Other targeting moieties include, but are not limited to, chemical groups that react with thiol, sulfhydryl or disulfide groups on cells, folate and other vitamins.

In particular examples, the targeting molecule is one that achieves or enhances targeting to tumor cells. There are multiple examples of receptors that can be used for targeted delivery to tumor cells (see WO 2001/036003), including the folate receptor (see e.g., Gottschalk, Gene Ther. 1994; 1(3):185-191), prolactin receptor (see e.g., U.S. Publication No. US20090317855) and transferrin receptor 2 (Calzolari et al., Blood Cells Mol Dis. 2009; 42(1):5-13; Calzolari et al. Blood Cells Mol Dis., 2009; 43(3):243-9). For example, the transferrin receptor is found overexpressed in at least 40% of human tumors and cell lines.

In addition, targeted delivery can be achieved by shielding or removing vector domains that demonstrate undesired binding potential to blood or non-target cells (e.g., natural receptor binding proteins in viral vectors, positive surface charge in non-viral vectors). For example, viral vectors can be engineered to eliminate gene transfer to non-target cells through native viral receptors, and redirected instead to specific receptors present on the desired target cell (Sharman et al., Adv Drug Deliv Rev. 2004; 56(1):53-76; Wickham, Nat Med. 2003; 9:135-139). Retargeting of viral vectors can be performed genetically, for example, by sequence integration of retargeting ligands into the genome of the vector.

b. Cell-Specific (e.g., Tumor-Specific) Expression

Any of the nucleic acid molecules provided herein can contain a cell- or tissue-specific promoter that is operably linked to the coding region of the gene encoding the chromophore-producing enzyme or enzymes. In such an example, the nucleic acid molecule can be delivered into several cell types, but transcription of the carried nucleic acid is under the control of specific promoter/enhancer elements that allow gene transcription only in select target cells (targeted transcription). Tissue and disease-specific promoters, including tumor-specific promoters, are known in the art (see e.g., Papadakis et al. (Current Gene Therapy (2004) 4:89-113). Generally if the nucleic acid molecules encodes more than one chromophore-producing enzyme, the enzymes can be under control of the same promoter separated by an internal ribosome binding site (IRES) such that both genes are expressed. In other examples, if the nucleic acid molecule encodes more than one chromophore-producing enzyme, the enzymes can be under the control of separate promoters. The separate promoters can be the same or different.

Exemplary cell-specific promoters include, for example, endothelial nitric oxide synthase (eNOS) promoter expressed in endothelial cells (Guillot, P.V. et al. (199) J. Clin. Invest. 103:799-805); vascular endothelial growth factor (VEGF) receptor (Flk-1) promoter expressed in endothelial cells (Kappel et al. (1999) Blood, 93:4282-4292); insulin promoter expressed in beta cells of the pancreas (Ray et al., J. Surg. Res. (1999) 84:199-203); promoter of gonadotropin-releasing hormone receptor gene expressed in cells of the hypothalamus (Albarracin et al. (1999) Endocrinology, 140:2415-2421); matrix metalloproteinase 9 promoter expressed in osteoclasts and keratinocytes (Munant et al., (1999) J. Biol. Chem., 274:5588-5596); promoter of parathyroid hormone receptor expressed in bone cells (Amizuma et al. (1999) J. Clin. Invest., 103:373-381); and dopamine beta-hydroxylase promoter expressed in noradrenergic neurons (Yang et al., (1998) J. Neurochem. 71:1813-1826).

Cell-specific promoters also include tumor-specific promoters. Tumor-specific promoters are known to those of skill in the art (see e.g., Hardcastle J et al. (2007) Current Cancer Drug Targets, 7: 181-189. These include, but are not limited to, c-erbB-2 oncogene (targeting to breast, pancreatic, gastric and ovarian cancers; see e.g., Hollywood D and Hust H (1993) EMBO J, 12:2369-2375); carcinoembryonic antigen (CEA) (targeting to lung and gastrointestinal malignancies, including colon, pancreatic and gastric cancer; see e.g., Thompson, J.A. et al. (1991) J. Clin. Lab. Anal., 5:344-366; Osaki T et al. (1994) Cancer Res., 54:5258-5261); DF3/MUC1 (targeting to breast cancer; see e.g., Abe M and Kufe D (1993) Proc. Natl. Acad. Sci. USA, 90:282-286; Manome Y et al. (1995) Gene Ther. 2:685, A051; Chen L et al. (1995) J. Clin. Invest., 96:2775-2782); prostate specific antigen (PSA) (targeting to prostate cancer; see e.g., Lundwall, A (1989) Biochem. Biophys. Res. Commun., 161:1151-1156); and alpha-fetoprotein (AFP) (targeting to hepatocellular carcinoma; see e.g., Arbuthnot P et al. (1995) Hepatology, 22:1788-1796; Ido et al. (1995) Cancer Res., 55:3105-3109); L-plastin (LP-P) (targeting to epithelial-derived tumors; see e.g., Chung et al. (1999) Cancer Gene Ther., 6:99-106); α-lactalbumin (ALA) (targeting to breast cancer; see e.g., Anderson et al. (2000) Cancer Gene Ther., 7:845-852); midkine (MK) (targeting to pancreatic cancer; see e.g., Yoshida et al. (2002) Anticancer Res., 22:117-120); cyclooxygenase-2 (COX-2) (targeting gastrointestinal cancer; see e.g., Yamamoto et al., (2001) Mol. Ther., 3:385-394; Wesseling et al. (2001) Cancer Gene Ther., 8:990-996); probasin (ARR2PB) (targeting prostate cancer; see e.g., Lowe et al. (2001) Gene Ther., 8:1363-1371; Rubinchik et al. (2002) Gene Ther., 8:247-253); hypoxic response elements (HRE) (not tissue specific; see e.g., Dachs et al. (2000) Eur. J. Cancer, 36:1649-1660; Ruan H et al. (2001) Curr Opin Invest Drugs, 2:839-843); hTERT (not tissue specific; see e.g., Gu et al. (2000) Cancer Res., 60:5359-5364; Koga et al. (2000) Hum. Gene Ther., 11:1397-1406; Lin et al. (2002) Cancer Res., 62:3620-3625; Majumdar et al. (2001) Gene Ther., 8:568-578; Fujiwara T et al. (2007) Current Cancer Drug Targets, 7: 191-201); promoters that target the angiogenic tumor vasculature, for example, flt-1, flk1/KDR, E-selectin, endoglin, ICAM-2, preproendothelin 1 (PPE-1) (see e.g., Jaggar et al. (1997) Hum. Gene Ther., 8:2239-2247; Walton et al. (1998) Anticancer Res., 18:1357-1360; Savontaus et al. (2002) Gene Ther., 9:972-979; Bauerschmitz et al. (2002) Cancer Res., 62:1271-1274; Velasco et al. (2001) Gene Ther., 8:897-904; Varda-Bloom et al. (2001)); prolactin (PRL) (targeting pituitary tumors; see e.g., Southgate et al. (2000) Endocrinology, 141:3493-3505); osteocalcin 2 (targeting osteosarcoma; see e.g., Barnett et al. (2002) Mol. Ther., 6:377-385); survivin (Hoffman, W.H. et al. (2002) J Biol. Chem., 277:3247-3257); CXCR4 tumor-specific promoters (Ulasov I V et al. (2007) Cancer Biology and Therapy, 65:675-689; Zhu, Z B et al. (2006) J Thorac. Oncol., 1:701-711); and human papilloma virus 16 (Delgado-Enciso et al. (2007) J of Gene Medicine).

Included among exemplary cell-specific and tumor-specific promoters include, but are not limited to the CMV promoter (SEQ ID NO:170); Telomerase promoter (SEQ ID NO:171); E2F-1 promoter, including modified or synthetic promoters (e.g., SEQ ID NO:172), Antigen 33 (A33) promoter (SEQ ID NO: 175) Cyclo-oxygenase-2 (COX-2) promoter (SEQ ID NO:176); Human carcinoembryonic antigen (CEA) promoter (SEQ ID NO: 177); Cyclin A (CycA) promoter (SEQ ID NO: 178); Cell division cycle 2 (Cdc2) promoter (SEQ ID NO: 179); Cell division cycle 25 (Cdc25) promoter (SEQ ID NO: 180); B-myb promoter (SEQ ID NO: 181); p107 promoter (SEQ ID NO:182); Tyrosine Kinase (TK) promoter (SEQ ID NO:183); DNA polymerase alpha promoter (SEQ ID NO:184); Histone 2A (H2A) promoter (SEQ ID NO:185); C-myc promoter (SEQ ID NO: 186) or a Synthetic cell cycle-dependent promoter (SEQ ID NO:187).

c. Oncolytic Viruses

In particular, the nucleic acid molecules encoding a chromophore-producing enzyme or enzymes (e.g., a melanin-producing enzyme or enzymes) is an oncolytic virus. Oncolytic viruses are characterized by their largely tumor cell specific replication, resulting in tumor cell lysis and efficient tumor regression. Oncolytic viruses effect treatment by colonizing or accumulating in tumor cells, including metastatic tumor cells such as circulating tumor cells, and replicating therein. They provide an effective weapon in the tumor treatment arsenal. Oncolytic viruses include Newcastle Disease virus, parvovirus, vaccinia virus, reovirus, measles virus, vesicular stomatitis virus (VSV), oncolytic adenoviruses and herpes viruses. In many cases, tumor selectivity is an inherent property of the virus, such as vaccinia viruses and other oncolytic viruses. Generally oncolytic viruses effect treatment by replicating in tumors or tumor cells resulting in lysis.

Oncolytic viruses for use in the methods provided here are well-known to one of skill in the art and include, for example, vesicular stomatitis virus, see, e.g., U.S. Pat. Nos. 7,731,974, 7,153,510, 6,653,103 and U.S. Pat. Pub. Nos. 2010/0178684, 2010/0172877, 2010/0113567, 2007/0098743, 20050260601, 20050220818 and EP Pat. Nos. 1385466, 1606411 and 1520175; herpes simplex virus, see, e.g., U.S. Pat. Nos. 7,897,146, 7731,952, 7,550,296, 7,537,924, 6,723,316, 6,428,968 and U.S. Pat. Pub. Nos. 2011/0177032, 2011/0158948, 2010/0092515, 2009/0274728, 2009/0285860, 2009/0215147, 2009/0010889, 2007/0110720, 2006/0039894 and 20040009604; retroviruses, see, e.g., U.S. Pat. Nos. 6,689,871, 6,635,472, 6,639,139, 5,851,529, 5,716,826, 5,716,613 and U.S. Pat. Pub. No. 20110212530; and adeno-associated viruses, see, e.g., U.S. Pat. Nos. 8,007,780, 7,968,340, 7,943,374, 7,906,111, 7,927,585, 7,811,814, 7,662,627, 7,241,447, 7,238,526, 7,172,893, 7,033,826, 7,001,765, 6,897,045, and 6,632,670.

Oncolytic viruses also include viruses that have been genetically altered to attenuate their virulence, to improve their safety profile, enhance their tumor specificity, and they have also been equipped with additional genes, for example cytotoxins, cytokines, prodrug converting enzymes to improve the overall efficacy of the viruses (see, e.g., Kim et al., (2009) Nat Rev Cancer 9:64-71; Garcia-Aragoncillo et al., (2010) Curr Opin Mol Ther 12:403-411; see U.S. Pat. Nos. 7,588,767, 7,588,771, 7,662,398 and 7,754,221 and U.S. Pat. Publ. Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650.

For example, other activities can be introduced and/or anti-tumor activity can be enhanced by including nucleic acid encoding a heterologous gene product that is a therapeutic and/or diagnostic agent or agents. In some examples, the oncolytic viruses provide oncolytic therapy of a tumor cell without the expression of a therapeutic gene. In other examples, the oncolytic viruses can express one or more genes whose products are useful for tumor therapy. For example, a virus can express proteins that cause cell death or whose products cause an anti-tumor immune response. Such genes can be considered therapeutic genes. A variety of therapeutic gene products, such as toxic or apoptotic proteins, or siRNA, are known in the art, and can be used with the oncolytic viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound. Exemplary therapeutic gene products are gene products selected from among an anticancer agent, an anti-metastatic agent, an antiangiogenic agent, an immunomodulatory molecule, an antigen, a cell matrix degradative gene, genes for tissue regeneration and reprogramming human somatic cells to pluripotency, and other genes described herein or known to one of skill in the art. In these examples, the tumor-specific replication process is capable of directly killing the infected tumor cells (oncolytic viruses) and/or strongly amplifying the copy number of the therapeutic gene carried by the viral vector.

Exemplary therapeutic genes that can be inserted into any oncolytic virus are described herein in Section E and exemplified with respect to vaccinia virus (e.g., LIVP and Western Reserve). It is understood that an oncolytic virus can be modified to include nucleic acid sequences encoding any of the therapeutic genes described in Section E or any known to one of skill in the art. The sequence of nucleotides encoding a gene is typically inserted into or in place of a non-essential gene or region in the genome of the virus.

Thus, oncolytic viruses herein, in addition to containing nucleic acid encoding a chromophore-producing enzyme(s), also include viruses that contain nucleic acid encoding another heterologous gene product that is a therapeutic and/or diagnostic agent or agents. Examples of such oncolytic viruses are viruses derived from the Lister strain, such as LIVP, including any containing nucleic acid encoding a heterologous gene product (e.g., GLV-1h68 and derivatives thereof). Such viruses, are further described in detail in Section E. Among other therapeutic vaccinia viruses are the virus designated VV-GM, which is a vaccinia virus that expresses GM-CSF described, for example, in U.S. Pat. No. 6,093,700, and the Wyeth strain vaccinia virus designated JX-594, which is a TK-deleted vaccinia virus that expresses GM-CSF (see, International PCT application No WO 2004/014314, U.S. Pat. No. 5,364,773; Mastrangelo et al. (1998) Cancer Gene Therapy 6:409-422; Kim et al. (2006) Molecular Therapeutics 14:361-370). Other oncolytic viruses include, but are not limited to, JX-954 (Parato et al. (2012) Mol. Ther., 20:749-58); ColoAd1 (Kuhn et al. (2008) PLoS One, 3:e2409; MV-CEA and MV-NIS (Msaouel et al. (2009) Curr. Opin. Mol. Ther., 11:43-53); Synco-B18R (Fu et al. (2012) Mol. Ther., 20:1871-81); OncoVEX GM-CSF (Kaufman et al. (2010) Future Oncol. 6:941-9), Reo-001 (Reolysin®, Galanis it al. (2012) Mol. Ther., 20:1998-2003); NTX-010 (Morton et al. (2010) Pediatr Blood Cancer, 55:295-303); and Coxsackieviruses A13, A15, A18, A20 and A21 (e.g., CAVATAK™, which is coxsackievirus A21.)

In addition, adenoviruses, such as the ONYX viruses and others, have been modified, such as by deletion of EA1 genes, so that they selectively replicate in cancerous cells, and, thus, are oncolytic. Adenoviruses also have been engineered to have modified tropism for tumor therapy and also as gene therapy vectors. Examples of such is ONYX-015, H101 and Ad5ΔCR (Hallden and Portella (2012) Expert Opin Ther Targets, 16:945-58) and TNFerade (McLoughlin et al. (2005) Ann. Surg. Oncol., 12:825-30). A conditionally replicative adenovirus, Oncorine®, has been approved in China.

Any virus, including any described above, can be modified to remove or disrupt native genes that cause disease and insert nucleic acid molecules encoding one or more chromophore-producing enzymes (e.g., one or more melanin-producing enzyme) using standard cloning methods known in the art and described elsewhere herein. For example, the sequence of nucleotides encoding a chromophore-producing enzyme is inserted into or in place of a non-essential gene or region in the genome of an unmodified oncolytic virus or is inserted into in or in place of nucleic acid encoding a heterologous gene product in the genome of an unmodified oncolytic virus. Any of the oncolytic viruses described above or in Section E further below, or otherwise known in the art, can be used as an unmodified virus herein for insertion of nucleic acid encoding a chromophore-producing enzyme.

E. Vaccinia Viruses

Vaccinia viruses are oncolytic viruses that possess a variety of features that make them particularly suitable for use in wound and cancer gene therapy. For example, vaccinia is a cytoplasmic virus, thus, it does not insert its genome into the host genome during its life cycle. Unlike many other viruses that require the host's transcription machinery, vaccinia virus can support its own gene expression in the host cell cytoplasm using enzymes encoded in the viral genome. Vaccinia viruses also have a broad host and cell type range. In particular, vaccinia viruses can accumulate in immunoprivileged cells or immunoprivileged tissues, including tumors and/or metastases, and also including wounded tissues and cells. Yet, unlike other oncolytic viruses, vaccinia virus can typically be cleared from the subject to whom the viruses are administered by activity of the subject's immune system, and hence are less toxic than other viruses such as adenoviruses. Thus, while the viruses can typically be cleared from the subject to whom the viruses are administered by activity of the subject's immune system, viruses can nevertheless accumulate, survive and proliferate in immunoprivileged cells and tissues such as tumors, because such immunoprivileged areas are isolated from the host's immune system.

Vaccinia viruses also can be easily modified by insertion of heterologous genes. This can result in the attenuation of the virus and/or permit delivery of therapeutic proteins. For example, vaccinia virus genome has a large carrying capacity for foreign genes, where up to 25 kb of exogenous DNA fragments (approximately 12% of the vaccinia genome size) can be inserted. The genomes of several of the vaccinia strains have been completely sequenced, and many essential and nonessential genes identified. Due to high sequence homology among different strains, genomic information from one vaccinia strain can be used for designing and generating modified viruses in other strains. Finally, the techniques for production of modified vaccinia strains by genetic engineering are well established (Moss, Curr. Opin. Genet. Dev. 3 (1993), 86-90; Broder and Earl, Mol. Biotechnol. 13 (1999), 223-245; Timiryasova et al., Biotechniques 31 (2001), 534-540).

Various vaccina viruses have been demonstrated to exhibit antitumor activities. In one study, for example, nude mice bearing nonmetastatic colon adenocarcinoma cells were systemically injected with a WR strain of vaccinia virus modified by having a vaccinia growth factor deletion and an enhanced green fluorescence protein inserted into the thymidine kinase locus. The virus was observed to have antitumor effect, including one complete response, despite a lack of exogenous therapeutic genes in the modified virus (McCart et al. (2001) Cancer Res 1:8751-8757). In another study, vaccinia melanoma oncolysate (VMO) was injected into sites near melanoma positive lymph nodes in a Phase III clinical trial of melanoma patients. As a control, New York City Board of Health strain vaccinia virus (VV) was administered to melanoma patients. The melanoma patients treated with VMO had a survival rate better than that for untreated patients, but similar to patients treated with the VV control (Kim et al. (2001) Surgical Oncol 10:53-59).

LIVP strains of vaccinia virus also have been used for the diagnosis and therapy of tumors, and for the treatment of wounded and inflamed tissues and cells (see e.g., Zhang et al. (2007) Surgery, 142:976-983; Lin et al. (2008) J. Clin. Endocrinol. Metab., 93:4403-7; Kelly et al. (2008) Hum gene There., 19:774-782; Yu et al. (2009) Mol Cancer Ther., 8:141-151; Yu et al. (2009) Mol Cancer, 8:45; U.S. Pat. No. 7,588,767; U.S. Pat. No. 8,052,968; and U.S. Publication No. US20040234455). For example, when intravenously administered, LIVP strains have been demonstrated to accumulate in internal tumors at various loci in vivo, and have been demonstrated to effectively treat human tumors of various tissue origin, including, but not limited to, breast tumors, thyroid tumors, pancreatic tumors, metastatic tumors of pleural mesothelioma, squamous cell carcinoma, lung carcinoma and ovarian tumors. LIVP strains of vaccinia, including attenuated forms thereof, exhibit less toxicity than WR strains of vaccinia virus, and results in increased and longer survival of treated tumor-bearing animal models (see e.g., U.S. Published Patent Appl. No. US20110293527).

Vaccinia is a cytoplasmic virus, thus, it does not insert its genome into the host genome during its life cycle. Vaccinia virus has a linear, double-stranded DNA genome of approximately 180,000 base pairs in length that is made up of a single continuous polynucleotide chain (Baroudy et al. (1982) Cell, 28:315-324). The structure is due to the presence of 10,000 base pair inverted terminal repeats (ITRs). The ITRs are involved in genome replication. Genome replication is believed to involve self-priming, leading to the formation of high molecular weight concatemers (isolated from infected cells) which are subsequently cleaved and repaired to make virus genomes (see, e.g., Traktman, P., Chapter 27, Poxvirus DNA Replication, pp. 775-798, in DNA Replication in Eukaryotic Cells, Cold Spring Harbor Laboratory Press (1996)). The genome contains approximately 250 genes. In general, the non-segmented, non-infectious genome is arranged such that centrally located genes are essential for virus replication (and are thus conserved), while genes near the two termini effect more peripheral functions such as host range and virulence. Vaccinia viruses practice differential gene expression by utilizing open reading frames (ORFs) arranged in sets that, as a general principle, do not overlap.

Vaccinia virus possesses a variety of features for use in cancer gene therapy and vaccination including broad host and cell type range, and low toxicity. For example, while most oncolytic viruses are natural pathogens, vaccinia virus has a unique history in its widespread application as a smallpox vaccine that has resulted in an established track record of safety in humans. Toxicities related to vaccinia administration occur in less than 0.1% of cases, and can be effectively addressed with immunoglobulin administration. In addition, vaccinia virus possesses a large carrying capacity for foreign genes (up to 25 kb of exogenous DNA fragments (approximately 12% of the vaccinia genome size) can be inserted into the vaccinia genome) and high sequence homology among different strains for designing and generating modified viruses in other strains. Techniques for production of modified vaccinia strains by genetic engineering are well established (Moss (1993) Curr. Opin. Genet. Dev. 3: 86-90; Broder and Earl (1999) Mol. Biotechnol. 13: 223-245; Timiryasova et al. (2001) Biotechniques 31: 534-540). Vaccinia virus strains have been shown to specifically colonize solid tumors, while not infecting other organs (see, e.g., Zhang et al. (2007) Cancer Res 67:10038-10046; Yu et al., (2004) Nat Biotech 22:313-320; Heo et al., (2011) Mol Ther 19:1170-1179; Liu et al. (2008) Mol Ther 16:1637-1642; Park et al., (2008) Lancet Oncol, 9:533-542).

A variety of vaccinia virus strains are available for modification by insertion of nucleic acid encoding melanin producing enzymes, including, but not limited to, Western Reserve (WR) (SEQ ID NO: 62), Copenhagen (SEQ ID NO: 63), Tashkent, Tian Tan, Lister, Wyeth, IHD-J, and IHD-W, Brighton, Ankara, MVA, Dairen I, LIPV, LC16M8, LC 16MO, LIVP, WR 65-16, and NYCBH. Exemplary known viruses are set forth in Table 3. Exemplary vaccinia viruses for insertion of heterologous nucleic acid encoding a chromophore-producing enzyme(s) for use in the methods provided herein include, but are not limited to, Lister strain or LIVP strain of vaccinia viruses, WR strains, or modified forms thereof. LIVP generally exhibits less virulence than the WR strain. Also, for example, a recombinant derivative of LIVP, designated GLV-1h68 (set forth in SEQ ID NO: 2; GenBank Acc. No. EU410304) and GLV-1h64 (set forth in SEQ ID NO: 189) exhibit tumor targeting properties and an improved safety profile compared to its parental LIVP strain (set forth in SEQ ID NO: 188) and the WR strain (Zhang et al. (2009) Mol. Genet. Genomics, 282:417-435).

TABLE 3

| | | Reference (e.g., GenBank |
|---|---|---|
| Name | Abbreviations | Accession No.) |
| Vaccinia virus strain | WR | AY243312 |
| Western Reserve | | |
| Vaccinia virus strain | COP | M35027 |
| Copenhagen | | |
| Vaccinia Lister major strain | LIST | AY678276 |
| Vaccinia Lister isolate | LC | AY678277 |
| LC16MO | | |
| Vaccinia Lister clone | VACV107 | DQ121394 |
| VACV107 | | |
| Vaccinia virus strain | ACAM | AY313847 |
| ACAM2000 | | |
| Vaccinia virus strain DUKE | DUKE | DQ439815; Li et al. (2006) |
| | | Virology J, 3: 88 |
| Vaccinia virus strain | MVA | U94848 |
| Ankara | | |
| Vaccinia virus Clone3 | CLONE3 | AY138848 |

1. Lister and LIVP Strains

Exemplary vaccinia viruses are Lister or LIVP vaccinia viruses. Lister (also referred to as Elstree) vaccinia virus is available from any of a variety of sources. For example, the Elstree vaccinia virus is available at the ATCC under Accession Number VR-1549. The Lister vaccinia strain has high transduction efficiency in tumor cells with high levels of gene expression.

The vaccinia virus provided in the compositions and methods herein containing a sequence of nucleotides encoding a chromophore-producing enzyme or enzymes (e.g., melanin-producing enzyme or enzymes) can be based on modifications to the Lister strain of vaccinia virus. LIVP is a vaccinia strain derived from Lister (ATCC Catalog No. VR-1549). As described elsewhere herein, the LIVP strain can be obtained from the Lister Institute of Viral Preparations, Moscow, Russia; the Microorganism Collection of FSRI SRC VB Vector; or can be obtained from the Moscow Ivanovsky Institute of Virology (C0355 K0602). The LIVP strain was used for vaccination throughout the world, particularly in India and Russia, and is widely available. LIVP and its production are described, for example, in U.S. Pat. Nos. 7,588,767, 7,588,771, 7,662,398 and 7,754,221 and U.S. Publication Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917, 2011/0064650; Zhang et al. (2009) Mol. Genet. Genomics, 282:417-435). A sequence of a parental genome of LIVP is set forth in SEQ ID NO: 188.

LIVP strains in the compositions provided herein also include clonal strains that are derived from LIVP and that can be present in a virus preparation propagated from LIVP. The LIVP clonal strains have a genome that differs from the parental sequence set forth in SEQ ID NO: 188. The clonal strains provided herein exhibit greater anti-tumorigenicity and/or reduced toxicity compared to the recombinant or modified virus strain designated GLV-1h68 (having a genome set forth in SEQ ID NO: 2).

The LIVP and clonal strains have a sequence of nucleotides that have at least 70%, such as at least 75%, 80%, 85% or 90% sequence identity to SEQ ID NO: 1 or 188. For example, the clonal strains have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9% or 100% sequence identity to SEQ ID NO: 1 or 188. Such LIVP clonal viruses include viruses that differ in one or more open reading frames (ORF) compared to the parental LIVP strain that has a sequence of amino acids set forth in SEQ ID NO: 1 or 188. The LIVP clonal virus strains provided herein can contain a nucleotide deletion or mutation in any one or more nucleotides in any ORF compared to SEQ ID NO: 1 or 188, or can contain an addition or insertion of viral DNA compared to SEQ ID NO: 1 or 188.

LIVP strains in the compositions provided herein include those that have a nucleotide sequence corresponding to nucleotides 2,256-181,114 of SEQ ID NO: 55, nucleotides 11,243-182,721 of SEQ ID NO: 56, nucleotides 6,264-181,390 of SEQ ID NO: 57, nucleotides 7,044-181,820 of SEQ ID NO: 58, nucleotides 6,674-181,409 of SEQ ID NO: 59, nucleotides 6,716-181,367 of SEQ ID NO: 60 or nucleotides 6,899-181,870 of SEQ ID NO: 61, or to a complement thereof. In some examples, the LIVP strain for use in the compositions and methods is a clonal strain of LIVP or a modified form thereof containing a sequence of nucleotides that has at least 97%, 98%, 99% or more sequence identity to a sequence of nucleotides 2,256-181,114 of SEQ ID NO: 55, nucleotides 11,243-182,721 of SEQ ID NO: 56, nucleotides 6,264-181,390 of SEQ ID NO: 57, nucleotides 7,044-181,820 of SEQ ID NO: 58, nucleotides 6,674-181,409 of SEQ ID NO: 59, nucleotides 6,716-181,367 of SEQ ID NO: 60 or nucleotides 6,899-181,870 of SEQ ID NO: 61. LIVP clonal strains provided herein generally also include terminal nucleotides corresponding to a left and/or right inverted terminal repeat (ITR). Exemplary LIVP strains include, but are not limited to, virus strains designated LIVP 1.1.1 having a genome containing a sequence of nucleotides set forth in SEQ ID NO: 55 or a sequence of nucleotides that exhibits at least 97% sequence identity to SEQ ID NO: 55; a virus strain designated LIVP 2.1.1 having a genome containing a sequence of nucleotides set forth in SEQ ID NO: 56 or a sequence of nucleotides that exhibits at least 97%, 98%, 99% or more sequence identity to SEQ ID NO: 56; a virus strain designated LIVP 4.1.1 having a genome containing a sequence of nucleotides set forth in SEQ ID NO: 57 or a sequence of nucleotides that exhibits at least 97%, 98%, 99% or more sequence identity to SEQ ID NO: 57; a virus strain designated LIVP 5.1.1 having a genome containing a sequence of nucleotides set forth in SEQ ID NO: 58 or a sequence of nucleotides that exhibits at least 97%, 98%, 99% or more sequence identity to SEQ ID NO: 58; a virus strain designated LIVP 6.1.1 having a sequence of nucleotides set forth in SEQ ID NO: 59 or a sequence of nucleotide that exhibits at least 97%, 98%, 99% or more sequence identity to SEQ ID NO: 59; a virus strain designated LIVP 7.1.1 having a genome containing a sequence of nucleotides set forth in SEQ ID NO: 60 or a sequence of nucleotides that exhibits at least 97%, 98%, 99% or more sequence identity to SEQ ID NO: 60; or a virus strain designated LIVP 8.1.1 having a genome containing a sequence of nucleotides set forth in SEQ ID NO: 61 or a sequence of nucleotides that exhibits at least 97%, 98%, 99% or more sequence identity to SEQ ID NO: 61.

3. Heterologous Nucleic Acid and Modified Viruses

The large genome size of poxviruses, such as the vaccinia viruses in the compositions provided herein, allows large inserts of heterologous DNA and/or multiple inserts of heterologous DNA to be incorporated into the genome (Smith and Moss (1983) Gene 25(1):21-28). Unmodified vaccinia viruses for insertion of coding sequences for a chromophore-producing enzyme or enzymes (e.g., a melanin-producing enzyme or enzymes), such as any described above, also can contain genes encoding other heterologous gene products. Hence, the unmodified viruses can be viruses that have been previously modified by insertion of heterologous DNA molecules. Thus, the vaccinia viruses in the compositions and methods provided herein can be modified by insertion of 1, 2, 3, 4, 5, 6, 7, 8, 9, 10 or more heterologous DNA molecules. Generally, the one or more heterologous DNA molecules are inserted into a non-essential region of the virus genome. For example, the one or more heterologous DNA molecules are inserted into a locus of the virus genome that is non-essential for replication in proliferating cells, such as tumor cells. Exemplary insertion sites are provided herein below and are known in the art.

In some examples, the virus can be modified to express an exogenous or heterologous gene. Exemplary exogenous gene products include proteins and RNA molecules. The modified viruses can express a therapeutic gene product, a detectable gene product, a gene product for manufacturing or harvesting, an antigenic gene product for antibody harvesting, or a viral gene product. The characteristics of such gene products are described herein and elsewhere.

In some examples, the viruses can be modified to express two or more gene products, such as 2, 3, 4, 5, 6, 7, 8, 9, 10 or more gene products, where any combination of the two or more gene products can be one or more detectable gene products, therapeutic gene products, gene products for manufacturing or harvesting or antigenic gene products for antibody harvesting or a viral gene product. In one example, a virus can be modified to express an anticancer gene product. In another example, a virus can be modified to express two or more gene products for detection or two or more therapeutic gene products. In some examples, one or more proteins involved in biosynthesis of a luciferase substrate can be expressed along with luciferase. When two or more exogenous genes are introduced, the genes can be regulated under the same or different regulatory sequences, and the genes can be inserted in the same or different regions of the viral genome, in a single or a plurality of genetic manipulation steps. In some examples, one gene, such as a gene encoding a detectable gene product, can be under the control of a constitutive promoter, while a second gene, such as a gene encoding a therapeutic gene product, can be under the control of an inducible promoter. Methods for inserting two or more genes into a virus are known in the art and can be readily performed for a wide variety of viruses using a wide variety of exogenous genes, regulatory sequences, and/or other nucleic acid sequences.

The heterologous DNA can be an exemplary gene, including any from the list of human genes and genetic disorders authored and edited by Dr. Victor A. McKusick and his colleagues at Johns Hopkins University and elsewhere, and developed for the World Wide Web by NCBI, the National Center for Biotechnology Information; online, Mendelian Inheritance in Man, OMIM™ Center for Medical Genetics, Johns Hopkins University (Baltimore, Md.), and National Center for Biotechnology Information, National Library of Medicine (Bethesda, Md.), 1999; and those available in public databases, such as PubMed and GenBank (see, e.g., (ncbi.nlm.nih.gov/entrez/query.fcgi?db=OMIM).

In particular, viruses provided herein can be modified to express an anti-tumor antibody, an anti-metastatic gene or metastasis suppressor genes; cell matrix degradative genes; hormones; growth factors; immune modulatory molecules, including cytokines, such as interleukins or interferons, chemokines, including CXC chemokines, costimulatory molecules; ribozymes; transporter proteins; antibodies or fragments thereof; antisense RNA; siRNA; microRNAs; protein ligands; a mitosis inhibitor protein; an antimitotic oligopeptide; an anti-cancer polypeptide; anti-cancer antibiotics; angiogenesis inhibitors; anti-angiogenic factors; tissue factors; a prodrug converting enzyme; genes for tissue regeneration and reprogramming human somatic cells to pluripotency; enzymes that modify a substrate to produce a detectable product or signal or are detectable by antibodies; viral attenuation factors; a superantigen; proteins that can bind a contrasting agent, chromophore, or a compound of ligand that can be detected; tumor suppressors; cytotoxic protein; cytostatic protein; genes for optical imaging or detection including luciferase, a fluorescent protein such as a green fluorescent protein (GFP) or GFP-like protein, a red fluorescent protein (RFP), a far-red fluorescent protein, a near-infrared fluorescent protein, a yellow fluorescent protein (YFP), an orange fluorescent protein (OFP), a cerulean fluorescent protein (CFP), or a blue fluorescent protein (BFP), and phycobiliproteins from certain cyanobacteria and eukaryotic algae, including phycoerythrins (red) and the phycocyanins (blue); genes for PET imaging; genes for MRI imaging; or genes to alter attenuation of the viruses.

a. Exemplary Modifications

Exemplary heterologous genes for modification of viruses herein are known in the art (see e.g., U.S. Pub. Nos. US2003-0059400, US2003-0228261, US2009-0117034, US2009-0098529, US2009-0053244, US2009-0081639 and US2009-0136917; U.S. Pat. Nos. 7,588,767 and 7,763,420; and International Pub. No. WO 2009/139921). Such modifications are in addition to the modification of the viruses herein to contain heterologous DNA encoding a chromophore-producing enzyme or enzymes, such as a melanin-producing enzyme or enzymes, for example any described above.

A non-limiting description of exemplary genes encoding heterologous proteins for modification of virus strains is set forth in the following table. The sequence of the gene and encoded proteins are known to one of skill in the art from the literature. Hence, provided herein are virus strains, including any of the clonal viruses provided herein, that contain nucleotides encoding any of the heterologous proteins listed in Table 4.

TABLE 4
Exemplary Genes and Gene Products

```
Detectable gene products
  Optical Imaging
  Luciferase
```

```
bacterial luciferase
luciferase (from Vibrio harveyi or Vibrio fischeri)
  luxA
  luxB
  luxC
  luxD
  luxE
  luxAB
  luxCD
  luxABCDE
firefly luciferase
Renilla luciferase from Renilla reniformis
Gaussia luciferase
luciferases found among marine arthropods
luciferases that catalyze the oxidation of Cypridina (Vargula) luciferin
luciferases that catalyze the oxidation of Coleoptera luciferin
```

luciferase photoproteins

aequorin photoprotein to which luciferin is non-covalently bound

click beetle luciferase

CBG99
(CBG99)mRFP1

Fusion Proteins

Ruc-GFP

Fluorescent Proteins
GFP

aequorin from Aequorea victoria
GFP from Aequorea victoria
GFP from Aequorea coerulescens
GFP from the anthozoan coelenterates Renilla reniformis and Renilla koellikeri (sea
pansies)
Emerald (Invitrogen, Carlsbad, CA)
EGFP (Clontech, Palo Alto, CA)
Azami-Green (MBL International, Woburn, MA)
Kaede (MBL International, Woburn, MA)
ZsGreen1 (Clontech, Palo Alto, CA)
CopGFP (Evrogen/Axxora, LLC, San Diego, CA)
Anthozoa reef coral
Anemonia sea anemone
Renilla sea pansy
Galaxea coral
Acropora brown coral
Trachyphyllia stony coral
Pectiniidae stony coral
GFP-like proteins

RFP

RFP from the corallimorph Discosoma (DsRed) (Matz et al. (1999) Nature
Biotechnology 17: 969-973)
Heteractis reef coral, Actinia or Entacmaea sea anemone
RFPs from Discosoma variants

mRFP1 (Wang et al. (2004) Proc. Natl. Acad. Sci. U.S.A. 101: 16745-9)
mCherry (Wang et al. (2004) PNAS USA. 101(48): 16745-9)
tdTomato (Wang et al. (2004) PNAS USA. 101 (48): 16745-9)
mStrawberry (Wang et al. (2004) PNAS USA. 101(48): 16745-9)
mTangerine (Wang et al. (2004) PNAS USA. 101(48): 16745-9)
DsRed2 (Clontech, Palo Alto, CA)
DsRed-T1 (Bevis and Click (2002) Nat. Biotechnol. 20: 83-87)
Anthomedusa J-Red (Evrogen)
Anemonia AsRed2 (Clontech, Palo Alto, CA)

far-red fluorescent protein

TurboFP635
mNeptune monomeric far-red fluorescent protein
Actinia AQ143 (Shkrob et al. (2005) Biochem J. 392(Pt 3): 649-54)
Entacmaea eqFP611 (Wiedenmann et al. (2002) PNAS USA. 99(18): 11646-51)
Discosoma variants

mPlum (Wang et al.. (2004) PNAS USA. 101(48): 16745-9)
mRasberry (Wang et al. (2004) PNAS USA. 101(48): 16745-9)
Heteractis HcRed1 and t-HcRed (Clontech, Palo Alto, CA)

IFP (infrared fluorescent protein)
near-infrared fluorescent protein
YFP

EYFP (Clontech, Palo Alto, CA)
YPet (Nguyen and Daugherty (2005) Nat Biotechnol. 23(3): 355-60)
Venus (Nagai et al. (2002) Nat. Biotechnol. 20(1): 87-90)
Zs Yellow (Clontech, Palo Alto, CA)
mCitrine (Wang et al. (2004) PNAS USA. 101(48): 16745-9)

OFP

cOFP (Strategene, La Jolla, CA)
mKO (MBL International, Woburn, MA)
mOrange (Wang et al. (2004) PNAS USA. 101(48): 16745-9)

CFP

Cerulean (Rizzo (2004) Nat Biotechnol. 22(4): 445-9)
mCFP (Wang et al. (2004) PNAS USA. 101(48): 16745-9)
AmCyan1 (Clontech, Palo Alto, CA)
MiCy (MBL International, Woburn, MA)
CyPet (Nguyen and Daugherty (2005) Nat Biotechnol. 23(3): 355-60)

BFP

EBFP (Clontech, Palo Alto, CA);
phycobiliproteins from certain cyanobacteria and eukaryotic algae, phycoerythrins
(red) and the phycocyanins (blue)

R-Phycoerythrin (R-PE)
B-Phycoerythrin (B-PE)
Y-Phycoerythrin (Y-PE
C-Phycocyanin (P-PC)
R-Phycocyanin (R-PC)
Phycoerythrin 566 (PE 566)
Phycoerythrocyanin (PEC)
Allophycocyanin (APC)
frp Flavin Reductase
CBP Coelenterazine-binding protein 1
PET imaging

Cyp11B1 transcript variant 1
Cyp11B1 transcript variant 2
Cyp11B2
AlstR
PEPR-1
LAT-4 (SLC43A2)
Cyp51 transcript variant 1
Cyp51 transcript variant 2

Transporter proteins
Solute carrier transporter protein families (SLC)

SLC1 solute carrier 1 transporter protein family
SLC1A1, SLC1A2, SLC1A3, SLC1A4, SLC1A5, SLC1A6, SLC1A7
SLC2 solute carrier 2 transporter protein family
SLC2A1, SLC2A2, SLC2A3, SLC2A4, SLC2A5, SLC2A6, SLC2A7, SLC2A8,
SLC2A9, SLC2A10, SLC2A11, SLC2A12, SLC2A13, SLC2A14)
SLC3 solute carrier 3 transporter protein family
SLC3A1, SLC3A2
SLC 4 solute carrier 4 transporter protein family
SLC4A1, SLC4A2, SLC4A3, SLC4A4, SLC4A5, SLC4A6, SLC4A7, SLC4A8,
SLC4A9, SLC4A10, SLC4A11
SLC5 solute carrier 5 transporter protein family
SLC5A1 sodium/glucose cotransporter 1
SLC5A2 sodium/glucose cotransporter 2
SLC5A3 sodium/myo-inositol cotransporter
SLC5A4 low affinity sodium-glucose cotransporter
SLC5A5 sodium/iodide cotransporter
SLC5A6 sodium-dependent multivitamin transporter
SLC5A7 high affinity choline transporter 1
SLC5A8 sodium-coupled monocarboxylate transporter 1
SLC5A9 sodium/glucose cotransporter 4
SLC5A10 sodium/glucose cotransporter 5, isoform 1
sodium/glucose cotransporter 5, isoform 2
sodium/glucose cotransporter 5, isoform 3
sodium/glucose cotransporter 5, isoform 4
SLC5A11 sodium/myo-inositol cotransporter 2, isoform 1
sodium/myo-inositol cotransporter 2, isoform 2
sodium/myo-inositol cotransporter 2, isoform 3
sodium/myo-inositol cotransporter 2, isoform 4
SLC5A12 sodium-coupled monocarboxylate transporter 2, isoform 1
sodium-coupled monocarboxylate transporter 2, isoform 2

Sodium Iodide Symporter (NIS)
hNIS (NM_000453)
hNIS (BC105049)
hNIS (BC105047)
hNIS (non-functional hNIS variant containing an additional 11 aa)

SLC6 solute carrier 6 transporter protein family
SLC6A1 sodium- and chloride-dependent GABA transporter 1
SLC6A2 norepinephrine transporter (sodium-dependent noradrenaline transporter)
SLC6A3 sodium-dependent dopamine transporter
SLC6A4 sodium-dependent serotonin transporter
SLC6A5 sodium- and chloride-dependent glycine transporter 1
SLC6A6 sodium- and chloride-dependent taurine transporter
SLC6A7 sodium-dependent proline transporter
SLC6A8 sodium- and chloride-dependent creatine transporter
SLC6A9 sodium- and chloride-dependent glycine transporter 1, isoform 1
sodium- and chloride-dependent glycine transporter 1, isoform 2
sodium- and chloride-dependent glycine transporter 1, isoform 3
SLC6A10 sodium- and chloride-dependent creatine transporter 2
SLC6A11 sodium- and chloride-dependent GABA transporter 3
SLC6A12 sodium- and chloride-dependent betaine transporter
SLC6A13 sodium- and chloride-dependent GABA transporter 2
SLC6A14 Sodium- and chloride-dependent neutral and basic amino acid transporter
B(0+)
SLC6A15 Orphan sodium- and chloride-dependent neurotransmitter transporter
NTT73
SLC6A16 Orphan sodium- and chloride-dependent neurotransmitter transporter
NTT5
SLC6A17 Orphan sodium- and chloride-dependent neurotransmitter transporter
NTT4
Sodium SLC6A18 Sodium- and chloride-dependent transporter XTRP2
SLC6A19 Sodium-dependent neutral amino acid transporter B(0)
SLC6A20 Sodium- and chloride-dependent transporter XTRP3
Norepinephrine Transporter (NET)

Human Net (hNET) transcript variant 1 (NM_001172504)
Human Net (hNET) transcript variant 2 (NM_001172501)
Human Net (hNET) transcript variant 3 (NM_001043)
Human Net (hNET) transcript variant 4 (NM_001172502)
Non-Human Net

SLC7 solute carrier 7 transporter protein family
SLC7A1, SLC7A2, SLC7A3, SLC7A4, SLC7A5, SLC7A6, SLC7A7, SLC7A8,
SLC7A9, SLC7A10, SLC7A11, SLC7A13, SLC7A14
SLC8 solute carrier 8 transporter protein family
SLC8A1, SLC8A2, SLC8A3
SLC9 solute carrier 9 transporter protein family
SLC9A1, SLC9A2, SLC9A3, SLC9A4, SLC9A5, SLC9A6, SLC9A7, SLC9A8,
SLC9A9, SLC9A10, SLC9A11
SLC10 solute carrier 10 transporter protein family
SLC10A1, SLC10A2, SLC10A3, SLC10A4, SLC10A5, SLC10A6, SLC10A7
SLC11 solute carrier 11 transporter protein family
SLC11A1
SCL11A2 or hDMT

SLC11A2 transcript variant 4
SLC11A2 transcript variant 1
SLC11A2 transcript variant 2
SLC11A2 transcript variant 3
SLC11A2 transcript variant 5
SLC11A2 transcript variant 6
SLC11A2 transcript variant 7

SLC12 solute carrier 12 transporter protein family
SLC12A1, SLC12A1, SLC12A2, SLC12A3, SLC12A4, SLC12A5, SLC12A6,
SLC12A7, SLC12A8, SLC12A9
SLC13 solute carrier 13 transporter protein family
SLC13A1, SLC13A2, SLC13A3, SLC13A4, SLC13A5
SLC 14 solute carrier 14 transporter protein family
SLC14A1, SLC14A2
SLC15 solute carrier 15 transporter protein family
SLC15A1, SLC15A2, SLC15A3, SLC15A4
SLC16 solute carrier 16 transporter protein family
SLC16A1, SLC16A2, SLC16A3, SLC16A4, SLC16A5, SLC16A6, SLC16A7,
SLC16A8, SLC16A9, SLC16A10, SLC16A11, SLC16A12, SLC16A13,
SLC16A14
SLC 17 solute carrier 17 transporter protein family
SLC17A1, SLC17A2, SLC17A3, SLC17A4, SLC17A5, SLC17A6, SLC17A7,
SLC17A8
SLC18 solute carrier 18 transporter protein family
SLC18A1, SLC18A2, SLC18A3
SLC19 solute carrier 19 transporter protein family
SLC19A1, SLC19A2, SLC19A3
SLC20 solute carrier 20 transporter protein family
SLC20A1, SLC20A2
SLC21 solute carrier 21 transporter protein family

subfamily 1; SLCO1A2, SLCO1B1, SLCO1B3, SLCO1B4,
SLCO1C1
subfamily 2; SLCO2A1, SLCO2B1
subfamily 3; SLCO3A1
subfamily 4; SLCO4A1, SLCO4C1
subfamily 5; SLCO5A1

SLC22 solute carrier 22 transporter protein family
SLC22A1, SLC22A2, SLC22A3, SLC22A4, SLC22A5, SLC22A6, SLC22A7,
SLC22A8, SLC22A9, SLC22A10, SLC22A11, SLC22A12, SLC22A13,
SLC22A14, SLC22A15, SLC22A16, SLC22A17, SLC22A18, SLC22A19,
SLC22A20
SLC23 solute carrier 23 transporter protein family
SLC23A1, SLC23A2, SLC23A3, SLC23A4
SLC24 solute carrier 24 transporter protein family
SLC24A1, SLC24A2, SLC24A3, SLC24A4, SLC24A5, SLC24A6
SLC25 solute carrier 25 transporter protein family
SLC25A1, SLC25A2, SLC25A3, SLC25A4, SLC25A5, SLC25A6, SLC25A7,
SLC25A8, SLC25A9, SLC25A10, SLC25A11, SLC25A12, SLC25A13,
SLC25A14, SLC25A15, SLC25A16, SLC25A17, SLC25A18, SLC25A19,
SLC25A20, SLC25A21, SLC25A22, SLC25A23, SLC25A24, SLC25A25,
SLC25A26, SLC25A27, SLC25A28, SLC25A29, SLC25A30, SLC25A31,
SLC25A32, SLC25A33, SLC25A34, SLC25A35, SLC25A36, SLC25A37,
SLC25A38, SLC25A39, SLC25A40, SLC25A41, SLC25A42, SLC25A43,
SLC25A44, SLC25A45, SLC25A46
SLC26 solute carrier 26 transporter protein family
SLC26A1, SLC26A2, SLC26A3, SLC26A4, SLC26A5, SLC26A6, SLC26A7,
SLC26A8, SLC26A9, SLC26A10, SLC26A11
SLC27 solute carrier 27 transporter protein family
SLC27A1, SLC27A2, SLC27A3, SLC27A4, SLC27A5, SLC27A6
SLC28 solute carrier 28 transporter protein family
SLC28A1, SLC28A2, SLC28A3
SLC29 solute carrier 29 transporter protein family
SLC29A1, SLC29A2, SLC29A3, SLC29A4
SLC30 solute carrier 30 transporter protein family
SLC30A1, SLC30A2, SLC30A3, SLC30A4, SLC30A5, SLC30A6, SLC30A7,
SLC30A8, SLC30A9, SLC30A10
SLC31 solute carrier 31 transporter protein family
SLC31A1
SLC32 solute carrier 32 transporter protein family
SLC32A1
SLC33 solute carrier 33 transporter protein family
SLC33A1
SLC34 solute carrier 34 transporter protein family
SLC34A1, SLC34A2, SLC34A3
SLC35 solute carrier 35 transporter protein family

subfamily A; SLC35A1, SLC35A2, SLC35A3, SLC35A4, SLC35A5
subfamily B; SLC35B1, SLC35B2, SLC35B3, SLC35B4
subfamily C; SLC35C1, SLC35C2
subfamily D; SLC35D1, SLC35D2, SLC35D3
subfamily E; SLC35E1, SLC35E2, SLC35E3, SLC35E4

SLC36 solute carrier 36 transporter protein family
SLC36A1, SLC36A2, SLC36A3, SLC36A4
SLC37 solute carrier 37 transporter protein family
SLC37A1, SLC37A2, SLC37A3, SLC37A4
SLC38 solute carrier 38 transporter protein family
SLC38A1, SLC38A2, SLC38A3, SLC38A4, SLC38A5, SLC38A6
SLC39 solute carrier 39 transporter protein family
SLC39A1, SLC39A2, SLC39A3, SLC39A4, SLC39A5, SLC39A6, SLC39A7,
SLC39A8, SLC39A9, SLC39A10, SLC39A11, SLC39A12, SLC39A13,
SLC39A14
SLC40 solute carrier 40 transporter protein family
SLC40A1
SLC41 solute carrier 41 transporter protein family
SLC41A1, SLC41A2, SLC41A3
SLC42 solute carrier 42 transporter protein family
RHAG, RhBG, RhCG
SLC43 solute carrier 43 transporter protein family
SLC43A1
SLC43A2
SLC43A3
SLC44 solute carrier 44 transporter protein family
SLC44A1, SLC44A2, SLC44A3, SLC44A4, SLC44A5
SLC45 solute carrier 45 transporter protein family
SLC45A1, SLC45A2, SLC54A3, SLC45A4
SLC46 solute carrier 46 transporter protein family
SLC46A1, SLC46A2
SLC47 solute carrier 47 transporter protein family
SLC47A1, SLC47A2

MRI Imaging

Human transferrin receptor
Mouse transferrin receptor
Human ferritin light chain (FTL)
Human ferritin heavy chain
FTL 498-199InsTC, a mutated form of the ferritin light chain
Bacterial ferritin

E. coli
E. coli strain K12
S. aureus strain MRSA252
S. aureus strain NCTC 8325
H. pylori B8

bacterioferritin
codon optimized bacterioferritin
MagA

Enzymes that modify a substrate to produce a detectable product or signal, or are
detectable by antibodies

alpha-amylase
alkaline phosphatase
secreted alkaline phosphatase
peroxidase
T4 lysozyme
oxidoreductase
pyrophosphatase

Therapeutic genes
therapeutic gene product
antigens

tumor specific antigens
tumor-associated antigens
tissue-specific antigens
bacterial antigens
viral antigens
yeast antigens
fungal antigens
protozoan antigens
parasite antigens
mitogens

an antibody or fragment thereof

virus-specific antibodies

antisense RNA
siRNA

siRNA directed against expression of a tumor-promoting gene

an oncogene
growth factor
angiogenesis promoting gene
a receptor

siRNA molecule directed against expression of any gene essential for cell growth,
cell replication or cell survival.
siRNA molecule directed against expression of any gene that stabilizes the cell
membrane or otherwise limits the number of tumor cell antigens released from the
tumor cell.

protein ligands
an antitumor oligopeptide
an antimitotic peptide

tubulysin,
phomopsin
hemiasterlin
taltobulin (HTI-286, 3)
cryptophycin

a mitosis inhibitor protein
an antimitotic oligopeptide
an anti-cancer polypeptide antibiotic
anti-cancer antibiotics

tissue factors

Tissue Factor (TF)
αvβ3-integrin RGD fusion protein

Immune modulatory molecules

GM-CSF
MCP-1 or CCL2 (Monocyte Chemoattractant Protein-1) Human
MCP-1 murine
IP-10 or Chemokine ligand 10 (CXCL10)
LIGHT
P60 or SEQSTM1 (Sequestosome 1 transcript variant 1)
P60 or SEQSTM1 (Sequestosome 1 transcript variant 3)
P60 or SEQSTM1 (Sequestosome 1 transcript variant 2)
OspF
OspG
STAT1alpha
STAT1beta

Interleukins

IL-18 (Interleukin-18)
IL-11 (Interleukin-11)
IL-6 (Interleukin-6)
SIL-6R-IL-6
interleukin-12
interleukin-1
interleukin-2
IL-24 (Interleukin-24)
IL-24 transcript variant 1
IL-24 transcript variant 4
IL-24 transcript variant 5
IL-4
IL-8
IL-10

chemokines

IP-10 (CXCL)
Thrombopoietin
members of the C-X-C and C-C chemokine families
RANTES
MIP1-alpha
MIP1-beta
MIP-2

CXC chemokines

GROα
GROβ (MIP-2)
GROγ
ENA-78
LDGF-PPBP
GCP-2
PF4
Mig
IP-10
SDF-1α/β
BUNZO/STRC33
I-TAC
BLC/BCA-1
MDC
TECK
TARC
HCC-1
HCC-4
DC-CK1
MIP-3α
MIP-3β
MCP-2
MCP-3 (Monocyte Chemoattractant Protein-3, CCL7)
MCP-4
MCP-5 (Monocyte Chemoattractant Protein-5; CCL12)
Eotaxin (CCL11)
Eotaxin-2/MPIF-2
I-309
MIP-5/HCC-2
MPIF-1
6Ckine
CTACK
MEC
lymphotactin
fractalkine

Immunoglobulin superfamily of cytokines

B7.1
B7.2.

Anti-angiogenic genes/angiogenesis inhibitors

Human plasminogen k5 domain (hK5)
PEDF (SERPINF1) (Human)
PEDF (mouse)
anti-VEGF single chain antibody (G6)
anti-DLL4 s.c. antibody GLAF-3
tTF-RGD (truncated human tissue factor protein fused to an RGD peptide)

viral attenuation factors

Interferons

IFN-γ
IFN-α
IFN-β

Antibody or scFv

Therapeutic antibodies (i.e. anticancer antibodies)

Rituximab (RITUXAN)
ADEPT
Trastuzumab (Herceptin)
Tositumomab (Bexxar)
Cetuximab (Erbitux)
Ibritumomab (90Y-Ibritumomab tiuxetan; Zevalin)
Alemtuzumab (Campath-1H)
Epratuzumab (Lymphocide)
Gemtuzumab ozogamicin (Mylotarg)
Bevacizumab (Avastin) and Edrecolomab (Panorex)
Infliximab

Metastasis suppressor genes

NM23 or NME1 Isoform a
NM23 or NME1 Isoform b

Anti-metastatic genes

E-Cad
Gelsolin
LKB1 (STK11)
RASSF1
RASSF2
RASSF3
RASSF4
RASSF5
RASSF6
RASSF7
RASSF8
Syk
TIMP-1 (Tissue Inhibitor of Metalloproteinase Type-1)
TIMP-2 (Tissue Inhibitor of Metalloproteinase Type-2)
TIMP-3 (Tissue Inhibitor of Metalloproteinase Type-3)
TIMP-4 (Tissue Inhibitor of Metalloproteinase Type-4)
BRMS-1
CRMP-1
CRSP3
CTGF
DRG1
KAI1
KiSS1 (kisspeptin)
kisspeptin fragments

kisspeptin-10
kisspeptin-13
kisspeptin-14
kisspeptin-54

Mkk4
Mkk6
Mkk7
RKIP
RHOGDI2
SSECKS
TXNIP/VDUP1

Cell matrix-degradative genes

Relaxin 1
hMMP9

Hormones

Human Erythropoietin (EPO)

MicroRNAs

pre-miRNA 181a (sequence inserted into viral genome)
miRNA 181a
mmu-miR-181a MIMAT0000210 mature miRNA 181a
pre-miRNA 126 (sequence inserted into the vial genome)
miRNA 126
hsa-miR-126 MI000471
hsa-miR-126 MIMAT0000445
pre-miRNA 335 (sequence inserted into the viral genome)
miRNA 335
hsa-miR-335 MI0000816
hsa-miR-335 MIMAT0000765

Genes for tissue regeneration and reprogramming Human somatic cells to pluripotency

nAG
Oct4
NANOG
Ngn (Neogenin 1) transcript variant 1
Ngn (Neogenin 1) transcript variant 2
Ngn (Neogenin 1) transcript variant 3
Ngn3
Pdx1
Mafa

Additional Genes

Myc-CTR1
FCU1
mMnSOD
HACE1
nppa1
GCP-2 (Granulocyte Chemotactic Protein-2, CXCL6)
hADH
Wildtype CDC6
Mut CDC6
GLAF-3 anti-DLL4 scFv
GLAF-4 anti-FAP (Fibroblast Activation Protein) scFv (Brocks et al., (2001) Mol.
Medicine 7(7): 461-469)
GLAF-5 anti-FAP scFv
BMP4
wildtype F14.5L

Other Proteins

WT1
p53
pseudomonas A endotoxin
diphtheria toxin
Arf or p16
Bax
Herpes simplex virus thymidine kinase
E. coli purine nucleoside phosphorylase
angiostatin
endostatin
Rb
BRCA1
cystic fibrosis transmembrane regulator (CFTR)
Factor VIII
low density lipoprotein receptor
alpha-galactosidase
beta-glucocerebrosidase
insulin
parathyroid hormone
alpha-1-antitrypsin
rsCD40L
Fas-ligand
TRAIL
TNF
microcin E492
xanthine guanine phosphoribosyltransferase (XGPRT)
E. coli guanine phosphoribosyltransferase (gpt)
hyperforin
endothelin-1 (ET-1)
connective tissue growth factor (CTGF)
vascular endothelial growth factor (VEGF)
cyclooxygenase
COX-2
cyclooxygenase-2 inhibitor
MPO (Myeloperoxidase)
Apo A1 (Apolipoprotein A1)
CRP (C Reactive Protein)
Fibrinogen
SAP (Serum Amyloid P)
FGF-basic (Fibroblast Growth Factor-basic)
PPAR-agonist
PE37/TGF-alpha fusion protein

Replacement of the A34R gene with another A34R gene from a different strain in order
to increase the EEV form of the virus

A34R from VACV IHD-J
A34R with a mutation at codon 151 (Lys 151 to Asp)
A34R with a mutation at codon 151 (Lys 151 to Glu)

Non-coding Sequence

Non-proteins
Non-coding nucleic acid

Ribozymes

Group I introns
Group II introns
RNaseP
hairpin ribozymes
hammerhead ribozymes

Prodrug converting enzymes

varicella zoster thymidine kinase
cytosine deaminase
purine nucleoside phosphorylase (e.g., from E. coli)
beta lactamase
carboxypeptidase G2
carboxypeptidase A
cytochrome P450

cytochrome P450-2B1
cytochrome P450-4B1

horseradish peroxidase
nitroreductase
rabbit carboxylesterase
mushroom tyrosinase
beta galactosidase (lacZ) (i.e., from E. coli)
beta glucuronidase (gusA)
thymidine phosphorylase
deoxycytidine kinase
linamerase

Proteins detectable by antibodies

chloramphenicol acetyl transferase
hGH

Viral attenuation factors

virus-specific antibodies

mucins
thrombospondin

tumor necrosis factors (TNFs)

TNFα

Superantigens
Toxins

diphtheria toxin
Pseudomonas exotoxin
Escherichia coli Shiga toxin
Shigella toxin
Escherichia coli Verotoxin 1
Toxic Shock Syndrome Toxin 1
Exfoliating Toxins (EXft)
Streptococcal Pyrogenic Exotoxin (SPE) A, B and C
Clostridial Perfringens Enterotoxin (CPET)
staphylococcal enterotoxins

SEA, SEB, SEC1, SEC2, SED, SEE and SEH

Mouse Mammary Tumor Virus proteins (MMTV)
Streptococcal M proteins
Listeria monocytogenes antigen p60
mycoplasma arthritis superantigens

Proteins that can bind a contrasting agent, chromophore, or a compound or ligand that
can be detected

siderophores

enterobactin
salmochelin
yersiniabactin
aerobactin

Growth Factors

platelet-derived growth factor (PDG-F)
keratinocyte growth factor (KGF)
insulin-like growth factor-1 (IGF-1)
insulin-like growth factor-binding proteins (IGFBPs)
transforming growth factor (TGF-alpha)
Growth factors for blood cells

Granulocyte Colony Stimulating Factor (G-CSF)

growth factors that can boost platelets

Other Groups

BAC (Bacterial Artificial Chromosome) encoding several or all proteins of a

specific pathway, e.g. wound healing-pathway

MAC (Mammalian Artificial Chromosome) encoding several or all proteins of a

specific pathway, e.g. woundhealing-pathway

tumor antigen
RNAi
ligand binding proteins
proteins that can induce a signal detectable by MRI
angiogenins
photosensitizing agents
anti-metabolites
signaling modulators
chemotherapeutic compounds
lipases
proteases
pro-apoptotic factors
anti-cancer vaccine

antigen vaccines
whole cell vaccines (i.e., dendritic cell vaccines)
DNA vaccines
anti-idiotype vaccines

tumor suppressors
cytotoxic protein
cytostatic proteins
costimulatory molecules

cytokines and chemokines

cancer growth inhibitors
gene therapy
BCG vaccine for bladder cancer

Proteins that interact with host cell proteins

i. Diagnostic or Reporter Gene Products

In some examples, the recombinant vaccinia viruses for insertion of heterologous DNA encoding a chromophore-producing enzyme or enzymes (e.g., a melanin-producing enzyme or enzymes) can express one or more additional genes whose products are detectable or whose products are capable of inducing a detectable signal, in addition to the melanin. In some examples, the viruses provided herein contain nucleic acid that encodes a detectable protein or a protein capable of inducing a detectable signal. Expression of such proteins allows detection of the virus in vitro and in vivo. A variety of detectable gene products, such as detectable proteins are known in the art, and can be used with the viruses provided herein.

Examples of such proteins are enzymes that can catalyze a detectable reaction or catalyze formation of a detectable product, such as, for example, luciferases, such as a click beetle luciferase, a Renilla luciferase, a firefly luciferase or beta-glucuronidase (GusA). Other examples of such proteins are proteins that emit a detectable signal, including fluorescent proteins, such as a green fluorescent protein (GFP) or a red fluorescent protein (RFP). A variety of DNA sequences encoding proteins that can emit a detectable signal or that can catalyze a detectable reaction, such as luminescent or fluorescent proteins, are known and can be used in the viruses and methods provided herein. Transformation and expression of these genes in viruses can permit detection of viral infection, for example, using a low light and/or fluorescence imaging camera.

Exemplary genes encoding light-emitting proteins include, for example, genes from bacterial luciferase from Vibrio harveyi (Belas et al., Science 218 (1982), 791-793), bacterial luciferase from Vibrio fischeri (Foran and Brown, Nucleic acids Res. 16 (1988), 177), firefly luciferase (de Wet et al., Mol. Cell. Biol. 7 (1987), 725-737), aequorin from Aequorea victoria (Prasher et al., Biochem. 26 (1987), 1326-1332), Renilla luciferase from Renilla reniformis (Lorenz et al, PNAS USA 88 (1991), 4438-4442). The luxA and luxB genes of bacterial luciferase can be fused to produce the fusion gene (Fab₂), which can be expressed to produce a fully functional luciferase protein (Escher et al., PNAS 86: 6528-6532 (1989)). In some examples, luciferases expressed by viruses can require exogenously added substrates such as decanal or coelenterazine for light emission. In other examples, viruses can express a complete lux operon, which can include proteins that can provide luciferase substrates such as decanal. For example, viruses containing the complete lux operon sequence, when injected intraperitoneally, intramuscularly, or intravenously, allowed the visualization and localization of microorganisms in live mice indicating that the luciferase light emission can penetrate the tissues and can be detected externally (Contag et al. (1995) Mol. Microbiol. 18: 593-603).

Exemplary fluorescent proteins include green fluorescent protein from Aequorea victoria (Prasher et al., Gene 111: 229-233 (1987)), and GFP variants and variants of GFP-like proteins. Such fluorescent proteins include monomeric, dimeric and tetrameric fluorescent proteins. Exemplary monomeric fluorescent proteins include, but are not limited to: violet fluorescent proteins, such as for example, Sirius; blue fluorescent proteins, such as for example, Azurite, EBFP, SBFP2, EBFP2, TagBFP; cyan fluorescent proteins, such as for example, mTurquoise, eCFP, Cerulean, SCFP, TagCFP, mTFP1; green fluorescent proteins, such as for example, GFP, mUkG1, aAG1, AcGFP1, TagGFP2, EGFP, mWasabi, EmGFP (Emerald); yellow fluorescent proteins, such as for example; TagYFP, EYFP, Topaz, SYFP2, YPet, Venus, Citrine; orange fluorescent proteins, such as for example, mKO, mKO2, mOrange, mOrange2, red fluorescent proteins, such as for example; TagRFP, TagRFPt, mStrawberry, mRuby, mCherry; far red fluorescent proteins, such as for example; mRasberry, mKate2, mPlum, and mNeptune; and fluorescent proteins having an increased stokes shift (i.e. >100 nm distance between excitation and emission spectra), such as for example, Sapphire, T-Sapphire, mAmetrine, and mKeima. Exemplary dimeric and tetrameric fluorescent proteins include, but are not limited to: AmCyanl, Midori-Ishi Cyan, copGFP (ppluGFP2), TurboGFP, ZsGreen, TurboYFP, ZsYellow1, TurboRFP, tdTomato, DsRed2, DsRed-Express, DsRed-Express2, DsRed-Max, AsRed2, TurboFP602, RFP611, Katushka (TurboFP635), Katushka2, and AQ143. Excitation and emission spectra for exemplary fluorescent proteins are well-known in the art (see also e.g., Chudakov et al. (2010) Physiol Rev 90, 1102-1163).

Exemplary detectable proteins also include proteins that can bind a contrasting agent, chromophore, or a compound or ligand that can be detected, such as a transferrin receptor or a ferritin; and reporter proteins, such as E. coli β-galactosidase, β-glucuronidase, xanthine-guanine phosphoribosyltransferase (gpt).

Exemplary detectable proteins are also gene products that can specifically bind a detectable compound, including, but not limited to receptors, metal binding proteins (e.g., siderophores, ferritins, transferrin receptors), ligand binding proteins, and antibodies. Other examples of detectable proteins include transporter proteins that can bind to and transport detectable molecules also are examples of detectable proteins. Such molecules can be used for detection of the virus, such as for applications involving imaging. Any of a variety of detectable compounds can be used, and can be imaged by any of a variety of known imaging methods. Exemplary compounds include receptor ligands and antigens for antibodies. The ligand can be labeled according to the imaging method to be used. Exemplary imaging methods include, but are not limited to, X-rays, magnetic resonance methods, such as magnetic resonance imaging (MRI) and magnetic resonance spectroscopy (MRS), and tomographic methods, including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography and ultrasonic tomography.

Labels appropriate for X-ray imaging are known in the art, and include, for example, Bismuth (III), Gold (III), Lanthanum (III) or Lead (II); a radioactive ion, such as ⁶⁷Copper, ⁶⁷Gallium, ⁶⁸Gallium, ¹¹¹Indium, ¹¹³Indium, ¹²³Iodine, ¹²⁵Iodine, ¹³¹Iodine, ¹⁹⁷Mercury, ²⁰³Mercury, ¹⁸⁶Rhenium, ¹⁸⁸Rhenium, ⁹⁷Rubidium, ¹⁰³Rubidium, ⁹⁹Technetium or ⁹⁰Yttrium; a nuclear magnetic spin-resonance isotope, such as Cobalt (II), Copper (II), Chromium (III), Dysprosium (III), Erbium (III), Gadolinium (III), Holmium (III), Iron (II), Iron (III), Manganese (II), Neodymium (III), Nickel (II), Samarium (III), Terbium (III), Vanadium (II) or Ytterbium UM; or rhodamine or fluorescein.

Labels appropriate for magnetic resonance imaging are known in the art, and include, for example, gadolinium chelates and iron oxides. Use of chelates in contrast agents is known in the art. Labels appropriate for tomographic imaging methods are known in the art, and include, for example, β-emitters such as ¹¹C, ¹³N, ¹⁵O or ⁶⁴Cu or γ-emitters such as ¹²³I. Other exemplary radionuclides that can, be used, for example, as tracers for PET include ⁵⁵Co, ⁶⁷Ga, ⁶⁸Ga, ⁶⁰Cu(II), ⁶⁷Cu(II), ⁵⁷Ni, ⁵²Fe and ¹⁸F (e.g., ¹⁸F-fluorodeoxyglucose (FDG)). Examples of useful radionuclide-labeled agents are a ⁶⁴Cu-labeled engineered antibody fragment (Wu et al. (2002) PNAS USA 97: 8495-8500), ⁶⁴Cu-labeled somatostatin (Lewis et al. (1999) J. Med. Chem. 42: 1341-1347), ⁶⁴Cu-pyruvaldehyde-bis (N4-methylthiosemicarbazone)(⁶⁴Cu-PTSM) (Adonai et al. (2002) PNAS USA 99: 3030-3035), ⁵²Fe-citrate (Leenders et al. (1994) J. Neural. Transm. Suppl. 43: 123-132), ⁵²Fe/^{52m}Mn-citrate (Calonder et al. (1999) J. Neurochem. 73: 2047-2055) and ⁵²Fe-labeled iron (III) hydroxide-sucrose complex (Beshara et al. (1999) Br. J. Haematol. 104: 288-295, 296-302).

Exemplary detectable proteins are transporter proteins that can bind to and transport detectable molecules, such as human epinephrine transporter (hNET) or sodium iodide symporter (NIS) that can bind to and transport detectable molecules, such as MIBG and other labeled molecules (e.g., Na¹²⁵I), into the cell.

The viruses can be modified for purposes of using the viruses for imaging, including for the purpose of dual imaging in vitro and/or in vivo to detect two or more detectable gene products, gene products that produce a detectable signal, gene products that can bind a detectable compound, or gene products that can bind other molecules to form a detectable product. In some examples, the two or more gene products are expressed by different viruses, whereas in other examples the two or more gene products are produced by the same virus. For example, a virus can express a gene product that emits a detectable signal and also express a gene product that catalyzes a detectable reaction. In other examples, a virus can express one or more gene products that emit a detectable signal, one or more gene products that catalyze a detectable reaction, one or more gene products that can bind a detectable compound or that can form a detectable product, or any combination thereof. Any combination of such gene products can be expressed by the viruses provided herein and can be used in combination with any of the methods provided herein. Imaging of such gene products can be performed, for example, by various imaging methods as described herein and known in the art (e.g., fluorescence imaging, MRI, PET, among many other methods of detection). Imaging of gene products also can be performed using the same method, whereby gene products are distinguished by their properties, such as by differences in wavelengths of light emitted. For example, a virus can express more than one fluorescent protein that differs in the wavelength of light emitted (e.g., a GFP and an RFP). In another non-limiting example, an RFP can be expressed with a luciferase. In yet other non-limiting examples, a fluorescent gene product can be expressed with a gene product, such as a ferritin or a transferrin receptor, used for magnetic resonance imaging. A virus expressing two or more detectable gene products or two or more viruses expressing two or more detectable gene products can be imaged in vitro or in vivo using such methods. In some examples the two or more gene products are expressed as a single polypeptide, such as a fusion protein. For example a fluorescent protein can be expressed as a fusion protein with a luciferase protein.

ii. Therapeutic Gene Products

The recombinant vaccinia viruses for insertion of heterologous DNA encoding a chromophore-producing enzyme or enzymes (e.g., a melanin-producing enzyme or enzymes) can contain a heterologous nucleic acid molecule that encodes one or more therapeutic gene products in addition to the chromophore-producing enzymes (melanin-producing enzyme(s)). Therapeutic gene products include products that cause cell death or cause an anti-tumor immune response. A variety of therapeutic gene products, such as toxic or apoptotic proteins, or siRNA, are known in the art, and can be used with the viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound.

Exemplary therapeutic gene products that can be expressed by the viruses provided herein include, but are not limited to, gene products (i.e., proteins and RNAs), including those useful for tumor therapy, such as, but not limited to, an anticancer agent, an antimetastatic agent, or an antiangiogenic agent. For example, exemplary proteins useful for tumor therapy include, but are not limited to, tumor suppressors, cytostatic proteins and costimulatory molecules, such as a cytokine, a chemokine, or other immunomodulatory molecules, an anticancer antibody, such as a single-chain antibody, antisense RNA, siRNA, prodrug converting enzyme, a toxin, a mitosis inhibitor protein, an antitumor oligopeptide, an anticancer polypeptide antibiotic, an angiogenesis inhibitor, or tissue factor. For example, a large number of therapeutic proteins that can be expressed for tumor treatment in the viruses and methods provided herein are known in the art, including, but not limited to, a transporter, a cell-surface receptor, a cytokine, a chemokine, an apoptotic protein, a mitosis inhibitor protein, an antimitotic oligopeptide, an antiangiogenic factor (e.g., hk5), angiogenesis inhibitors (e.g., plasminogen kringle 5 domain, anti-vascular endothelial growth factor (VEGF) scAb, tTF-RGD, truncated human tissue factor-αᵥβ₃-integrin RGD peptide fusion protein), anticancer antibodies, such as a single-chain antibody (e.g., an antitumor antibody or an antiangiogenic antibody, such as an anti-VEGF antibody or an anti-epidermal growth factor receptor (EGFR) antibody), a toxin, a tumor antigen, a prodrug converting enzyme, a ribozyme, RNAi, and siRNA.

Additional therapeutic gene products that can be expressed by the oncolytic reporter viruses include, but are not limited to, cell matrix degradative genes, such as but not limited to, relaxin-1 and MMP9, and genes for tissue regeneration and reprogramming human somatic cells to pluripotency, such as but not limited to, nAG, Oct4, NANOS, Neogenin-1, Ngn3, Pdx1 and Mafa.

Costimulatory molecules for the methods provided herein include any molecules which are capable of enhancing immune responses to an antigen/pathogen in vivo and/or in vitro. Costimulatory molecules also encompass any molecules which promote the activation, proliferation, differentiation, maturation or maintenance of lymphocytes and/or other cells whose function is important or essential for immune responses.

An exemplary, non-limiting list of therapeutic proteins includes tumor growth suppressors such as IL-24, WT1, p53, pseudomonas A endotoxin, diphtheria toxin, Arf, Bax, HSV TK, E. coli purine nucleoside phosphorylase, angiostatin and endostatin, p16, Rb, BRCA1, cystic fibrosis transmembrane regulator (CFTR), Factor VIII, low density lipoprotein receptor, beta-galactosidase, alpha-galactosidase, beta-glucocerebrosidase, insulin, parathyroid hormone, alpha-1-antitrypsin, rsCD40L, Fas-ligand, TRAIL, TNF, antibodies, microcin E492, diphtheria toxin, Pseudomonas exotoxin, Escherichia coli Shiga toxin, Escherichia coli Verotoxin 1, and hyperforin. Exemplary cytokines include, but are not limited to, chemokines and classical cytokines, such as the interleukins, including for example, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factor (GM-CSF), erythropoietin and exemplary chemokines including, but not limited to CXC chemokines such as IL-8 GROα, GROβ, GROγ, ENA-78, LDGF-PBP, GCP-2, PF4, Mig, IP-10, SDF-1α/β, BUNZO/STRC33, I-TAC, BLC/BCA-1; CC chemokines such as MIP-1α, MIP-1β, MDC, TECK, TARC, RANTES, HCC-1, HCC-4, DC-CK1, MIP-3α, MIP-3β, MCP-1, MCP-2, MCP-3, MCP-4, Eotaxin, Eotaxin-2/MPIF-2, I-309, MIP-5/HCC-2, MPIF-1, 6Ckine, CTACK, MEC; lymphotactin; and fractalkine. Other exemplary costimulatory molecules include immunoglobulin superfamily of cytokines, such as B7.1, B7.2.

Exemplary therapeutic proteins that can be expressed by the viruses provided herein and used in the methods provided herein include, but are not limited to, erythropoietin (e.g., SEQ ID NO: 195), an anti-VEGF single chain antibody (e.g., SEQ ID NO: 196), a plasminogen K5 domain (e.g., SEQ ID NO: 197), a human tissue factor-αvβ3-integrin RGD fusion protein (e.g., SEQ ID NO: 173), interleukin-24 (e.g., SEQ ID NO: 174), or immune stimulators, such as SIL-6-SIL-6 receptor fusion protein (e.g., SEQ ID NO: 74).

In some examples, the viruses provided herein can express one or more therapeutic gene products that are proteins that convert a less active compound into a compound that causes tumor cell death. Exemplary methods of conversion of such a prodrug compound include enzymatic conversion and photolytic conversion. A large variety of protein/compound pairs are known in the art, and include, but are not limited to, Herpes simplex virus thymidine kinase/ganciclovir, Herpes simplex virus thymidine kinase/(E)-5-(2-bromovinyl)-2′-deoxyuridine (BVDU), varicella zoster thymidine kinase/ganciclovir, varicella zoster thymidine kinase/BVDU, varicella zoster thymidine kinase/(E)-5-(2-bromovinyl)-1-beta-D-arabinofuranosyluracil (BVaraU), cytosine deaminase/5-fluorouracil, cytosine deaminase/5-fluorocytosine, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid (CMDA), carboxypeptidase A/methotrexate-phenylamine, cytochrome P450/acetaminophen, cytochrome P450-2B1/cyclophosphamide, cytochrome P450-4B1/2-aminoanthracene, 4-ipomeanol, horseradish peroxidase/indole-3-acetic acid, nitroreductase/CB 1954, rabbit carboxylesterase/7-ethyl-10-[4-(1-piperidino)-1-piperidino]carbonyloxy-camptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin glucuronide, thymidine phosphorylase/5′-deoxy-5-fluorouridine, deoxycytidine kinase/cytosine arabinoside, and linamerase/linamarin.

Other therapeutic gene products that can be expressed by the viruses provided herein include siRNA and microRNA molecules. The siRNA and/or microRNA molecule can be directed against expression of a tumor-promoting gene, such as, but not limited to, an oncogene, growth factor, angiogenesis promoting gene, or a receptor. The siRNA and/or microRNA molecule also can be directed against expression of any gene essential for cell growth, cell replication or cell survival. The siRNA and/or microRNA molecule also can be directed against expression of any gene that stabilizes the cell membrane or otherwise limits the number of tumor cell antigens released from the tumor cell. Design of an siRNA or microRNA can be readily determined according to the selected target of the siRNA; methods of siRNA and microRNA design and down-regulation of genes are known in the art, as exemplified in U.S. Pat. Pub. Nos. 2003-0198627 and 2007-0044164, and Zeng et al., Molecular Cell 9:1327-1333 (2002).

Therapeutic gene products include viral attenuation factors, such as antiviral proteins. Antiviral proteins or peptides can be expressed by the viruses provided herein. Expression of antiviral proteins or peptides can control viral pathogenicity. Exemplary viral attenuation factors include, but are not limited to, virus-specific antibodies, mucins, thrombospondin, and soluble proteins such as cytokines, including, but not limited to TNFα, interferons (for example IFNα, IFNβ, or IFNγ) and interleukins (for example IL-1, IL-12 or IL-18).

Another exemplary therapeutic gene product that can be expressed by the viruses provided herein is a protein ligand, such as antitumor oligopeptide. Antitumor oligopeptides are short protein peptides with high affinity and specificity to tumors. Such oligopeptides could be enriched and identified using tumor-associated phage libraries (Akita et al. (2006) Cancer Sci. 97(10):1075-1081). These oligopeptides have been shown to enhance chemotherapy (U.S. Pat. No. 4,912,199). The oligopeptides can be expressed by the viruses provided herein. Expression of the oligopeptides can elicit anticancer activities on their own or in combination with other chemotherapeutic agents. An exemplary group of antitumor oligopeptides is antimitotic peptides, including, but not limited to, tubulysin (Khalil et al. (2006) Chembiochem. 7(4):678-683), phomopsin, hemiasterlin, taltobulin (HTI-286, 3), and cryptophycin. Tubulysin is from myxobacteria and can induce depletion of cell microtubules and trigger the apoptotic process. The antimitotic peptides can be expressed by the viruses provide herein and elicit anticancer activities on their own or in combination with other therapeutic modalities.

Another exemplary therapeutic gene product that can be expressed by the viruses provided herein is an anti-metastatic agent that inhibits one or more steps of the metastatic cascade. The encoded anti-metastatic agents include agents that inhibit invasion of local tissue, inhibit intravasation into the bloodstream or lymphatics, inhibit cell survival and transport through the bloodstream or lymphatics as emboli or potentially single cells, inhibit cell lodging in microvasculature at the secondary site, inhibit growth into microscopic lesions and subsequently into overt metastatic lesions, and/or inhibit metastasis formation and growth within the primary tumor, where the inhibition of metastasis formation is not a consequence of inhibition of primary tumor growth.

Exemplary anti-metastatic agents expressed by the viruses provided herein can directly or indirectly inhibit one or more steps of the metastatic cascade. Exemplary anti-metastatic agents include, but are not limited to, the following: BRMS-1 (Breast Cancer Metastasis Suppressor 1), CRMP-1 (Collapsin Response Mediator Protein-1), CRSP-3 (Cofactor Required for Sp1 transcriptional activation subunit 3), CTGF (Connective Tissue Growth Factor), DRG-1 (Developmentally-regulated GTP-binding protein 1), E-Cad (E-cadherin), gelsolin, KAI1, KiSS1 (Kisspeptin 1/Metastin), kispeptin-10, kispeptin-13, kispeptin-14, kispeptin-54, LKB1 (STK11 (serine/threonine kinase 11)), JNKK1/MKK4 (c-Jun-NH2-Kinase Kinase/Mitogen activated Kinase Kinase 4), MKK6 (mitogen activated kinase kinase 6), MKK7 (mitogen activated kinase kinase 7), Nm23 (NDP Kinase A), RASSF1-8 (Ras association (RalGDS/AF-6) domain family members), RKIP (Raf kinase inhibitor protein), RhoGDI2 (Rho GDP dissociation inhibitor 2), SSECKS (src-suppressed C-kinase substrate), Syk, TIMP-1 (Tissue inhibitor of metalloproteinase-1), TIMP-2 (Tissue inhibitor of metalloproteinase-2), TIMP-3 (Tissue inhibitor of metalloproteinase-3), TIMP-4 (Tissue inhibitor of metalloproteinase-4), TXNIP/VDUP1 (Thioredoxin-interacting protein). Such list of anti-metastatic agents is not meant to be limiting. Any gene product that can suppress metastasis formation via a mechanism that is independent of inhibition of growth within the primary tumor is encompassed by the designation of an anti-metastatic agent or metastasis suppressor and can be expressed by a virus as provided herein. One of skill in the art can identify anti-metastatic genes and can construct a virus expressing one or more anti-metastatic genes for therapy.

Another exemplary therapeutic gene product that can be expressed by the viruses provided herein is a protein that sequesters molecules or nutrients needed for tumor growth. For example, the virus can express one or more proteins that bind iron, transport iron, or store iron, or a combination thereof. Increased iron uptake and/or storage by expression of such proteins not only, increases contrast for visualization and detection of a tumor or tissue in which the virus accumulates, but also depletes iron from the tumor environment. Iron depletion from the tumor environment removes a vital nutrient from the tumors, thereby deregulating iron hemostasis in tumor cells and delaying tumor progression and/or killing the tumor.

Additionally, iron, or other labeled metals, can be administered to a tumor-bearing subject, either alone, or in a conjugated form. An iron conjugate can include, for example, iron conjugated to an imaging moiety or a therapeutic agent. In some cases, the imaging moiety and therapeutic agent are the same, e.g., a radionuclide. Internalization of iron in the tumor, wound, area of inflammation or infection allows the internalization of iron alone, a supplemental imaging moiety, or a therapeutic agent (which can deliver cytotoxicity specifically to tumor cells or deliver the therapeutic agent for treatment of the wound, area of inflammation or infection). These methods can be combined with any of the other methods provided herein.

The administered virus also can be modified to stimulate humoral and/or cellular immune response in the subject, such as the induction of cytotoxic T lymphocytes responses. For example, the virus can provide prophylactic and therapeutic effects against a tumor infected by the virus or other infectious diseases, by rejection of cells from tumors or lesions using viruses that express immunoreactive antigens (Earl et al., Science 234: 728-831 (1986); Lathe et al., Nature (London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol.3: 495-500 (1996)), or other therapeutic proteins.

For example, the viruses provided herein can be modified to express one or more antigens. Sustained release of the antigen can result in an immune response by the viral-infected host, in which the host can develop antibodies against the antigen and/or the host can develop an immune response against cells expressing the antigen. Exemplary antigens include, but are not limited to, tumor specific antigens, tumor-associated antigens, tissue-specific antigens, bacterial antigens, viral antigens, yeast antigens, fungal antigens, protozoan antigens, parasite antigens and mitogens. Superantigens are antigens that can activate a large immune response, often brought about by a large response of T cells. A variety of superantigens are known in the art including, but not limited to, diphtheria toxin, staphylococcal enterotoxins (SEA, SEB, SEC1, SEC2, SED, SEE and SEH), Toxic Shock Syndrome Toxin 1, Exfoliating Toxins (EXft), Streptococcal Pyrogenic Exotoxin A, B and C (SPE A, B and C), Mouse Mammary Tumor Virus proteins (MMTV), Streptococcal M proteins, Clostridial Perfringens Enterotoxin (CPET), Listeria monocytogenes antigen p60, and mycoplasma arthritis superantigens.

Since many superantigens also are toxins, if expression of a virus of reduced toxicity is desired, the superantigen can be modified to retain at least some of its superantigenicity while reducing its toxicity, resulting in a compound such as a toxoid. A variety of recombinant superantigens and toxoids of superantigens are known in the art, and can readily be expressed in the viruses provided herein. Exemplary toxoids include toxoids of diphtheria toxin, as exemplified in U.S. Pat. No. 6,455,673 and toxoids of Staphylococcal enterotoxins, as exemplified in U.S. Pat. Pub. No. 2003-0009015.

iii. Modifications to Alter Attenuation of the Viruses

The toxicity of the viruses can be modulated. For example, viruses provided herein can be attenuated by addition, deletion and/or modification of nucleic acid in the viral genome. In particular, viruses can be attenuated by increasing transcriptional or translational load. In one example, the virus is attenuated by addition of heterologous nucleic acid that contains an open reading frame that encodes one or more gene products (e.g., a diagnostic gene product or a therapeutic gene product as described above). In another example, the virus is attenuated by modification of heterologous nucleic acid that contains an open reading frame that encodes one or more gene products. In a further example, the heterologous nucleic acid is modified by increasing the length of the open reading frame, removal of all or part of the open reading frame or replacement of all or part of the open reading frame. Such modifications can affect viral toxicity by disruption of one or more viral genes or by increasing or decreasing the transcriptional and/or translational load on the virus (see, e.g., International Patent Publication No. WO 2008/100292).

In another example, the virus can be attenuated by modification or replacement of one or more promoters contained in the virus. Such promoters can be replaced by stronger or weaker promoters, where replacement results in a change in the attenuation of the virus. In one example, a promoter of a virus provided herein is replaced with a natural promoter. In one example, a promoter of a virus provided herein is replaced with a synthetic promoter. Exemplary promoters that can replace a promoter contained in a virus can be a viral promoter, such as a vaccinia viral promoter, and can include a vaccinia early, intermediate, early/late or late promoter. Additional exemplary viral promoters are provided herein and known in the art and can be used to replace a promoter contained in a virus.

In another example, the virus is attenuated by modification of a heterologous nucleic acid contained in the virus by removal of all or a portion of a first heterologous nucleic acid molecule and replacement by a second heterologous nucleic acid molecule, where replacement changes the level of attenuation of the virus. The second heterologous nucleic acid molecule can contain a sequence of nucleotides that encodes a protein or can be a non-coding nucleic acid molecule. In some examples, the second heterologous nucleic acid molecule contains an open reading frame operably linked to a promoter. The second heterologous nucleic acid molecule can contain one or more open reading frames or one or more promoters. Further, the one or more promoters of the second heterologous nucleic acid molecule can be one or more stronger promoters or one or more weaker promoters, or can be a combination or both.

Attenuated vaccinia viruses are known in the art and are described, for example, in U.S. Patent Pub. Nos. US 2005-0031643 (now U.S. Pat. Nos. 7,588,767, 7,588,771 and 7,662,398), US 2008-0193373, US 2009-0098529, US 2009-0053244, US 2009-0155287, US 2009-0081639, US 2009-0117034 and US 2009-0136917, and International Patent Pub. Nos. WO 2005/047458, WO 2008/100292 and WO 2008/150496.

Viruses provided herein also can contain a modification that alters its infectivity or resistance to neutralizing antibodies. In one non-limiting example, deletion of the A35R gene in an vaccinia LIVP strain can decrease the infectivity of the virus. In some examples, the viruses provided herein can be modified to contain a deletion of the A35R gene. Exemplary methods for generating such viruses are described in PCT Publication No. WO2008/100292, which describes vaccinia LIVP viruses GLV-1j87, GLV-1j88 and GLV-1j89, which contain deletion of the A35R gene.

In another non-limiting example, replacement of viral coat proteins (e.g., A34R, which encodes a viral coat glycoprotein) with coat proteins from either more virulent or less virulent virus strains can increase or decrease the clearance of the virus from the subject. In one example, the A34R gene in a vaccinia LIVP strain can be replaced with the A34R gene from vaccinia IHD-J strain. Such replacement can increase the extracellular enveloped virus (EEV) form of vaccinia virus and can increase the resistance of the virus to neutralizing antibodies.

b. Control of Heterologous Gene Expression

In some examples, the heterologous nucleic acid, including heterologous nucleic acid encoding a chromophore-producing enzyme(s), also can contain one or more regulatory sequences to regulate expression of an open reading frame encoding, the heterologous RNA, and/or protein. Suitable regulatory sequences which, for example, are functional in a mammalian host cell are well-known in the art. Expression also can be influenced by one or more proteins or RNA molecules expressed by the virus. Gene regulatory elements, such as promoters and enhancers, possess cell-type specific activities and can be activated by certain induction factors (e.g., hormones, growth factors, cytokines, cytostatic agents, irradiation, heat shock) via responsive elements. Controlled and restricted expression of these genes can be achieved using such regulatory elements as internal promoters to drive the expression of therapeutic genes in viral vector constructs.

For example, the one or more heterologous nucleic acid molecules can be operably linked to a promoter for expression of the heterologous RNA and/or protein. For example, a heterologous nucleic acid that is operably linked to a promoter is also called an expression cassette. Hence, viruses provided herein can have the ability to express one or more heterologous genes. Gene expression can include expression of a protein encoded by a gene and/or expression of an RNA molecule encoded by a gene. In some embodiments, the viruses provided herein can express exogenous genes at levels high enough that permit harvesting products of the exogenous genes from the tumor. Exemplary promoters for the expression of heterologous genes are known in the art. The heterologous nucleic acid can be operatively linked to a native promoter or a heterologous promoter that is not native to the virus. Any suitable promoters, including synthetic and naturally-occurring and modified promoters, can be used. Exemplary promoters include synthetic promoters, including synthetic viral and animal promoters. Native promoters or heterologous promoters include, but are not limited to, viral promoters, such as vaccinia virus and adenovirus promoters.

In one example, the promoter is a poxvirus promoter, such as, for example, a vaccinia virus promoter. Vaccinia viral promoters for the expression of one or more heterologous genes can be synthetic or natural promoters, and include vaccinia early, intermediate, early/late and late promoters. Exemplary vaccinia viral promoters for controlling heterologous gene expression include, but are not limited to, P_{7.5k}, P₁₁ₖ, P_{SE}, P_{SEL}, P_{SL}, HSR, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b or K1 promoters. Other viral promoters include, but are not limited to, adenovirus late promoter, Cowpox ATI promoter, or T7 promoter. Strong late promoters can be used to achieve high levels of expression of the heterologous genes. Early and intermediate-stage promoters also can be used. In one example, the promoters contain early and late promoter elements, for example, the vaccinia virus early/late promoter P_{7.5k}, vaccinia late promoter P₁₁ₖ, a synthetic early/late vaccinia P_{SEL} promoter (Patel et al., (1988) Proc. Natl. Acad. Sci. USA 85: 9431-9435; Davison and Moss, (1989) J Mol Biol 210: 749-769; Davison et al. (1990) Nucleic Acids Res. 18: 4285-4286; Chakrabarti et al. (1997), BioTechniques 23: 1094-1097). The viruses provided herein can exhibit differences in characteristics, such as attenuation, as a result of using a stronger promoter versus a weaker promoter. For example, in vaccinia, synthetic early/late and late promoters are relatively strong promoters, whereas vaccinia synthetic early, P_{7.5k} early/late, P_{7.5k} early, and P₂₈ late promoters are relatively weaker promoters (see e.g., Chakrabarti et al. (1997) BioTechniques 23(6) 1094-1097). Combinations of different promoters can be used to express different gene products in the same virus or two different viruses.

Expression of heterologous genes can be controlled by a constitutive promoter, or by an inducible promoter. For example, gene expression can be made inducible using a tetracycline-regulated promoter, whereby transcription is reversibly turned on or off in the presence of tetracycline or one of its derivative (e.g., doxycycline). In such a system, in the absence of an inducer, a tetracycline repressor (TetR) binds to the tet operator (tetO) to repress the activity of the promoter placed near the operator. In the presence of an inducer that binds to TetR, a conformational change occurs that prevents TetR from remaining bound to the operator, thereby permitting gene transcription.

In other examples, organ or tissue-specific expression can be controlled by regulatory sequences. In order to achieve expression only in the target organ or tissue, for example, a tumor to be treated, the foreign nucleotide sequence can be linked to a tissue specific promoter and used for gene therapy. Such promoters are well-known to those skilled in the art (see, e.g., Zimmermann et al., Neuron 12: 11-24 (1994); Vidal et al., EMBO J. 9: 833-840 (1990); Mayford et al., Cell 81: 891-904 (1995); and Pinkert et al., Genes & Dev. 1: 268-76 (1987)).

As is known in the art, regulatory sequences can permit constitutive expression of the exogenous gene or can permit inducible expression of the exogenous gene. Further, the regulatory sequence can permit control of the level of expression of the exogenous gene. In some examples, such as gene product manufacture and harvesting, the regulatory sequence can result in constitutive, high levels of gene expression. In some examples, such as anti-(gene product) antibody harvesting, the regulatory sequence can result in constitutive, lower levels of gene expression. In tumor therapy examples, a therapeutic protein can be under the control of an internally inducible promoter or an externally inducible promoter.

Hence, expression of heterologous genes can be controlled by a constitutive promoter or by an inducible promoter. Inducible promoters can be used to provide tissue specific expression of the heterologous gene or can be inducible by the addition of a regulatory molecule to provide temporal specific induction of the promoter. In some examples, inducible expression can be under the control of cellular or other factors present in a tumor cell or present in a virus-infected tumor cell. In further examples, inducible expression can be under the control of an administrable substance, including IPTG, RU486 or other known induction compounds. Additional regulatory sequences can be used to control the expression of the one or more heterologous genes inserted the virus. Any of a variety of regulatory sequences are available to one skilled in the art according to known factors and design preferences.

c. Exemplary Modified or Recombinant Viruses (Parental Strains)

Modified or recombinant vaccinia strains containing heterologous nucleic acid encoding a gene product or products have been or can be generated from any of a variety of vaccinia virus strains, including, but not limited to, Western Reserve (WR) (SEQ ID NO: 62), Copenhagen (SEQ ID NO: 63), Tashkent, Tian Tan, Lister, Wyeth, IHD-J, and IHD-W, Brighton, Ankara, MVA, Dairen I, LIPV, LC16M8, LC16MO, LIVP, WR 65-16, NYCBH. Such strains, or modified strains thereof, can be used as the parental or starting strain for insertion of heterologous DNA encoding a chromophore-producing enzyme or enzymes (e.g., a melanin-producing enzyme or enzymes), such as any described above, for use in the methods herein. The chromophore-producing enzyme or enzymes can replace the existing heterologous nucleic acid in the parental strain, or can be in addition to other introduced heterologous nucleic acid.

For example, recombinant vaccinia viruses, such as LIVP viruses, have been generated and are known in the art. Exemplary modified or recombinant vaccinia viruses provided herein for insertion of heterologous DNA encoding a chromophore-producing enzyme or enzymes (e.g., a melanin-producing enzyme or enzymes), such as any set forth above, are those derived from the Lister strain, and in particular the attenuated Lister strain LIVP. The modified LIVP viruses can be modified by insertion, deletion or amino acid replacement of heterologous nucleic acid compared to an LIVP strain having a genome set forth in any one of SEQ ID NOS: 1, 188 or 55-61, or having a genome that exhibits at least 97%, 98%, 99% or more sequence identity to any of SEQ ID NOS: 1, 188 or 55-61.

For example, known modified or recombinant LIVP viruses include GLV-1h68 or derivatives thereof. GLV-1h68 (also named RVGL21, SEQ ID NO: 2; described in U.S. Pat. Pub. No. 2005-0031643, now U.S. Pat. Nos. 7,588,767, 7,588,771, 7,662,398) is an attenuated virus of the LIVP strain containing a genome set forth in SEQ ID NO: 188 that contains DNA insertions in gene loci F14.5L (also designated in LIVP as F3) gene locus, thymidine kinase (TK) gene locus, and hemagglutinin (HA) gene locus with expression cassettes encoding detectable marker proteins. Specifically, GLV-1h68 contains an expression cassette containing a Ruc-GFP cDNA molecule (a fusion of DNA encoding Renilla luciferase and DNA encoding GFP) under the control of a vaccinia synthetic early/late promoter P_{SEL} ((P_{SEL})Ruc-GFP) inserted into the F14.5L gene locus; an expression cassette containing a DNA molecule encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) inserted into the HA gene locus.

Other recombinant LIVP viruses are derived from GLV-1h68 and contain heterologous DNA that encodes a gene product or products (see e.g., U.S. Pub. Nos. US2003-0059400, US2003-0228261, US2007-0202572, US2007-0212727, US2009-0117034, US2009-0098529, U52009-0053244, US2009-0155287, US2009-0081639, US2009-0136917, US2009-0162288, US2010-0062016, US2010-0233078 and US2010-0196325; U.S. Pat. Nos. 7,588,767, 7,588,771, 7,662,398 and 7,754,221 and 7,763,420; and International Pub. No. WO 2009/139921). Examples of such recombinant viruses include those set forth in Table 5, including but not limited to, GLV-1h64 (set forth in SEQ ID NO: 189); GLV-1h188 (SEQ ID NO: 190), GLV-1h189 (SEQ ID NO: 191), GLV-1h190 (SEQ ID NO: 192), GLV-1h253 (SEQ ID NO: 193), and GLV-1h254 (SEQ ID NO: 3); GLV-1h311 (SEQ ID NO:65); GLV-1h312 (SEQ ID NO:66); GLV-1h330 (SEQ ID NO:210); or GLV-1h354 (SEQ ID NO:211).

Modified vaccinia viruses also include viruses that are modified by introduction of heterologous nucleic acid into an LIVP strain containing a genome set forth in any of SEQ ID NO: 55-61, or a genome that exhibits at least 97%, 98%, 99% or more sequence identity to any of SEQ ID NOS:55-61. For example, an exemplary modified vaccinia virus is a virus that is modified by insertion, deletion or replacement of heterologous nucleic acid compared to an LIVP strain having a genome set forth in SEQ ID NO: 55. An example of such as strain is GLV-2b372, which contains TurboFP635 (Far-red fluorescent protein “katushka”; set forth in SEQ ID NO: 50) under the control of the vaccinia synthetic early/late promoter at the TK locus. The genome of GLV-2b372 has the sequence of nucleotides set forth in SEQ ID NO: 194.

Exemplary modified or recombinant vaccinia viruses provided herein for insertion of heterologous DNA encoding a chromophore-producing enzyme or enzymes (e.g., a melanin-producing enzyme or enzymes), such as any set forth above, also include those derived from the WR strain. Examples of such recombinant viruses include those set forth in Table 5, including but not limited to, GLV-0b348 (set forth in SEQ ID NO: 201); GLV-0b358 (SEQ ID NO: 202), or GLV-0b365 (SEQ ID NO:203).

Table 5 sets forth exemplary viruses, the reference or parental vaccinia virus (e.g., LIVP set forth in SEQ ID NO: 1 or 188 or GLV-1h68 set forth in SEQ ID NO: 2) and the resulting genotype. The exemplary modifications of the Lister strain can be adapted to other vaccinia viruses (e.g., Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, NYCBH). Any of these viruses, and other oncolytic viruses known in the art, can be further modified to encode a chromophore-producing enzyme or enzymes.

TABLE 5
Recombinant Viruses

| | | Genotype | | | | |
|---|---|---|---|---|---|---|
| Virus | Parent | | | A56R (HA | | |
| Name | Virus | F14.5L | J2R (TK locus) | locus) | A34R | A35R |

LIVP- & GLV-1h68-derived Virus Strains

| | | | | | | |
|---|---|---|---|---|---|---|
| GLV-1h68 | LIVP | (PSEL)Ruc-GFP | (PSEL)rTrfR- | (P11)gusA | wt | wt |
| | | | (P7.5)lacZ | | | |
| GLV-1i69 | GLV-1h68 | (PSEL)Ruc-GFP | (PSEL)rTrfR- | (P11)gusA | A34R from | wt |
| | | | (P7.5)lacZ | | IHD-J | |
| GLV-1h70 | GLV-1h68 | (PSEL)Ruc-GFP | (PSEL)rTrfR- | ko | wt | wt |
| | | | (P7.5)lacZ | | | |
| GLV-1h71 | GLV-1h68 | ko | (PSEL)rTrfR- | (P11)gusA | wt | wt |
| | | | (P7.5)lacZ | | | |
| GLV-1h72 | GLV-1h68 | (PSEL)Ruc-GFP | ko | (P11)gusA | wt | wt |
| GLV-1h73 | GLV-1h70 | ko | (PSEL)rTrfR- | ko | wt | wt |
| | | | (P7.5)lacZ | | | |
| GLV-1h74 | GLV-1h73 | ko | ko | ko | wt | wt |
| GLV-1h76 | GLV-1h68 | (PSEL)Ruc-GFP | (PSE)GM-CSF | (P11)gusA | wt | wt |
| GLV-1h77 | GLV-1h68 | (PSEL)Ruc-GFP | (PSEL)GM-CSF | (P11)gusA | wt | wt |
| GLV-1h78 | GLV-1h68 | (PSEL)Ruc-GFP | (PSL)GM-CSF | (P11)gusA | wt | wt |
| GLV-1h79 | GLV-1h68 | (PSEL)Ruc-GFP | (PSEL)mMCP-1 | (P11)gusA | wt | wt |
| GLV-1h80 | GLV-1h68 | (PSEL)Ruc-GFP | (PSL)mMCP-1 | (P11)gusA | wt | wt |
| GLV-1h81 | GLV-1h68 | (PSEL)Ruc-GFP | (PSEL)rTrfR- | (PSEL)hk5 | wt | wt |
| | | | (P7.5)lacZ | | | |
| GLV-1h82 | GLV-1h22 | (PSEL)Ruc-GFP | (PSEL)TrfR- | (PSEL)ftn | wt | wt |
| | | | (P7.5)lacZ | | | |
| GLV-1h83 | GLV-1h68 | (PSEL)Ruc-GFP | (PSEL)rTrfR- | (PSEL)ftn | wt | wt |
| | | | (P7.5)lacZ | | | |
| GLV-1h84 | GLV-1h68 | ko | (PSEL)CBG99- | ko | wt | wt |
| | | | mRFP1 | | | |
| GLV-1h85 | GLV-1h72 | ko | ko | (P11)gusA | wt | wt |
| GLV-1h86 | GLV-1h72 | (PSEL)Ruc-GFP | ko | ko | wt | wt |
| GLV-1j87 | GLV-1h68 | (PSEL)Ruc-GFP | (PSEL)rTrfR- | (P11)gusA | wt | ko |
| | | | (P7.5)lacZ | | | |
| GLV-1j88 | GLV-1h73 | ko | (PSEL)rTrfR- | ko | wt | ko |
| | | | (P7.5)lacZ | | | |
| GLV-1j89 | GLV-1h74 | ko | ko | ko | wt | ko |
| GLV-1h90 | GLV-1h68 | (PSEL)Ruc-GFP | (PSEL)rTrfR- | (PSE)sIL- | wt | wt |
| | | | (P7.5)lacZ | 6R/IL-6 | | |
| GLV-1h91 | GLV-1h68 | (PSEL)Ruc-GFP | (PSEL)rTrfR- | (PSEL)sIL- | wt | wt |
| | | | (P7.5)lacZ | 6R/IL-6 | | |
| GLV-1h92 | GLV-1h68 | (PSEL)Ruc-GFP | (PSEL)rTrfR- | (PSL)sIL- | wt | wt |
| | | | (P7.5)lacZ | 6R/IL-6 | | |
| GLV-1h93 | GLV-1h68 | (PSEL)Ruc-GFP | (PSEL)rTrfR- | (PSE)FCU1 | wt | wt |
| | | | (P7.5)lacZ | | | |
| GLV-1h94 | GLV-1h68 | (PSEL)Ruc-GFP | (PSEL)rTrfR- | (PSEL)FCU1 | wt | wt |
| | | | (P7.5)lacZ | | | |
| GLV-1h95 | GLV-1h68 | (PSEL)Ruc-GFP | (PSEL)rTrfR- | (PSL)FCU1 | wt | wt |
| | | | (P7.5)lacZ | | | |
| GLV-1h96 | GLV-1h68 | (PSE)IL-24 | (PSEL)rTrfR- | (P11)gusA | wt | wt |
| | | | (P7.5)lacZ | | | |
| GLV-1h97 | GLV-1h68 | (PSEL)IL-24 | (PSEL)rTrfR- | (P11)gusA | wt | wt |
| | | | (P7.5)lacZ | | | |
| GLV-1h98 | GLV-1h68 | (PSL)IL-24 | (PSEL)rTrfR- | (P11)gusA | wt | wt |
| | | | (P7.5)lacZ | | | |
| GLV-1h99 | GLV-1h68 | (PSE)hNET | (PSEL)rTrfR- | (P11)gusA | wt | wt |
| | | | (P7.5)lacZ | | | |
| GLV- | GLV-1h68 | (PSEL)Ruc-GFP | (PSE)hNET | (P11)gusA | wt | wt |
| 1h100 | | | | | | |
| GLV- | GLV-1h68 | (PSEL)Ruc-GFP | (PSL)hNET | (P11)gusA | wt | wt |
| 1h101 | | | | | | |
| GLV- | GLV-1h68 | (PSEL)Ruc-GFP | (PSEL)rTrfR- | (PSE)hDMT | wt | wt |
| 1h102 | | | (P7.5)lacZ | | | |
| GLV- | GLV-1h68 | (PSEL)Ruc-GFP | (PSL)hMCP1 | (P11)gusA | wt | wt |
| 1h103 | | | | | | |
| GLV- | GLV-1h68 | (PSEL)Ruc-GFP | (PSE)tTF-RGD | (P11)gusA | wt | wt |
| 1h104 | | | | | | |
| GLV- | GLV-1h68 | (PSEL)Ruc-GFP | (PSE/L)tTF-RGD | (P11)gusA | wt | wt |
| 1h105 | | | | | | |
| GLV- | GLV-1h68 | (PSEL)Ruc-GFP | (PSL)tTF-RGD | (P11)gusA | wt | wt |
| 1h106 | | | | | | |
| GLV- | GLV-1h68 | (PSEL)Ruc-GFP | (PSE)G6-FLAG | (P11)gusA | wt | wt |
| 1h107 | | | | | | |
| GLV- | GLV-1h68 | (PSEL)Ruc-GFP | (PSEL)G6-FLAG | (P11)gusA | wt | wt |
| 1h108 | | | | | | |
| GLV- | GLV-1h68 | (PSEL)Ruc-GFP | (PSL)G6-FLAG | (P11)gusA | wt | wt |
| 1h109 | | | | | | |
| GLV- | GLV-1h68 | (PSEL)Ruc-GFP | (PSEL)rTrfR- | (PSE)bfr | wt | wt |
| 1h110 | | | (P7.5)lacZ | | | |
| GLV- | GLV-1h68 | (PSEL)Ruc-GFP | (PSEL)rTrfR- | (PSEL)bfr | wt | wt |
| 1h111 | | | (P7.5)lacZ | | | |
| GLV- | GLV-1h68 | (PSEL)Ruc-GFP | (PSEL)rTrfR- | (PSL)bfr | wt | wt |
| 1h112 | | | (P7.5)lacZ | | | |
| GLV- | GLV-1h68 | (PSEL)Ruc-GFP | (PSEL)rTrfR- | (PSEL)bfrₒₚₜ | wt | wt |
| 1h113 | | | (P7.5)lacZ | | | |
| GLV- | GLV-1h68 | (PSEL)Ruc-GFP | (PSEL)rTrfR- | (PSE)mtr | wt | wt |
| 1h114 | | | (P7.5)lacZ | | | |
| GLV- | GLV-1h68 | (PSEL)Ruc-GFP | (PSEL)rTrfR- | (PSEL)mtr | wt | wt |
| 1h115 | | | (P7.5)lacZ | | | |
| GLV- | GLV-1h68 | (PSEL)Ruc-GFP | (PSE)mMnSOD | (P11)gusA | wt | wt |
| 1h116 | | | | | | |
| GLV- | GLV-1h68 | (PSEL)Ruc-GFP | (PSEL)mMnSOD | (P11)gusA | wt | wt |
| 1h117 | | | | | | |
| GLV- | GLV-1h68 | (PSEL)Ruc-GFP | (PSL)mMnSOD | (P11)gusA | wt | wt |
| 1h118 | | | | | | |
| GLV- | GLV-1h68 | (PSEL)Ruc-GFP | (PSE)mIP-10 | (P11)gusA | wt | wt |
| 1h119 | | | | | | |
| GLV- | GLV-1h68 | (PSEL)Ruc-GFP | (PSEL)mIP-10 | (P11)gusA | wt | wt |
| 1h120 | | | | | | |
| GLV- | GLV-1h68 | (PSEL)Ruc-GFP | (PSL)mIP-10 | (P11)gusA | wt | wt |
| 1h121 | | | | | | |
| GLV- | GLV-1h68 | (PSEL)Ruc-GFP | (PSE)mLIGHT | (P11)gusA | wt | wt |
| 1h122 | | | | | | |
| GLV- | GLV-1h68 | (PSEL)Ruc-GFP | (PSE/L)mLIGHT | (P11)gusA | wt | wt |
| 1h123 | | | | | | |
| GLV- | GLV-1h68 | (PSEL)Ruc-GFP | (PSL)mLIGHT | (P11)gusA | wt | wt |
| 1h124 | | | | | | |
| GLV- | GLV-1h68 | (PSEL)Ruc-GFP | (PSE)CBP | (P11)gusA | wt | wt |
| 1h125 | | | | | | |
| GLV- | GLV-1h68 | (PSEL)Ruc-GFP | (PSEL)CBP | (P11)gusA | wt | wt |
| 1h126 | | | | | | |
| GLV- | GLV-1h68 | (PSEL)Ruc-GFP | (PSL)CBP | (P11)gusA | wt | wt |
| 1h127 | | | | | | |
| GLV- | GLV-1h68 | (PSEL)Ruc-GFP | (PSE)P60 | (P11)gusA | wt | wt |
| 1h128 | | | | | | |
| GLV- | GLV-1h68 | (PSEL)Ruc-GFP | (PSEL)P60 | (P11)gusA | wt | wt |
| 1h129 | | | | | | |
| GLV- | GLV-1h68 | (PSEL)Ruc-GFP | (PSL)P60 | (P11)gusA | wt | wt |
| 1h130 | | | | | | |
| GLV- | GLV-1h68 | (PSEL)Ruc-GFP | (PSE)hFLH | (P11)gusA | wt | wt |
| 1h131 | | | | | | |
| GLV- | GLV-1h68 | (PSEL)Ruc-GFP | (PSEL)hFLH | (P11)gusA | wt | wt |
| 1h132 | | | | | | |
| GLV- | GLV-1h68 | (PSEL)Ruc-GFP | (PSL)hFLH | (P11)gusA | wt | wt |
| 1h133 | | | | | | |
| GLV- | GLV-1h68 | (PSEL)CBG99- | (PSEL)rTrfR- | (P11)gusA | wt | wt |
| 1h134 | | mRFP1 | (P7.5)lacZ | | | |
| GLV- | GLV-1h68 | wt | (PSEL)rTrfR- | (P11)gusA | wt | wt |
| 1e135 | | | (P7.5)lacZ | | | |
| GLV- | GLV-1h68 | (PSEL)Ruc-GFP | (PSE)PEDF | (P11)gusA | wt | wt |
| 1h136 | | | | | | |
| GLV- | GLV-1h68 | (PSEL)Ruc-GFP | (PSEL)PEDF | (P11)gusA | wt | wt |
| 1h137 | | | | | | |
| GLV- | GLV-1h68 | (PSEL)Ruc-GFP | (PSL)PEDF | (P11)gusA | wt | wt |
| 1h138 | | | | | | |
| GLV- | GLV-1h68 | (PSEL)Ruc-GFP | (PSEL)rTrfR- | (PSE)hNET | wt | wt |
| 1h139 | | | (P7.5)lacZ | | | |
| GLV- | GLV-1h68 | (PSEL)Ruc-GFP | (PSE)CYP11B1 | (P11)gusA | wt | wt |
| 1h140 | | | | | | |
| GLV- | GLV-1h68 | (PSEL)Ruc-GFP | (PSEL)CYP11B1 | (P11)gusA | wt | wt |
| 1h141 | | | | | | |
| GLV- | GLV-1h68 | (PSEL)Ruc-GFP | (PSL)CYP11B1 | (P11)gusA | wt | wt |
| 1h142 | | | | | | |
| GLV- | GLV-1h68 | (PSEL)Ruc-GFP | (PSE)CYP11B2 | (P11)gusA | wt | wt |
| 1h143 | | | | | | |
| GLV- | GLV-1h68 | (PSEL)Ruc-GFP | (PSEL)CYP11B2 | (P11)gusA | wt | wt |
| 1h144 | | | | | | |
| GLV- | GLV-1h68 | (PSEL)Ruc-GFP | (PSL)CYP11B2 | (P11)gusA | wt | wt |
| 1h145 | | | | | | |
| GLV- | GLV- | (PSEL)Ruc-GFP | (PSE)hNET | (PSE)IL-24 | wt | wt |
| 1h146 | 1h100 | | | | | |
| GLV- | GLV-1h68 | (PSEL)Ruc-GFP | (PSE)HACE1 | (P11)gusA | wt | wt |
| 1h147 | | | | | | |
| GLV- | GLV-1h68 | (PSEL)Ruc-GFP | (PSEL)HACE1 | (P11)gusA | wt | wt |
| 1h148 | | | | | | |
| GLV- | GLV-1h68 | (PSEL)Ruc-GFP | (PSL)HACE1 | (P11)gusA | wt | wt |
| 1h149 | | | | | | |
| GLV- | GLV- | (PSEL)Ruc-GFP | (PSL)hNET | (PSE)IL-24 | wt | wt |
| 1h150 | 1h101 | | | | | |
| GLV- | GLV-1h68 | (PSEL) Ruc-GFP | (PSE/L)TfR- | (PSE)hNIS | wt | wt |
| 1h151 | | | (P7.5)lacZ | | | |
| GLV- | GLV-1h68 | (PSEL) Ruc-GFP | (PSEL)TfR- | (PSE)hNISa | wt | wt |
| 1h153 | | | (P7.5)lacZ | | | |
| GLV- | GLV-1h22 | (PSEL) Ruc-GFP | (PSEL)TfR- | (PSEL)bfrₒₚₜ | wt | wt |
| 1h154 | | | (P7.5)lacZ | | | |
| GLV- | GLV-1h22 | (PSEL) Ruc-GFP | (PSEL)TfR- | (PSEL)hFH | wt | wt |
| 1h155 | | | (P7.5)lacZ | | | |
| GLV- | GLV- | (PSEL) Ruc-GFP | (PSEL)mtr | (PSEL)bfrₒₚₜ | wt | wt |
| 1h156 | 1h113 | | | | | |
| GLV- | GLV-1h68 | (PSEL) Ruc-GFP | (PSEL)mtr | (PSEL)hFH | wt | wt |
| 1h157 | | | | | | |
| GLV- | GLV-1h68 | (PSEL) Ruc-GFP | (PSEL)TfR- | (PSEL)G6- | wt | wt |
| 1h158 | | | (P7.5)lacZ | scAb | | |
| GLV- | GLV-1h68 | (PSEL) Ruc-GFP | (PSEL)TfR- | (PSL)G6- | wt | wt |
| 1h159 | | | (P7.5)lacZ | scAb | | |
| GLV- | GLV-1h68 | (PSEL)Ruc-GFP | (PSEL)luxAB | (P11)gusA | wt | wt |
| 1h160 | | | | | | |
| GLV- | GLV-1h68 | (PSEL)Ruc-GFP | (PSEL)TfR- | (PSEL)luxCD | wt | wt |
| 1h161 | | | (P7.5)lacZ | | | |
| GLV- | GLV-1h68 | (PSEL)luxE | (PSEL)rTrfR- | (P11)gusA | wt | wt |
| 1h162 | | | (P7.5)lacZ | | | |
| GLV- | GLV- | (PSEL)Ruc-GFP | (PSE)hNET | (PSEL)G6- | wt | wt |
| 1h163 | 1h100 | | | scAb | | |
| GLV- | GLV- | (PSEL)Ruc-GFP | (PSE)hNET | (PSEL)G6- | wt | wt |
| 1h164 | 1h100 | | | scAb | | |
| GLV- | GLV-1h68 | (PSEL)Ruc-GFP | (PSE)nAG | (P11)gusA | wt | wt |
| 1h165 | | | | | | |
| GLV- | GLV-1h68 | (PSEL)Ruc-GFP | (PSEL)NAG | (P11)gusA | wt | wt |
| 1h166 | | | | | | |
| GLV- | GLV-1h68 | (PSEL)Ruc-GFP | (PSL)nAG | (P11)gusA | wt | wt |
| 1h167 | | | | | | |
| GLV- | GLV-1h68 | (PSEL)Ruc-GFP | (PSE)RLN | (P11)gusA | wt | wt |
| 1h168 | | | | | | |
| GLV- | GLV-1h68 | (PSEL)Ruc-GFP | (PSEL)RLN | (P11)gusA | wt | wt |
| 1h169 | | | | | | |
| GLV- | GLV-1h68 | (PSEL)Ruc-GFP | (PSL)RLN | (P11)gusA | wt | wt |
| 1h170 | | | | | | |
| GLV- | GLV-1h68 | (PSEL)Ruc-GFP | (PSE)NM23A | (P11)gusA | wt | wt |
| 1h171 | | | | | | |
| GLV- | GLV-1h68 | (PSEL)Ruc-GFP | (PSEL)NM23A | (P11)gusA | wt | wt |
| 1h172 | | | | | | |
| GLV- | GLV-1h68 | (PSEL)Ruc-GFP | (PSL)NM23 | (P11)gusA | wt | wt |
| 1h173 | | | | | | |
| GLV- | GLV-1h68 | (PSEL)Ruc-GFP | (PSE)NPPA1 | (P11)gusA | wt | wt |
| 1h174 | | | | | | |
| GLV- | GLV-1h68 | (PSEL)Ruc-GFP | (PSEL)NPPA1 | (P11)gusA | wt | wt |
| 1h175 | | | | | | |
| GLV- | GLV-1h68 | (PSEL)Ruc-GFP | (PSL)NPPA1 | (P11)gusA | wt | wt |
| 1h176 | | | | | | |
| GLV- | GLV-1h68 | (PSEL)Ruc-GFP | (PSE)STAT1α | (P11)gusA | wt | wt |
| 1h177 | | | | | | |
| GLV- | GLV-1h68 | (PSEL)Ruc-GFP | (PSEL)STAT1α | (P11)gusA | wt | wt |
| 1h178 | | | | | | |
| GLV- | GLV-1h68 | (PSEL)Ruc-GFP | (PSL)STAT1α | (P11)gusA | wt | wt |
| 1h179 | | | | | | |
| GLV- | GLV-1h68 | (PSEL)Ruc-GFP | (PSE)CPG2 | (P11)gusA | wt | wt |
| 1h180 | | | | | | |
| GLV- | GLV-1h68 | (PSEL)Ruc-GFP | (PSEL)CPG2 | (P11)gusA | wt | wt |
| 1h181 | | | | | | |
| GLV- | GLV-1h68 | (PSEL)Ruc-GFP | (PSL)CPG2 | (P11)gusA | wt | wt |
| 1h182 | | | | | | |
| GLV- | GLV-1h68 | (PSEL)Ruc-GFP | (PSE)Ecad | (P11)gusA | wt | wt |
| 1h183 | | | | | | |
| GLV- | GLV-1h68 | (PSEL) Ruc-GFP | (PSE/L)TfR- | (PSE)magA | wt | wt |
| 1h184 | | | (P7.5)lacZ | | | |
| GLV- | GLV-1h68 | (PSEL)Ruc-GFP | (PSL)Ecad | (P11)gusA | wt | wt |
| 1h185 | | | | | | |
| GLV- | GLV-1h68 | (PSEL) Ruc-GFP | (PSEL)TfR- | (PSEL)FTL | wt | wt |
| 1h186 | | | (P7.5)lacZ | 498- | | |
| | | | | 499InsTC | | |
| GLV- | GLV-1h68 | (PSEL) Ruc-GFP | (PSEL)TfR- | (PSEL)FTL | wt | wt |
| 1h187 | | | (P7.5)lacZ | | | |
| GLV- | GLV-1h68 | (PSEL) Ruc-GFP | (PSEL)TfR- | (PSE)FUKW | wt | wt |
| 1h188 | | | (P7.5)lacZ | | | |
| GLV- | GLV-1h68 | (PSEL) Ruc-GFP | (PSEL)TfR- | (PSEL)FUKW | wt | wt |
| 1h189 | | | (P7.5)lacZ | | | |
| GLV- | GLV-1h68 | (PSEL) Ruc-GFP | (PSEL)TfR- | (PSL)FUKW | wt | wt |
| 1h190 | | | (P7.5)lacZ | | | |
| GLV- | GLV-1h68 | (PSEL)Ruc-GFP | (PSE)STAT1β | (P11)gusA | wt | wt |
| 1h191 | | | | | | |
| GLV- | GLV-1h68 | (PSEL)Ruc-GFP | (PSEL)STAT1β | (P11)gusA | wt | wt |
| 1h192 | | | | | | |
| GLV- | GLV-1h68 | (PSEL)Ruc-GFP | (PSL)STAT1β | (P11)gusA | wt | wt |
| 1h193 | | | | | | |
| GLV- | GLV- | (PSE)luxE | (PSEL)TfR- | (PSEL)luxCD | wt | wt |
| 1h194 | 1h161 | | (P7.5)lacZ | | | |
| GLV- | GLV- | (PSEL)Ruc-GFP | (PSE)luxAB | (PSEL)luxCD | wt | wt |
| 1h195 | 1h161 | | | | | |
| GLV- | GLV-1h68 | (PSEL)Ruc-GFP | (PSE)181a | (P11)gusA | wt | wt |
| 1h196 | | | | | | |
| GLV- | GLV-1h68 | | | | | |
| 1h197 | | | | | | |
| GLV- | GLV-1h68 | (PSEL)Ruc-GFP | (PSL)181a | (P11)gusA | wt | wt |
| 1h198 | | | | | | |
| GLV- | GLV-1h68 | (PSEL)Ruc-GFP | (PSE)335 | (P11)gusA | wt | wt |
| 1h199 | | | | | | |
| GLV- | GLV-1h68 | (PSEL)Ruc-GFP | (PSL)335 | (P11)gusA | wt | wt |
| 1h201 | | | | | | |
| GLV- | GLV-1h68 | | | | | |
| 1h202 | | | | | | |
| GLV- | GLV-1h68 | (PSEL)Ruc-GFP | (PSEL)126 | (P11)gusA | wt | wt |
| 1h203 | | | | | | |
| GLV- | GLV-1h68 | | | | | |
| 1h204 | | | | | | |
| GLV- | GLV-1h68 | (PSEL) Ruc-GFP | (PSEL)TfR- | (PSE)NAN | wt | wt |
| 1h205 | | | (P7.5)lacZ | OG | | |
| GLV- | GLV-1h68 | (PSEL) Ruc-GFP | (PSEL)TfR- | (PSE)Oct4 | wt | wt |
| 1h208 | | | (P7.5)lacZ | | | |
| GLV- | GLV-1h68 | (PSEL)Ruc-GFP | (P7.5E)hEPO | (P11)gusA | wt | wt |
| 1h210 | | | | | | |
| GLV- | GLV-1h68 | (PSEL)Ruc-GFP | (PSE)hEPO | (P11)gusA | wt | wt |
| 1h211 | | | | | | |
| GLV- | GLV-1h68 | (PSEL)Ruc-GFP | (PSEL)hEPO | (P11)gusA | wt | wt |
| 1h212 | | | | | | |
| GLV- | GLV-1h68 | (PSEL)Ruc-GFP | (PSL)hEPO | (P11)gusA | wt | wt |
| 1h213 | | | | | | |
| GLV- | GLV-1h68 | (PSEL)Ruc-GFP | (PSE)OspF | (P11)gusA | wt | wt |
| 1h214 | | | | | | |
| GLV- | GLV-1h68 | (PSEL)Ruc-GFP | (PSE)OspG | (P11)gusA | wt | wt |
| 1h215 | | | | | | |
| GLV- | GLV-1h68 | (PSEL)Ruc-GFP | (PSEL)OspG | (P11)gusA | wt | wt |
| 1h216 | | | | | | |
| GLV- | GLV-1h68 | (PSEL)Ruc-GFP | (PSL)OspG | (P11)gusA | wt | wt |
| 1h217 | | | | | | |
| GLV- | GLV-1h84 | ko | (PSEL)CBG99- | (PSE)RLN | wt | wt |
| 1h218 | | | mRFP1 | | | |
| GLV- | GLV-1h84 | ko | (PSEL)CBG99- | (PSEL)RLN | wt | wt |
| 1h219 | | | mRFP1 | | | |
| GLV- | GLV-1h84 | ko | (PSEL)CBG99- | (PSL)RLN | wt | wt |
| 1h220 | | | mRFP1 | | | |
| GLV- | GLV- | (PSE)luxE | (PSEL)luxAB | (P11)gusA | wt | wt |
| 1h221 | 1h160 | | | | | |
| GLV- | GLV-1h68 | (PSEL)Ruc-GFP | (PSE)Ngn3 | (P11)gusA | wt | wt |
| 1h222 | | | | | | |
| GLV- | GLV-1h68 | (PSEL)Ruc-GFP | (PSEL)Ngn3 | (P11)gusA | wt | wt |
| 1h223 | | | | | | |
| GLV- | GLV-1h68 | (PSEL)Ruc-GFP | (PSL)Ngn3 | (P11)gusA | wt | wt |
| 1h224 | | | | | | |
| GLV- | GLV-1h68 | (PSEL)Ruc-GFP | (PSE)hADH | (P11)gusA | wt | wt |
| 1h225 | | | | | | |
| GLV- | GLV-1h68 | (PSEL)Ruc-GFP | (PSEL)hADH | (P11)gusA | wt | wt |
| 1h226 | | | | | | |
| GLV- | GLV-1h68 | (PSEL)Ruc-GFP | (PSL)hADH | (P11)gusA | wt | wt |
| 1h227 | | | | | | |
| GLV- | GLV- | (PSE)luxE | (PSE)luxAB | (PSEL)luxCD | wt | wt |
| 1h228 | 1h194 | | | | | |
| GLV- | GLV- | (PSEL)luxE | (PSE)luxAB | (PSEL)luxCD | wt | wt |
| 1h229 | 1h195 | | | | | |
| GLV- | GLV-1h68 | (PSEL)Ruc-GFP | (PSE)Myc-CTR1 | (P11)gusA | wt | wt |
| 1h230 | | | | | | |
| GLV- | GLV-1h68 | (PSEL)Ruc-GFP | (PSL)Myc-CTR1 | (P11)gusA | wt | wt |
| 1h231 | | | | | | |
| GLV- | GLV-1h68 | (PSEL)Ruc-GFP | (PSE)CTR1 | (P11)gusA | wt | wt |
| 1h232 | | | | | | |
| GLV- | GLV-1h68 | (PSEL)Ruc-GFP | (PSE)mPEDF | (P11)gusA | wt | wt |
| 1h233 | | | | | | |
| GLV- | GLV-1h68 | (PSEL)Ruc-GFP | (PSEL)mPEDF | (P11)gusA | wt | wt |
| 1h234 | | | | | | |
| GLV- | GLV-1h68 | (PSEL)Ruc-GFP | (PSL)mPEDF | (P11)gusA | wt | wt |
| 1h235 | | | | | | |
| GLV- | GLV-1h73 | (PSEL)Ruc-GFP | rtfr(PEL) | (PSE)WTC | wt | wt |
| 1h236 | | | (P7.5)lacZ | DC6 | | |
| GLV- | GLV-1h73 | (PSEL)Ruc-GFP | rtfr(PEL) | (PSE)MutC | wt | wt |
| 1h237 | | | (P7.5)lacZ | DC6 | | |
| GLV- | GLV-1h68 | (PSEL) Ruc-GFP | (PSEL)TfR- | (PSL)CBG9 | wt | wt |
| 1h238 | | | (P7.5)lacZ | 9-mRFP1 | | |
| GLV- | GLV-1h68 | (PSEL)Ruc-GFP | (PSE)GLAF-3 | (P11)gusA | wt | wt |
| 1h239 | | | | | | |
| GLV- | GLV-1h68 | (PSEL)Ruc-GFP | (PSEL)GLAF-3 | (P11)gusA | wt | wt |
| 1h240 | | | | | | |
| GLV- | GLV-1h68 | (PSEL)Ruc-GFP | (PSL)GLAF-3 | (P11)gusA | wt | wt |
| 1h241 | | | | | | |
| GLV- | GLV-1h68 | (PSEL)Ruc-GFP | (PE)luxABCDE | (P11)gusA | wt | wt |
| 1h242 | | | | | | |
| GLV- | GLV- | (PSEL)Ruc-GFP | (PE)luxABCDE | (PSE)frp | wt | wt |
| 1h243 | 1h242 | | | | | |
| GLV- | GLV- | (PSEL) Ruc-GFP | (PSE)hNISa | (PSEL)FUKW | wt | wt |
| 1h244 | 1h189 | | | | | |
| GLV- | GLV- | (PSEL) Ruc-GFP | (PSEL)hNISa | (PSEL)FUKW | wt | wt |
| 1h245 | 1h189 | | | | | |
| GLV- | GLV- | (PSEL) Ruc-GFP | (PSL)hNISa | (PSEL)FUKW | wt | wt |
| 1h246 | 1h189 | | | | | |
| GLV- | GLV-1h68 | (PSEL) Ruc-GFP | (PSEL)TfR- | (PSE)IFP | wt | wt |
| 1h247 | | | (P7.5)lacZ | | | |
| GLV- | GLV-1h168 | (PSEL) Ruc-GFP | (PSEL)TfR | (PSEL)IFP | wt | wt |
| 1h248 | | | (P7.5)lacZ | | | |
| GLV- | GLV-1h68 | (PSEL) Ruc-GFP | (PSEL)TfR- | (PSL)IFP | wt | wt |
| 1h249 | | | (P7.5)lacZ | | | |
| GLV- | GLV- | (PSEL) Ruc-GFP | (PSEL)TfR- | (PSL)FUKW | wt | wt |
| 1h250 | 1h190 | | (P7.5)lacZ | | | |
| GLV- | GLV-1h68 | (PSE)hNISa | (PSEL)rTrfR- | (P11)gusA | wt | wt |
| 1h251 | | | (P7.5)lacZ | | | |
| GLV- | GLV-1h68 | (PSEL)Ruc-GFP | (PSE)hNISa | (P11)gusA | wt | wt |
| 1h252 | | | | | | |
| GLV- | GLV-1h71 | ko | (PSEL)TfR- | (PSE)FUKW | wt | wt |
| 1h253 | | | (P7.5)lacZ | | | |
| GLV- | GLV-1h71 | ko | (PSEL)TfR- | (PSL)FUKW | wt | wt |
| 1h254 | | | (P7.5)lacZ | | | |
| GLV- | GLV-1h68 | (PSEL)Ruc-GFP | (PSE)hMMP9 | (P11)gusA | wt | wt |
| 1h255 | | | | | | |
| GLV- | GLV-1h68 | (PSEL)Ruc-GFP | (PSL)hMMP9 | (P11)gusA | wt | wt |
| 1h256 | | | | | | |
| GLV- | GLV-1h68 | (PSEL) Ruc-GFP | (PSEL)TfR- | (PSE)mNeptune | wt | wt |
| 1h257 | | | (P7.5)lacZ | | | |
| GLV- | GLV-1h68 | (PSEL) Ruc-GFP | (PSEL)TfR- | (PSEL)mNeptune | wt | wt |
| 1h258 | | | (P7.5)lacZ | | | |
| GLV- | GLV-1h68 | (PSEL) Ruc-GFP | (PSEL)TfR- | (PSL)mNeptune | wt | wt |
| 1h259 | | | (P7.5)lacZ | | | |
| GLV- | GLV-1h68 | (PSE)mNeptune | (PSEL)rTrfR- | (P11)gusA | wt | wt |
| 1h260 | | | (P7.5)lacZ | | | |
| GLV- | GLV-1h68 | (PSEL)mNeptune | (PSEL)rTrfR- | (P11)gusA | wt | wt |
| 1h261 | | | (P7.5)lacZ | | | |
| GLV- | GLV-1h68 | (PSL)mNeptune | (PSEL)rTrfR- | (P11)gusA | wt | wt |
| 1h262 | | | (P7.5)lacZ | | | |
| GLV- | GLV- | (PSE)mNeptune | (PSE)hNET | (PSL)G6- | wt | wt |
| 1h263 | 1h164 | | | scAb | | |
| GLV- | GLV- | (PSEL)mNeptune | (PSE)hNET | (PSL)G6- | wt | wt |
| 1h264 | 1h164 | | | scAb | | |
| GLV- | GLV- | (PSL)mNeptune | (PSE)hNET | (PSL)G6- | wt | wt |
| 1h265 | 1h164 | | | scAb | | |
| GLV- | GLV- | (PSEL) Ruc-GFP | (PSE)AlstR | (PSEL)FUKW | wt | wt |
| 1h266 | 1h189 | | | | | |
| GLV- | GLV- | (PSEL) Ruc-GFP | (PSEL)AlstR | (PSEL)FUKW | wt | wt |
| 1h267 | 1h189 | | | | | |
| GLV- | GLV- | (PSEL) Ruc-GFP | (PSL)AlstR | (PSEL)FUKW | wt | wt |
| 1h268 | 1h189 | | | | | |
| GLV- | GLV- | (PSEL) Ruc-GFP | (PSE)PEPR1 | (PSEL)FUKW | wt | wt |
| 1h269 | 1h189 | | | | | |
| GLV- | GLV- | (PSEL) Ruc-GFP | (PSEL)PEPR1 | (PSEL)FUKW | wt | wt |
| 1h270 | 1h189 | | | | | |
| GLV- | GLV- | (PSEL) Ruc-GFP | (PSL)PEPR1 | (PSEL)FUKW | wt | wt |
| 1h271 | 1h189 | | | | | |
| GLV- | GLV- | (PSEL) Ruc-GFP | (PSE)LAT4 | (PSEL)FUKW | wt | wt |
| 1h272 | 1h189 | | | | | |
| GLV- | GLV- | (PSEL) Ruc-GFP | (PSEL)LAT4 | (PSEL)FUKW | wt | wt |
| 1h273 | 1h189 | | | | | |
| GLV- | GLV- | (PSEL) Ruc-GFP | (PSL)LAT4 | (PSEL)FUKW | wt | wt |
| 1h274 | 1h189 | | | | | |
| GLV- | GLV- | (PSEL) Ruc-GFP | (PSE)Cyp51 | (PSEL)FUKW | wt | wt |
| 1h275 | 1h189 | | | | | |
| GLV- | GLV- | (PSEL) Ruc-GFP | (PSEL)Cyp51 | (PSEL)FUKW | wt | wt |
| 1h276 | 1h189 | | | | | |
| GLV- | GLV- | (PSEL) Ruc-GFP | (PSL)Cyp51 | (PSEL)FUKW | wt | wt |
| 1h277 | 1h189 | | | | | |
| GLV- | GLV- | (PSEL)Ruc-GFP | (PSE)BMP4 | (PSEL)FUKW | wt | wt |
| 1h284 | 1h189 | | | | | |
| GLV- | GLV- | (PSEL)Ruc-GFP | (PSEL)BMP4 | (PSEL)FUKW | wt | wt |
| 1h285 | 1h189 | | | | | |
| GLV- | GLV- | (PSEL)Ruc-GFP | (PSL)BMP4 | (PSEL)FUKW | wt | wt |
| 1h286 | 1h189 | | | | | |
| GLV- | GLV-1h68 | (P_{SEL})Ruc-GFP | (P_{SL})tetO-CBG99- | (P₁₁ₖ)gusA | wt | wt |
| 1h311 | | | mRFP | | | |
| GLV- | GLV- | (P_{7.5k})-TetR | (P_{SL})tetO-CBG99- | (P₁₁ₖ)gusA | wt | wt |
| 1h312 | 1h311 | | mRFP | | | |
| GLV- | GLV-1h68 | (P_{7.5})tetR | (P_{SEL})rTrfR- | (P₁₁ₖ)gusA | wt | wt |
| 1h330 | | | (P_{7.5k})LacZ | | | |
| GLV- | GLV- | (P_{SEL})tetR | (P_{SL})tetO-CBG99- | (P₁₁ₖ)gusA | wt | wt |
| 1h354 | 1h311 | | mRFP1 | | | |

LIVP 1.1.1-Derived Virus Strains

| | | | | | | |
|---|---|---|---|---|---|---|
| GLV- | LIVP 1.1.1 | wt | (P_{SEL})FUKW | wt | wt | wt |
| 2b372 | | | | | | |

WR-Derived Strains

| | | | | | | |
|---|---|---|---|---|---|---|
| GLV- | WR | wt | (P_{SL})-tetO-CBG99- | wt | wt | wt |
| 0b348 | | | mRFP1 | | | |
| GLV- | GLV- | wt | (P_{SE})EFNA1 | wt | wt | wt |
| 0b358 | 0b348 | | | | | |
| GLV- | GLV- | wt | (P_{SE})EFNA1 | (P_{SEL})FUKW | wt | wt |
| 0e365 | 0b358 | | | | | |

d. Exemplary Vaccinia Viruses Encoding a Chromophore-Producing Enzyme or Enzymes

Exemplary vaccinia viruses provided herein, for use in the methods and methods of use herein, are vaccinia viruses that contain nucleic acid encoding a chromophore-producing enzyme that is a tyrosinase enzyme. The vaccinia virus can be any vaccinia virus, such as any known in the art or described above. In particular examples, the vaccinia virus is a Lister strain such as an LIVP virus, a clonal strain thereof or a derivative thereof that has been previously modified by insertion of heterologous DNA encoding a therapeutic product or reporter. The vaccinia virus also can be a WR strain, a clonal strain thereof or a derivative thereof that has been previously modified by insertion of heterologous DNA encoding a therapeutic product or reporter. Hence, any of the vaccinia viruses, modified viruses or recombinant viruses described above can be adapted to contain a sequence of nucleotides encoding a tyrosinase enzyme.

The vaccinia virus can be modified by insertion of nucleotides encoding a tyrosinase enzyme, such as any described above. For example, the virus is modified by insertion of a sequence of nucleotides encoding a tyrosinase enzyme having the sequence of amino acids set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence or a sequence of amino acids that exhibits at least 75% sequence identity to any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence. For example, the nucleic acid molecule encodes a tyrosinase enzyme having a sequence of amino acids that exhibits at least 80%, 85%, 86%, 87%, 88%, 89%, 90%, 91%, 92%, 93%, 94%, 95%, 96%, 97%, 98%, 99% or more sequence identity to any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence. The inserted nucleotide sequence can contain the sequence of nucleotides set forth in any of SEQ ID NOS: 6, 80, 83, 86, 88, 90, 92, 94, 96, 98, 100, 102, 104, 106, 108, 110, 112, 114, 116 or 118 or a sequence of nucleotides that exhibits at least 70% sequence identity to any of SEQ ID NOS: 6, 80, 83, 86, 88, 90, 92, 94, 96, 98, 100, 102, 104, 106, 108, 110, 112, 114, 116 or 118, for example, a sequence of nucleotides that exhibits at least 75%, 80%, 85%, 86%, 87%, 88%, 89%, 90%, 91%, 92%, 93%, 94%, 95%, 96%, 97%, 98%, 99% or more sequence identity to any SEQ ID NOS: 6, 80, 83, 86, 88, 90, 92, 94, 96, 98, 100, 102, 104, 106, 108, 110, 112, 114, 116 or 118.

The nucleic acid molecule encoding the tyrosinase enzyme can be operably linked to a promoter for expression of the heterologous RNA and/or protein. The promoter can be any promoter, such as any described herein above or known in the art. In particular examples herein, the promoter is a strong promoter in order to achieve high levels of expression and overproduction of tyrosinase in cells such that eumelanin is produced over pheomelanin. For example, the promoter is a late viral promoter, a retroviral LTR or other strong eukaryotic promoter. Examples of such promoters are adenoviral major late promoter, vaccinia synthetic early-late promoter (P_{SEL}), vaccinia synthetic late promoter (P_{SL}), simian virus (SV40) promoter, cytomegalovirus (CMV) promoter, respiratory syncytial virus (RSV) promoter, human elongation factor 1α-subunit (EF1-1α) promoter, a ubiquitin C promoter (Ubc), a phosphoglycerate kinase-1 (PGK) promoter, small nuclear RNA U1b promoter or glucose 6-phosphate dehydrogenase promoter. In particular, the encoded tyrosinase enzyme is under control of the vaccinia synthetic early-late promoter (P_{SEL}; SEQ ID NO:169) or the vaccinia synthetic early promoter (P_{SE}; SEQ ID NO:168), and generally under control of the vaccinia synthetic early-late promoter (P_{SEL}; SEQ ID NO:169). Examples of such viruses include, for example, GLV-1h326 (SEQ ID NO:69), GLV-1h327 (SEQ ID NO:70), GLV-1h459 (SEQ ID NO: 213), GLV-1h460 (SEQ ID NO:214), GLV-1h461 (SEQ ID NO:215), GLV-1h462 (SEQ ID NO:216), GLV-2b482 (SEQ ID NO:200) or GLV-0e407 (SEQ ID NO:205).

The promoter operatively linked to the tyrosinase enzyme or other chromophore-producing enzyme also can be regulated to be inducible in order to regulate gene expression of the chromophore-producing enzyme. In particular examples herein, a tetracycline-inducible gene expression system is employed such that the promoter controlling expression of the chromophore-producing enzyme (e.g., tyrosinase) bears a tet operon (tetO) that can be controlled by a tetR in the absence of an inducer. In the presence of an inducer, such as tetracycline or a derivative of tetracycline (e.g., doxycycline, oxytetracycline, minocycline or other derivative), transcription can be induced. Examples of such viruses are GLV-1h461 (SEQ ID NO:215) or GLV-1h462 (SEQ ID NO: 216).

The vaccinia virus also can be modified by insertion of a sequence of nucleotides encoding a further accessory melanin-producing enzyme. In such examples, the accessory melanin-producing enzyme is one that contributes to the production of eumelanin in a host cell into which it is expressed. Examples of such enzymes are tyrosinase-related protein 1 and dopachrome tautomerase as described herein above. The sequences encoding the accessory melanin-producing enzyme are operatively linked to a promoter for expression thereof. The promoter can be the same promoter or a different promoter than that controlling expression of the tyrosinase enzyme. In some examples, the sequences encoding the tyrosinase enzyme and the accessory melanin-producing enzyme or enzymes can be driven under control of the same promoter. The sequences can be separated by an internal ribosome entry site (IRES).

Thus, in some examples, a vaccinia virus, such as a Lister virus (e.g., LIVP) contains a sequence of nucleotides encoding a tyrosinase-related protein 1 (TRP-1; Tyrp1) enzyme having the sequence of amino acids set forth in any of SEQ ID NOS: 20, 120, 121, 123, 124, 126, 127, 129, 131, 133, 135, 137, 139, 141, 142 or 144, or a mature form thereof lacking the signal sequence, or a sequence of amino acids that exhibits at least 75% or more sequence identity to any of SEQ ID NOS: 20, 120, 121, 123, 124, 126, 127, 129, 131, 133, 135, 137, 139, 141, 142 or 144 or the mature form thereof lacking the signal sequence, such as a sequence of amino acids that exhibits at least 80%, 85%, 86%, 87%, 88%, 89%, 90%, 91%, 92%, 93%, 94%, 95%, 96%, 97%, 98%, 99% or more sequence identity to any of SEQ ID NOS: 20, 120, 121, 123, 124, 126, 127, 129, 131, 133, 135, 137, 139, 141, 142 or 144, or a mature form thereof lacking the signal sequence. For example, the nucleotide sequence can be any set forth in any of SEQ ID NOS: 19, 119, 122, 125, 128, 130, 132, 134, 136, 138, 140, 143 or 145 or a sequence of nucleotides that exhibits at least 70% sequence identity to any of SEQ ID NOS: 19, 119, 122, 125, 128, 130, 132, 134, 136, 138, 140, 143 or 145, such as at least 75%, 80%, 85%, 86%, 87%, 88%, 89%, 90%, 91%, 92%, 93%, 94%, 95%, 96%, 97%, 98%, 99% or more sequence identity to any SEQ ID NOS: 19, 119, 122, 125, 128, 130, 132, 134, 136, 138, 140, 143 or 145. Examples of such viruses are GLV-1h310 (SEQ ID NO:64), GLV-1h322 (SEQ ID NO:67); GLV-1h323 (SEQ ID NO:206); GLV-1h324 (SEQ ID NO:68), GLV-1h458 (SEQ ID NO: 212); GLV-2b452 (SEQ ID NO:198), GLV-2b453 (SEQ ID NO:199) or GLV-0e406 (SEQ ID NO:204).

In other examples, a vaccinia virus, such as a Lister virus (e.g., LIVP) contains a sequence of nucleotides encoding a DCT enzyme or an enzymatically active portion thereof or an enzymatically active variant thereof, such as a DCT enzyme having the sequence of amino acids set forth in any of SEQ ID NOS: 30, 146, 147, 148, 150, 152, 154, 156, 158, 160, 162, 164 or 166, or a mature form thereof lacking the signal sequence, or a sequence of amino acids that exhibits at least 75% or more sequence identity to any of SEQ ID NOS: 30, 146, 147, 148, 150, 152, 154, 156, 158, 160, 162, 164 or 166 or the mature form thereof lacking the signal sequence, such as at least 80%, 85%, 86%, 87%, 88%, 89%, 90%, 91%, 92%, 93%, 94%, 95%, 96%, 97%, 98%, 99% or more sequence identity to any of SEQ ID NOS: 30, 146, 147, 148, 150, 152, 154, 156, 158, 160, 162, 164 or 166, or a mature form thereof lacking the signal sequence. The nucleotide sequence can be any set forth in SEQ ID NOS: 29, 149, 151, 153, 155, 157, 159, 161, 163, 165 or 167 or a sequence of nucleotides that exhibits at least 70% sequence identity to any of SEQ ID NOS: 29, 149, 151, 153, 155, 157, 159, 161, 163, 165 or 167, such as at least 75%, 80%, 85%, 86%, 87%, 88%, 89%, 90%, 91%, 92%, 93%, 94%, 95%, 96%, 97%, 98%, 99% or more sequence identity to any SEQ ID NOS: 29, 149, 151, 153, 155, 157, 159, 161, 163, 165 or 167.

The genotypes of exemplary vaccinia viruses encoding a tyrosinase enzyme are set forth in Table 6. Among such viruses are those that also encode tyrosinase-related protein 1 (TRP-1; Tyrp1). Gene transcription of the enzyme is controlled by a promoter operatively linked to the nucleic acid encoding the tyrosinase enzyme (i.e., mTyr) and/or tyrosinase-related protein 1 (i.e., Tyrp1). In some cases, a tetracycline-inducible gene expression system is included. Hence, provided herein for use in the methods herein are vaccinia viruses having a genome containing the sequence of nucleotides set forth in any of SEQ ID NOS: 64, 67-70, 198-200, 204-206 or 212-216, or a sequence of nucleotides that exhibits at least 75% sequence identity to any of SEQ ID NOS: 64, 67-70, 198-200, 204-206 or 212-216 such as at least 80%, 81%, 82%, 83%, 84%, 85%, 86%, 87%, 88%, 89%, 90%, 91%, 92%, 93%, 94%, 95%, 96%, 97%, 98%, 99% or more sequence identity to any of SEQ ID NOS: 64, 67-70, 198-200, 204-206 or 212-216.

TABLE 6
Tyrosinase and Tyrosinase/Tyrosinase-Related Protein 1 - Expressing Virus
Strains

| | | | | | | |
|---|---|---|---|---|---|---|
| | SEQ | Vaccinia | | | | |
| virus | ID | virus | parent | F14.5L | | |
| name | NO: | type | strain | locus | TK locus | HA locus |

tyrosinase-expressing virus strains

| | | | | | | |
|---|---|---|---|---|---|---|
| GLV- | 69 | LIVP | GLV- | (P_{SEL})Ruc- | (P_{SE})mTyr | (P₁₁ₖ)gusA |
| 1h326 | | | 1h311 | GFP | | |
| GLV- | 70 | LIVP | GLV- | (P_{SEL})Ruc- | (P_{SEL})mTyr | (P₁₁ₖ)gusA |
| 1h327 | | | 1h311 | GFP | | |
| GLV- | 213 | LIVP | GLV- | (P_{SE})hNISa | (P_{SEL})mTyr | (P₁₁ₖ)gusA |
| 1h459 | | | 1h327 | | | |
| GLV- | 214 | LIVP | GLV- | (P_{SEL})Ruc- | (P_{SL})tetO-mTyr | (P₁₁ₖ)gusA |
| 1h460 | | | 1h311 | GFP | | |
| GLV- | 215 | LIVP | GLV- | (P_{7.5})tetR | (P_{SL})tetO-mTyr | (P₁₁ₖ)gusA |
| 1h461 | | | 1h330 | | | |
| GLV- | 216 | LIVP | GLV- | (P_{SEL})tetR | (P_{SL})tetO-mTyr | (P₁₁ₖ)gusA |
| 1h462 | | | 1h354 | | | |
| GLV- | 200 | LIVP | GLV- | wt | (P_{SEL})mTyr | wt |
| 2b482 | | | 2b372 | | | |
| GLV- | 205 | WR | GLV- | wt | (P_{SEL})mTyr | (P_{SEL})FUKW |
| 0e407 | | | 0e365 | | | |

tyrosinase- and tyrosinase-related protein 1- expressing virus strains

| | | | | | | |
|---|---|---|---|---|---|---|
| GLV- | 64 | LIVP | GLV- | (knock-out) | (P_{SEL})Tyrp1- | (P_{SL})TurboFP635 |
| 1h310 | | | 1h254 | | (P_{SEL})mTyr | |
| GLV- | 67 | LIVP | GLV- | (P_{SEL})Ruc- | (P_{SE})Tyrp1- | (P₁₁ₖ)gusA |
| 1h322 | | | 1h311 | GFP | (P_{SE})mTyr | |
| GLV- | 206 | LIVP | GLV- | (P_{SEL})Ruc- | (P_{SEL})Tyrp1- | (P₁₁ₖ)gusA |
| 1h323 | | | 1h311 | GFP | (P_{SEL})mTyr | |
| GLV- | 68 | LIVP | GLV- | (P_{SEL})Ruc- | (P_{SE})Tyrp1- | (P₁₁ₖ)gusA |
| 1h324 | | | 1h311 | GFP | (P_{SEL})mTyr | |
| GLV- | | LIVP | GLV- | (P_{SEL})Ruc- | (P_{SEL})Tyrp1- | (P₁₁ₖ)gusA |
| 1h325 | | | 1h311 | GFP | (P_{SE})mTyr | |
| GLV- | 212 | LIVP | GLV- | (P_{SE})hNISa | (P_{SE})Tyrp1- | (P₁₁ₖ)gusA |
| 1h458 | | | 1h324 | | (P_{SEL})mTyr | |
| GLV- | 198 | LIVP | GLV- | wt | (P_{SEL})Tyrp1- | wt |
| 2b452 | | | 2b372 | | (P_{SEL})mTyr | |
| GLV- | 199 | LIVP | GLV- | wt | (P_{SEL})mTyr | wt |
| 2b453 | | | 2b372 | | | |
| GLV- | 204 | WR | GLV- | wt | (P_{SE})Tyrp1- | (P_{SEL})FUKW |
| 0e406 | | | 0e365 | | (P_{SEL})mTyr | |

e. Methods of Generating Modified Viruses

The viruses for use in the compositions and methods herein can be modified by insertion, deletion, replacement or mutation as described herein, for example insertion or replacement of heterologous nucleic acid, using standard methodologies well-known in the art for modifying viruses. Methods for modification include, for example, in vitro recombination techniques, synthetic methods, direct cloning, and in vivo recombination methods as described, for example, in Sambrook et al., Molecular Cloning: A Laboratory Manual, 2nd edition, Cold Spring Harbor Laboratory Press, cold Spring Harbor N.Y. (1989), and in the Examples disclosed herein.

For example, generation of recombinant viruses, including recombinant vaccinia virus, is well-known in the art, and typically involves the generation of gene cassettes or transfer vectors using standard molecular biology techniques (see, e.g., U.S. Pat. No. 7,588,767 and US2009-0053244-A1, which describe exemplary methods of generating recombinant LIVP vaccinia viruses). Such techniques include various nucleic acid manipulation techniques, nucleic acid transfer protocols, nucleic acid amplification protocols, and other molecular biology techniques known in the art.

For example, point mutations or small insertions or deletions can be introduced into a gene of interest through the use of oligonucleotide mediated site-directed mutagenesis. In another example, homologous recombination can be used to introduce a mutation in the nucleic acid sequence or insertion or deletion of a nucleic acid molecule into a target sequence of interest. In some examples, mutations, insertions or deletions of nucleic acid in a particular gene can be selected for using a positive or negative selection pressure (see, e.g., Current Techniques in Molecular Biology, (Ed. Ausubel, et al.)).

Nucleic acid amplification protocols include, but are not limited to, polymerase chain reaction (PCR), or amplification via viruses or organisms, such as, but not limited to, bacteria, yeast, insect or mammalian cells. Use of nucleic acid tools such as plasmids, vectors, promoters and other regulating sequences, are well-known in the art for a large variety of viruses and cellular organisms.

Nucleic acid transfer protocols include calcium chloride transformation/transfection, electroporation, liposome mediated nucleic acid transfer, N-[1-(2,3-dioloyloxy)propyl]-N,N,N-trimethylammonium methylsulfate meditated transformation, and others. Further a large variety of nucleic acid tools are available from many different sources, including various commercial sources. One skilled in the art will be readily able to select the appropriate tools and methods for genetic modifications of any particular virus according to the knowledge in the art and design choice.

Hence, any of a variety of modifications can be readily accomplished using standard molecular biological methods known in the art. The modifications will typically be one or more truncations, deletions, mutations or insertions of the viral genome. In one example, the modification can be specifically directed to a particular sequence in the viral genome. The modifications can be directed to any of a variety of regions of the viral genome, including, but not limited to, a regulatory sequence, a gene-encoding sequence, an intergenic sequence, a sequence without a known role, or a non-essential region of the viral genome. Any of a variety of regions of viral genomes that are available for modification are readily known in the art for many viruses, including LIVP.

Heterologous nucleic acid molecules are typically inserted into the viral genome in an intergenic region or in a locus that encodes a nonessential viral gene product. Insertion of heterologous nucleic acid at such sites generally does not significantly affect viral infection or replication in the target tissue. Exemplary insertion sites are known in the art and include, but are not limited to, J2R (thymidine kinase (TK)), A56R (hemagglutinin (HA)), F14.5L, vaccinia growth factor (VGF), A35R, N1L, E2L/E3L, K1L/K2L, superoxide dismutase locus, 7.5K, C7-K1L (host range gene region), B13R+B14R (hemorrhagic region), A26L (A type inclusion body region (ATI)) or I4L (large subunit, ribonucleotide reductase) gene loci. Insertion sites for the viruses provided herein also include sites that correspond to intragenic regions described in other poxviruses such as Modified Vaccinia Ankara (MVA) virus (exemplary sites set forth in U.S. Pat. No. 7,550,147), NYVAC (exemplary sites set forth in U.S. Pat. No. 5,762,938).

Methods for the generation of recombinant viruses using recombinant DNA techniques are well-known in the art (e.g., see U.S. Pat. Nos. 4,769,330; 4,603,112; 4,722,848; 4,215,051; 5,110,587; 5,174,993; 5,922,576; 6,319,703; 5,719,054; 6,429,001; 6,589,531; 6,573,090; 6,800,288; 7,045,313; He et al. (1998) PNAS 95(5): 2509-2514; Racaniello et al., (1981) Science 214: 916-919; and Hruby et al., (1990) Clin Micro Rev. 3:153-170). Methods for the generation of recombinant vaccinia viruses are well-known in the art (e.g., see Hruby et al., (1990) Clin Micro Rev. 3:153-170, U.S. Pat. Pub. No. 2005-0031643, now U.S. Pat. Nos. 7,588,767, 7,588,771, 7,662,398 and U.S. Pat. No. 7,045,313).

For example, generating a recombinant vaccinia virus that expresses a heterologous gene product typically includes the use of a recombination plasmid which contains the heterologous nucleic acid, optionally operably linked to a promoter, with vaccinia virus DNA sequences flanking the heterologous nucleic acid to facilitate homologous recombination and insertion of the gene into the viral genome. Generally, the viral DNA flanking the heterologous gene is complementary to a non-essential segment of vaccinia virus DNA, such that the gene is inserted into a non-essential location. The recombination plasmid can be grown in and purified from Escherichia coli and introduced into suitable host cells, such as, for example, but not limited to, CV-1, BSC-40, BSC-1 and TK-143 cells. The transfected cells are then superinfected with vaccinia virus which initiates a replication cycle. The heterologous DNA can be incorporated into the vaccinia viral genome through homologous recombination, and packaged into infection progeny. The recombinant viruses can be identified by methods known in the art, such as by detection of the expression of the heterologous gene product, or by using positive or negative selection methods (U.S. Pat. No. 7,045,313).

In another example, the recombinant vaccinia virus that expresses a heterologous gene product can be generated by direct cloning (see, e.g., U.S. Pat. No. 6,265,183 and Scheiflinger et al. (1992) Proc. Natl. Acad. Sci. USA 89: 9977-9981). In such methods, the heterologous nucleic acid, optionally operably linked to a promoter, is flanked by restriction endonuclease cleavage sites for insertion into a unique restriction endonuclease site in the target virus. The virus DNA is purified using standard techniques and is cleaved with the sequence-specific restriction endonuclease, where the sequence is a unique site in the virus genome. Any unique site in the virus genome can be employed provided that modification at the site does not interfere with viral replication. For example, in vaccinia virus strain LIVP, the NotI restriction site is located in the ORF encoding the F14.5L gene with unknown function (Mikryukov et al., Biotekhnologiya 4: 442-449 (1988)). Table 7 provides a summary of unique restriction sites contained in exemplary LIVP strains and designates the nucleotide position of each. Such LIVP strains can be modified herein by direct cloning and insertion of heterologous DNA into the site or sites. Generally, insertion is in a site that is located in a non-essential region of the virus genome. For example, exemplary modifications herein include insertion of a foreign DNA sequence into the NotI digested virus DNA.

TABLE 7
Unique restriction endonuclease cleavage sites in LIVP clonal isolates

| Restriction Enzyme/ | | | | | | | | | LIVP |
|---|---|---|---|---|---|---|---|---|---|
| Site | | 1.1.1 | 2.1.1 | 4.1.1 | 5.1.1 | 6.1.1 | 7.1.1 | 8.1.1 | Parental |
| Name/ | SEQ | (SEQ ID | (SEQ ID | (SEQ ID | (SEQ ID | (SEQ ID | (SEQ ID | (SEQ ID | (SEQ ID |
| sequence | ID NO | NO: 55) | NO: 56) | NO: 57) | NO: 58) | NO: 59) | NO: 60) | NO: 61) | NO: 188) |
| SbfI | 238 | 40033/ | 40756/ | 39977/ | 40576/ | 40177/ | 40213/ | 40493/ | 38630/ |
| CCTGCAGG | | 40029 | 40752 | 39973 | 40572 | 40173 | 40209 | 40489 | 38626 |
| NotI | 239 | 42989/ | 43712/ | 42933/ | 43532/ | 43133/ | 43169/ | 43449/ | 41586/ |
| GCGGCCGC | | 42998 | 43716 | 42937 | 43536 | 43137 | 43173 | 43453 | 41590 |
| SgrAI | 240 | 114365/ | 115107/ | 114308/ | 114924/ | 114489/ | 114548/ | 114845/ | 112975/ |
| CRCCGGYG | | 114369 | 115111 | 114312 | 114928 | 114493 | 114552 | 114849 | 112979 |
| SmaI | 241 | 159260 | NA | NA | NA | NA | NA | NA | NA |
| CCCGGG | | | | | | | | | |
| TspMI | 241 | 159258/ | NA | NA | NA | NA | NA | NA | NA |
| CCCGGG | | 159262 | | | | | | | |
| XmaI | 241 | 159258/ | NA | NA | NA | NA | NA | NA | NA |
| CCCGGG | | 159262 | | | | | | | |
| ApaI | 242 | 180516/ | NA | 180377/ | 181027/ | 180638/ | 180596/ | 180972/ | NA |
| GGGCCC | | 180512 | | 180373 | 181023 | 180634 | 180592 | 180968 | |
| PspOMI | 242 | 180512/ | NA | 180373/ | 181023/ | 180634/ | 180592/ | 180968/ | NA |
| GGGCCC | | 180516 | | 180377 | 181027 | 180638 | 180596 | 180972 | |

In some examples, the virus genomic DNA is first modified by homologous recombination to introduce one or more unique restriction sites in the virus (see, e.g., Mackett et al. (1984) J. Virol. 857-864). Following cleavage with the restriction endonuclease, the cleaved DNA is optionally treated with a phosphatase to remove a phosphate moiety from an end of the DNA segment that is produced by cleavage with the endonuclease. Typically, a plasmid vector is generated that contains the heterologous DNA for insertion flanked by the restriction sites. Prior to insertion into the virus, the heterologous DNA is excised from the plasmid by cleavage with the sequence specific restriction endonuclease. The heterologous DNA is then ligated to the cleaved viral DNA and is packaged in a permissive cell line by infection of the cells with a helper virus, such as, but not limited to a fowlpox virus or a PUV-inactivated helper vaccinia virus, and transfection of the ligated DNA into the infected cells.

In some examples, the methods involve homologous recombination and/or use of unique restriction sites in the virus. For example, a recombinant LIVP vaccinia virus with an insertion, for example, in the F14.5L gene (e.g., in the Not I restriction site of an LIVP isolate), can be prepared by the following steps: (a) generating (i) a vaccinia shuttle/transfer plasmid containing the modification (e.g., a gene expression cassette or a modified F14.5L gene) inserted at a restriction site, X (e.g., Not 1), where the restriction site in the vector is flanked by parental virus sequences of the target insertion site and (ii) an LIVP virus DNA digested at restriction site X (e.g., Not I) and optionally dephosphorylated; (b) infecting cells with PUV-inactivated helper vaccinia virus and transfecting the infected host cells with a mixture of the constructs of (i) and (ii) of step a; and (c) isolating the recombinant vaccinia viruses from the transfectants. One skilled in the art knows how to perform such methods (see, e.g., Timiryasova et al. (Biotechniques 31: 534-540 (2001)). Typically, the restriction site X is a unique restriction site in the virus as described above.

In one example, the methods include introducing into the viruses one or more genetic modifications, followed by screening the viruses for properties reflective of the modification or for other desired properties. In some examples, the modification can be fully or partially random, whereupon selection of any particular modified virus can be determined according to the desired properties of the modified the virus.

3. Methods of Producing Viruses

Viruses in the compositions and methods provided herein can be produced by methods known to one of skill in the art. Typically, the virus is propagated in host cells, quantified and prepared for storage before finally being prepared in the compositions described herein. The virus can be propagated in suitable host cells to enlarge the stock, the concentration of which is then determined. In some examples, the infectious titer is determined, such as by plaque assay. The total number of viral particles also can be determined. The viruses are stored in conditions that promote stability and integrity of the virus, such that loss of infectivity over time is minimized. In some examples, a large amount of virus is produced and stored in small aliquots of known concentration that can be used for multiple procedures over an extended period of time. Conditions that are most suitable for various viruses will differ, and are known in the art, but typically include freezing or drying, such as by lyophilization. The viruses can be stored at a concentration of 10⁵-10¹⁰ pfu/mL, for example, 10⁷-10⁹ pfu/mL, such as at least or about or 10⁶ pfu/mL, 10⁷ pfu/mL, 10⁸ pfu/mL or 10⁹ pfu/mL. Immediately prior to preparing compositions provided herein, the stored viruses can be reconstituted (if dried for storage) and diluted in an appropriate medium or solution.

The following sections provide exemplary methods that can be used for the production and preparation of viruses for use in preparing viruses in the compositions provided herein.

a. Host Cells for Propagation

Virus strains can be propagated in an appropriate host cell. Such cells can be a group of a single type of cells or a mixture of different types of cells. Host cells can include cultured cell lines, primary cells, and proliferative cells. These host cells can include any of a variety of animal cells, such as mammalian, avian and insect cells and tissues that are susceptible to the virus, such as vaccinia virus, infection, including chicken embryo, rabbit, hamster, and monkey kidney cells. Suitable host cells include, but are not limited to, hematopoietic cells (hematopoietic stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts, and cell lines including, for example, CV-1, BSC40, Vero, and BSC-1, and human HeLa cells. Typically, viruses are propagated in cell lines that that can be grown in monolayers or in suspension. For example, exemplary cell lines for the propagation of vaccinia viruses include, but are not limited to, CV-1, BSC40, Vero, BGM, BSC-1 and RK-13 cells. Purification of the cultured strain from the system can be effected using standard methods.

b. Concentration Determination

The concentration of virus in a solution, or virus titer, can be determined by a variety of methods known in the art. In some methods, a determination of the number of infectious virus particles is made (typically termed plaque forming units (PFU)), while in other methods, a determination of the total number of viral particles, either infectious or not, is made. Methods that calculate the number of infectious virions include, but are not limited to, the plaque assay, in which titrations of the virus are grown on cell monolayers and the number of plaques is counted after several days to several weeks, and the endpoint dilution method, which determines the titer within a certain range, such as one log. Methods that determine the total number of viral particles, including infectious and non-infectious particles, include, but are not limited to, immunohistochemical staining methods that utilize antibodies that recognize a viral antigen and which can be visualized by microscopy or FACS analysis; optical absorbance, such as at 260 nm; and measurement of viral nucleic acid, such as by PCR, RT-PCR, or quantitation by labeling with a fluorescent dye.

c. Storage Methods

Once the virus has been purified (or to a desired purity) and the titer has been determined, the virus can be stored in conditions which optimally maintain its infectious integrity. Typically, viruses are stored in the dark, because light serves to inactivate the viruses over time. Viral stability in storage is usually dependent upon temperatures. Although some viruses are thermostable, most viruses are not stable for more than a day at room temperature, exhibiting reduced viability (Newman et al., (2003) J. Inf. Dis. 187:1319-1322). Vaccinia virus is generally stable at refrigerated temperatures, and can be stored in solution at 4° C., frozen at, for example −20° C., −70° C. or −80° C., or lyophilized with little loss of viability (Newman et al., (2003) J. Inf Dis. 187:1319-1322, Hruby et al., (1990) Clin. Microb. Rev. 3:153-170). Methods and conditions suitable for the storage of particular viruses are known in the art, and can be used to store the viruses used in the methods presented herein.

For short-term storage of viruses, for example, 1 day, 2 days, 4 days or 7 days, temperatures of approximately 4° C. are generally recommended. For long-term storage, most viruses can be kept at −20° C., −70° C. or −80° C. When frozen in a simple solution such as PBS or Tris solution (20 mM Tris pH 8.0, 200 NaCl, 2-3% glycerol or sucrose) at these temperatures, the virus can be stable for 6 months to a year, or even longer. Repeated freeze-thaw cycles are generally avoided, however, since it can cause a decrease in viral titer. The virus also can be frozen in media containing other supplements in the storage solution which can further preserve the integrity of the virus. For example, the addition of serum or bovine serum albumin (BSA) to a viral solution stored at −80° C. can help retain virus viability for longer periods of time and through several freeze-thaw cycles.

In other examples, the virus sample is dried for long-term storage at ambient temperatures. Viruses can be dried using various techniques including, but not limited to, freeze-drying, foam-drying, spray-drying and desiccation. Water is a reactant in nearly all of the destructive pathways that degrade viruses in storage. Further, water acts as a plasticizer, which allows unfolding and aggregation of proteins. Since water is a participant in almost all degradation pathways, reduction of the aqueous solution of viruses to a dry powder provides an alternative composition methodology to enhance the stability of such samples.

Lyophilization, or freeze-drying, is a drying technique used for storing viruses (see, e.g., Cryole et al., (1998) Pharm. Dev. Technol., 3(3), 973-383). There are three stages to freeze-drying; freezing, primary drying and secondary drying. During these stages, the material is rapidly frozen and dehydrated under high vacuum. Once lyophilized, the dried virus can be stored for long periods of time at ambient temperatures, and reconstituted with an aqueous solution when needed. Various stabilizers can be included in the solution prior to freeze-drying to enhance the preservation of the virus. For example, it is known that high molecular weight structural additives, such as serum, serum albumin or gelatin, aid in preventing viral aggregation during freezing, and provide structural and nutritional support in the lyophilized or dried state. Amino acids such as arginine and glutamate, sugars, such as trehalose, and alcohols such as mannitol, sorbitol and inositol, can enhance the preservation of viral infectivity during lyophilization and in the lyophilized state. When added to the viral solution prior to lyophilization, urea and ascorbic acid can stabilize the hydration state and maintain osmotic balance during the dehydration period. Typically, a relatively constant pH of about 7.0 is maintained throughout lyophilization.

Other methods for the storage of viruses at ambient, refrigerated or freezing temperatures are known in the art, and include, but are not limited to, those described in U.S. Pat. Nos. 5,149,653; 6,165,779; 6,255,289; 6,664,099; 6,872,357; and 7,091,030; and in U.S. Pat. Pub. Nos. 2003-0153065, 2004-003841 and 2005-0032044.

d. Preparation of Virus

Immediately prior to use, the virus can be prepared at an appropriate concentration in suitable media, and can be maintained at a cool temperature, such as on ice, until use. If the virus was lyophilized or otherwise dried for storage, then it can be reconstituted in an appropriate aqueous solution. The aqueous solution in which the virus is prepared is typically the medium used in the assay (e.g., DMEM or RPMI) or one that is compatible, such as a buffered saline solution (e.g., PBS, TBS, Hepes-buffered saline (HBS) solution). For pharmaceutical applications, the virus can be immediately prepared or reconstituted in a pharmaceutical solution. Numerous pharmaceutically acceptable solutions for use are well-known in the art (see e.g., Remington's Pharmaceutical Sciences (18^{th} edition) ed. A. Gennaro, 1990, Mack Publishing Co., Easton, Pa.). In one example, the viruses can be diluted in a physiologically acceptable solution, such as sterile saline or sterile buffered saline, with or without an adjuvant or carrier. In other examples, the pharmaceutical solution can contain a component that provides viscosity (e.g., glycerol) and/or component that has bactericidal properties (e.g., phenol). The virus can be reconstituted or diluted to provide the desired concentration or amount. The particular concentration can be empirically determined by one of skill in the art depending on the particular application.

4. Anti-Tumorigenicity and Efficacy

Viruses for use in the compositions and methods provided herein can be tested for parameters indicative of its anti-tumorigenic property. Generally, the parameters selected for are desirable for the treatment of proliferative diseases and disorders, including the treatment of a tumor or metastasis. For example, a virus can destroy tumor cells by replicating such that continual amplification of the virus results in infection of adjacent cells and their subsequent destruction. Oncolytic viruses also exhibit anti-tumorigenicity by expression of proteins that are cytotoxic to cancer cells. In further examples, viruses can exhibit anti-tumorigenicity by initiating specific and nonspecific anti-tumor immune responses, for example, the initiation of cytokine expression from infected cells (e.g., TNF) or through a specific response (e.g., CTL response). Hence, any of the above parameters can be assessed as indicative of anti-tumorigenicity of a virus.

For example, the isolated virus is tested in one or more in vitro and/or in vivo assays that assess infectivity, viral nucleic acid replication, virus production, viral gene expression from tumor cells, effects on the host cell, cytotoxicity of tumor cells, tumor cell selectivity, tumor cell-type selectivity, specific and non-specific immune response, and therapeutic efficacy. Parameters indicative of anti-tumorigenicity can be assessed in vitro or in vivo. In particular examples, anti-tumorigenicity is assessed in vivo. In vivo parameters of anti-tumorigenicity include, but are not limited to, a desirable therapeutic index in an animal model of cancer, release of tumor antigens and preferential accumulation of the virus in tumor tissues following administration. Exemplary assays or methods to assess such parameters are described below.

a. Tumor-Associated Replication Indicator

Viruses in compositions provided herein can be tested for replication and/or infectivity in tumor cells. The replication indicator that is measured is any parameter from which the level or amount or relative amount of viral replication, typically within a day of administration to the tumor cells, can be assessed or inferred. In some examples, replication can be assessed by measurement of a viral replication indicator, such as, for example, viral titer (i.e., as assessed by the number of plaques produced in a plaque assay) or the changes in viral gene expression or host gene expression (see, e.g., U.S. Patent Pub. No. 2009-0136917). For example, replication can be determined by infecting or introducing the test virus into a tumor cell and assessing a replication indicator at a particular time or as a function of time. This can be compared to a predetermined standard, for example the parental virus preparation or mixture or other reference strain (e.g., recombinant virus), or compared to other test strains or controls. Viruses can be tested to assess selective replication in tumor cells compared to normal cells.

Assays to assess replication can be performed on cell lysates of cells infected in vitro with any of the provided vaccinia viruses. Exemplary cells that can be used for such replication assays include, but are not limited to, various tumor cell lines, primary tissues or cells as well as tumor cells such as from a biopsy. For example, a tissue or cell sample can be obtained (e.g., biopsy) from a subject (e.g., human or non-human animal subject), and the sample can be infected with one or more types of virus compositions. In other examples, tumor cell lines can be used. Tumor cell lines are known and available to one of skill in the art, for example, from the American Type Culture Collection (ATTC; Manassas, Va.) or from the European Collection of Cell Cultures (ECACC). Tumor cell lines also are available from the Division of Cancer Treatment and Diagnosis (DCTD) Tumor Repository (National Cancer Institute/National Institute of Health; dtp.nih.gov/index.html.). Exemplary tumor cell lines include human and other animal cells lines, including, but are not limited to, DU145 human prostate carcinoma cells, LNCaP human prostate cancer cells, MCF-7 human breast cancer cells, MRC-5 human lung fibroblast cells, MDA-MB-438 human breast cancer cells, MDA-MB-231 human breast carcinoma cells, PC3 human prostate cancer cells, T47D human breast cancer cells, THP-1 human acute myeloid leukemia cells, U87 human glioblastoma cells, SH-SY5Y human neuroblastoma cells, Saos-2 human cells, A549 human lung carcinoma cells, A2780 human ovarian carcinoma cells, HCT 116 human colon cells, HT-29 human colon cells, SW260 human colon cells, HT-180 human fibrosarcoma, MIA PaCa-2 human pancreatic carcinoma cells, PANC-1 human pancreatic cells, CMT 64 C57BL/6 mouse cell, JC mouse mammary cells, TIB-75 mouse hepatic cells, CT26 WT mouse colon carcinoma cells, MC-38 mouse adenocarcinoma cells, B16-F10 mouse melanoma cells, 4T1 murine mammary carcinoma cells and hamster pancreatic tumor HP-1 cells.

For example, cells or cell lines can be seeded onto wells of a plate. Virus compositions can then be added and allowed to infect the cells. At the end of the infection, the media can be changed to remove any residual virus and the cells further incubated. Then, the cells can be scraped into the media and collected. Cells can be lysed, for example, by freeze-thaw and/or sonication methods, to obtain virus-containing lysates. The extent of replication can be measured, such as by determination of viral titer or expression of genes as described further below. It is understood that the extent and degree of replication and/or infectivity efficiency of a virus will differ between various tumor cell types.

Assays to assess replication also can be performed on tumor-harvested virus propagated in vivo upon infection of tumor-bearing animals. Such an assay is a measure of the accumulation of the virus in tumor tissues. As discussed below, tumors can be established in animals by implantation of different tumor cell types. For example, tumor-bearing animals can be administered (e.g., topically or via intravenous administration or other route of administration) with a vaccinia virus, virus propagated in tumors and virus or tumor extracted therefrom. The extent of replication can be measured, such as by determination of viral titer or expression of genes, as described further below.

In one example, cell culture supernatants or cell lysates from the infected cells or tumor cell extracts can be obtained following infection and subjected to assays to measure viral titer. For example, a standard plaque assay can be used. The plaque assay can indicate the biological activity in different cell types, including different tumor cell types. Titration of virus by plaque assay is known to one of skill in the art. In one example of a plaque assay, supernatants or cells lysates of tumors or cells infected with the virus is harvested and plaque assays can be performed. Typically, serial dilutions of the virus supernatant or lysate is made in the range of 10⁻² (1:100) to 10⁻¹⁰, and in particular from 10⁻⁵ to 10⁻¹⁰. Diluted virus is added to a monolayer of cells, for example, monolayers of permissive cell line, such as, for example, CV-1, Vero, BHK, RK13 or HEK-293 cell lines, and incubated with virus. In some examples, the plaque assay can be performed directly on a monolayer of tumor cells provided that the tumor cells can form a monolayer. Following incubation, an agarose overlay is added to the monolayer of cells without dislodging the cells, and the plate is further incubated until plaques become visible. A dye or color stain solution that is taken up by healthy cells but not dead cells, such as neutral red, is added to each of the wells or plate. After incubation, the dye or stain is removed such that the plaques are observed to be clear, while non-lysed cells remain stained. Titer (pfu/mL) is calculated by counting the number of plaques in the well and dividing by the dilution factor (d) and the volume (V) of diluted virus added to the well (# plaques/d×V). The virus yield can be converted to pfu/cell by dividing the total amount of virus present in the sample by the number of cells originally infected in the sample.

Other indicators of replication also can be assessed. For example, expression of viral genes, tumor proteins and/or housekeeping genes that are correlated with viral replication and/or infectivity in tumor cells can be assessed (see e.g., U.S. Patent Pub. No. 2009-0136917). For example, expression of housekeeping genes or other genes in tumor cells associated with virus replication and infectivity can be assessed (U.S. Patent Pub. No. 2009-0136917). In some examples, expression of a plurality of such genes, such as housekeeping genes, whose expression increases in tumor cells upon infection with virus are assessed. Examples of such genes that can be assessed include expression of one or more genes encoding a protein selected from among IL-18 (Interleukin-18), MCP-5 (Monocyte Chemoattractant Protein-5; CCL12), IL-11 (Interleukin-11), MCP-1 (Monocyte Chemoattractant Protein-1), MPO (Myeloperoxidase), Apo A1 (Apolipoprotein A1), TIMP-1 (Tissue Inhibitor of Metalloproteinase Type-1), CRP (C Reactive Protein), Fibrinogen, MMP-9 (Matrix Metalloproteinase-9), Eotaxin (CCL11), GCP-2 (Granulocyte Chemotactic Protein-2; CXCL6), IL-6 (Interleukin-6), Tissue Factor (TF), SAP (Serum Amyloid P), FGF-basic (Fibroblast Growth Factor-basic), MCP-3 (Monocyte Chemoattractant Protein-3; CCL7), IP-10 (CXCL 10), MIP-2, Thrombopoietin, Cancer antigen 125, CD40, CD40 ligand, ENA-78, Ferritin, IL-12p40, IL-12p70, IL-16, MMP-2, PAI-1, TNF RII, TNF-beta and VCAM-1.

In another example, expression of a plurality of genes, such as housekeeping genes, whose expression decreases in tumor cells upon infection with virus are assessed. Examples of such genes include one or more genes encoding a protein selected from among MIP-1beta (Macrophage Inflammatory Protein-1beta), MDC (Macrophage-Derived Chemokine; CCL22), MIP-1 alpha (Macrophage Inflammatory Protein-1alpha; CCL3), KC/GROalpha (Melanoma Growth Stimulatory Activity Protein), VEGF (Vascular Endothelial Cell Growth Factor), Endothelin-1, MIP-3 beta (Macrophage Inflammatory Protein-3 beta; Exodus-3 or ELC), Beta-2 microglobulin, IL-5 (Interleukin-5), IL-1 alpha (Interleukin-1 alpha), EGF (Epidermal Growth Factor), Lymphotactin (XCL1), GM-CSF (Granulocyte Macrophage-Colony Stimulating Factor), MIP-1 gamma (Macrophage Inflammatory Protein-1 gamma; CCL4), IL-1beta (Interleukin-1 beta), BDNF (Brain-derived neutrophic factor), Cancer antigen 19-9, Carcinoembryonic antigen, C reactive protein, EGF, Fatty acid binding protein, Factor VII, Growth hormone, IL-1 alpha, IL-1 beta, IL-1 ra, IL-7, IL-8, MDC, Prostatic acid phosphatase, Prostate specific antigen, free, Stem cell factor, Tissue factor, TNF-alpha, VEGF and Von Willebrand factor.

Gene expression can be assayed after contacting a tumor sample with the virus for a period of time in vitro or in vivo and measuring the level of expression of one or more housekeeping genes or other genes. Any method known in the art can be used for assessing the expression of genes in a tumor can be employed. For example, methods for measuring protein expression levels which can be used include, but are not limited to, microarray analysis, ELISA assays, Western blotting, or any other technique for the quantitation of specific proteins. For RNA levels, examples of techniques which can be used include microarray analysis, quantitative PCR, Northern hybridization, or any other technique for the quantitation of specific nucleic acids. In some examples, a difference in expression of the same marker between the contacted and non-contacted biological samples of about less than 2-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold or greater than about 100-fold is indicative of specific replication and/or infectivity of a tumor cell.

For in vitro tests, varying doses/multiplicity of infection (MOI; ratio of virus to cell) of the virus can be assessed in order to assess the rate of viral infection and virus production at different infection levels. Viruses that exhibit a high rate of replication at a lower MOI are generally desirable for therapy of a proliferative disorder or disease. For example, cells can be infected at an MOI of at or between 0.1 to 10, such as 0.5 to 5, for example, 0.5 to 2, for example, an MOI of at or at least 0.25, 0.5, 1, 1.5, 2 or more.

In any of the examples herein of assessing replication or infectivity of a virus, tumor cell selectivity of the virus also can be assessed. For example, normal cells and tumor cells can be infected with the vaccinia virus composition followed by assessment of replication and or infectivity using any of the assays described herein or known to one of skill in the art. For example, measurement of viral titer by plaque assay or by expression of genes as described can be determined in virally-infected tumor cells versus virally-infected normal cells. Normal or non-transformed cells include, but are not limited to, MRC-5 lung fibroblast cells, Beas-2B bronchial epithelial cells, normal human bronchial epithelial (NHBE) cells, small airway bronchial epithelial cells(SAEC). Tumor cells include any described herein or known to one of skill in the art and include, but are not limited to, A2780, A549, HCT 116, HT 1080, LNCaP or SW620 cells. In some examples, paired tumor and non-tumor cell lines can be infected with virus and compared. Exemplary corresponding or paired tumor and non-tumor cell lines are known to one of skill in the art (see e.g., Gazdar et al. (1998) Int. J. Cancer, 78:766-774, Theodore et al. (2010) Int. J Oncology, 37:1477-1482; Niedbala et al. (2001) Radiation Research, 155:297-303). In other examples, tumors infected in vivo can be harvested and can be compared to normal cells or tissues that also are extracted from the same infected animal. Infection and replication of virus in normal cells and tumor cells can be assessed and compared. The therapeutic index of the virus can be determined by the ratio of replication in the tumor cell compared to the normal cell (e.g., virus produced per cell; pfu/cell).

b. Cytotoxicity

Viruses can be tested to determine if they are cytotoxic or kill tumor cells. For example, viruses can eliminate tumor cells via induction of cell death and/or lysis of the tumor cell (i.e., oncolysis). The cell killing activity of the virus can be assessed by a variety of techniques known in the art including, but not limited to, cytotoxicity/cell viability assays that can be employed to measure cell necrosis and/or apoptosis following virus infection, such as MTT (3-(4,5-dimethylthiazol-2-yl)-2,5-diphenyltetrazolium bromide) assays and other related tetrazolium salt based assays (e.g., XTT, MTS or WST), ATP assays, apoptosis assays, such as TUNEL staining of infected cells, DNA fragmentation assays, DNA laddering assays, and cytochrome C release assays. Such assays are well-known to one of skill in the art.

For example, viability of virally-infected cells can be assessed. Various tumor cell lines, for example any described above or known to one of skill in the art, can be seeded in a 96-well plate (e.g., at or about 5,000 cells/well) or other size well-plate and grown overnight, and then can be infected with serial dilution of virus compositions. For example, various MOI of the virus can be tested. MOI can range from, for example, 1000 to 0.0001, such as 100 to 0.001 or 10 to 0.01. It is within the level of one of skill in the art to empirically select or determine an appropriate MOI range in which to use. Once infected, the cells can be incubated for a period of time before assessment of cytotoxicity. For example, samples for assessment of cytotoxicity are typically obtained at selected time points following virus infection of the cells, such as, for example, 1 hour, 2 hours, 4 hours, 8 hours, 16 hours, 24 hours, 1.5 days. 2 days, 2.5 days, 3 days, 4 days, 5 days, 6 days or more. One of skill in the art can select appropriate time points for assessment of viral replication based on the relative infectivity of the virus compared to other known virus strains. Generally, infection is allowed to proceed at least 12 hours, 24 hours, 36 hours, 48 hours, 72 hours, 84 hours, 96 hours or more.

Following infection for the designated period, media is replaced and viability of the cells is determined based on any assay or procedure known to one of skill in the art. Exemplary assays to assess viability are colorimetric assays that permit visualization of cells based on metabolic activity and measure the reducing potential of the tetrazolium salt to a colored formazan product (e.g., MTT assay, MTS assay or XTT assay). Other redox assays include assays that measure the ability of cells to convert a redox dye resazurin to a fluorescent end product resorufin (McMillian et al. (2002) Cell Biol. Toxicology, 18:157-173; CellTiter-Blue™ Cell Viability Assay, Promega). In other examples, viability can be assessed using a CASY cell counting technology, which is an electric field multi-channel cell counting system based on existence of a transmitted electric field through injured or dead cells as compared to normal cells (e.g., CASY® Model TT; Roche Innovatis AG). Additional examples include, but are not limited to, trypan blue or propidium iodide dye exclusion assay, measurement of lactate dehydrogenase (LDH; see e.g., LDH Cytotoxicity Detection Kit, Clontech, Cat. #630117), sulforhodamine B (SRB) assay (e.g., CytoScan™ SRB Cytotoxicity Assay, GBiosciences, Cat. No. 786-213, WST assay (e.g., CytoScan™ WST-1 Cell Proliferation Assay, GBiosciences, Cat No. 786-212), clonogenic assay and luciferase-based ATP-based assays (e.g., CelTiter-Glo™ Luminescent Cell Viability Assay; Promega).

Generally, the assays are performed using various controls. For example, any assay to assess viability generally is performed with untreated wells containing cells only (e.g., 100% viable) as well as cell-free wells (0% viable). Also, in addition to the testing vaccinia virus compositions provided, other control viruses can be tested. For example, a reference virus strain, for example, a known attenuated recombinant strain can be tested. GLV-1h68, or a derivative thereof, containing inserted heterologous genes is an example of such a strain. In examples where virus is added as a control, the MOI range of virus that is used is the same as the tested virus composition.

A virus exhibits a cytopathic effect if it is determined to exhibit a reduction in cell viability relative to an untreated well containing cells only (100% viable). In other examples, a virus exhibits a cytopathic effect if it is determined to exhibit a reduction in cell viability relative to the viability of cells in a well-treated with a control or reference virus that is not oncolytic. In a further example, a virus exhibits a cytopathic effect if it is determined to exhibit a similar or greater effect on cell viability relative to the viability of cells in a well-treated with a known reference attenuated virus strain, such as an attenuated recombinant virus (e.g., GLV-1h68 or derivative thereof).

c. Tumor Growth

Viruses can be tested to determine if it causes shrinkage of tumor size and/or delays tumor progression. Tumor size can be assessed in vivo in tumor-bearing human or animal models treated with virus. Tumor shrinkage or tumor size can be assessed by various assays known in art, such as, by weight, volume or physical measurement.

Tumor-bearing animal models can be generated. In vivo tumors can be generated by any known method, including xenograft tumors generated by inoculating or implanting tumor cells (e.g., by subcutaneous injection) into an immunodeficient rodent, syngeneic tumors models generated by inoculating (e.g., by subcutaneous injection) a mouse or rat tumor cell line into the corresponding immunocompetent mouse or rat strain, metastatic tumors generated by metastasis of a primary tumor implanted in the animal model, allograft tumors generated by the implantation of tumor cells into same species as the origin of the tumor cells, and spontaneous tumors generated by genetic manipulation of the animal. The tumor models can be generated orthotopically by injection of the tumor cells into the tissue or organ of their origin, for example, implantation of breast tumor cells into a mouse mammary fat pad. Any of the above models provide a consistent and reproducible tool for evaluating tumor cell growth, as well as permitting easy access to assess the mass of the tumor.

In particular examples, xenograft models or syngenic models are used. For example, tumors can be established by subcutaneous injection at the right armpit with a cell suspension (e.g., 1×10⁶ to 5×10⁶ cells/animal) of different tumor cell types into immunocompetent hosts (syngeneic) or immunodeficient hosts (e.g., nude or SCID mice; xenograft). Exemplary human tumor xenograft models in mice, such as nude or SCID mice, include, but are not limited to, human lung carcinoma (A549 cells, ATCC No. CCL-185); human breast tumor (GI-101A cells, Rathinavelu et al., Cancer Biochem. Biophys., 17:133-146 (1999)); human ovarian carcinoma (OVCAR-3 cells, ATCC No. HTB-161); human pancreatic carcinoma (PANC-1 cells, ATCC No. CRL-1469 and MIA PaCa-2 cells, ATCC No. CRL-1420); DU145 cells (human prostate cancer cells, ATCC No. HTB-81); human prostate cancer (PC-3 cells, ATCC# CRL-1435); colon carcinoma (HT-29 cells); human melanoma (888-MEL cells, 1858-MEL cells or 1936-MEL cells; see e.g., Wang et al., (2006) J. Invest. Dermatol. 126:1372-1377); and human fibrosarcoma (HT-1080 cells, ATCC No. CCL-121,) and human mesothelioma (MSTO-211H cells). Exemplary rat tumor xenograft models in mice include, but are not limited to, glioma tumor (C6 cells; ATCC No. CCL-107). Exemplary mouse tumor homograft models include, but are not limited to, mouse melanoma (B16-F10 cells; ATCC No. CRL-6475). Exemplary cat tumor xenograft models in mice include, but are not limited to, feline fibrosarcoma (FC77.T cells; ATCC No. CRL-6105). Exemplary dog tumor xenograft models in mice include, but are not limited to, canine osteosarcoma (D17 cells; ATCC No. CCL-183). Non-limiting examples of human xenograft models and syngeneic tumor models are set forth in the Tables 8 and 9 below.

TABLE 8
Human Tumor Xenograft Models

| Tumor Type | Cell Line Name |
|---|---|
| Adenoid cystic | ACC-2 |
| carcinoma | |
| Bladder carcinoma | EJ |
| Bladder carcinoma | T24 |
| Breast carcinoma | BCaP-37 |
| Breast carcinoma | MX-1 |
| Cervical carcinoma | SiHa |
| Cervical carcinoma | HeLa |
| Colon carcinoma | Ls-174-T |
| Colon carcinoma | CL187 |
| Colon carcinoma | HCT-116 |
| Colon carcinoma | SW116 |
| Gastric carcinoma | MGC-803 |
| Gastric carcinoma | SGC-7901 |
| Gastric carcinoma | BGC-823 |
| Kidney carcinoma | Ketr-3 |
| Leukemia | HL-60 |
| Liver carcinoma | Bel-7402 |
| Liver carcinoma | HepG-2 |
| Liver carcinoma | QGY-7701 |
| Liver carcinoma | SMMC7721 |
| Lung carcinoma | A549 |
| Lung carcinoma | NCI-H460 |
| Melanoma | A375 |
| Melanoma | M14 |
| Melanoma | MV3 |
| Ovary carcinoma | A2780 |
| Pancreatic carcinoma | BXPC-3 |
| Prostate carcinoma | PC-3M |
| Tongue carcinoma | Tca-8113 |

TABLE 9
Syngeneic Mouse Tumor Model

| Tumor Type | Cell Line Name | Strain of Mice |
|---|---|---|
| Cervical carcinoma | U14 | ICR |
| Liver carcinoma | H22 | ICR |
| Lung carcinoma | Lewis | C57BL6 |
| Melanoma | B16F1, B16F10, B16BL6 | C57BL6 |
| Sarcoma | S180 | ICR |

Tumor size and volume can be monitored based on techniques known to one of skill in the art. For example, tumor size and volume can be monitored by radiography, ultrasound imaging, necropsy, by use of calipers, by microCT or by ¹⁸F-FDG-PET. Tumor size also can be assessed visually. In particular examples, tumor size (diameter) is measured directly using calipers. In other examples, tumor volume can be measured using an average of measurements of tumor diameter (D) obtained by caliper or ultrasound assessments. The volume can be determined from the formula V=D³×π/6 (for diameter measured using calipers) or V=D²×d×π/6 (for diameter measured using ultrasound where d is the depth or thickness). For example, caliper measurements can be made of the tumor length (l) and width (w) and tumor volume calculated as length×width²×0.52. In another example, microCT scans can be used to measure tumor volume (see e.g., Huang et al. (2009) PNAS, 106:3426-3430). In such an example, mice can be injected with Optiray Pharmacy ioversol injection 74% contrast medium (e.g., 741 mg of ioversol/mL), mice anesthetized, and CT scanning done using a MicroCat 1A scanner or other similar scanner (e.g., IMTek) (40 kV, 600 μA, 196 rotation steps, total angle or rotation=196). The images can be reconstructed using software (e.g., RVA3 software program; ImTek). Tumor volumes can be determined by using available software (e.g., Amira 3.1 software; Mercury Computer Systems).

Once the implanted tumors reach a predetermined size or volume, the models can be used for treatment with virus. The exact final tumor volume can be empirically determined and is a function of the particular type of tumor as well as the end-point of the analysis. Generally, mice are sacrificed if the tumor volume is greater than 3 cm³.

Tumor-bearing animals are infected with virus. The route of administration for infection can be any desired route of administration, for example, intravenous or topical. Other routes also can be employed, for example, intraperitoneal, such as subcutaneous, or can be intratumoral. The vaccinia can be administered at varying dosages. For example, the virus can be administered to tumor-bearing animals at or between about 1×10⁴ to 1×10⁸ pfu, such as 1×10⁵ to 1×10⁷ pfu, for example at least or about or 1×10⁶, 2×10⁶, 3×10⁶, 4×10⁶ or 5×10⁶ pfu. Progressing tumors are visualized and tumor size and tumor volume can be measured using any technique known to one of skill in the art. For example, tumor volume or tumor size can be measured using any of the techniques described herein. Tumor volume and size can be assessed or measured at periodic intervals over a period of time following virus infections, such as, for example, every hour, every 6 hours, every 12 hours, every 24 hours, every 36 hours, every 2 days, every 3 days, every 4 days, every 5 days, every 6 days, every 7-days, every week, every 3 weeks, every month or more post-infection. A graph of the median change in tumor volume over time can be made and the total area under the curve (AUC) can be calculated. A therapeutic index also can be calculated using the formula AUC_{untreated animals}−AUC_{virus-treated animals}/AUC_{untreated}×100.

In additional examples, tumors can be harvested from the animals and weighed. In further examples, the harvested tumors can be lysed. For example, lysis of tumors can be by freeze-thaw of the harvested tumor several times (e.g., at least 2 times, 3 times or 4 times) shortly after removal of the tumor from the animal. For example, the tumor is lysed by 3 freeze-thaw cycles within 2 hours of removal. The virus in the tumor lysates can be tittered as described above and the amount of virus in each tumor sample determined. In some examples, the virus titer can be expressed as tissue culture infectious dose normalized to the tissue weight (TCID₅₀/mg tissue). In particular examples, the effect of the virus on other organs or tissues in the animal can be assessed. For example, other organs can be harvested from the animals, weighed and/or lysed for viral titer determination.

Generally, tumor-bearing animals generated in the same manner, at the same time and with the same type of tumor cells are used as controls. Such control tumor-bearing animals include those that remain untreated (not infected with virus). Additional control animals can include those infected with a reference virus strain. An example of such a strain is GLV-1h68 or a derivative thereof containing inserted heterologous genes. Comparison of tumor size or volume can be made at any predetermined time post-infection, and can be empirically determined by one of skill in the art. In some examples, a comparison can be made at the day in which the untreated control is sacrificed. In other examples, analysis of the total AUC can be made, and AUC values compared as an indicator of the size and volume of the tumor over the time period of infection. A decrease in tumor size, volume or weight compared to control treated or untreated tumor-bearing animals means that the virus itself is mediating tumor regression or shrinkage or that the virus is mediating delayed tumor progression compared to control treated or untreated tumor-bearing animals. Tumor shrinkage or delay in tumor progression are parameters indicative of anti-tumorigenicity.

5. Toxicity/Safety

Virus provided herein can be tested for parameters indicative of its toxicity/safety property. Viruses can be toxic to their hosts by manufacturing one or more compounds that worsen the health condition of the host. Toxicity to the host can be manifested in any of a variety of manners, including septic shock, neurological effects, or muscular effects. Typically, vaccinia virus exhibits minimal to no toxicity to a host, such that the host does not die or become severely ill from the toxic effects of the virus. For example, the viruses are not toxic or exhibit minimal toxicity if a host typically has no significant long-term effect from the presence of the viruses in the host, beyond any effect on tumorous, metastatic or necrotic organs or tissues. For example, minimal toxicity can be a minor fever or minor infection, which lasts for less than about a month, and following the fever or infection, the host experiences no adverse effects resultant from the fever or infection. In another example, the minimal toxicity can be measured as an unintentional decline in body weight of about 5% or less for the host after administration of the virus. In other examples, the virus has no toxicity to the host.

Parameters indicative of toxicity or safety of a virus can be tested in vitro or in vivo. Typically, assessment is in vivo. Exemplary methods include administration of the virus to a subject (e.g., animal model) and assessment of one or more properties associated with toxicity including, but not limited to, survival of the subject, decrease in body weight, existence of side effects such as fever, rash or other allergy, fatigue or abdominal pain, induction of an immune response in the subject, tissue distribution of the virus, amount of tumor antigens that are released and decreased rate of pock formation. Hence, any of the above parameters can be assessed as indicative of toxicity/safety of a virus.

As above, subjects (e.g., animals such as tumor-bearing animal models) are infected with virus. The route of administration for infection can be any desired route of administration, for example, intravenous or topical. Other routes also can be employed, for example, intraperitoneal, such as subcutaneous, or can be intratumoral. The virus can be administered at varying dosages. For example, the virus can be administered to tumor-bearing animals at or between about 1×10⁴ to 1×10⁸ pfu, such as 1×10⁵ to 1×10⁷ pfu, for example at least or about or 1×10⁶, 2×10⁶, 3×10⁶, 4×10⁶ or 5×10⁶ pfu. For humans, the virus can be administered at or between about 1×10⁷ to 1×10¹⁴ pfu, such as 1×10⁷ to 1×10¹⁰ pfu or 1×10⁹ to 1×10¹⁰ pfu, for example at least or about 1×10⁹, 2×10⁹, 3×10⁹, 4×10⁹, or 5×10⁹ pfu. Parameters indicative of toxicity such as the survival and weight of the subject can be monitored over time. For example, survival and weight can be monitored at periodic intervals over a period of time following virus infections, such as, for example, every hour, every 6 hours, every 12 hours, every 24 hours, every 36 hours, every 2 days, every 3 days, every 4 days, every 5 days, every 6 days, every 7-days, every week, every 3 weeks, every month or more post-infection.

Generally, control subjects (e.g., animal models such as tumor-bearing animal models) are similarly monitored. Such control subjects include those that remain untreated (not infected with virus). Additional controls animals can include those infected with a reference virus strain. GLV-1h68, or a derivative thereof, containing inserted heterologous genes is an example of such a strain.

### F. PHARMACEUTICAL COMPOSITIONS, COMBINATIONS AND KITS

Provided herein are pharmaceutical compositions, combinations and kits containing a virus provided herein. Pharmaceutical compositions can include a virus provided herein and a pharmaceutical carrier. Combinations can include, for example, two or more viruses, a virus and a detectable compound, a virus and a therapeutic compound, a virus and a viral expression modulating compound, or any combination thereof. Kits can include one or more pharmaceutical compositions or combinations provided herein, and one or more components, such as instructions for use, a device for administering the pharmaceutical composition or combination to a subject, a device for administering a therapeutic or diagnostic compound to a subject or a device for detecting a virus in a subject.

A virus contained in a pharmaceutical composition, combination or kit can include any virus provided herein. The pharmaceutical compositions, combinations or kits can include one or more additional viruses that can be selected from a viruses provided herein, or other therapeutic or diagnostic virus, such as any oncolytic virus provided herein.

1. Pharmaceutical Compositions

Provided herein are pharmaceutical compositions containing a virus provided herein and a suitable pharmaceutical carrier. A pharmaceutically acceptable carrier includes a solid, semi-solid or liquid material that acts as a vehicle carrier or medium for the virus. Pharmaceutical compositions provided herein can be formulated in various forms, for example in solid, semi-solid, aqueous, liquid, powder or lyophilized form. Exemplary pharmaceutical compositions containing a virus provided herein include, but are not limited to, sterile injectable solutions, sterile packaged powders, eye drops, tablets, pills, powders, lozenges, sachets, cachets, elixirs, suspensions, emulsions, solutions, syrups, aerosols (as a solid or in a liquid medium), ointments, soft and hard gelatin capsules, and suppositories.

Examples of suitable pharmaceutical carriers are known in the art and include, but are not limited to, water, buffers, saline solutions, phosphate buffered saline solutions, various types of wetting agents, sterile solutions, alcohols, gum arabic, vegetable oils, benzyl alcohols, gelatin, glycerin, carbohydrates, such as lactose, sucrose, dextrose, amylose or starch, sorbitol, mannitol, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preserving agents, analgesic agents, binders, disintegrants, coloring, diluents, excipients, extenders, glidants, solubilizers, stabilizers, tonicity agents, vehicles, viscosity agents, flavoring agents, sweetening agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as, but not limited to, crystalline cellulose, microcrystalline cellulose, citric acid, dextrin, liquid glucose, lactic acid, lactose, magnesium chloride, potassium metaphosphate, starch, among others. Such carriers and/or additives can be formulated by conventional methods and can be administered to the subject at a suitable dose. Stabilizing agents such as lipids, nuclease inhibitors, polymers, and chelating agents can preserve the compositions from degradation within the body. Other suitable formulations for use in a pharmaceutical composition can be found, for example, in Remington: The Science and Practice of Pharmacy (2005, Twenty-first edition, Gennaro & Gennaro, eds., Lippencott Williams and Wilkins).

Pharmaceutical formulations that include a virus provided herein for injection or mucosal delivery typically include aqueous solutions of the virus provided in a suitable buffer for injection or mucosal administration or lyophilized forms of the virus for reconstitution in a suitable buffer for injection or mucosal administration. Such formulations optionally can contain one or more pharmaceutically acceptable carriers and/or additives as described herein or known in the art. Liquid compositions for oral administration generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Pharmaceutical compositions provided herein can be formulated to provide quick, sustained or delayed released of a virus as described herein by employing procedures known in the art. For preparing solid compositions such as tablets, a virus provided herein is mixed with a pharmaceutical carrier to form a solid composition. Optionally, tablets or pills are coated or otherwise compounded to provide a dosage form affording the advantage of prolonged action in the subject. For example, a tablet or pill contains an inner dosage and an outer dosage component, the latter being in the form of an envelope over the former. The two components can be separated by an enteric layer, for example, which serves to resist disintegration in the stomach and permit the inner component to pass intact into the duodenum or to be delayed in release. A variety of materials are used for such enteric layers or coatings, including, for example, a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. These liquid or solid compositions optionally can contain suitable pharmaceutically acceptable excipients and/or additives as described herein or known in the art. Such compositions are administered, for example, by the oral or nasal respiratory route for local or systemic effect. Compositions in pharmaceutically acceptable solvents are nebulized by use of inert gases. Nebulized solutions are inhaled, for example, directly from the nebulizing device, from an attached face mask tent, or from an intermittent positive pressure breathing machine. Solution, suspension, or powder compositions are administered, orally or nasally, for example, from devices which deliver the formulation in an appropriate manner such as, for example, use of an inhaler.

Pharmaceutical compositions provided herein can be formulated for transdermal delivery via a transdermal delivery devices (“patches”). Such transdermal patches are used to provide continuous or discontinuous infusion of a virus provided herein. The construction and use of transdermal patches for the delivery of pharmaceutical agents are performed according to methods known in the art (see, for example, U.S. Pat. No. 5,023,252). Such patches are constructed for continuous, pulsatile, or on-demand delivery of a virus provided herein.

Colloidal dispersion systems that can be used for delivery of viruses include macromolecule complexes, nanocapsules, microspheres, beads and lipid-based systems including oil-in-water emulsions (mixed), micelles, liposomes and lipoplexes. An exemplary colloidal system is a liposome. Organ-specific or cell-specific liposomes can be used in order to achieve delivery only to the desired tissue. The targeting of liposomes can be carried out by the person skilled in the art by applying commonly known methods. This targeting includes passive targeting (utilizing the natural tendency of the liposomes to distribute to cells of the reticuloendothelial system (RES) in organs which contain sinusoidal capillaries) or active targeting (for example, by coupling the liposome to a specific ligand, for example, an antibody, a receptor, sugar, glycolipid and protein by methods known to those of skill in the art). Monoclonal antibodies can be used to target liposomes to specific tissues, for example, tumor tissue, via specific cell-surface ligands.

2. Host Cells

Host cells that contain a virus provided herein are provided. Such cells can be employed in vitro use or in vivo use, for example, as described in the diagnostic or therapeutic methods provided herein. The host cells can be a group of a single type of cells or a mixture of different types of cells. Host cells can include cultured cell lines, primary cells and proliferative cells. The host cells can include any of a variety of animal cells, such as mammalian, avian and insect cells and tissues that are susceptible to infection by the virus, including, but not limited to, human, primate, rodent (e.g., mouse, rate, hamster, or rabbit) and chicken embryo cells. Suitable host cells include, but are not limited to, hematopoietic cells (hematopoietic stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts, tumor cells and cell lines including, for example, CV-1, BSC40, Vero, BSC40 and BSC-1, and human HeLa cells. Methods for infecting and/or transforming host cells, phenotypically selecting infected cells or transformants, and other such methods are known in the art.

3. Combinations

Provided are combinations of a virus provided herein and a second agent, such as a second virus or other therapeutic or diagnostic agent. A combination can include a virus provided herein with one or more additional viruses, including, for example, one or more additional diagnostic or therapeutic viruses. A combination can contain pharmaceutical compositions containing a virus provided herein or host cells containing a virus as described herein. A combination also can include any virus or reagent for effecting treatment or diagnosis in accord with the methods provided herein such as, for example, an antiviral or chemotherapeutic agent. Combinations also can contain a compound used for the modulation of gene expression from endogenous or heterologous genes encoded by the virus.

Combinations provided herein can contain a virus and a therapeutic compound. Therapeutic compounds for the compositions provided herein can be, for example, an anti-cancer or chemotherapeutic compound. Exemplary therapeutic compounds include, for example, cytokines, growth factors, photosensitizing agents, radionuclides, toxins, siRNA molecules, enzyme/pro E drug pairs, anti-metabolites, signaling modulators, anti-cancer antibiotics, anti-cancer antibodies, angiogenesis inhibitors, chemotherapeutic compounds, antimetastatic compounds or a combination of any thereof.

Viruses provided herein can be combined with an anti-cancer compound, such as a platinum coordination complex. Exemplary platinum coordination complexes include, for example, cisplatin, carboplatin, oxaliplatin, DWA2114R, NK121, IS3 295, and 254-S. Exemplary chemotherapeutic agents also include, but are not limited to, methotrexate, vincristine, adriamycin, non-sugar containing chloroethylnitrosoureas, 5-fluorouracil, mitomycin C, bleomycin, doxorubicin, dacarbazine, taxol, fragyline, Meglamine GLA, valrubicin, carmustine, polifeprosan, MM1270, BAY 12-9566, RAS farnesyl transferase inhibitor, farnesyl transferase inhibitor, MMP, MTA/LY231514, lometrexol/LY264618, Glamolec, CI-994, TNP-470, Hycamtin/topotecan, PKC412, Valspodar/PSC833, Novantrone/mitoxantrone, Metaret/suramin, BB-94/batimastat, E7070, BCH-4556, CS-682, 9-AC, AG3340, AG3433, Incel/VX-710, VX-853, ZD0101, IS1641, ODN 698, TA 2516/marimastat, BB2516/marimastat, CDP 845, D2163, PD183805, DX8951f, Lemonal DP 2202, FK 317, picibanil/OK-432, valrubicin/AD 32, strontium-89/Metastron, Temodal/temozolomide, Yewtaxan/paclitaxel, Taxol/paclitaxel, Paxex/paclitaxel, Cyclopax/oral paclitaxel, Xeloda/capecitabine, Furtulon/doxifluridine, oral taxoids, SPU-077/cisplatin, HMR 1275/flavopiridol, CP-358 (774)/EGFR, CP-609 (754)/RAS oncogene inhibitor, BMS-182751/oral platinum, UFT(Tegafur/Uracil), Ergamisol/levamisole, Campto/levamisole, Eniluracil/776C85/5FU enhancer, Camptosar/irinotecan, Tomudex/raltitrexed, Leustatin/cladribine, Caelyx/liposomal doxorubicin, Myocet/liposomal doxorubicin, Doxil/liposomal doxorubicin, Evacet/liposomal doxorubicin, Fludara/fludarabine, Pharmorubicin/epirubicin, DepoCyt, ZD1839, LU 79553/Bis-Naphthalimide, LU 103793/Dolastatin, Gemzar/gemcitabine, ZD 0473/AnorMED, YM 116, Iodine seeds, CDK4 and CDK2 inhibitors, PARP inhibitors, D4809/dexifosfamide, Ifex/Mesnex/ifosfamide, Vumon/teniposide, Paraplatin/carboplatin, Platinol/cisplatin, VePesid/Eposin/Etopophos/etoposide, ZD 9331, Taxotere/docetaxel, prodrugs of guanine arabinoside, taxane analogs, nitrosoureas, alkylating agents such as melphalan and cyclophosphamide, aminoglutethimide, asparaginase, busulfan, carboplatin, chlorambucil, cytarabine HCl, dactinomycin, daunorubicin HCl, estramustine phosphate sodium, etoposide (VP16-213), floxuridine, fluorouracil (5-FU), flutamide, hydroxyurea (hydroxycarbamide), ifosfamide, interferon alfa-2a, interferon alfa-2b, leuprolide acetate (LHRH-releasing factor analogue), lomustine (CCNU), mechlorethamine HCl (nitrogen mustard), mercaptopurine, mesna, mitotane (o,p′-DDD), mitoxantrone HCl, octreotide, plicamycin, procarbazine HCl, streptozocin, tamoxifen citrate, thioguanine, thiotepa, vinblastine sulfate, amsacrine (m-AMSA), azacitidine, erythropoietin, hexamethylmelamine (HMM), interleukin 2, mitoguazone (methyl-GAG; methyl glyoxal bis-guanylhydrazone; MGBG), pentostatin (2′deoxycoformycin), semustine (methyl-CCNU), teniposide (VM-26) and vindesine sulfate. Additional exemplary therapeutic compounds for the use in pharmaceutical compositions and combinations provided herein can be found elsewhere herein (see e.g., Section I for exemplary cytokines, growth factors, photosensitizing agents, radionuclides, toxins, siRNA molecules, enzyme/pro-drug pairs, anti-metabolites, signaling modulators, anti-cancer antibiotics, anti-cancer antibodies, angiogenesis inhibitors, and chemotherapeutic compounds).

In some examples, the combination can include additional therapeutic compounds such as, for example, compounds that are substrates for enzymes encoded and expressed by the virus, or other therapeutic compounds provided herein or known in the art to act in.concert with a virus. For example, the virus can express an enzyme that converts a prodrug into an active chemotherapy drug for killing the cancer cell. Hence, combinations provided herein can contain a therapeutic compound, such as a prodrug. An exemplary virus/therapeutic compound combination can include a virus encoding Herpes simplex virus thymidine kinase with the prodrug ganciclovir. Additional exemplary enzyme/pro-drug pairs, for the use in combinations provided include, but are not limited to, varicella zoster thymidine kinase/ganciclovir, cytosine deaminase/5-fluorouracil, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid, cytochrome P450/acetaminophen, horseradish peroxidase/indole-3-acetic acid, nitroreductase/CB 1954, rabbit carboxylesterase/7-ethyl-10-[4-(1-piperidino)-1-piperidino]carbonyloxycamptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin-glucuronide, thymidine phosphorylase/5′-deoxy-5-fluorouridine, deoxycytidine kinase/cytosine arabinoside, beta-lactamase and linamerase/linamarin. Additional exemplary prodrugs, for the use in combinations can also be found elsewhere herein (see e.g., Section I). Any of a variety of known combinations provided herein or otherwise known in the art can be included in the combinations provided herein.

In some examples, the combination can include compounds that can kill or inhibit viral growth or toxicity. Such compounds can be used to alleviate one or more adverse side effects that can result from viral infection (see, e.g., U.S. Patent Pub. No. US 2009-016228-A1). Combinations provided herein can contain antibiotic, antifungal, anti-parasitic or antiviral compounds for treatment of infections. In some examples, the antiviral compound is a chemotherapeutic agent that inhibits viral growth or toxicity.

Exemplary antibiotics which can be included in a combination with a virus provided herein include, but are not limited to, ceftazidime, cefepime, imipenem, aminoglycoside, vancomycin and antipseudomonal β-lactam. Exemplary antifungal agents which can be included in a combination with a virus provided herein include, but are not limited to, amphotericin B, dapsone, fluconazole, flucytosine, griseofulvin, itraconazole, ketoconazole, miconazole, clotrimazole, nystatin, and combinations thereof. Exemplary antiviral agents can be included in a combination with a virus provided herein include, but are not limited to, cidofovir, alkoxyalkyl esters of cidofovir (CDV), cyclic CDV, and (S)-9-(3-hydroxy-2 phosphonylmethoxypropyl)adenine, 5-(dimethoxymethyl)-2′-deoxyuridine, isatin-beta-thiosemicarbazone, N-methanocarbathymidine, brivudine, 7-deazaneplanocin A, ST-246, Gleevec, 2′-beta-fluoro-2′,3′-dideoxyadenosine, indinavir, nelfinavir, ritonavir, nevirapine, AZT, ddI, ddC, and combinations thereof. Typically, combinations with an antiviral agent contain an antiviral agent known to be effective against the virus of the combination. For example, combinations can contain a vaccinia virus with an antiviral compound, such as cidofovir, alkoxyalkyl esters of cidofovir, ganciclovir, acyclovir, ST-246, Gleevec, and derivatives thereof.

In some examples, the combination can include a detectable compound. A detectable compound can include, for example, a ligand, substrate or other compound that can interact with and/or bind specifically to a protein or RNA encoded and expressed by the virus, and can provide a detectable signal, such as a signal detectable by tomographic, spectroscopic, magnetic resonance, or other known techniques. In some examples, the protein or RNA is an exogenous protein or RNA. In some examples, the protein or RNA expressed by the virus modifies the detectable compound where the modified compound emits a detectable signal. Exemplary detectable compounds can be, or can contain, an imaging agent such as a magnetic resonance, ultrasound or tomographic imaging agent, including a radionuclide. The detectable compound can include any of a variety of compounds as provided elsewhere herein or are otherwise known in the art. Exemplary proteins that can be expressed by the virus and a detectable compound combinations employed for detection include, but are not limited to luciferase and luciferin, β-galactosidase and (4,7,10-tri(acetic acid)-1-(2-β-galactopyranosylethoxy)-1,4,7,10-tetraazacyclododecane) gadolinium (Egad), and other combinations known in the art.

In some examples, the combination can include a gene expression modulating compound that regulates expression of one or more genes encoded by the virus. Compounds that modulate gene expression are known in the art, and include, but are not limited to, transcriptional activators, inducers, transcriptional suppressors, RNA polymerase inhibitors and RNA binding compounds such as siRNA or ribozymes. Any of a variety of gene expression modulating compounds known in the art can be included in the combinations provided herein. Typically, the gene expression modulating compound included with a virus in the combinations provided herein will be a compound that can bind, inhibit or react with one or more compounds, active in gene expression such as a transcription factor or RNA of the virus of the combination. An exemplary virus/expression modulator combinations can be a virus encoding a chimeric transcription factor complex having a mutant human progesterone receptor fused to a yeast GALA DNA-binding domain an activation domain of the herpes simplex virus protein VP16 and also containing a synthetic promoter containing a series of GALA recognition sequences upstream of the adenovirus major late EIB TATA box, where the compound can be RU486 (see, e.g., Yu et al., (2002) Mol Genet Genomics 268:169-178). A variety of other virus/expression modulator combinations known in the art also can be included in the combinations provided herein.

In some examples, the combination can contain nanoparticles. Nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen.

In some examples, the combination can contain one or more additional therapeutic and/or diagnostic viruses or other therapeutic and/or diagnostic microorganism (e.g., therapeutic and/or diagnostic bacteria) for diagnosis or treatment. Exemplary therapeutic and/or diagnostic viruses are known in the art and include, but are not limited to, therapeutic and/or diagnostic poxviruses, herpesviruses, adenoviruses, adeno-associated viruses, and reoviruses. Exemplary oncolytic viruses are described herein above.

4. Kits

The viruses, cells, pharmaceutical compositions or combinations provided herein can be packaged as kits. Kits can optionally include one or more components such as instructions for use, devices and additional reagents, and components, such as tubes, containers and syringes for practice of the methods. Exemplary kits can include a virus provided herein, and can optionally include instructions for use, a device for detecting a virus in a subject, a device for administering the virus to a subject, or a device for administering an additional agent or compound to a subject.

In one example, a kit can contain instructions. Instructions typically include a tangible expression describing the virus and, optionally, other components included in the kit, and methods for administration, including methods for determining the proper state of the subject, the proper dosage amount, and the proper administration method, for administering the virus. Instructions also can include guidance for monitoring the subject over the duration of the treatment.

In another example, a kit can contain a device for detecting a virus in a subject. Devices for detecting a virus in a subject can include a low light imaging device for detecting light, for example, emitted from luciferase, or fluoresced from fluorescent protein, such as a green or red fluorescent protein, a magnetic resonance measuring device such as an MRI or NMR device, a tomographic scanner, such as a PET, CT, CAT, SPECT or other related scanner, an ultrasound device, or other device that can be used to detect a protein expressed by the virus within the subject. Typically, the device of the kit will be able to detect one or more proteins expressed by the virus of the kit. Any of a variety of kits containing viruses and detection devices can be included in the kits provided herein, for example, a virus expressing luciferase and a low light imager or a virus expressing fluorescent protein, such as a green or red fluorescent protein, and a low light imager.

Kits provided herein also can include a device for administering a virus to a subject. Any of a variety of devices known in the art for administering medications, pharmaceutical compositions and vaccines can be included in the kits provided herein. Exemplary devices include, but are not limited to, a hypodermic needle, an intravenous needle, a catheter, a needle-less injection device, an inhaler and a liquid dispenser, such as an eyedropper. For example, a virus to be delivered systemically, for example, by intravenous injection, can be included in a kit with a hypodermic needle and syringe. Typically, the device for administering a virus of the kit will be compatible with the virus of the kit; for example, a needle-less injection device such as a high pressure injection device can be included in kits with viruses not damaged by high pressure injection, but is typically not included in kits with viruses damaged by high pressure injection.

Kits provided herein also can include a device for administering an additional agent or compound to a subject. Any of a variety of devices known in the art for administering medications to a subject can be included in the kits provided herein. Exemplary devices inclUde, but are not limited to, a hypodermic needle, an intravenous needle, a catheter, a needle-less injection device, an inhaler and a liquid dispenser, such as an eyedropper. Typically the device for administering the compound of the kit will be compatible with the desired method of administration of the compound. For example, a compound to be delivered systemically or subcutaneously can be included in a kit with a hypodermic needle and syringe.

The kits provided herein also can include any device for applying energy to a subject, such as electromagnetic energy. Such devices include, but are not limited to, a laser, light-emitting diodes, fluorescent lamps, dichroic lamps, and a light box. Kits also can include devices to effect internal exposure of energy to a subject, such as an endoscope or fiber optic catheter.

### G. THERAPEUTIC, DIAGNOSTIC AND MONITORING METHODS

The methods provided herein can be used for diagnostic, monitoring and/or therapeutic procedures. The initial step in the methods provided herein, involves administering a nucleic acid encoding one or more chromophore-producing enzymes to a subject, resulting in production of the chromophore(s) in a cell or tissue that is to be treated, detected and/or monitored. As described elsewhere herein, any expression vector, such as an oncolytic virus, for example LIVP virus, including derivatives thereof (described elsewhere herein), can be modified to contain nucleic acid encoding one or more chromophore-producing enzymes (e.g., Tyrosinase and/or Tyrosinase-related protein-1) and administered a subject, resulting in translation of the chromophore-producing enzyme(s) and production of the chromophore(s) in a target cell or tissue. Following nucleic acid delivery and chromophore accumulation, the chromophore-containing cell or tissue is exposed to one or more forms of energy, for example, electro-magnetic, acoustic, and/or magnetic energy, which is absorbed by the accumulated chromophore and utilized for therapeutic, diagnostic, and or monitoring applications.

The time between nucleic acid delivery and energy application depends on the desired application. Generally, timing is a function of the delivery agent and also the targeted tissue/cells. Energy should be applied/administered after a sufficient time for the delivered nucleic acid to be expressed and for the chromophore to be produced. For example, for therapeutic applications, the energy application can occur within 4 hr to 3 months after the nucleic acid(s) encoding one or more chromophore-producing enzymes are delivered to a subject, for example, within 6 hr to 2 months, 12 hr to 1 month, 18 hr to 2 weeks, typically 24 hr to 1 week or 1, 2, 3, 4, 5, 6, or 7 days post nucleic acid delivery. In some examples, for example, when circulating cells are the target of the therapeutic application, external energy application occurs within 4 to 36 hr after nucleic acid delivery, for example 12-24 hr, typically 18-24 hr after nucleic acid delivery, such as 18, 19, 20, 21, 22, 23, or 24 hr after nucleic acid delivery. Typically, for viruses, the energy is applied 24 hours, 48 hrs, 72 hours, 96 hours, 5 days, 6 days, 7 days, or 8 days after delivery of the nucleic acid.

The energy can be applied one time, repeatedly and/or intermittently after administration of the nucleic acid molecule. The energy also can be applied a plurality of times. Once nucleic acid treatment commences, and is repeated, each (nucleic acid delivery and energy treatment) can be administered at scheduled intervals. For example, treatment can be effected every day, every other day, once a week or once a month. In addition, since the energy treatment can be combined with detection, administration can be empirically determined as tumor tissue/cells are eliminated.

For example, application of therapeutic energy administration can occur in a single dose or in multiple doses, such as 2, 3, 4, 5, 6, 7 or more doses, for example, in one or more cycles of administration, over a period of time. In some examples, one or more forms of energy can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle. The dose of energy applied need not be the same on consecutive days, nor in subsequent cycles. The type of energy applied (e.g., electro-magnetic, acoustic, and/or magnetic energy) also can vary between administrations. Generally, a cycle of administration is 7 days, 14 days, 21 days or 28 days. Depending on the responsiveness or prognosis of the patient, the cycle of administration is repeated over the course of several months or years.

For diagnostic and monitoring methods, described herein, chromophore-containing cells or tissues can be detected for example, beginning at 6 hr to 1 week after nucleic acid delivery, for example 12-72 hr, 16-48, typically 18-48 hr, such as 18, 20, 24, 30, 36, 42, or 48 hr after nucleic acid delivery. Detection of chromophore-containing cells or tissues can be repeated at any time after the initial detection, for example, for monitoring applications. In some examples the chromophore-containing cells or tissues are detected and/or monitored following a single administration of the nucleic acid encoding the chromophore-producing enzyme(s). In other examples, booster administrations of the nucleic acid can be administered between detection procedures.

In some examples diagnostic/detection methods are combined with therapeutic methods described herein. For example, diagnostic/detection methods can be used prior to therapeutic methods to detect the cell(s) or tissue(s) to be treated and direct application of the therapeutic treatment. In other examples, detection methods, described herein, can be used to monitor the efficacy of the therapeutic methods. In some examples, the detection methods can be used to monitor the effects of applied therapeutic methods and to diagnose additional cells or tissues for therapeutic treatment that may arise over the course of the treatment regimen.

For therapeutic methods described herein, energy, for example, electromagnetic energy, is applied to the cell or tissue containing the chromophore product, resulting in local production of heat and/or toxic chemicals (e.g., reactive oxygen species) which effect destruction of the host cell or tissue, or sensitize the cell or tissue for additional therapeutic treatment(s). For diagnostic methods described herein, the properties of the chromophore enhance detection and/or diagnosis of the chromophore-producing host cell or tissue using one or more diagnostic imaging methods, for example ultrasound, magnetic resonance, and/or optoacoustic imaging methods. The chromophore-containing cell or tissue of a subject also can be monitored using any one or more of the diagnostic imaging methods, for example before, during, and/or following one or more therapeutic treatments, including one or more therapeutic treatments exploiting the chromophore product as described herein.

The therapeutic, diagnostic, and/or monitoring methods described herein, can be applied to any proliferative disorders or hyperproliferative disorders, including any disorders involving abnormal proliferation of cells. Such disorders include, but are not limited to, neoplastic diseases; inflammatory responses and disorders, including wound healing responses; psoriasis; restenosis; macular degeneration; diabetic retinopathies; endometriosis; benign prostatic hypertrophy; benign prostatic hyperplasia; hypertrophic scarring; cirrhosis; proliferative vitreoretinopathy; retinopathy of prematurity; and autoimmune or immunoproliferative diseases or disorders, e.g., inflammatory bowel disease, rheumatoid arthritis, multiple sclerosis, systemic lupus erythematosus (SLE) and vascular hyperproliferation secondary to retinal hypoxia or vasculitis.

Exemplary neoplastic diseases, for which the therapeutic, diagnostic, and/or monitoring methods described herein can be applied, include carcinoma, sarcoma, lymphoma or leukemia. In particular, the methods provided herein can effect treatment and/or detection of solid tumors (e.g., sarcomas, carcinomas or epithelial tumors or lymphomas) and cancers, other than solid tumors, such as leukemia and metastatic disease. For example, exemplary tumors, cancers or neoplastic diseases include, for example, carcinoma of the tongue, mouth, throat, stomach, cecum, colon, rectum, breast, ovary, uterus, thyroid, adrenal cortex, lung, kidney, prostate, pancreas, a melanoma, a basal cell carcinoma of the skin, a leukemia, a lymphoma, or an osteosarcoma.

In particular, examples of solid tumors include, but are not limited to, sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Cancers amenable to the treatment and detection methods described herein also include cancers that metastasize. It is understood by those in the art that metastasis is the spread of cells from a primary tumor to a noncontiguous site, usually via the bloodstream or lymphatics, which results in the establishment of a secondary tumor growth. Examples of cancers contemplated for treatment include, but are not limited to melanoma, including choroidal and cutaneous melanoma; bladder, non-small cell lung, small cell lung, lung, head, neck, breast, pancreatic, gum, tongue, prostate, renal, bone, testicular, ovarian, cervical, gastrointestinal lymphoma, brain, or colon cancer; hepatocarcinoma; retinoblastoma; mesothelioma; astrocytoma; glioblastoma; neuroblastoma; and any other tumors or neoplasms that are metastasized or at risk of metastasis.

The subject of the methods provided herein can be any subject, such as an animal or plant subject, including mammal or avian species. For example, the animal subject can be a human or non-human animal including, but not limited to, domesticated and farm animals, such as a pig, cow, a goat, sheep, horse, cat, or dog. In particular examples, the animal subject is a human subject.

1. Delivery of Nucleic Acid Encoding Melanin-Producing Enzymes and Melanin Biosynthesis

Any mode of administering exogenous nucleic acid to a can be employed to deliver a nucleic acid encoding a chromophore producing enzyme, such as a melanin-producing enzyme, to a subject, provided the mode of administration permits the nucleic acid to enter a target cell or tissue, for example, a tumor or metastasis. For example, viral or non-viral delivery methods of nucleic acid delivery can be used. The nucleic acid molecule can be cloned into any type of expression vector known to one of skill in the art, including plasmids, cosmids, artificial chromosomes, such as human artificial chromosomes, bacterial artificial chromosomes, yeast artificial chromosomes, and viral vectors.

a. Methods of Nucleic Acid Delivery

The nucleic acid molecule can be delivered or administered to a subject locally or systemically. For example, the nucleic acid molecule can be delivered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, topically, ocularly, vaginally, or rectally. Typically, administration is intravenous. In some examples, administration of the nucleic acid can be effected directly into the target tissue to be treated, such as locally into a body cavity. To increase the frequency of delivery into cells into a subject, the nucleic acid, such as a non-viral vector or DNA, can be delivered in the presence of a physical method to facilitate entry into cells selected from among electroporation, sonoporation, hydrodynamic, pressure, ultrasound and gene gun.

For example, several methods exist whereby an expression vector can be introduced into a target cell or tissue, including but not limited to transfection, microinjection, ballistic DNA injection (e.g., gene gun), and infection, any of which can be used in the methods provided herein. Methodologies of transfection of non-viral expression vectors are known in the art and including, but limited to, calcium phosphate, electroporation, heat shock, magnetofection, and the use of cationic lipids such as Lipofectamine™, FuGene®, Lipofectin™, Optifect™ and others known to one of skill in the art. One of skill in the art can determine which cell type should be transfected or infected, the type of expression vector to use, and by which method the expression vector is introduced, based on empirical determination.

In examples where the expression vector is a virus, any mode of viral delivery can be used. Modes of viral administration can include, but are not limited to, systemic, parenteral, intravenous, intraperitoneal, subcutaneous, intramuscular, transdermal, intradermal, intra-arterial (e.g., hepatic artery infusion), intravesicular perfusion, intrapleural, intraarticular, topical, intratumoral, intralesional, endoscopic, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, ocular or topical administration.

One skilled in the art can select any mode of administration compatible with the subject and the nucleic acid and that also is likely to result in the nucleic acid reaching and entering the target cell-type or tissue, e.g., tumors and/or metastases. The route of administration can be selected by one skilled in the art according to any of a variety of factors, including the nature of the disease, the properties of the target cell or tissue, and the nature of the nucleic acid, for example, the particular virus to be administered.

Provided herein are methods of systemically administering a vaccinia virus, such as any provided herein, to treat a proliferative or inflammatory disease or condition. In particular, the condition is associated with immunoprivileged cells or tissues. A disease or condition associated with immunoprivileged cells or tissues includes, for example, proliferative disorders or conditions, including the treatment (such as inhibition) of cancerous cells, neoplasms, tumors, metastases, cancer stem cells, and other immunoprivileged cells or tissues, such as wounds and wounded or inflamed tissues. In particular examples of such methods, compositions provided herein are administered by intravenous administration.

To decrease susceptibility of the administered nucleic acid to degradation by serum proteases following systemic administration, and improve stability in vivo, the nucleic acid can be condensed or encapsulated within vectors using viral capsids, lipids, cationic polymers, or protein polymers, such as silk-elastin like protein polymers (SELP). For example, a vaccinia virus, modified to contain nucleic acid encoding a chromophore-producing enzyme, can be encapsulated in a protein polymer and delivered to a subject by systemic administration (e.g., intravenous administration) or topical administration directly to the surface of a wound or lesion or other surface of a subject. For example, administration to the target site can be performed, for example, by systemic administration by injection into an artery or by topical administration by direct application onto a surface or by surface application of a coated device (e.g., bandage). Exemplary methods of delivery of such vaccinia in protein-polymer compositions is described in U.S. application Ser. No. 61/742,895, any of which can be used in the methods herein. In some examples, a diagnostic or therapeutic agent as described elsewhere herein also can be similarly administered.

The compositions provided herein can be administered by a single injection, by multiple injections, or continuously. For example, the compositions can be administered by slow infusion including using an intravenous pump, syringe pump, intravenous drip or slow injection. For example, continuous administration of the compositions can occur over the course of minutes to hours, such as between or about between 1 minute to 1 hour, such as between 20 and 60 minutes.

b. Dosages and Dosage Regimes

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic or other drugs, being administered concurrently. In examples where the nucleic acid is a virus, dosage levels can be affected by the infectivity of the virus and the nature of the virus, in addition to the factors above, as can be determined by one skilled in the art. The dosages of administered nucleic acid or vector can be determined by one of skill in the art. Considerations in determining the dosage can include the cell type and/or tissue to which the nucleic acid is to be administered, the rate of translation of the chromophore-producing enzyme, the stability (half-life) of the chromophore-producing enzyme, the promoter used to express the transgene, the biosynthesis pathway of the chromophore produced by the expressed transgene, the particular delivered agent, the method of delivery and other factors.

Where the delivered agent is non-viral nucleic acid, an effective dosage amount of nucleic acid (i.e., DNA or RNA) is in the range of from about 0.005 mg/kg body weight to about 50 mg/kg body weight. Generally, the dosage is in a range of or of about from 0.005 mg/kg to about or 20 mg/kg, and more generally in a range of or of about 0.05 mg/kg to about or 5 mg/kg. For example, for non-viral nucleic acid (e.g., plasmid, naked DNA, siRNA, shRNA or antisense nucleic acid), 0.01 mg to 2000 mg is delivered, such as 0.05 mg to 1500 mg, 1 mg to 1000 mg, 10 mg to 1500 mg, or 100 mg to 1000 mg.

In the present methods, appropriate minimum dosage levels and dosage regimes of viruses in the compositions herein can be levels sufficient for the virus to survive, grow and replicate in a tumor, metastasis or other wound or lesion. Generally, the virus is administered in an amount that is at least or about or 1×10⁵ pfu. Exemplary minimum levels for administering a virus to a 65 kg human can include at least about 1×10⁵ plaque forming units (pfu), at least about 5×10⁵ pfu, at least about 1×10⁶ pfu, at least about 5×10⁶ pfu, at least about 1×10⁷ pfu, at least about 1×10⁸ pfu, at least about 1×10⁹ pfu, or at least about 1×10¹⁰ pfu. For example, the virus is administered in an amount that is at least or about or is 1×10⁵ pfu, 1×10⁶ pfu, 1×10⁷ pfu, 1×10⁸ pfu, 1×10⁹ pfu, 1×10¹⁰ pfu, 1×10¹¹ pfu, 1×10¹²pfu, 1×10¹³ pfu, or 1×10¹⁴ pfu at least one time over a cycle of administration.

In the dosage regime, the amount of virus can be delivered as a single administration or multiple times over a cycle of administration. Hence, the methods provided herein can include a single administration of a virus to a subject or multiple administrations of a virus to a subject. In some examples, a single administration is sufficient to establish a virus in a tumor, where the virus can proliferate and can cause or enhance an anti-tumor response in the subject; such methods do not require additional administrations of a virus in order to cause or enhance an anti-tumor response in a subject, which can result, for example in inhibition of tumor growth, inhibition of metastasis growth or formation, reduction in tumor or size, elimination of a tumor or metastasis, inhibition or prevention of recurrence of a neoplastic disease or new tumor formation, or other cancer therapeutic effects.

In other examples, the virus can be administered on different occasions, separated in time typically by at least one day. For example, the compositions can be administered two times, three time, four times, five times, or six times or more, with one day or more, two days or more, one week or more, or one month or more time between administrations. Separate administrations can increase the likelihood of delivering a virus to a tumor or metastasis, where a previous administration has been ineffective in delivering a virus to a tumor or metastasis. Separate administrations can increase the locations on a tumor or metastasis where virus proliferation can occur or can otherwise increase the titer of virus accumulated in the tumor, which can increase the scale of release of antigens or other compounds from the tumor in eliciting or enhancing a host's anti-tumor immune response, and also can, optionally, increase the level of virus-based tumor lysis or tumor cell death. Separate administrations of a virus can further extend a subject's immune response against viral antigens, which can extend the host's immune response to tumors or metastases in which viruses have accumulated, and can increase the likelihood of a host mounting an anti-tumor immune response.

When separate administrations are performed, each administration can be a dosage amount that is the same or different relative to other administration dosage amounts. In one example, all administration dosage amounts are the same. In other examples, a first dosage amount can be a larger dosage amount than one or more subsequent dosage amounts, for example, at least 10× larger, at least 100× larger, or at least 1000× larger than subsequent dosage amounts. In one example of a method of separate administrations in which the first dosage amount is greater than one or more subsequent dosage amounts, all subsequent dosage amounts can be the same or smaller amount relative to the first administration.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a virus, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of tumor growth or inhibition of tumor growth, appearance of new metastases or inhibition of metastasis, the subject's anti-virus antibody titer, the subject's anti-tumor antibody titer, the overall health of the subject, the weight of the subject, the presence of virus solely in tumor and/or metastases, the presence of virus in normal tissues or organs.

The time period between administrations can be any of a variety of time periods. The time period between administrations can be a function of any of a variety of factors, including monitoring steps, as described in relation to the number of administrations, the time period for a subject to mount an immune response, the time period for a subject to clear the virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month.

In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

For example, an amount of virus is administered two times, three times, four times, five times, six times or seven times over a cycle of administration. The amount of virus can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle. Generally, the cycle of administration is 7 days, 14 days, 21 days or 28 days. Depending on the responsiveness or prognosis of the patient the cycle of administration is repeated over the course of several months or years.

Generally, appropriate maximum dosage levels or dosage regimes of viruses are levels that are not toxic to the host, levels that do not cause splenomegaly of 3 times or more, levels that do not result in colonies or plaques in normal tissues or organs after about 1 day or after about 3 days or after about 7 days.

2. Therapeutic Methods

The methods provided herein can be used for the treatment of any proliferative disorders or hyperproliferative disorders, including any disorders involving abnormal proliferation of cells, such as neoplastic diseases, inflammatory responses and disorders, which warrant removal of particular cells or tissues while preserving healthy cells and tissues. In particular, provided herein are methods of treating cancerous cells, neoplasms, tumors, metastases and cancer stem cells by delivering nucleic acid encoding a chromophore-producing enzyme (e.g., a melanin producing enzyme such as a tyrosinase or tyrosinase-related protein-1) followed by transferring energy to the chromophore resulting in the destruction of the chromophore-containing host cell or tissue. In some examples provided herein, viruses (e.g., the oncolytic viruses) containing the chromophore-producing enzyme(s) preferentially accumulate in tumors or metastases, resulting in chromophore (e.g., melanin) production in the targeted tissues.

In some examples, the administration alone of an oncolytic virus, containing nucleic acid encoding one or more melanin producing enzyme(s), results in a slowing of tumor growth, and in some cases an inhibition in tumor growth. As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005-0031643, now U.S. Pat. Nos. 7,588,767, 7,588,771, 7,662,398), which provide and exemplify the GLV-1h68 virus and derivatives thereof. Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophil count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production.

In the methods provided herein, the administration of a nucleic acid encoding a chromophore-producing enzyme (e.g., a melanin-producing enzyme) is followed by application of an energy source (e.g., electromagnetic energy, ultrasonic waves, or other conduction-based energy source), resulting in a transfer of energy to the chromophore (e.g., melanin). The absorption of the applied energy by the chromophore results in a release of thermal energy and/or the production of toxic products (e.g., ROS products), resulting in destruction of the host cell or tissue. In some examples, the tissue to be treated is a tumor and the transfer of energy from an energy source to the chromophore results in a decrease in tumor volume, including elimination or eradication of the tumor. In some examples, administration of an oncolytic virus containing nucleic acid encoding a melanin-producing enzyme results in selective infection of tumor cells and production of melanin within the tumor. Application of an energy source, for example a heat source, such as a wavelength of electromagnetic energy in the near infrared range, after melanin is produced in the tumor, results in a decrease in tumor volume, including elimination or eradication of the tumor.

Any form of energy which can be absorbed by the chromophore produced in the target cell or tissue, or precursors thereof, can be used in the methods provided herein, provided the energy can be absorbed by the chromophore product and converted in to a form of energy that can be used for therapeutic treatment. For example, chemical energy, electric energy, radiant energy, electromagnetic energy, nuclear energy, magnetic energy, elastic energy, sound energy, mechanical energy and luminous energy that can be absorbed by the chromophore product and used to generate a heat or chemical-based therapy to a target cell or tissue can be used.

In some examples, electromagnetic energy is applied as a phototherapeutic heating source to selectively induce toxicity or injury to melanin-producing cells and tissues, for example, cancer cells and tumors. Phototherapeutic tissue injury can be invoked by photothermal, photomechanical, or photochemical mechanisms, or a combination thereof. Photothermal mechanisms result from the conversion of light energy into thermal energy (heat). Photomechanical mechanisms result from rapid tissue heating, with very short, high energy pulses resulting in tissue rupture. Examples of photomechanical injury include photoablation (removal of tissue) and photoacoustics (shock waves). Photochemical mechanisms cause target molecules (e.g., photosensitizers) to start light-induced chemical reactions (e.g., photodynamic therapy (PDT)). The mechanism of tissue injury is a function of the density of power of the light source and the time of exposure. High power treatments for brief time periods (e.g., greater than 10⁶ W·cm²) for short periods of time (e.g., less than 10 μs) results in photomechanical tissue destruction.

One or more of the energy absorbing therapeutic methods described herein also can be employed in combination therapies, including with chemotherapy, radiotherapy and biological therapy.

a. Heat Therapies: Hyperthermia and Thermotherapy

In examples of the methods provided herein, energy is applied to a cell or tissue containing a chromophore, produced as a result of delivery of nucleic acid(s) encoding one or more chromophore-producing enzymes, resulting in the local production of heat which promotes or causes selective destruction of the cell or tissue hosting the chromophore. The degree to which the treated tissue is heated and the duration of the energy application determine the extent to which the chromophore-containing tissue is damaged. Elevating the temperature of a patient's tissue in the range of approximately 40-47° C., typically 41-42° C. (104-117° F., typically 106° F.) is called hyperthermia. At hyperthermic temperatures, heat shock pathways are activated. While some cellular components are damaged at hyperthermic temperatures, healthy cells and tissues are typically capable of recovery following exposure to temperatures within this range. When cells are exposed to higher temperatures, for example, temperatures exceeding approximately 50° C., the treatment is termed thermotherapy. Even brief exposure to thermotherapeutic temperatures results in irreparable damage and indiscriminant cell death, i.e., healthy and diseased cells and tissues are equally susceptible.

Several types of energy sources can be employed to produce the heat therapies described herein. For example, external or internal heating devices scan be used for application of local, regional, or whole-body heat therapy. Local heat therapies involve the application of heat to a small area, such as a tumor. Heat can be generated externally with high-frequency waves targeting a tumor from a device outside the body. Internal heating methods can employ a sterile probe, including thin, heated wires or hollow tubes filled with warm water, implanted microwave antennae and radiofrequency electrodes.

A patient's organ or a limb can be heated using regional hyperthermia therapy. This can be achieved with devices that produce high energy, such as magnets. Alternatively, some of the patient's blood can be removed and heated before being perfused into an area that will be internally heated. Whole-body heating also can be implemented in cases where cancer has spread throughout the body. Warm-water blankets, hot wax, inductive coils, and thermal chambers can be used for this purpose.

The heat therapies described herein results in cell or tissue damage or destruction. The types and extent of molecular and cellular damage caused depend on the duration and degree of heating. For example, extended heating, exceeding hyperthermic temperatures, such as temperatures of or of about 47° C. (e.g., thermotherapeutic temperatures), or temperatures of or exceeding 100° C., leads to increasing degrees of biological response and destruction. A list of some of the biological responses to varying degrees of heat treatment are set forth below in Table 10.

TABLE 10
Biological Responses to Heat Treatment

| | | |
|---|---|---|
| | | Increased heat |
| | | sensitivity of |
| Temper- | | tumors over |
| ature | Biological Effects | normal tissue |

hyperthermic temperatures

| | | |
|---|---|---|
| 40-44° C. | Protein conformational changes | yes |
| | Activation of heat shock pathway | |
| | Increased pore size/permeability | |
| | Increased cell sensitivity to radiation | |
| | Increased blood perfusion in normal tissues | |
| | Decreased blood perfusion in tumors | |
| 45-47° C. | Cytoskeleton reorganization | no |
| | Increased membrane fluidity | |
| | Inhibition of DNA, RNA, protein synthesis | |
| | hyperthermic killing | |

thermotherapeutic temperatures

| | | |
|---|---|---|
| 47-50° C. | Protein denaturation | no |
| ≧50° C. | Reduction of enzymatic activity | no |
| ≧60° C. | Irreversible denaturation of proteins | no |
| | Coagulation of collagens | |
| | Membrane rupture | |

vaporizing temperatures

| | | |
|---|---|---|
| 100° C. | Boiling of tissue water | no |
| | Formation of extracellular vacuoles | |
| | Cell shrinkage | |
| ≧100° C. | Breaking of vacuoles | no |
| | Carbonization | |
| ≧300° C. | Tissue thermoablation | no |
| | Charring | |

As set forth in forth in Table 10, treatment under hyperthermic conditions results in structural changes in protein conformation. This is due to breaking of hydrogen bonds and disruption of ionic interactions between macromolecules, including transmembrane proteins which are important for maintaining membrane integrity. Cell death, as a result of hyperthermia can be a combination of apoptotic cell death and necrosis. Thus, the extent of hyperthermic damage cannot be fully assessed until about 24-72 hr after treatment (Thomsen, (1991) Photochem Photobiol. 53(6):825-35).

At hyperthermic temperatures of 42-44° C., tumors are more sensitive than normal tissue (Dickson and Calderwood, “Thermosensitivity of neoplastic tissues in vivo,” in Hyperthermia in cancer therapy, FK Strom ed., (Boston:Hall Medical Publishers, 1983), pp. 63-140). Thus, clinical hyperthermia has been applied in this temperature range to induce cell death selectively of tumor cells while preserving the surrounding normal tissue. In addition, to exploit the hypersensitivity of tumors exposed to heat in this range, thermotherapy can be used in combination with other cancer treatments such as ionizing radiation, photodynamic therapies and drug therapies.

When temperatures exceed approximately 50° C., the treatment is termed thermotherapy. Thermotherapy facilitates rapid necrotic tissue destruction. There is no selectivity in heat sensitivity between neoplastic and healthy tissues at thermotherapeutic temperatures. Thus, thermotherapeutic treatments require precise targets. At temperatures at or exceeding 100° C., water in the tissue boils, resulting in trapped water vapor that leads to mechanical destruction from coalescing vapor bubbles and tissue dehydration. Continued heating results in burning, producing carbonized and/or charred tissue. Tissue destruction from vaporization and carbonization, at or above 100° C. can be vast and unpredictable. Thus for most applications of the heat therapies provided herein, heating the target cell(s) or tissue(s) to temperatures less than 100° C. is preferred.

In the methods provided herein, the target internal temperature of a tissue targeted for heat therapy, for example a chromophore-containing tumor, is approximately 42 to 99° C., or more, for a period of about 10 sec to 2 hr, such as 50 to 80° C. for a period of 30 sec to 20 min, for example, 1 to 10 min, such as 1, 2, 3, 4, 5, 6, 7, 8, 9 or 10 min. For some embodiments, the target internal temperature of the tissue targeted for heat therapy is up to or about 47° C. and maintained for a period of 2 min to 2 hr, for example 1 min to 1 hr, such as about 42 to 47° C. for a period of 2 min to 2 hr, 5 min to 1.5 hr, 15 min to 1 hr, 15, 20 min, 25, min, 30 min 45 min or 1 hr, 42 to 44° C. for a period of 5 min to 2 hr, or 45 to 47° C. for a period of 2 to 30 min, 2 to 15 min, 2 to 10 min, such as 2, 3, 4, 5, 6, 7, 8, 9 or 10 min.

In the methods provided herein, the healthy, non-targeted tissue (e.g., tissue not expressing the chromophore product) is not substantially damaged. For example, during the course of application of the methods provided herein, tissue not expressing the chromophore, with a 0 to 4 cm margin from the edge target tissue (e.g., a tumor), preferably a 1-2 cm margin from the edge of the target tissue does not exceed 44° C., preferably 42° C., more preferably less than 42° C., such as 40° C.

In some examples herein, measures optionally can be taken to reduce blood flow to a chromophore-expressing target tissue, for example to reduce convective heat loss from the target tissue during application of heat therapy, as described herein or to sensitize the tissue for heat therapy (see e.g., Sturesson et al., Lasers Surg Med. 43(1):29-35 (2011)). For example, a clamp, applying pressure adjacent to the region targeted for therapy, or an occlusion catheter, can isolate a region or segment of a tissue or organ from adjacent tissue(s) and from the surrounding vasculature and restrict blood flow to the target tissue. In some examples, the step of isolating a target tissue from the general circulation can be aided by imaging devices, such as ultrasonography, computed tomography or magnetic resonance imaging. In one example, an intra-operative ultrasound can be used to locate vascular patterns and other anatomical features of the tissue or organ in order to aid in blocking blood flow. In some examples, suction or flushing blood from the target tissue can be performed, in addition to isolation from surrounding vasculature, for example, prior to administration of the energy source to limit competing energy absorbance by biological chromophores present in blood (e.g., hemoglobin), and thereby optimize energy transfer to the chromophore product in the target tissue.

Methods of thermometry can optionally be employed in the methods provided herein to measure the temperature of the target tissue, for example, before, during and/or after application of the photothermal therapy. For example, the temperature of the target tissue is monitored during and/or immediately after treatment, preferably during treatment, to ensure proper dosage of the phototherapy. In some examples, the energy source of the phototherapy can be connected to a thermostat that can maintain a temperature over the course of the treatment. In other examples, the energy source is coupled to a thermistor, for example, such that the resistance increases when the target tissue reaches a pre-defined degree of temperature or after a pre-defined degree is maintained for a pre-determined amount of time. In other examples, a thermocouple is used, such as a fine-wire thermocouple to monitor and maintain a pre-determined temperature for a prescribed period of time.

Thus, in the methods described herein, one of skill in the art can determine the optimal irradiation parameters to achieve the desired biological effects in the treated tissue, while taking into account the temperature gradient and the resulting biological effects of the surrounding tissue(s).

i. Photothermal Therapy Methods

In the photothermal therapy methods provided herein, energy from electromagnetic radiation (light energy) is converted to thermal energy following absorbance of the applied electromagnetic radiation by one or more chromophores in a targeted cell or tissue, as a result of delivery of one or more chromophore-containing enzyme(s) the cell or tissue. Electromagnetic radiation is generally classified by wavelength (λ) as gamma rays, x-rays, ultraviolet (UV) light, visible light, infrared light, microwaves and radio waves. The wavelength of electromagnetic radiation is inversely proportional to the wave frequency, which is proportional to the photon energy. Table 11 below sets forth these three properties for the broad classifications of electromagnetic radiation.

TABLE 11
Wavelengths, frequencies, and energies of electromagnetic radiation

| Class | Wavelength | Frequency (Hz) | Photon Energy (eV) |
|---|---|---|---|
| Gamma ray | less than 0.01 nm | more than 10 EHZ | 100 keV-300+ GeV |
| X-Ray | 0.01 nm to 10 nm | 30 EHz-30 PHZ | 120 eV to 120 keV |
| Ultraviolet | 10 nm-390 nm | 30 PHZ-790 THz | 3 eV to 124 eV |
| Visible | 390 nm-750 nm | 790 THz-405 THz | 1.7 eV-3.3 eV |
| Infrared | 750 nm-1 mm | 405 THz-300 GHz | 1.24 meV-1.7 eV |
| Microwave | 1 mm-1 meter | 300 GHz-300 MHz | 1.24 μeV-1.24 meV |
| Radio | 1 mm-100,000 km | 300 GHz-3 Hz | 12.4 feV-1.24 meV |

Optical radiation is part of the electromagnetic radiation spectrum, ranging from 10 nm (in the UV range) to 1 mm (in the IR range). Many photobiology and photomedicine applications employ wavelengths within this range. The International Commission on Illumination (CIE) has subdivided this range into seven classes based on wavelength: UVC (100-280 nm), UVB (280-315 nm), UVA (315-400), light/visible radiation (400-780 nm), 1R-A (780-1400 nm), IR-B (1400-3000 nm), and IR-C (3000 nm-1 mm).

For biological applications, the dose (e.g., J/cm²) of electromagnetic radiation (light) is the product of the irradiance of the applied electromagnetic energy (i.e., power per unit area, e.g., W/cm²) and the exposure time (e.g., s). The exposure dose determines the biological effect of the exposure. Thus, for application of electromagnetic radiation in the methods provided herein, the same dose (with same effect) can be achieved by a high intensity of light applied for a short time or a lower intensity of light applied for a longer time. However, because chromophores absorb different wavelengths to different extents, the threshold exposure of a chromophore is wavelength dependent. For example, the threshold fluence for a wavelength that is better absorbed than another will be less than that for a wavelength that is less well-absorbed. The threshold fluence for a given chromophore can be determined by one skilled in the art using the absorbance spectrum of the chromophore and empirical determination.

Sources of electromagnetic energy include lasers, light-emitting diodes, fluorescent lamps, antennae, dichroic lamps, and a light box. Any wavelength, or range of wavelengths, of electromagnetic radiation can be used in the methods provided herein, provided that the energy from the electromagnetic radiation application is absorbed by the chromophore product(s), produced in a cell or tissue following administration of a nucleic acid encoding one or more chromophore-producing enzymes, and converted into thermal energy by way of heat dissipation.

a) Laser Light Therapy

In the methods described herein, laser light therapy can be used as an energy source for absorbance-based thermotherapeutic treatments. For example a laser can be used to transmit electromagnetic radiation to be absorbed by a chromophore-containing target cell or tissue, resulting from delivery of one or more nucleic acid molecules encoding one or more chromophore-producing enzyme(s) to the target tissue, which converts the electromagnetic energy into thermal energy and produces heat within the target cell or tissue.

i) Parameters

Any type of laser, known to one of skill in the art, can be used for the laser-based thermotherapy methods provided herein, including gas lasers, chemical and excimer lasers, solid-state lasers, fiber lasers, photonic crystal lasers, semiconductor lasers, dye lasers, and free electron lasers, which are named based on the type of gain medium used. Typically, solid-state lasers or fiber lasers, for example fiber optics, are employed in the methods provided herein.

Selection of the laser source depends on the parameters, selected by one of skill in the art for laser therapy. Parameters of a laser energy source include the wavelength, mean power (mW), irradiance (W/cm²), and dose (J/cm²) of the electromagnetic energy to be administered, the size of the area(s) to be treated, region of the tissue to be treated, for example external vs. interstitial applications, the beam shape (focused/collimated), including the numeric aperture if a focused beam is selected, beam waist, and beam area (mm²),

The optical power of the lasers used herein can be continuous or pulsed. In some examples, energy is emitted from the laser in continuous wave (CW) mode. In some examples pulsed lasers are used to transmit electromagnetic energy to a chromophore-containing cell or tissue. Pulsed laser operation can be achieved, for example, using Q-switching, mode-locking, or pulsed pumping techniques, depending on the desired output. In some examples, the pulse rate is modulated to regulate the amount of power delivered in a pulse.

Typically, lasers are engineered or tuned to transmit coherent monochromatic light (i.e., waves of a single wavelength that are in phase). Table 12 below sets forth several of the commonly used lasers and their corresponding wavelengths.

TABLE 12
Wavelengths of Common Lasers

| Laser Type | Wavelength (nm) |
|---|---|
| Argon Fluoride (ArF) | 193 |
| Xenon Chloride (XeCl) | 308 and 459 |
| Helium Cadmium (HeCd) | 325-442 |
| Nitrogen (N) | 337.1 nm |
| Xenon Fluoride (XeF) | 353 and 459 |
| Rhodamine 6G | 450-650 |
| Copper Vapor | 511 and 578 |
| Argon | 514.5 and 488 |
| Helium Neon (HeNe) | 543, 594, 612, and 632.8 |
| frequency doubled Nd: YAG | 532 and 266 |
| Laser Diodes | 630-950 |
| Krypton (Kr) | 647.1-676.4 |
| Ti: Sapphire | 690-960 |
| Ruby | 694.3 |
| Alexandrite | 720-780 |
| Ytterbium (Yb) | 1030-1120 |
| Neodymiun: YAG (Nd: YAG) | 1064 and 1319 |
| Hydrogen Fluoride (HF) | 2600-3000 |
| Carbon Monoxide (CO) | 5000-6000 |
| Carbon Dioxide (CO₂) | 10600 |

Emitted laser light can be collimated (parallel waves) or focused. Collimated beams permit exposure to more area and typically are held stationary during treatment. In examples where the area of the target tissue is larger than the laser beam, multiple overlapping exposures can be administered to cover the entire area of the target tissue. Focused beams can produce dramatically increased effective irradiance in a treated tissue, compared to collimated beams. Because focused beams have a smaller exposure area (beam waist, e.g., on the order of μm) compared to collimated beams, focused beams typically are scanned, for example by rastering, across the surface of the tissue to be treated (see e.g., Krause et al., Arch Opthalmol. 121:357-363 (2003).

In some examples, a stationary collimated beam is used to transmit electromagnetic energy at a desired wavelength for a predetermined amount of time to a chromophore-containing tissue, for example a melanin-containing tissue, to invoke a therapeutic heat response. In other examples, the surface of a chromophore-containing tissue is exposed to focused laser light at a desired wavelength for a predetermined amount of time using rastered scanning.

Any wavelength of electromagnetic radiation can be applied to a chromophore-containing tissue, by a laser source, provided that that chromophore is capable of absorbing the electromagnetic radiation at the applied wavelength and converting the applied energy into thermal energy, raising the temperature of the tissue to a predetermined degree. Several factors contribute to the selection of the wavelength to be applied to the chromophore-containing tissue in the methods provided herein. Such factors include, but are not limited to, the absorbance spectrum of the chromophore product contained within the target tissue, the thermal response of the chromophore to the selected wavelength, the absorbance properties of the tissue(s) and cells surrounding the target tissue at the selected wavelength(s), the extent of heating desired, the proximity of the energy source to the target tissue, the method of application, for example whether the source is external or internal to the target tissue, the extent of blood flow to the target tissue, and other parameters of the laser source.

ii) Laser Positioning

In the methods provided herein, electromagnetic energy emitted from a laser can be applied by invasive or non-invasive means. For example, the applied energy can be external or internal to the chromophore-containing tissue. In some examples using an external laser application, the laser can be positioned external to the subject, for example in direct contact with, or close to, the chromophore containing tissue to be treated, for example on the surface of a subject.

In other examples the laser light can be directed transdermally to the chromophore-containing target tissue, for example to a target tissue that underlies the skin of the subject, such as a subepidermal or subcutaneous tumor. For transdermal applications, the optical properties of the skin should be taken into consideration when selecting the wavelength to be applied. For example, wavelengths capable of penetrating skin, for example wavelengths of 600 to 1500 nm, such as 600 to 1200 nm, or 700 to 900 nm, such as 808 nm should be selected for maximum penetrance. Therefore, in some transdermal applications of the methods provided herein require accumulation of a chromophore product (e.g., melanin) in the target tissue that is capable of absorbing a sufficient amount of electromagnetic energy at the wavelengths provided above (i.e., wavelengths of or of about 600 to 1500 nm) to effect thermal energy emission in sufficient amounts to achieve hyperthermic or thermotherapeutic temperatures in the target tissue.

In other examples, electromagnetic energy can be emitted transdermally by two laser sources, of the same wavelength, within the range of 600 to 1500 nm, such as 600 to 1200 nm, for example 700 to 900 nm, such as 800 nm, 805 nm or 808 nm, that are positioned, or focused, for example, by a lens, so that the laser beams intersect (e.g., are confocal) at or within the target tissue, resulting in elevated electromagnetic energy levels in a focused volume at the point of laser beam intersection at or within the target tissue (see e.g., U.S. Pat. Nos. 5,829,448 and 7,036,516, incorporated herein by reference). In these examples, a chromophore product manufactured in the target tissue can be capable of absorbing a sufficient amount of electromagnetic energy at or about half of the wavelengths emitted by the individual lasers (i.e., at wavelengths of or of about 300 to 800 nm, such as 320 to 480 nm, for example about 400 nm) to effect thermal energy emission in sufficient amounts to achieve hyperthermic or thermotherapeutic temperatures in the target tissue.

In a particular example, external electromagnetic energy at a wavelength in the range of 600 to 1500 nm, such as 600 to 1200 nm, or 700 to 900 nm, such as 808 nm, from a laser source, such as a laser diode, such as a 2.0 W laser diode, is applied to a chromophore containing tissue, for example a melanin-containing tumor, such as a sub-cutaneous melanin-containing tumor at an energy level sufficient to increase the temperature of the target tissue for example by up to 30° C., such as by 3 to 30° C., for example 10 to 30° C., resulting in destruction of the target tissue. In this example, the electromagnetic energy can be applied for 10 seconds to 10 minutes, for example 30 seconds to 5 minutes, preferably 1 to 2 minutes.

In other examples, minimally invasive laser electromagnetic energy sources are used. In some examples, endoscopic laser sources can be inserted into a cavity of a subject, for example to position the energy source (i.e., laser) in proximity to an internal target tissue, for example a chromophore product-containing tumor, such as a melanin containing tumor. For endoscopic applications, similar considerations should be taken into account as for transdermal applications. For example, the laser source and wavelength should be selected to permit transmission of the electromagnetic energy through the lining of the cavity and to the chromophore-containing target tissue to effect heat therapy.

In some examples, fiber lasers, for example, fiber optics are inserted interstitially into the chromophore-containing target tissue so that the electromagnetic energy can be emitted directly into the target tissue, such as a chromophore-containing tumor. In some examples, imaging methods, such as ultrasound or magnetic resonance imaging, can be used to aid in the placement of the fiber optic within the target tissue. Because the fiber optic is placed interstitially, within the target tissue, electromagnetic energy is emitted directly into the chromophore product-containing target tissue, and any wavelength absorbed by the chromophore product can be selected and applied to the target tissue to effect heat therapy. Typically, multimode optical fibers, capable of emitting high-power light are used for interstitial laser therapy applications. In some examples, one fiber optic is inserted into the chromophore-containing target tissue. In other examples more than one fiber optic is inserted into the chromophore-containing tissue, for example, 2, 3, 4, or more fiber optics, depending on the size of the tissue to be treated.

In some examples, the interstitial laser heat therapy methods described herein employs a plane cut optical fiber to emit electromagnetic energy, for example, from a Nd:YAG laser, for example, at a power level of about 2 to 3 W, for example for a period of up to 30 min, for example 5-30 min, into a chromophore-expressing target tissue, such as a tumor, for example a tumor up to approximately 1.5 cm in diameter. In these examples, treatment should cease when the temperature at the border of the tumor reaches for example 50 to 60° C. or after a temperature of 45° C. is maintained at the border for approximately 5 to 30 min, for example, 15 min.

In another example, target tissues (e.g., a chromophore product-containing tumor) are subjected to interstitial laser thermotherapy using an optical fiber that is constructed with a sculpted fiber tip, for example a fiber tip fabricated from silica core/silica clad fiber, a silica core and polymer clad fiber, or a material that conducts heat well, such as artificial sapphire. In some examples, the manufactured tip is a continuous, integral part of the optical fiber to eliminate an interface, and any coupling losses or surface contamination between the tip and fiber. In other examples, the tip is attached to the optical fiber, for example, by a collar, such as a metal collar.

The fiber tip can be manufactured in a variety of shapes and sizes. For example, the fiber tip can be a taper or cone, convex or concave lens, diffuser, a side-fire tip or an angled end. In particular, the optical fiber can be equipped with a diffusing tip to increase the light emitting area of the optical fiber, so that more surface area of the chromophore product-containing target tissue can absorb the applied electromagnetic energy. The fiber tip optionally can be covered with a scattering coating, as described in van Hillegersberg et al., Lasers Surg. Med.14:124-138 (1994).

Methods employing an interstitial optical fiber for heat therapy also can employ a laser cooling mechanism to prevent undesired vaporization and carbonization at the tip of the energy source. Such cooling methods have been described in the art (see e.g., Doewlatshahi et al., Lasers Surg. Med. 12:159-164 (1992); Orth et al., Lasers Surg. Med. 20:149-156 (1997); Sturesson and Andersson-Engels, Phys. Med. Biol. 41:445-463 (1996); Sturesson and Andersson-Engels, Med. Phys. 24:461-470 (1997)).

In the methods provided herein, a chromophore-containing tissue (e.g., a melanin-containing tumor) can be treated by heat therapy, internally, by applying electromagnetic energy using an interstitial fiber optic. The type of laser used and the wavelengths emitted depend on the chromophore produced in the target tissue. In some examples, the chromophore product is melanin. In some examples, a melanin-containing tissue, for example a melanin-containing tumor is treated by interstitial laser heat therapy, and the wavelength selected is selected from the range of 500 to 1500 nm, such as 500 to 1200 nm, 600-100 nm, such as 805 nm, 808 nm, 1047 nm, or 1064 nm. The exposure time and power (i.e., the dosage) can be empirically determined based on the particular application. In some examples, a power of 0.1 to 3 W, such as 0.5 to 2 W power are used for a period of 30 sec to 30 min, such as 1 to 15 minutes, such as 5, 10, or 15 minutes. In some examples, temperatures at the border or within a 1 or 2 cm margin of the target tissue are maintained at temperatures less than 50° C., such as less than 47° C., preferably less than 45° C., such as 40 to 44° C.

In some examples, mathematical modeling can be used to predict the electromagnetic energy absorption of the chromophore product and target tissue, and generate maps of the heat distribution for different modes of treatment to aid in the planning of a treatment regimen (e.g., Sturesson and Andersson-Engels, Phys Med Biol. 40(12):2037-52 (1995); Fasano et al, Applied Mathematical Modeling 34(12):3831-3840 (2010)).

b) Microwave-Based Therapy

In other examples, non-invasive and invasive applications of microwaves can be used to induce heat therapy, for example hyperthermia and thermotherapy, in chromophore-containing target tissues. Microwave energy can be applied to treat superficial and deep-seated tumors using heat therapy (see for example, U.S. Pat. No. 6,181,970; Lindholm et al., Int. J. Hyperthermia 11:337-355, 1995; Vargas et al., (2004) Ann Surg Oncol. 11(2):139-46. Dooley et al., (2010) Ann Surg Oncol. 17(4):1076-93; Bicher et al., in Hyperthermic Oncology, vol. 1, J. Overgaard, ed. (London: Taylor and Francis, 1984), pp. 575-578). For example, one or more antennae, together with a radiating element, can be configured to transmit microwave wavelengths of electromagnetic energy toward a target tissue, and positioned proximal to the tissue to be treated, for example by endoscopic positioning, for heat therapy (see U.S. Pat. No. 6,181,970). Proximal positioning of the microwave generating device is important, because the electromagnetic energy at microwave wavelengths is substantially absorbed by water and lipid molecules in tissue. In some examples a second antenna can be positioned, for example in a body passage, to receive the transmitted microwave energy from the first antenna after passing through the target tissue.

While microwaves are substantially absorbed by water and lipid molecules in targeted biological tissue, the presence of other chromophores (e.g., melanin or other chromophore product) in the targeted tissue, which also absorb microwaves and produce heat, also can contribute to the induction of hyperthermia. In some examples, electromagnetic energy at microwave wavelengths, for example at wavelengths ranging from 3 to 100 cm (corresponding to frequencies of 10 GHz and 0.3 GHz, respectively) at a power range of 100 mW to 150 W, can be applied to a chromophore-containing tissue to increase the temperature of the treated tissue to hyperthermic or thermotherapeutic temperatures.

c) Red/Infrared Light Therapy

In some examples of the heat therapy methods described herein, infrared light therapy can be used to increase the temperature of chromophore-expressing cells, for example chromophore-expressing circulatory cells, such as chromophore-expressing activated lymphocytes. In one example, nucleic acid encoding a chromophore-producing enzyme is delivered selectively to activated leukocytes, such as activated monocytes or T lymphocytes, for example through the use of interleukin-2 receptors (IL-2Rs). Approximately 12 to 48 hr after nucleic acid delivery, for example, 12 to 24 hr after nucleic acid delivery, preferably 18 to 24 hr after nucleic acid delivery, such as 18, 19, 20, 21, 22, 23 or 24 hr after nucleic acid delivery, red light, for example broad spectrum red light or narrow spectrum red light is applied, for example transdermally, to promote destruction of the target cells by heat therapy.

In some examples, the wavelengths used are in the range of about 600-900 nm, typically in the range of 600-790 nm, preferably about 630-710 nm, such as 690-780 nm. Doses of irradiation at these wavelengths can range from 1-160 J/cm², for example 5-25 J/cm².

ii. Ultrasound Heat Therapy

Focused ultrasound (FUS), or high intensity ultrasound (HIUS) also can be used to apply focused sonic energy to produce local heat in chromophore-containing target tissues, for example, melanin-containing tumors as a form of heat therapy, such as hyperthermia or thermotherapy. In the methods provided here in, ultrasound waves can be absorbed by a chromophore product (e.g., melanin) that is manufactured within a target tissue following administration of one or more nucleic acids encoding one or more chromophore producing enzyme(s). This method can be used to non-invasively treat deep-seated tumors (see e.g., Lele, in Hyperthermia in cancer therapy, FK Strom, ed., (London: Taylor and Francis, 1984), pp. 333-367).

In some examples, focused ultrasound using high-frequencies (e.g., 1-4 MHz) are used to transmit sonic energy to be absorbed by the chromophore product, thereby creating a heating zone, typically on the order of millimeters. For large target areas, for example centimeter-sized tumors, multiple overlapping sonication applications can be employed (Malinen et al., (2005) Phys Med Biol. 50(15):3473-90; Zippel and Papa, (2005) Breast Cancer. 12(1):32-38; Ho et al., (2007) Phys. Med. Biol. 52:4584-4599).

In other examples, a lower frequency of ultrasound, such as 1 kHz can be absorbed by the chromophore product and used to heat larger areas with a single sonication application (Bakker et al., (2009) Phys. Med. Biol. 54:3201-3215).

In some examples, the chromophore product is melanin, which can absorb acoustic waves and produce heat and can be used to supplement ultrasound-induced hyperthermia and thermotherapy. Melanin, for example, is a well-established potent absorber of sound waves (Kono et al., (1979) J. Appl. Phys. 50, 1236), and increased accumulation of melanin in target cells/tissues can enhance ultrasound heat therapies, to achieve hyperthermic and thermotherapeutic temperatures of target tissues, for example deep seated tissues, such as internal tumors.

FUS and HIUS procedures described herein can be coupled with an imaging procedure, such as magnetic resonance imaging (MRI) or diagnostic sonography (ultrasound), to facilitate treatment planning and assist with targeting. Increased accumulation of a chromophore, such as melanin in a target cell or tissue, resulting from the methods described herein, also can be exploited to enhance visualization of the chromophore-containing target cells by diagnostic imaging (described elsewhere herein) when used alone or in combination with ultrasounds thermotherapy (e.g., FUS or HIUS) or other thermotherapy procedures described herein.

iii. Magnetic Conduction Methods

Alternating magnetic fields also can produce energy that can be absorbed by a chromophore product and converted to thermal energy to produce a form of heat therapy. For example, a chromophore-containing target tissue can be treated by the methods of magnetic heat treatment provided herein, provided the chromophore product is responsive to an alternating magnetic field and is capable of converting the energy absorbed from the alternating magnetic field into thermal energy. Many chromophores, such as melanin, are chelating agents and are thus responsive to application of an alternating magnetic field. Thus, in some examples, a target tissue, which produces melanin following uptake of one or more nucleic acids encoding one or more melanin producing enzyme(s) is exposed to energy resulting from an alternating magnetic field, which results in the production of heat in the target tissue. In some examples, an alternating magnetic field applicator applies an alternating magnetic field with an oscillation frequency of 80 to 300 kHz, typically, 90 to 150 kHz, more typically approximately 100 kHz; with an intensity ranging from about 2 to 40 kA/m, for example about 2 to 31 kA/m, preferably 2 to 15 kA/m.

In some examples, the subject can be administered a composition containing a metal ion to be chelated by the metal-chelating chromophore product to enhance the absorbance and thermal emission of the chromophore product upon exposure to an alternating magnetic field, prior to the initiation of magnetic heat therapy. In other examples, the target tissue can be perfused or injected with a metal containing composition. Exemplary compositions include compositions containing gadolinium (Gd), iron (Fe), or manganese (Mn). In some examples, a gadolinium containing composition can be administered, such as gadoterate (Dotarem), gadodiamide (Omniscan), gadobenate (MultiHance), gadopentetate (Magnevist, Magnegita, Gado-MRT ratiopharm), gadoteridol (ProHance), gadoversetamide (OptiMARK), gadoxetate (Primovist), gadobutrol (Gadovist), or gadofosveset (Ablavar, formerly Vasovist), gadoxetate (Eovist). In other examples an iron (Fe) containing solution can be administered, such as an iron oxide or iron platinum solution. Exemplary iron containing solutions include Feridex I.V. (Endorem or ferumoxide), Resovist (Cliavist), Sinerem (Combidex), Lumirem (Gastromark), Clariscan™ (PEG-fero, Feruglose). In other examples, a solution containing manganese can be administered, such as manganese chelates. Metal containing compositions can be administered systemically, for example by intravenous or oral administration, or locally, for example by local injection.

b. Photodynamic Therapy

The basis of photodynamic therapy (PDT) is the interaction of light with photosensitive agents to produce an energy transfer and a local chemical effect. PDT relies on administration of a light sensitive compound (i.e., photosensitizer) that is activated by the appropriate wavelength of light, in the presence of oxygen, resulting in the production of toxic reactive oxygen species (ROS) which elicit cellular and tissue destruction, for example, by apoptosis, necrosis and/or vascular damage.

In the methods provided herein, a chromophore product and/or chemical precursors in the biosynthetic pathway thereof, produced in a target tissue as a result of administration of one or more nucleic acids encoding one or more chromophore-producing enzyme, are utilized as photosensitizers for PDT applications. In some examples, the photosensitizer(s) is/are chromophore product melanin and/or the reactive precursors are 5-S-cysteinylDOPA (5-SCD), dopasemiquinone, 5,6-dihydroxyindole (DHI) and/or 5,6-dihydroxyindole-2-carboxylic acid (DHICA), for methods of PDT. In some examples, melanin acts as a photosensitizer that generates ROS in target cell(s) or tissues upon ultraviolet irradiation. In some examples, melanin contributes to ROS production and PDT following exposure to UV irradiation, for exposure to electromagnetic radiation at a wavelength within the range of 100 to 400 nm, 280 to 315 or 315 to 400 nm. In some examples the dose of irradiation is 1 to 200 kJ/m², for example 1250 J/m² for wavelengths in the range of approximately 280 to 315 nm or 100 kJ/m² for wavelengths in the range of 315-340 nm. In some examples, a laser, light-emitting diode, fluorescent lamp, dichroic lamp, or a light box can be used as a light source for PDT. In other examples, UV light is delivered to the target tissue using an endoscope or fiber optic catheter. In a preferred example, a laser is used as the UV light source, for example an interstitial laser, such as a fiber optic is used as the UV light source.

In other examples, electromagnetic energy can be emitted transdermally by two laser sources, of the same wavelength, within the tissue penetrating range of approximately 600 to 1500 nm, such as approximately 600 to 1200 nm, for example approximately 700 to 900 nm or approximately 600 to 800 nm, such as approximately 600, 650, 700, 750, 800, 805 or 808 nm, that are positioned, or focused, for example, by a lens, so that the laser beams intersect (e.g., are confocal) at or within the target tissue, resulting in elevated (approximately double) electromagnetic energy levels in a focused volume at the point of laser beam intersection at or within the target tissue (see e.g., U.S. Pat. Nos. 5,829,448 and 7,036,516, incorporated herein by reference). In these examples, a chromophore product manufactured in the target tissue can be capable of absorbing a sufficient amount of electromagnetic energy at or about half of the wavelengths emitted by the individual lasers (i.e., at wavelengths of or of about 300 to 800 nm or about 320 to 480 nm, for example about 300, 325, 350, 375 or 400 nm) to effect PDT in the target tissue.

3. Methods of Detection

The methods provided herein can include methods of detection of a chromophore-containing target tissue. Any medical detection method, for example medical imaging method can be used for diagnostic or monitoring purposes. For example, X-rays, mammography, ultrasound, computed tomography (CT), magnetic resonance imaging (MRI), nuclear imaging, such as planar gamma imaging, single photon emission computed tomography (SPECT) imaging; positron emission tomography (PET), photoacoustic imaging and optoacoustic imaging can be used as a part of the therapeutic methods provided herein.

In some examples, imaging methods can be used to detect and diagnose tissues to be used as target tissues for the methods provided herein. In other examples, imaging methods can be used to monitor the treatment and/or progress of target tissues for the methods provided herein. In particular examples, the chromophore product (e.g., melanin), produced in a cell or target tissue, as a result of delivery of one or more nucleic acids encoding one or more chromophore-producing enzyme(s) (e.g., tyrosinase or tyrosinase-related protein-1) can enhance the detectability of the target tissue using the imaging methods provided herein.

a. Photoacoustic Tomography

In some examples, photoacoustic tomography is used as a detection method of target tissues in the methods provided herein, and the chromophore product manufactured in the target cell(s) or tissue(s) can enhance the detection of the target cell(s) or tissue(s) in vivo. For example, the chromophore product (e.g., melanin) can absorb non-ionizing laser pulses, for example radio frequency pulses, and convert the applied electromagnetic energy into heat, resulting in transient thermoelastic expansion and thus wideband (e.g., MHz) ultrasonic emission. The generated ultrasonic waves are then detected by ultrasonic transducers to form images. The presence of the chromophore product in the target tissue increases the absorbance properties of the target tissue, enhancing the contrast and facilitating distinction between the target cell(s) or tissue(s) and the surrounding cell(s) or tissue(s) (see e.g., Xu and L. H. Wang (2006). Review of Scientific Instruments. 77 (4): 041101; Filonov et al., (2012) Angew. Chem. Int. Ed. 51:1448-1451; Paproski et al., Biomedical Optics Express. 2(4):771-780, incorporated herein by reference; and Example 7 below). For some applications, detection of chromophore-containing tissues by photoacoustic imaging can utilize endoscopic techniques (Yang et al., (2009) Optics Letters, 34(10):1591-1593). In some examples photoacoustic tomography (e.g., photoacoustic endoscopy) can be combined with one or more therapeutic methods, such as the therapeutic methods described herein, for example, ultrasound methods (Yang et al., (2012) Nature Medicine. 18:1297-1302).

b. Multispectral Optoacoustic Tomography

The methods provided herein also can include multispectral optoacoustic tomography (MSOT) of chromophore-containing target cell(s) or tissue(s) Like photoacoustic imaging, MSOT relies on detection of ultrasonic signals following absorption of electromagnetic energy, typically in the near infrared range (e.g., 680-950 nm) by tissue components. For MSOT, images are acquired using several electromagnetic wavelengths (for example at least 3 wavelengths, such as at least 5 wavelengths, at least 10 wavelengths up to the continuum of wavelengths in the near infrared region), and reconstructed to produce an acoustic pressure distribution map of the imaged cell or tissue (Razansky et al., Optics Letters 32(19): 2891-2893).

Acoustic fingerprints of known, naturally occurring chromophores within the tissue imaged, for example oxy- or deoxy-melanin can be resolved and algorithms can be generated to subtract these signals from the reconstructed image (Glatz et al., (2011) Opt Express 19(4):3175-3184) and generate an image that is constructed from resolved chromophore product-specific signals. In some examples, the chromophore product, expressed in the target tissue subjected to MSOT is melanin. As described above, melanin can increase the contrast for photoacoustic applications and enhance the detection of a melanin-containing cell or tissue by MSOT (see e.g., Example 10 herein)

c. Magnetic Resonance

The methods provided herein also can include detection of a chromophore-containing target tissue by magnetic resonance (MR) imaging, also called MRI. Because the presence of metal in a tissue can increase contrast of an MRI through shortening the T1 relaxation time, accumulation of chromophore(s), or precursors thereof, which chelate metal ions can enhance the contrast of MR images and thereby facilitate and enhance detection of the chromophore-containing target cell(s) or tissue(s). In some examples, melanin is the metal-chelating chromophore that, together with metal-chelating precursors (e.g., DHI and DHICA), can accumulate in target cells or tissues, increase the contrast of MR images, and enhance detection of melanin-containing cells or tissues, which can include, for example, tumors or tumor cells, including metastases (see e.g., U.S. Pat. No. 5,310,539, Paproski et al., Biomedical Optics Express. 2(4):771-780; Weissleder et al. (1997) Radiology. 204(2):425-429; Enochs et al., (1997) Radiology. 204(2):417-423, incorporated herein by reference, and Examples 6 and 7 herein below).

In some examples, the subject can be administered a composition containing a metal ion to be chelated by the metal-chelating chromophore product (e.g., melanin) to enhance MRI detection of the target cell(s) or tissue(s). In other examples, the chelating chromophore (e.g., melanin)-containing target tissue can be perfused or injected with a metal containing composition. Exemplary compositions include compositions containing gadolinium (Gd), iron (Fe), or manganese (Mn). In some examples, a gadolinium containing composition can be administered, such as gadoterate (Dotarem), gadodiamide (Omniscan), gadobenate (MultiHance), gadopentetate (Magnevist, Magnegita, Gado-MRT ratiopharm), gadoteridol (ProHance), gadoversetamide (OptiMARK), gadoxetate (Primovist), gadobutrol (Gadovist), or gadofosveset (Ablavar, formerly Vasovist), gadoxetate (Eovist). In other examples an iron (Fe) containing solution can be administered, such as an iron oxide or iron platinum solution. Exemplary iron containing solutions include Feridex I.V. (Endorem or ferumoxide), Resovist (Cliavist), Sinerem (Combidex), Lumirem (Gastromark), Clariscan™ (PEG-fero, Feruglose). In other examples, a solution containing manganese can be administered, such as manganese chelates. Metal containing compositions can be administered systemically, for example by intravenous or oral administration, or locally, for example by local injection.

4. Monitoring

The methods provided herein can further include one or more steps of monitoring the subject, monitoring the tumor, and/or monitoring the virus administered to the subject. Any of a variety of monitoring steps can be included in the methods provided herein, including, but not limited to, monitoring tumor size, monitoring anti-(tumor antigen) antibody titer, monitoring the presence and/or size of metastases, monitoring the subject's lymph nodes, monitoring the subject's weight or other health indicators including blood or urine markers, monitoring anti-(viral antigen) antibody titer, monitoring viral expression of a detectable gene product, and directly monitoring viral titer in a tumor, tissue or organ of a subject.

The purpose of the monitoring can be for assessing the health state of the subject or the progress of therapeutic treatment of the subject, or can be for determining whether or not further administration of the same or a different virus is warranted, or for determining when or whether or not to administer a compound to the subject where the compound can act to increase the efficacy of the therapeutic method, or the compound can act to decrease the pathogenicity of the virus administered to the subject.

a. Monitoring Gene Expression

In some examples, the methods provided herein can include monitoring one or more genes expressed as a result of nucleic acid delivery, for example virally expressed genes. The nucleic acids delivered in the methods herein can express one or more detectable products, including but not limited to, detectable proteins (e.g., fluorescent proteins), pigments, and/or compounds proteins that induce a detectable signal (e.g., pigments such as melanin that can be detected). The target cells/tissue of the nucleic acid delivery step of the methods provided herein can thus be imaged by one more optical or non-optical imaging methods.

Measurement of a detectable gene product expressed by an expression vector, for example a virus, can provide an accurate determination of the level of expression vector (e.g., virus) present in the subject. The detectable gene product can be measured by methods, such as for example, by imaging methods including, but not limited to, magnetic resonance, fluorescence, and tomographic methods, can determine the localization of the delivered nucleic acid in a subject. Accordingly, the methods provided herein that include monitoring a detectable gene or chromophore product can be used to determine the presence or absence of the administered nucleic acid in one or more organs or tissues of a subject, and/or the presence or absence of the administered nucleic acid (e.g., virus) in a tumor or metastases of a subject. Further, the methods provided herein that include monitoring a detectable viral gene product can be used to determine the titer of virus present in one or more organs, tissues, tumors or metastases.

Since methods provided herein can be used to monitor the amount of viruses at any particular location in a subject, the methods that include monitoring the localization and/or titer of viruses in a subject can be performed at multiple time points, and, accordingly can determine the rate of viral replication in a subject, including the rate of viral replication in one or more organs or tissues of a subject. Accordingly, the methods of monitoring a viral gene product can be used for determining the replication competence of a virus.

The methods provided herein also can be used to quantitate the amount of virus present in a variety of organs or tissues, and tumors or metastases, and can thereby indicate the degree of preferential accumulation of the virus in a subject. Accordingly, the viral gene product monitoring methods provided herein can be used in methods of determining the ability of a virus to accumulate in tumor or metastases in preference to normal tissues or organs.

Since the viruses used in the methods provided herein can accumulate in an entire tumor or can accumulate at multiple sites in a tumor, and can also accumulate in metastases, the methods provided herein for monitoring a viral gene product can be used to determine the size of a tumor or the number of metastases that are present in a subject. Monitoring such presence of viral gene product in tumor or metastasis over a range of time can be used to assess changes in the tumor or metastasis, including growth or shrinking of a tumor, or development of new metastases or disappearance of metastases, and also can be used to determine the rate of growth or shrinking of a tumor, or development of new metastases or disappearance of metastases, or the change in the rate of growth or shrinking of a tumor, or development of new metastases or disappearance of metastases. Accordingly, the methods of monitoring a viral gene product can be used for monitoring a neoplastic disease in a subject, or for determining the efficacy of treatment of a neoplastic disease, by determining rate of growth or shrinking of a tumor, or development of new metastases or disappearance of metastases, or the change in the rate of growth or shrinking of a tumor, or development of new metastases or disappearance of metastases.

Any of a variety of detectable proteins can be detected in the monitoring methods provided herein. An exemplary, non-limiting list of such detectable proteins includes any of a variety of fluorescent proteins (e.g., green or red fluorescent proteins), any of a variety of luciferases, transferrin or other iron binding proteins; or receptors, binding proteins, and antibodies, where a compound that specifically binds the receptor, binding protein or antibody can be a detectable agent or can be labeled with a detectable substance (e.g., a radionuclide or imaging agent); or transporter proteins (e.g., hNET or hNIS) that can bind to and transport detectable molecules into the cell. Viruses expressing a detectable protein can be detected by a combination of the methods provided herein and known in the art.

Viruses expressing more than one detectable protein or two or more viruses expressing various detectable protein can be detected and distinguished by dual imaging methods. For example, a virus expressing a fluorescent protein and an iron binding protein can be detected in vitro or in vivo by low light fluorescence imaging and magnetic resonance, respectively. In another example, a virus expressing two or more fluorescent proteins can be detected by fluorescence imaging at different wavelengths. In vivo dual imaging can be performed on a subject that has been administered a virus expressing two or more detectable gene products or two or more viruses each expressing one or more detectable gene products.

b. Monitoring Tumor Size

Also provided herein are methods of monitoring tumor and/or metastasis size and location. Tumor and or metastasis size can be monitored by any of a variety of methods known in the art, including external assessment methods or tomographic or magnetic imaging methods, such as the detection methods described herein. In addition, methods provided herein, for example, monitoring gene expression (e.g., viral gene expression), can be used for monitoring tumor and/or metastasis size.

Monitoring size over several time points can provide information regarding the efficacy of the therapeutic methods provided herein. In addition, monitoring the increase or decrease in size of a tumor or metastasis, and can also provide information regarding the presence (i.e., detection and/or diagnosis) of additional tumors and/or metastases in the subject. Monitoring tumor size over several time points can provide information regarding the development of a neoplastic disease in a subject, including the efficacy of treatments of a neoplastic disease in a subject, such as the treatments provided herein.

c. Monitoring Antibody Titer

The methods provided herein also can include monitoring the antibody titer in a subject, including antibodies produced in response to administration of a virus to a subject. For example, the viruses administered in the methods provided herein can elicit an immune response to endogenous viral antigens. The viruses administered in the methods provided herein also can elicit an immune response to exogenous genes expressed by a virus. The viruses administered in the methods provided herein also can elicit an immune response to tumor antigens. Monitoring antibody titer against viral antigens, viral expressed exogenous gene products, or tumor antigens can be used in methods of monitoring the toxicity of a virus, monitoring the efficacy of treatment methods, or monitoring the level of gene product or antibodies for production and/or harvesting.

In one example, monitoring antibody titer can be used to monitor the toxicity of a virus. Antibody titer against a virus can vary over the time period after administration of the virus to the subject, where at some particular time points, a low anti-(viral antigen) antibody titer can indicate a higher toxicity, while at other time points a high anti-(viral antigen) antibody titer can indicate a higher toxicity. The viruses used in the methods provided herein can be immunogenic, and can therefore elicit an immune response soon after administering the virus to the subject.

Generally, a virus against which a subject's immune system can quickly mount a strong immune response can be a virus that has low toxicity when the subject's immune system can remove the virus from all normal organs or tissues. Thus, in some examples, a high antibody titer against viral antigens soon after administering the virus to a subject can indicate low toxicity of a virus. In contrast, a virus that is not highly immunogenic can infect a host organism without eliciting a strong immune response, which can result in a higher toxicity of the virus to the host. Accordingly, in some examples, a high antibody titer against viral antigens soon after administering the virus to a subject can indicate low toxicity of a virus.

In other examples, monitoring antibody titer can be used to monitor the efficacy of treatment methods. In the methods provided herein, antibody titer, such as anti-(tumor antigen) antibody titer, can indicate the efficacy of a therapeutic method such as a therapeutic method to treat neoplastic disease. Therapeutic methods provided herein can include causing or enhancing an immune response against a tumor and/or metastasis. Thus, by monitoring the anti-(tumor antigen) antibody titer, it is possible to monitor the efficacy of a therapeutic method in causing or enhancing an immune response against a tumor and/or metastasis.

The therapeutic methods provided herein also can include administering to a subject a virus that can accumulate in a tumor and can cause or enhance an anti-tumor immune response. Accordingly, it is possible to monitor the ability of a host to mount an immune response against viruses accumulated in a tumor or metastasis, which can indicate that a subject has also mounted an anti-tumor immune response, or can indicate that a subject is likely to mount an anti-tumor immune response, or can indicate that a subject is capable of mounting an anti-tumor immune response.

In other examples, monitoring antibody titer can be used for monitoring the level of gene product or antibodies for production and/or harvesting. As provided herein, methods can be used for producing proteins, RNA molecules or other compounds by expressing an exogenous gene in a virus that has accumulated in a tumor. Further provided herein are methods for producing antibodies against a protein, RNA molecule or other compound produced by exogenous gene expression of a virus that has accumulated in a tumor. Monitoring antibody titer against the protein, RNA molecule or other compound can indicate the level of production of the protein, RNA molecule or other compound by the tumor-accumulated virus, and also can directly indicate the level of antibodies specific for such a protein, RNA molecule or other compound.

d. Monitoring General Health Diagnostics

The methods provided herein also can include methods of monitoring the health of a subject. Some of the methods provided herein are therapeutic methods, including neoplastic disease therapeutic methods. Monitoring the health of a subject can be used to determine the efficacy of the therapeutic method, as is known in the art. The methods provided herein also can include a step of administering to a subject a virus. Monitoring the health of a subject can be used to determine the pathogenicity of a virus administered to a subject. Any of a variety of health diagnostic methods for monitoring disease such as neoplastic disease, infectious disease, or immune-related disease can be monitored, as is known in the art. For example, the weight, blood pressure, pulse, breathing, color, temperature or other observable state of a subject can indicate the health of a subject. In addition, the presence or absence or level of one or more components in a sample from a subject can indicate the health of a subject. Typical samples can include blood and urine samples, where the presence or absence or level of one or more components can be determined by performing, for example, a blood panel or a urine panel diagnostic test. Exemplary components indicative of a subject's health include, but are not limited to, white blood cell count, hematocrit, or reactive protein concentration.

e. Monitoring Coordinated with Treatment

Also provided herein are methods of monitoring a therapy, for example, a heat therapy or photodynamic therapy applied to a chromophore-producing target cell or tissue, as described in the methods provided herein, and therapeutic decisions can be based on the results of the monitoring. The methods provided herein include a first step of administering a nucleic acid to a subject encoding a chromophore-producing enzyme, for example, a virus that encodes a melanin-producing enzyme, such as tyrosinase or tyrosinase-related protein-1, that can preferentially accumulates in a target cell or tissue (e.g., a tumor and/or metastasis), wherein the chromophore-producing enzyme contributes to the biosynthesis of a chromophore product(e.g., melanin). A subsequent step of the therapeutic methods provided herein includes administering a form of energy, such as electromagnetic, acoustic, or magnetic energy, that is absorbed by the chromophore product and converted into heat energy or chemical energy, causing or enhancing destruction of or sensitizing destruction of its host cell or tissue.

Such therapeutic methods can include a variety of steps including multiple administrations of a nucleic acid (e.g., a particular virus), administration of a second nucleic acid (e.g., a second virus), administration of a therapeutic compound, or administration of one or more additional therapeutic methods. Determination of the amount, timing or type of nucleic acid (e.g., a virus) to administer to the subject can be based on one or more results from monitoring the subject and/or target cell or tissue. In addition, determination of the dose, timing or type of energy application, for example as a part of a therapeutic treatment, can be based on one or more results from monitoring the subject and/or target cell or tissue. For example, the antibody titer in a subject can be used to determine whether or not it is desirable to administer a nucleic acid (e.g., a virus), the quantity of nucleic acid (e.g., a virus) to administer, and the type of nucleic acid (e.g., a virus) to administer, where, for example, an unchanged tumor volume can indicate the desirability of administering additional nucleic acid (e.g., virus) encoding a chromophore-producing enzyme, a nucleic acid (e.g., a virus) encoding a different chromophore-producing enzyme that can produce the same or different chromophore product.

In another example, monitoring can determine whether or not a nucleic acid has accumulated in a target tissue (e.g., a tumor or metastasis of a subject), whether a delivered nucleic acid expresses the chromophore-producing enzyme encoded by the nucleic acid, and/or whether the chromophore-producing enzyme leads to sufficient production of the chromophore. Upon such determinations, a decision can be made to further administer additional nucleic acid or a different nucleic acid to the subject.

In another example, monitoring a detectable expressed transgene product can be used to determine whether or not it is desirable to administer additional nucleic acid, the quantity of nucleic acid to administer, and the type of nucleic acid to administer. Such monitoring methods also can be used to determine whether or not a therapeutic method is effective, whether or not a therapeutic method is pathogenic to the subject, whether or not the nucleic acid (e.g., virus) has accumulated in a target tissue (e.g., tumor or metastasis), and whether or not the nucleic acid (e.g., virus) has accumulated in normal tissues or organs.

In another example, the overall health state of a subject can be used to determine whether or not it is desirable to a administer a nucleic acid or therapeutic treatment provided herein, the dose of nucleic acid or therapy (i.e., energy) to administer, and the type of nucleic acid or therapy (i.e., energy) to administer, where, for example, determining that the subject or target tissue is healthy can indicate the desirability of administering additional nucleic acid (e.g., a virus) or a different nucleic acid, or can indicate the desirability of administering additional therapy (i.e., energy) or an alternative therapy.

In another example, monitoring can indicate that a nucleic acid (e.g., a virus) is pathogenic to a subject. In such instances, a decision can be made to terminate administration of the virus to the subject, to administer lower levels of the virus to the subject, to administer a different virus to a subject, or to administer to a subject a compound that reduces the pathogenicity of the virus. In one example, administration of a virus that is determined to be pathogenic can be terminated. In another example, the dosage amount of a virus that is determined to be pathogenic can be decreased for subsequent administration.

In one example, a healthy target tissue, following therapeutic treatment, can indicate the desirability for administering additional therapy (i.e., energy), for example administering a stronger dose of energy or administering energy at a different wavelength or administering a range of energies. In another example, a healthy tissue, following therapeutic treatment within a cycle of therapeutic treatments can indicate the desirability for increasing the frequency of therapeutic applications. In another example, a healthy target tissue, following therapeutic treatment, can indicate the desirability for administering an alternative therapeutic treatment, such as a therapeutic provided herein or other therapeutic treatment known in the art. In another example, a healthy target tissue, following therapeutic treatment, can indicate the desirability for administering a combination of therapeutic treatments, including one or more therapeutic treatments provided herein or other therapeutic treatments known in the art. Based on such determinations, the desirability and form of further therapeutic methods can be derived.

In one example, determination of whether or not a therapeutic method is effective can be used to derive further therapeutic methods. Any of a variety of methods of monitoring can be used to determine whether or not a therapeutic method is effective, as provided herein or otherwise known in the art. If monitoring methods indicate that the therapeutic method is effective, a decision can be made to maintain the current course of therapy, which can include further administrations of a nucleic acid, or a decision can be made that no further administrations are required. If monitoring methods indicate that a therapeutic method is ineffective, the monitoring results can indicate whether or not a course of treatment should be discontinued (e.g., when a nucleic acid (e.g., a virus) is pathogenic to the subject or if the subject experiences unwanted side-effects from the treatment administered), or changed (e.g., when a therapeutic method has no effect on a chromophore-expressing target tissue), or increased in frequency or amount (e.g., when a chromophore-expressing target tissue is slightly affected by the therapeutic treatment).

### H. COMBINATION THERAPY

The methods provided herein also can be used in combination therapies. For example, therapeutic methods provided herein can be combined with perfusion, direct injection or local application of the area with an additional therapy, such as an anti-cancer therapy. Such treatment can be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. The treatments provided herein and treatments used in combination with those provided herein can be of varying dosages.

In some examples, the subject to be treated using the methods provided herein also can be undergoing secondary treatment, for example, for a tumor, cancer, wound, hyperproliferative surface lesion, or autoimmune disorder. For example, the methods herein can include combination therapy with a secondary anti-cancer therapy. Examples of such therapy include, but are not limited to, surgical therapy, chemotherapy, radiotherapy, immunotherapy, treatment with another therapeutic substance or agent and/or administration with another therapeutic virus.

In some examples, the target tissue is a tumor and treatment of the target tumor using a therapeutic method provided herein, followed by application of an additional therapy, for example, a second therapy described herein or a therapy set forth below, can cause a reduction in tumor volume, can cause tumor growth to stop or be delayed or can cause the tumor to be eliminated from the subject. The status of tumors, cancers and metastasis following treatment can be monitored using any of the methods provided herein and known in the art, including the monitoring methods described herein.

In some examples, target tissues, such as tumors, cancers and metastases can be monotherapy-resistant such as, for example, one that does not respond to a single therapy, but that does respond to therapy with a combination of therapies, for example heat therapy combined with a therapeutic agent (e.g., anti-cancer agent) or radiotherapy. The therapies to be used in combination can be administered simultaneously, sequentially or intermittently with the therapies provided herein. Combined treatment should be individualized for each subject.

a. Surgical Procedures

In some examples, a subject can be subjected to surgical procedures in addition to receiving treatment for a hyperproliferative disease or disorder, by one of the methods provided herein. Surgical procedures that can be used in combination with the methods provided herein include preventative surgery, diagnostic surgery, cryoreductive surgery, or cryosurgery, for example by slow freezing, rapid freezing, or defreezing. Surgical procedures can follow or precede a therapeutic treatment provided in the methods herein. One of skill in the art can determine the time needed for a subject to recover from heat therapy or a surgical procedure before commencing with the second procedure.

b. Radiotherapy

Radiation therapy (also called radiotherapy) has become a foremost choice of treatment for a majority of cancer patients. The wide use of radiation treatment stems from the ability of gamma-irradiation to induce irreversible damage in targeted cells with the preservation of normal tissue function. Ionizing radiation triggers apoptosis, the intrinsic cellular death machinery in cancer cells, and the activation of apoptosis seems to be the principal mode by which cancer cells die following exposure to ionizing radiation.

In some examples, a subject can be treated with radiotherapy in combination with the therapeutic methods provided herein. Modalities of radiotherapy include curative, adjuvant, and palliative radiotherapy, such as brachytherapy, metabolic, and radiotherapy. Factors to take into account when combining the therapeutic methods provided herein with radiotherapy include the pericellular environment, histological type, and the location of the tumor, including the radiosensitivity of surrounding healthy tissue(s). In addition, the properties of the chromophore manufactured in the target tissue as a part of the methods provided herein also should be taken into account. For example, a chromophore that can interfere with radiotherapy can alter the application, for example the dosage, of the radiotherapy or can be incompatible with radiotherapy.

In some examples, heat therapy, such as by the methods provided herein, sensitizes the chromophore-containing target tissue, rendering the chromophore-containing target tissue more susceptible to radiotherapy. For example, in some combination therapies, application of heat therapy, such as interstitial laser-induced heat therapy methods provided herein, can destroy or sensitize the target cells or tissue, especially poorly vascularized cells and tissues. Application of ionizing radiation, following heat therapy, can then be used to eradicate cells from the well-vascularized cells, for example cells at the tumor surface.

In some examples, radiotherapy follows within hours (e.g., within 3 to 10 hr) of a heat therapy, for example. In some examples, radiotherapy follows the heat (or photodynamic) therapy 1 day after heat therapy. In some examples, radiotherapy follows 2 days to 1 week, or more, after heat therapy. One of skill in the art can determine when to administer the therapeutic agent subsequent to the prior therapeutic treatment, for example, in vivo, in animal models. In some examples, it is preferable to administer radiotherapy prior to heat therapy, for example at 1 to 6 hr after radiotherapy, such as 3 to 4 hr after radiotreatment.

c. Therapeutic Compounds

Any therapeutic or anti-cancer agent can be used as a second therapeutic or anti-cancer agent in the combined cancer treatment methods provided herein. The methods can include administering one or more therapeutic compounds to the subject in addition to administering a therapy provided herein to a subject. Therapeutic compounds can act independently, or in conjunction with the therapy or method of detection, to achieve therapeutic effects, for example, anti-tumor effects. Therapeutic compounds or agents also include those that are immunotherapeutic compounds. Therapeutic compounds to be administered can be any of those provided herein or in the art. Therapeutic compounds that can act independently include any of a variety of known chemotherapeutic compounds that can inhibit tumor growth, inhibit metastasis growth and/or formation, decrease the size of a tumor or metastasis, eliminate a tumor or metastasis, without reducing the ability of a nucleic acid (e.g., a virus containing one or more nucleic acids encoding one or more chromophore-producing enzymes) to accumulate in a target tissue, such as a tumor, or the replication thereof.

Typically, a target chromophore-containing target cell(s) or tissue(s) is treated using a therapeutic method described herein to effect optimal destruction or sensitivity of the target cell(s) or tissue(s), prior to administration of a therapeutically effective amount of another therapeutic agent, for example an anti-cancer agent, such as a chemotherapeutic agent (e.g., cisplatin). In one example, the subsequent therapeutic agent is administered once daily for five consecutive days. One of skill in the art can determine when to administer the therapeutic agent subsequent to the prior therapeutic treatment, for example, in vivo, in animal models.

Therapeutic compounds or agents include, but are not limited to, chemotherapeutic agents, nanoparticles, siRNA molecules, enzyme/pro-drug pairs, photosensitizing agents, toxins, a radionuclide, an angiogenesis inhibitor, a mitosis inhibitor protein (e.g., cdc6), an antitumor oligopeptide (e.g., antimitotic oligopeptides, high affinity tumor-selective binding peptides), a signaling modulator, anti-cancer antibiotics, or a combination thereof.

Exemplary photosensitizing agents include, but are not limited to, for example, indocyanine green, toluidine blue, aminolevulinic acid, texaphyrins, benzoporphyrins, phenothiazines, phthalocyanines, porphyrins such as sodium porfimer, chlorins such as tetra(m-hydroxyphenyl)chlorin or tin(IV) chlorin e6, purpurins such as tin ethyl etiopurpurin, purpurinimides, bacteriochlorins, pheophorbides, pyropheophorbides or cationic dyes. In one example, a vaccinia virus, such as a vaccinia virus provided herein, is administered to a subject having a tumor, cancer or metastasis in combination with a photosensitizing agent.

Radionuclides can be used for diagnosis and/or for treatment, depending upon the radionuclide, amount and application. Radionuclides include, but are not limited to, for example, a compound or molecule containing ³²Phosphorus, ⁶⁰Cobalt, ⁹⁰Yttrium, ⁹⁹Technitium, ¹⁰³Palladium, ¹⁰⁶Ruthenium, ¹¹¹Indium, ¹¹⁷Lutetium, ¹²⁵Iodine, ¹³¹Iodine, ¹³⁷Cesium, ¹⁵³Samarium, ¹⁸⁶Rhenium, ¹⁸⁸Rhenium, ¹⁹²Iridium, ¹⁹⁸Gold, ²¹¹Astatine, ²¹²Bismuth or ²¹³Bismuth. In one example, a vaccinia virus, such as a vaccinia virus provided herein, is administered to a subject having a tumor, cancer or metastasis in combination with a radionuclide.

Toxins include, but are not limited to, chemotherapeutic compounds such as, but not limited to, 5-fluorouridine, calicheamicin and maytansine. Signaling modulators include, but are not limited to, for example, inhibitors of macrophage inhibitory factor, toll-like receptor agonists and stat3 inhibitors. In one example, a vaccinia virus, such as a vaccinia virus provided herein, is administered to a subject having a tumor, cancer or metastasis in combination with a toxin or a signaling modulator.

Chemotherapeutic compounds include, but are not limited to, alkylating agents such as thiotepa and cyclophosphamide; alkyl sulfonates such as busulfan, improsulfan and piposulfan; aziridines such as benzodepa, carboquone, meturedepa and uredepa; ethylenimine and methylmelamines, including altretamine, triethylenemelamine, triethylenephosphoramide, triethylenethiophosphoramide and trimethylmelamine nitrogen mustards such as chlorambucil, chlornaphazine, chlorophosphamide, estramustine, ifosfamide, mechlorethamine, mechlorethamine oxide hydrochloride, melphalan, novobiocin, phenesterine, prednimustine, trofosfamide, uracil mustard; nitrosoureas such as carmustine, chlorozotocin, fotemustine, lomustine, nimustine, ranimustine; antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defosfamide; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, e.g., paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; Navelbine; Novantrone; teniposide; daunomycin; aminopterin; Xeloda; ibandronate; CPT11; topoisomerase inhibitor RFS 2000; difluoromethylornithine (DMFO); retinoic acid; esperamycins; capecitabine; and pharmaceutically acceptable salts, acids or derivatives of any of the above.

Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston); and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods.

Chemotherapeutic agents also include new classes of targeted chemotherapeutic agents such as, for example, imatinib (sold by Novartis under the trade name Gleevec in the United States), gefitinib (developed by AstraZeneca under the trade name Iressa) and erlotinib. Particular chemotherapeutic agents include, but are not limited to, cisplatin, carboplatin, oxaliplatin, DWA2114R, NK121, IS 3 295, and 254-S vincristine, prednisone, doxorubicin and L-asparaginase; mechlorethamine, vincristine, procarbazine and prednisone (MOPP), cyclophosphamide, vincristine, procarbazine and prednisone (C-MOPP), bleomycin, vinblastine, gemcitabine and 5-fluorouracil. Exemplary chemotherapeutic agents are, for example, cisplatin, carboplatin, oxaliplatin, DWA2114R, NK121, IS 3 295, and 254-S.

Exemplary anti-cancer antibiotics include, but are not limited to, anthracyclines such as doxorubicin hydrochloride (adriamycin), idarubicin hydrochloride, daunorubicin hydrochloride, aclarubicin hydrochloride, epirubicin hydrochloride and pirarubicin hydrochloride, phleomycins such as phleomycin and peplomycin sulfate, mitomycins such as mitomycin C, actinomycins such as actinomycin D, zinostatin stimalamer and polypeptides such as neocarzinostatin.

Anti-cancer antibodies include, but are not limited to, Rituximab, ADEPT, Trastuzumab (Herceptin), Tositumomab (Bexxar), Cetuximab (Erbitux), Ibritumomab (Zevalin), Alemtuzumab (Campath-1H), Epratuzumab (Lymphocide), Gemtuzumab ozogamicin (Mylotarg), Bevacimab (Avastin), Tarceva (Erlotinib), SUTENT (sunitinib malate), Panorex (Edrecolomab), RITUXAN (Rituximab), Zevalin (90Y-ibritumomab tiuxetan), Mylotarg (Gemtuzumab Ozogamicin) and Campath (Alemtuzumab).

Cancer growth inhibitors use cell-signaling molecules which control the growth and multiplication of cells, such as cancer cells. Drugs that block these signaling molecules can stop cancers from growing and dividing. Cancer growth inhibitors include drugs that block tyrosine kinases (i.e. tyrosine kinase inhibitors; TKIs) or that inhibit the proteasome. Examples of TKIs include, but are not limited to, Erlotinib (Tarceva, OSI-774), Iressa (Gefitinib, ZD 1839), Imatinib (Glivec, STI 571) and Bortezomib (Velcade).

In one example, nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen. In one example, a vaccinia virus in protein polymer composition, such as any provided herein, is administered to a subject having a tumor, cancer or metastasis in combination with a nanoparticle carrying any of the therapeutic agents provided herein.

Therapeutic compounds also include, but are not limited to, compounds that exert an immunotherapeutic effect, stimulate or suppress the immune system, carry a therapeutic compound, or a combination thereof. Such therapeutic compounds include, but are not limited to, anti-cancer antibodies, siRNA molecules and compounds that suppress the immune system (i.e. immunosuppressors, immunosuppressive agents). Exemplary immunosuppressive agents include, but are not limited to, glucocorticoids, alkylating agents, antimetabolites, cytokines and growth factors (e.g., interferons) and immunosuppressive antibodies (e.g., anti-CD3 and anti-IL-2 receptor antibodies). For example, immunosuppressive agents include biological response modifiers, such as monoclonal antibodies (mAbs), cancer vaccines, growth factors for blood cells, cancer growth inhibitors, anti-angiogenic factors, interferon alpha, interleukin-2 (IL-2), gene therapy and BCG vaccine for bladder cancer.

Cytokines and growth factors include, but are not limited to, interleukins, such as, for example, interleukins (e.g., interleukin-1, interleukin-2, interleukin-6 and interleukin-12), tumor necrosis factors, such as tumor necrosis factor alpha (TNF-α), interferons such as interferon gamma (IFN-γ) or interferon alpha (IFN-α), Granulocyte Colony Stimulating Factor (G-CSF; also called filgrastim (Neupogen) or lenograstim (Granocyte)), Granulocyte and Macrophage Colony Stimulating Factor (GM-CSF; also called molgramostim), angiogenins, erythropoietin (EPO) and tissue factors.

Cancer vaccines include, for example, antigen vaccines, whole cell vaccines, dendritic cell vaccines, DNA vaccines and anti-idiotype vaccines. Antigen vaccines are vaccines made from tumor-associated antigens in, or produced by, cancer cells. Antigen vaccines stimulate a subject's immune system to attack the cancer. Whole cell vaccines are vaccines that use the whole cancer cell, not just a specific antigen from it, to make the vaccine. The vaccine is made from a subject's own cancer cells, another subject's cancer cells or cancer cells grown in a laboratory. The cells are treated in the laboratory, usually with radiation, so that they can't grow, and are administered to the subject via injection or through an intravenous drip into the bloodstream so they can stimulate the immune system to attack the cancer.

One type of whole cell vaccine is a dendritic cell vaccine, which helps the immune system recognize and attack abnormal cells, such as cancer cells. Dendritic cell vaccines are made by growing dendritic cells alongside the cancer cells in the lab. The vaccine is administered to stimulate the immune system to attack the cancer.

Anti-idiotype vaccines are vaccines that stimulate the body to make antibodies against cancer cells. Cancer cells make some tumor-associated antigens that the immune system recognizes as foreign. But because cancer cells are similar to non-cancer cells, the immune system can respond weakly. DNA vaccines boost the immune response. DNA vaccines are made from DNA from cancer cells that carry the genes for the tumor-associated antigens. When a DNA vaccine is injected, it enables the cells of the immune system to recognize the tumor-associated antigens, and activates the cells in the immune system (i.e., breaking tolerance).

The dose scheme of the combination therapy administered is such that the combination of the two or more therapeutic modalities is therapeutically effective. Dosages will vary in accordance with such factors as the age, health, sex, size and weight of the patient, the route of administration, the toxicity of the drugs, frequency of treatment and the relative susceptibilities of the cancer to each of the therapeutic modalities. For combination therapies with additional therapeutic agents provided herein (e.g., chemotherapeutic compounds), dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, for example, tumor size, and other viruses, treatments, or compounds, such as other chemotherapeutic drugs, being administered concurrently). As will be understood by one of skill in the art, the optimal treatment regimen will vary and it is within the scope of the treatment methods to evaluate the status of the disease under treatment and the general health of the patient prior to, and following one or more cycles of combination therapy in order to determine the optimal therapeutic combination.

### I. EXAMPLES

The following examples are included for illustrative purposes only and are not intended to limit the scope of the invention.

### Example 1

### mTyr, Tyrp1, and DCT Expression in HeLa Cells and Detection of Melanin Production

In this example, lentiviral expression vectors were constructed for the expression of mouse Tyrosinase (mTyr), human tyrosinase related protein 1 (Tyrp1) and human Dopachrome tautomerase/tyrosinase related protein 2 (DCT). The mTyr, Tyrp, and DCT proteins were expressed in HeLa cells by transfection with the expression vectors. Detection of melanin production was performed by visual inspection, measurement of optical density/absorbance, and magnetic resonance imaging (MRI).

### A. Construction of Expression Plasmids

Expression vectors driving expression of mTyr (SEQ ID NO: 6 (cDNA); SEQ ID NO: 7 (protein)), Tyrp 1 (SEQ ID NO: 19 (cDNA); SEQ ID NO: 20 (protein)), and DCT (SEQ ID NO: 29 (cDNA); SEQ ID NO: 30 (protein)), under the regulation of the CMV promoter, were constructed using the Multisite Gateway® system (Invitrogen), including the Gateway® vector pLENTI6/V5-DEST (Invitrogen, SEQ ID NO: 4). pLENTI6/V5-DEST is a lentiviral expression vector for lentiviral-based expression of a target gene in dividing and non-dividing mammalian cells., and contains the blasticidin selection marker for stable selection in mammalian cells.

To obtain Tyrp1 (SEQ ID NO: 19) and DCT (SEQ ID NO: 29) cDNAs, mRNA was isolated from 1936-Mel (NIH) human melanoma cell lines, using RNeasy Mini kit (Qiagen), and reverse transcribed using Revertaid cDNA Synthesis Kit (Fermentas). Double-stranded cDNAs, corresponding to Tyrp1 and DCT, were then amplified by PCR using Phusion High-fidelity DNA polymerase (Finnzymes) and forward and reverse primers attB1-TYRP1-for (SEQ ID NO: 35) and attB2-TYRP1-rev (SEQ ID NO: 36), respectively for TYRP1, and attB1-DCT-for (SEQ ID NO: 37) and attB2-DCT-rev (SEQ ID NO: 38), respectively for DCT (see Table 13 below). The forward primers used each contained attB1 sites for Gateway® cloning and a Kozak sequence for transcription initiation. The reverse primers each contained attB2 sites for Gateway® cloning. The initial denaturation step of the PCR reaction occurred at 98° C. for 60 seconds (s), followed by 30 cycles of 98° C. for 10 s, 58° C. for 15 s, and 72° C. for 35 s. The reaction was concluded with a final elongation step at 72° C. for 5 min before cooling down to 4° C.

cDNA encoding mTyr was obtained by PCR from the pTRETyBSnew plasmid (Gimenez et al., 2004) using forward primer attB1-mTYR-for (SEQ ID NO: 33) and reverse primer attB2-mTYR-rev (SEQ ID NO: 34) and the Phusion High-fidelity DNA polymerase (Finnzymes). The attB1-mTYR-for primer contains an attB1 site and the attB2-mTYR-rev contains an attB2 site for Gateway® cloning. The PCR reaction commenced with an initial denaturation step at 98° C. for 90 s, followed by 30 cycles of 98° C. for 20 s, 62° C. for 20 s, and 72° C. for 40 s and a final elongation step at 72° C. for 5 min before cooling down to 4° C.

Each PCR product was BP-cloned into donor vector pDONR221 (Invitrogen; SEQ ID NO: 5) to generate pENTR221-mTyr (SEQ ID NO: 8), pENTR221-TYRP1 (SEQ ID NO: 23), and pENTR221-DCT (SEQ ID NO: 31), each of which contain attP sites for recombination with pLENTI6N5-DEST. Recombination was carried out via LR-reaction according to the manufacturer's instructions, using half of all reagents/volumes described in the manual, to generate pLENTI6N5-DCT (SEQ ID NO: 32), pLENTI6/V5-Tyrp1 (SEQ ID NO: 24), pLENTI6N5-mTyr (SEQ ID NO: 9). Inserted sequences in resulting plasmids were confirmed by sequencing.

TABLE 3

| Primer | |
|---|---|
| name: | Sequence (5′-->3′) |
| attB1- | ggggacaagtttgtacaaaaaagcaggctAGCTTAGTGTAAAACAGGCTGAG |
| mTYR-for | (SEQ ID NO: 33) |
| attB2- | ggggaccactttgtacaagaaagctgggtAATTCCGGAAATTCACAATGAAGTAAG |
| mTYR-rev | (SEQ ID NO: 34) |
| attB1- | ggggacaagtttgtacaaaaaagcaggctgccaccgccATGAGTGCTCCTAAACTCCTCT |
| TYRP1-for | (SEQ ID NO: 35) |
| attB2- | ggggaccactttgtacaagaaagctgggtTTGTTAGACCACAGACTGATTAGG |
| TYRP1-rev | (SEQ ID NO: 36) |
| attB1-DCT- | ggggacaagtttgtacaaaaaagcaggctgccaccgccATGAGCCCCCTTTGG |
| for | (SEQ ID NO: 37) |
| attB2-DCT- | ggggaccactttgtacaagaaagctgggtAGCACCCTAGGCTTCTTCTGTG |
| rev | (SEQ ID NO: 38) |

CAPITAL letters = transgene sequence;
underline = Kozak sequence

### B. Visual Detection of Melanin Production in Transfected HeLa Cells

HeLa cells seeded into 24-well plates were grown to about 50% confluency (approximately 1×10⁵ cells/well) and transfected with 0.5 μg of plasmids pLENTI6N5-DCT, pLENTI6N5-Tyrp1, pLENTI6/V5-mTyr (or mixtures thereof) or control plasmids, expressing the reporter constructs EGFP (pLENTI6/V5-EGFP; SEQ ID NO: 71), mNeptune (pLENTI6/V5-mNeptune; SEQ ID NO: 72), or lacZ (pLENTI6N5-GW/lacZ; SEQ ID NO: 73) using GeneCellin™ (Bio Cell Challenge, Paris, France) according to the manufacturer's instructions. The transfected cells were then harvested at predetermined time points following transfection for analysis.

In a first experiment, HeLa cells were transfected with DCT alone, Tyrp 1 alone, mTyr alone, mTyr/DCT, mTyr/Tyrp1, or mTyr/Tyrp1/DCT. Control cells were mock transfected cells or cells transfected with EGFP or EGFP/mNeptune. The transfection efficiency was determined to be about 70% for single- and dual-plasmid transfection by flow cytometry of pLENTI-EGFP and pLENTI-EGFP+pLENTI-mNeptune transfected cells respectively. The cells were harvested 8 days post-transfection. Harvested samples were pelleted by centrifugation in microfuge tubes and photographed using a digital camera for visual assessment. Expression of mTyr alone, mTyr/DCT, mTyr/Tyrp1, and mTyr/Tyrp1/DCT all resulted in production of a dark pigment visible by eye in the cell pellet. In this experiment, the production of pigment in samples expressing DCT alone or Tyrp 1 alone was unclear.

In a further experiment, HeLa cells transfected with Tyrp1/DCT in addition to cells transfected with the construct combinations of the first experiment, to further examine the effects of DCT and Tyrp1 expression. Control samples for the second experiment included mock transfected cells and EGFP-transfected cells. In this experiment, HeLa cells were plated in 6-well plates and were transfected with 2.0 μg DNA upon becoming 50% confluent (approximately 5×10⁵ cells/well). Transfected HeLa cells were harvested 2 days post-transfection, and the transfection efficiency was determined to be >95% (determined only for single plasmid). Cells were pelleted by centrifugation, resuspended in a small volume (20 μL) of PBS, and dropped onto the lid of a 96-well plate, whereupon they were photographed using a digital camera for visual inspection.

In this experiment, production of melanin was observed only in cells expressing mTyr. Co-transfection of mTyr and DCT had no observable effect on melanin expression, compared to mTyr alone. In contrast, co-expression of mTyr and Tyrp 1 resulted in a pigment that appeared darker and blacker in color compared to expression of mTyr alone. The dark pigmentation indicates that co-expression of mTyr with Tyrp1 results in increased production of eumelanin.

### C. Absorbance Detection of Melanin Production in Transfected HeLa Cells

In this experiment, melanin production in HeLa cells was quantified based on changes in absorption rates following transfection with the melanin-producing enzymes, mTyr, Tyrp 1 and DCT, or combinations thereof. HeLa cells were transfected with vectors expressing DCT alone, Tyrp1 alone, mTyr alone, Tyrp1/DCT, mTyr/DCT, mTyr/Tyrp1 or mTyr/Tyrp1/DCT. Control samples included mock and lacZ transfected cells. HeLa cells were plated in a 24-well plated and transfected as described in part B, above, and harvested 2 days later. Cells were harvested from single wells (about 2×10⁵ cells) and resuspended in 100 μL ddH₂O containing 0.5 μL Benzonase. The cells were disrupted by ultrasound treatment (3×30 s at 80%), using a Sonifier Branson 450 (Heinemann, Schwäbisch Gmünd, Germany), followed by incubation at room temperature for 20 minutes. 10 μL 0.1% Triton X-100 in ddH2O was then added. After centrifugation (13,000 rpm for 3 min), the pigments were found in the pellet which was then resuspended in 100 μL ddH₂O. Of this suspension, 75 μL were added to a 384-well plate and the optical density/absorbance from 230 nm to 1000 nm was determined for each sample in 10 nm steps (bandwidth 230-310 nm: 5 nm, 320-1000 nm: 9 nm) using an Infinite 200 PRO NanoQuant Absorbance Reader (Tecan). Results were plotted as wavelength (nm) versus absorption (OD) and separately as wavelength (nm) versus absorption relative to lacZ-transfected cells.

The expression of mTyr resulted in production of pigments which resulted in high absorption of light over the whole tested spectrum, with the highest change in the visible spectrum observed at 450 nm. Co-expression of mTyr with Tyrp1 resulted in higher absorption (especially in the near infrared wavelengths). Additional expression of DCT with mTyr with Tyrp1 resulted in reduced absorption compared to mTyr with Tyrp1, but increased absorption compared to mTyr alone. Co-expression of DCT with mTyr resulted in reduced absorption compared to mTyr alone. The reduced absorption with the addition of DCT may be a consequence of reduced amounts of mTyr and/or Tyrp1 proteins, as the amount of each individual plasmid DNA is lower to achieve the same total amount of plasmid DNA for transfection.

### D. MRI Detection of Melanin Production in Transfected HeLa Cells

Melanin is associated with high signal intensity on T1-weighted MRI images due to shortened T1-relaxation times. In this experiment, changes in T1-relaxation times were examined in HeLa cells transfected with expression plasmids containing EGFP (pLENTI6N5-EGFP; SEQ ID NO: 71), mTyr (pLENTI6/V5-mTyr; SEQ ID NO: 9), Tyrp1 (pLENTI6N5-Tyrp1; SEQ ID NO: 24) transgenes, or combinations thereof.

HeLa cells were seeded in T175 flasks and transfected 3 days later with 20 μg total pLENTI6N5 plasmid DNA containing a 1:1 mixture of Tyrp1/EGFP, mTyr/EGFP, or Tyrp1/mTyr, or EGFP alone, using GeneCellin™ (20 mL total volume, 20 μg DNA dissolved in 0.5 mL medium with addition of 60 μL GeneCellin™ solution). The medium was supplemented with 250 μM ferric citrate. Three days after transfection, the cells were harvested by removing the medium, washing with 10 mL Hank's Balanced Salt Solution (HBSS), trypsinizing (5 mL Trypsin solution (PAA)) for 10 min at 37° C., adding 8 ml PBS (PAA) containing Ca²⁺ and Mg²⁺, centrifugation (1400 rpm for 5 min), and washing with 12 ml PBS. Harvested cells were counted and 3×10⁷ cells were pelleted by centrifugation at 1400 rpm for 5 min and then resuspended in 300 μL PBS. MRI-glass tubes (5 mm diameter) were then filled with the cell suspension, which were centrifuged at low speed before inversion recovery snapshot FLASH MR imaging was performed. Images were collected, using a Bruker Avance 500 equipped with an 11.7 T (500 MHz) vertical bore magnet at MR microscopy resolution with the following parameters: 40 echoes, 4 segments, TR=3,992 ms, TE=1,167 ms, flip angle α=4.5°, matrix size=128×128, FOV=20×20 mm² (with a resolution of 156.25×156.25 mm²), slice thickness=6 mm.

The experimentally determined T1 relaxation times were 1812±40 ms for EGFP, 1850±8 ms for mTyr/EGFP, 1899±6 ms for Tyrp1/EGFP and 1032±2 ms for mTyr/Tyrp 1. These results indicate that co-expression of mTyr and Tyrp 1, which results in increased eumelanin production (see Examples 1B and 1C), significantly reduces T1 relaxation time, which in turn produces greater contrast on images produced by MRI.

### Example 2

### Construction of Modified LIVP Vaccinia Viruses

Recombinant vaccinia viruses containing heterologous DNA were generated by insertion of one or more transgenes, from a transfer vector, into one or more loci of a starting vaccinia virus strain via homologous recombination, using methods described herein and known to those of skill in the art (see, e.g., Falkner and Moss (1990) J. Virol. 64:3108-2111; Chakrabarti et al. (1985) Mol. Cell Biol. 5:3403-3409; and U.S. Pat. No. 4,722,848). In these methods, the existing target gene in the starting vaccinia virus genome was replaced by an interrupted copy of the gene contained in the transfer vector through two crossover events: a first crossover event of homologous recombination between the vaccinia virus genome and the transfer vector and a second crossover event of homologous recombination between direct repeats within the target locus. The interrupted version of the target gene in the transfer vector contains the insertion DNA flanked on each side by DNA corresponding to the left portion of the target gene and right portion of the target gene, respectively. The transfer vector also contains a dominant selection marker, e.g., the E. coli guanine phosphoribosyltransferase (gpt) gene, under the control of a vaccinia virus early promoter (e.g., P7.5kE). Including such a marker in the vector enables a transient dominant selection process to identify recombinant virus grown under selective pressure that has incorporated the transfer vector into its genome. Because the marker gene is not stably integrated into the genome, it is deleted from the genome in a second crossover event that occurs when selection is removed. Thus, the final recombinant virus contains the interrupted version of the target gene as a disruption of the target loci, but does not retain the selectable marker from the transfer vector.

Modified vaccinia viruses described herein were generated by replacing nucleic acid or inserting nucleic acid in the F14.5L (also referred to as F3; see U.S. Pat. No. 7,588,767), thymidine kinase (TK), and hemagglutinin (HA) gene loci in the vaccinia virus genome. The heterologous DNA inserted was an expression cassette containing one or two genes of interest, or a reporter protein-encoding DNA, operably linked to a promoter, in the correct or reverse orientation to a vaccinia virus promoter.

Exemplary modified viruses are set forth in Table 14 below. Details of the experimental procedures to generate the modified viruses are provided in the sections that follow. These methods can be adapted to modify any of the viruses described herein to express tyrosinase and/or tyrosinase related protein-1 for production of melanin in infected cells. Also, the tyrosinase and/or tyrosinase related protein-1 encoding nucleic acid can be inserted into loci other than those exemplified, particularly into any non-essential locus.

TABLE 14
Summary of modified vaccinia viruses

| | SEQ | | | | |
|---|---|---|---|---|---|
| virus | ID | parent | F14.5L | TK | HA |
| name | NO: | strain | locus | locus | locus |
| GLV- | 2 | LIVP | (P_{SEL})Ruc- | (P_{SEL})rTrfR- | (P₁₁ₖ)gusA |
| 1h68 | | | GFP | (P_{7.5k})LacZ | |
| GLV- | | GLV- | (knock-out) | (P_{SEL})rTrfR- | (P₁₁ₖ)gusA |
| 1h71 | | 1h68 | | (P_{7.5k})LacZ | |
| GLV- | 3 | GLV- | (knock-out) | (P_{SEL})rTrfR- | (P_{SL})TurboFP635 |
| 1h254 | | 1h71 | | (P_{7.5k})LacZ | |
| GLV- | 64 | GLV- | (knock-out) | (P_{SEL})Tyrp1- | (P_{SL})TurboFP635 |
| 1h310 | | 1h254 | | (P_{SEL})mTyr | |
| GLV- | 65 | GLV- | (P_{SEL})Ruc- | (P_{SL})tetO- | (P₁₁ₖ)gusA |
| 1h311 | | 1h68 | CFP | CBG99- | |
| | | | | mRFP | |
| GLV- | 66 | GLV- | (P_{7.5k})-TetR | (P_{SL})tetO- | (P₁₁ₖ)gusA |
| 1h312 | | 1h311 | | CBG99- | |
| | | | | mRFP | |
| GLV- | 67 | GLV- | (P_{SEL})Ruc- | (P_{SE})Tyrp1- | (P₁₁ₖ)gusA |
| 1h322 | | 1h311 | GFP | (P_{SE})mTyr | |
| GLV- | 206 | GLV- | (P_{SEL})Ruc- | (P_{SEL})Tyrp1- | (P₁₁ₖ)gusA |
| 1h323 | | 1h311 | GFP | (P_{SEL})mTyr | |
| GLV- | 68 | GLV- | (P_{SEL})Ruc- | (P_{SE})Tyrp1- | (P₁₁ₖ)gusA |
| 1h324 | | 1h311 | GFP | (P_{SEL})mTyr | |
| GLV- | | GLV- | (P_{SEL})Ruc- | (P_{SEL})Tyrp1- | (P₁₁ₖ)gusA |
| 1h325 | | 1h311 | GFP | (P_{SE})mTyr | |
| GLV- | 69 | GLV- | (P_{SEL})Ruc- | (P_{SE})mTyr | (P₁₁ₖ)gusA |
| 1h326 | | 1h311 | GFP | | |
| GLV- | 70 | GLV- | (P_{SEL})Ruc- | (P_{SEL})mTyr | (P₁₁ₖ)gusA |
| 1h327 | | 1h311 | GFP | | |
| GLV- | | GLV- | (P_{SEL})Ruc- | (P_{SE})Tyrp1 | (P₁₁ₖ)gusA |
| 1h328 | | 1h311 | GFP | | |
| GLV- | | GLV- | (P_{SEL})Ruc- | (P_{SEL})Tyrp1 | (P₁₁ₖ)gusA |
| 1h329 | | 1h311 | GFP | | |
| GLV- | 210 | GLV- | (P_{7.5})tetR | (P_{SEL})rTrfR- | (P₁₁ₖ)gusA |
| 1h330 | | 1h68 | | (P_{7.5k})LacZ | |
| GLV- | 211 | GLV- | (P_{SEL})tetR | (P_{SL})tetO- | (P₁₁ₖ)gusA |
| 1h354 | | 1h311 | | CBG99- | |
| | | | | mRFP1 | |
| GLV- | 212 | GLV- | (P_{SE})hNISa | (P_{SE})Tyrp1- | (P₁₁ₖ)gusA |
| 1h458 | | 1h324 | | (P_{SEL})mTyr | |
| GLV- | 213 | GLV- | (P_{SE})hNISa | (P_{SEL})mTyr | (P₁₁ₖ)gusA |
| 1h459 | | 1h327 | | | |
| GLV- | 214 | GLV- | (P_{SEL})Ruc- | (P_{SL})tetO- | (P₁₁ₖ)gusA |
| 1h460 | | 1h311 | GFP | mTyr | |
| GLV- | 215 | GLV- | (P_{7.5})tetR | (P_{SL})tetO- | (P₁₁ₖ)gusA |
| 1h461 | | 1h330 | | mTyr | |
| GLV- | 216 | GLV- | (P_{SEL})tetR | (P_{SL})tetO- | (P₁₁ₖ)gusA |
| 1h462 | | 1h354 | | mTyr | |

### A. Generation of the GLV-1h68 Starting/Parental Strain

Modified vaccinia viruses were generated from the starting vaccinia virus (VV) strain GLV-1h68 (also named RVGL21 and GL-ONC1, SEQ ID NO: 2). This genetically engineered strain, which has been described in U.S. Pat. No. 7,588,767, was prepared from the vaccinia virus strain designated LIVP (a vaccinia virus strain, originally derived by adapting the Lister strain (ATCC Catalog No. VR-1549) to calf skin (Research Institute of Viral Preparations, Moscow, Russia, Al'tshtein et al. (1983) Dokl. Akad. Nauk USSR 285:696-699)). The LIVP strain (having a genome containing the sequence set forth in SEQ ID NO: 1), from which GLV-1h68 was generated, contains a mutation in the coding sequence of the TK gene, whereby a substitution of a guanine nucleotide with a thymidine nucleotide (nucleotide position 80207 of SEQ ID NO: 1) introduces a premature STOP codon within the coding sequence. The genome of LIVP also is set forth in SEQ ID NO:188, which represents the de novo parental sequence of GLV-1h68 without the insertions as described in Zhang et al. (2009) Mol. Genet. Genomics, 282:417-435.

As described in U.S. Pat. No. 7,588,767 (see Example 1), GLV-1h68 was generated by inserting expression cassettes encoding detectable marker proteins into the F14.5L (also designated in LIVP as F3), thymidine kinase (TK), and hemagglutinin (HA) gene loci of the vaccinia virus LIVP strain. Specifically, an expression cassette, containing a Ruc-GFP cDNA, a fusion of DNA encoding Renilla luciferase and DNA encoding GFP, under the control of a vaccinia synthetic early/late promoter (P_{SEL}) was inserted into the F14.5L gene; an expression cassette containing DNA encoding beta-galactosidase (β-gal) under the control of the vaccinia early/late promoter, P7.5k, and DNA encoding a rat transferrin receptor positioned in reverse orientation relative to an SEL promoter (and therefore not expressed), was inserted into the TK gene; and an expression cassette containing DNA encoding β-glucuronidase (gusA) under the control of the vaccinia late promoter, P11k, was inserted into the HA gene.

GLV-1h68 contains an expression cassette containing a Ruc-GFP cDNA molecule (a fusion of DNA encoding Renilla luciferase and DNA encoding GFP; SEQ ID NO: 78 (cDNA); SEQ ID NO: 79 (protein)) under the control of a vaccinia synthetic early/late promoter P_{SEL} ((P_{SEL})Ruc-GFP) inserted into the F14.5L gene locus; an expression cassette containing a DNA molecule encoding beta-galactosidase under the control of the vaccinia early/late promoter P_{7.5k} ((P_{7.5k})LacZ), DNA encoding a rat transferrin receptor positioned in the reverse orientation for transcription relative to the vaccinia synthetic early/late promoter P_{SEL} ((P_{SEL})rTrfR) inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) inserted into the HA gene locus. The genome of GLV-1h68 has the sequence of nucleotides set forth in SEQ ID NO:2. GLV-1h68 was used as a parental strain for the generation of additional modified viruses, described in the following sections.

### B. Generation of Other Starting Strains

i. GLV-1h71

GLV-1h71 was generated by insertion of a short non-coding DNA fragment containing BamHI and HindIII sites (SEQ ID NO: 41) into the F14.5L locus of starting strain GLV-1h68 (SEQ ID NO: 2) thereby deleting the Ruc-GFP fusion gene expression cassette at the F14.5L locus of GLV-1h68. Thus, in strain GLV-1h71, the vaccinia F14.5L gene is interrupted within the coding sequence by a short non-coding DNA fragment. GLV-1h71 was used as a starting strain to generate GLV-1h254.

ii. GLV-1h254

GLV-1h254 (SEQ ID NO: 3) was generated by insertion of an expression cassette encoding a TurboFP635 polypeptide (SEQ ID NO: 49 (cDNA); SEQ ID NO: 50 (protein)), a far-red mutant of the red fluorescent protein also known as “Katushka” from sea anemone Entacmaea quadricolor, under the control of the vaccinia P_{SL} promoter into the HA locus of starting strain GLV-1h71 thereby deleting the gusA expression cassette at the HA locus of starting GLV-1h71. Thus, in strain GLV-1h254 (SEQ ID NO: 3), the vaccinia HA gene is interrupted within the coding sequence by (P_{SL})TurboFP635. GLV-1h254 was used as the starting strain for the generation of GLV-1h310.

iii. GLV-1h330

The cDNA encoding tetR (Tetracycline repressor, codon optimized for expression in mammalian system), was PCR amplified from the lentiviral vector system designated FH1tUTG (Herold et al., (2008) PNAS 105(47):18507-18512) as the template with primers tetR-5 (5′-CTCGAG(Xho I) ATGTCCAGACTGGACAAGAGCAA-3′; SEQ ID NO:53) and tetR-3 (5′-GGATCC(BamH I) TCAAGAGCCAGACTCACATTTCAGCTGC-3′; SEQ ID NO:54). The PCR product was gel-purified, and cloned into the pCR-Blunt II-TOPO vector (SEQ ID NO: 52) using Zero Blunt TOPO PCR Cloning Kit (Invitrogen). The resulting construct pCRII-tetR1 was sequence confirmed. The tetR cDNA was then released from pCRII-tetR1 with Xho I and BamH I, and subcloned into vaccinia shuttle vector pSC65 (Earl et al., “Preparation of cell cultures and vaccinia virus stocks. In: Ausubel et al, editors. Current protocols in molecular biology. Vol 2. New York (N.Y.): Greene Publishing Associates and Wiley Interscience; 1991. p. 16.16.1-16.16.7) with the same cuts, replacing lacZ cDNA with tetR and thus placing tetR under the control of vaccinia virus promoter p7.5. The p7.5-tetR cDNA was then released from the pSC65-tetR construct with Hind III and BamH I, and subcloned into the transfer vector for the F14.5 locus (pNCVVf14.51T) with the same cuts. The resulting construct F14.5L-p7.5-tetR1 (SEQ ID NO: 77) was sequence confirmed, and used for homologous recombination at the F14.5L locus of GLV-1h68 (SEQ ID NO: 2) to generate GLV-1h330 (SEQ ID NO: 210).

iv. Generation of Vaccinia Viruses Carrying Inducible Tetracycline (TetO) System

Modified viruses, expressing a tetracycline-inducible reporter construct or a reporter construct repressed by tetracycline or a derivative thereof, were generated by homologous recombination with the GLV-1h68 parental strain generated in part A above. These viruses, designated GLV-1h311 (SEQ ID NO: 65) and GLV-1h312 (SEQ ID NO: 66), respectively, were used as control constructs in the Examples below. GLV-1h311 (SEQ ID NO: 65) was also used as a parental strain for the construction of additional modified viruses.

a. GLV-1h311

GLV-1h311 (SEQ ID NO: 65) was generated by replacing the (P_{SEL})rTrfR-(P_{7.5k})Lacz insertion in the TK locus of GLV-1h68 with an expression cassette containing (CBG99)-2A-mRFP1 (SEQ ID NO: 44), a fusion protein of a green-emitting Chroma-Luc luciferase (CBG99) linked with monomeric red fluorescent protein (mRFP), by a “self-cleaving” 2A peptide under the regulation of a tet operator (tetO) sequence and the vaccinia virus SL promoter. The cDNA encoding (CBG99)-2A-mRFP was PCR amplified using plasmid pCRTK-(P_{SEL})mRFP (SEQ ID NO: 46; described in U.S. Pat. No. 8,052,968) as the template, with primers tetO-mRFP-5 (5′-GTCGAC(SalI) TCCCTATCAGTGATAGAGAATGGTGAAGCGTGAGAAAAATGTCATC-3′; SEQ ID NO: 47) and mRFP-3 (5′-TTAATTAA(PacI) TTAGGCGCCGGTGGAGT-3′; SEQ ID NO: 48). The forward tetO-mRFP-5 primer (SEQ ID NO: 47), introduced a tet operator (tetO) sequence (TCCCTATCAGTGATAGAGA; SEQ ID NO: 51) at the 5′ end of (CBG99)mRFP.

The PCR product was gel-purified, and cloned into the pCR-Blunt II-TOPO vector (SEQ ID NO: 52) using Zero Blunt TOPO PCR Cloning Kit (Invitrogen). The resulting construct, pCRII-tetO-(CBG99)mRFP, was sequence confirmed. The tetO-(CBG99)mRFP cDNA (SEQ ID NO: 45) was then released from pCRII-tetO-(CBG99)mRFP with SalI and PacI, and subcloned into SalI/PacI-digested TK-(P_{SL})mLIGHT1 (SEQ ID NO: 207), replacing the mLIGHT cDNA in the vector. The resulting construct, TK-(P_{SL})tetO-(CBG99)mRFP was sequence confirmed and used to make recombinant virus GLV-1h311 (SEQ ID NO: 65), inserting (P_{SL})tetO-(CBG99)mRFP in the TK locus, by homologous recombination with parental virus GLV-1h68 (SEQ ID NO: 2).

b. GLV-1h312

GLV-1h312 was generated by replacing the (P_{SEL})Ruc-GFP insert within the F14.5L locus of GLV-1h311 (SEQ ID NO: 65) with an expression cassette expressing tetracycline repressor (tetR) under the control of the vaccinia early/late promoter, P7.5k. F14.5L-p7.5-tetR1 (SEQ ID NO: 77), generated as described in Example 2B(iii) above, was recombined with GLV-1h311 (SEQ ID NO: 65) to insert (P_{7.5k})TetR in the F14.5 locus, thereby generating GLV-1h312 (SEQ ID NO: 66).

c. GLV-1h354

The tetR cDNA was released from F14.5L-p7.5-tetR1 (SEQ ID NO:77) with Xho I and Sal I digestion, gel purified, and ligated with the F14.5L-SEL fragment of Sal I-digested F-SEL-CBG99-mRFP2. The resulting construct, FSEL-tetR1 (SEQ ID NO: 223) was sequence confirmed and used for homologous recombination at the F14.5L locus of GLV-1h311 (SEQ ID NO: 65) to generate GLV-1h354 (SEQ ID NO: 211).

C. Generation of Vaccinia Viruses Encoding mTyr (Tyrosinase)

i. GLV-1h460, GLV-1h461, and GLV-1h462 ((P_{SL})tetO-mTyr)

A tetO-mTyr fragment was PCR amplified from TK-SE-mTyr-1 (SEQ ID NO: 12) using primers SalI-tetO-mTyr (5′-GTCGAC(SalI) TCCCTATCAGTGATAGAGAATGTTCTTGGCTGTTTTGTATTGCCTTC-3′; SEQ ID NO: 230) and mTyr-3 (5′-TTAATTAA(PacI) TCACAGATGGCTCTGATACAGC-3′; SEQ ID NO: 11). The purified PCR product was then cloned with the pCR Blunt II TOPO kit (Invitrogen). The resulting pCRII-mTyr plasmid was digested with SalI and Pad and ligated with SalI and PacI-digested TK-SL fragment of the TK-(P_{SL})mLIGHT1 transfer vector (SEQ ID NO: 207). The resulting construct, TK-SL-tetO-mTyr (SEQ ID NO: 227), was sequence confirmed and used for homologous recombination at the TK locus of GLV-1h311 (SEQ ID NO: 65), GLV-1h330 (SEQ ID NO: 210) and GLV-1h354 (SEQ ID NO: 211) to generate GLV-1h460 (SEQ ID NO: 214), GLV-1h461 (SEQ ID NO: 215) and GLV-1h462 (SEQ ID NO: 216), respectively.

ii. GLV-1h326, GLV-1h327, and GLV-1h459

The insertion within the TK locus of GLV-1h311 was replaced with an expression cassette containing cDNA encoding mouse Tyrosinase (mTyr) under the regulation of a vaccinia virus SE (weaker) or SEL(stronger) promoter to generate GLV-1h326 and GLV-1h327, respectively. The (P_{SEL})Ruc-GFP insert in the F15.5L locus of GLV-1h327 was then replaced with a cassette containing the transgene encoding human sodium iodide symporter (hNIS) under the regulation of the P_{SE} promoter to generate GLV-1h459.

a. GLV-1h326 and GLV-1h327

The cDNA encoding mTyr was PCR amplified using the plasmid pLENTI6/V5-mTyr as the template with primers mTyr-5 (5′-GTCGAC(SalI) CACCATGTTCTTGGCTGTTTTGTATTGCCT-3′; SEQ ID NO: 10) and mTyr-3 (5′-TTAATTAA(PacI) TCACAGATGGCTCTGATACAGC-3′; SEQ ID NO: 11). The PCR product was gel-purified, and cloned into the pCR-Blunt II-TOPO vector (Invitrogen; SEQ ID NO: 52) using Zero Blunt TOPO PCR Cloning Kit (Invitrogen). The resulting construct, pCRII-mTyr-1, was confirmed by sequencing. The mTyr encoding DNA was then released from pCRII-mTyr-1 by SalI/PacI digestion and subcloned into TK-(P_{SE})mLIGHT2 (SEQ ID NO: 76) and TK-(P_{SEL})mLIGHT1 (SEQ ID NO: 75), which were likewise digested, replacing the mLIGHT-encoding cDNA in both vectors. The resulting constructs, TK-(P_{SE})mTyr-1 (SEQ ID NO: 12) and TK-(P_{SEL})mTyr-1 (SEQ ID NO: 13), were sequence confirmed and then recombined with parental virus GLV-1h311 (SEQ ID NO: 65), exchanging the expression cassettes in the TK locus, to create recombinant viruses GLV-1h326 (SEQ ID NO: 69) and GLV-1h327 (SEQ ID NO: 70), respectively.

b. GLV-1h459

GLV-1h459 was generated by replacing the (P_{SEL})Ruc-GFP expression cassette in the F14.5L locus of GLV-1h327 with a cassette containing the transgene encoding human sodium iodide symporter (hNIS) under the regulation of the P_{SE} promoter. Expression of hNIS facilitates deep tissue imaging without altering the oncolytic or replication capabilities of vaccinia virus (U.S. Patent Publ. No. 2009-0117034; Haddad et al., (2011) J. Transl. Med. 9:36).

hNIS cDNA was PCR amplified using human cDNA clone TC124097 (SLC5A5) from OriGene as the template with following primers: hNIS-5 (5′-GTCGAC(SalI) CACCATGGAGGCCGTGGAGACCGG-3′, SEQ ID NO: 234) and hNIS-3 (5′-TTAATTAA (PacI)TCAGAGGTTTGTCTCCTGCTGGTCTCGA-3′, SEQ ID NO: 235). The PCR product was gel-purified, and cloned into the pCR-Blunt H-TOPO vector (SEQ ID NO: 52) using Zero Blunt TOPO PCR Cloning Kit (Invitrogen). The resulting construct, pCRII-hNIS-1, was sequenced and found to contain an extra 33-bp segment in the middle of the coding sequence, representing an alternative splicing product for hNIS. To remove this extra 33-bp segment, two additional primers, hNIS-a5 (5′-CCCAGACCAGTACATGCCTCTGCTGGTGCTG-3′; SEQ ID NO: 236) and hNIS-a3 (5′-GAGGCATGTACTGGTCTGGGGCAGAGATGC-3′; SEQ ID NO: 237) were designed to flank the segment.

The pCRII-hNIS-1 construct was then used as the template for two subsequent PCR amplifications: 1) using hNIS-5 (SEQ ID NO: 234) paired with hNIS-a3 (SEQ ID NO: 237) and 2) using hNIS-a5 (SEQ ID NO: 236) paired with hNIS-3 (SEQ ID NO: 235). The respective PCR products were then mixed and used as the templates in one reaction with the primer pair hNIS-5 (SEQ ID NO: 234) and hNIS-3 (SEQ ID NO: 235). The final PCR product was cloned into the pCR-Blunt II-TOPO vector (SEQ ID NO: 52) and designated pCRIIhNISa-2, Sequencing of pCRIIhNISa-2 confirmed the sequence of the hNISa transgene was identical to the SLC5A5 sequence in GenBank (accession number NM_{—}000453; SEQ ID NO: 226). The sequence-confirmed hNISa cDNA was then released from pCRII-hNISa-2 with Sal I and Pac I and subcloned into HA-SE-RLN-7 with the same cuts, replacing the RLN cDNA. The resulting construct HA-SE-hNISa-2 was confirmed by sequencing.

HA-SE-hNISa-2 was then used as the template for PCR, using primers FSEin-5 (5′-TCGTGTCTGTAAGCTT(HindIII) AAAAATTGAAAAACTAGCGTCTTTTTTTGC-3′; (SEQ ID NO: 208) and FinhNISa-3 (5′-GGATCC(BamHI) TGCGAAGCTTTCCAGACATTGTTGAATTAGATCGAT-3′; SEQ ID NO: 209) to amplify SE-hNISa cDNA. The SE-hNISa PCR product was gel purified, and cloned, using the In-Fusion PCR Cloning System (Clontech), into the gel-purified F14.5L transfer vector of HindIII/BamHI digested F14.5L-SE-hNET. The resulting construct, FSE-hNISa (SEQ ID NO: 224), was sequence confirmed and used for homologous recombination at the F14.5L locus of GLV-1h327 (SEQ ID NO: 70) to generate GLV-1h459 (SEQ ID NO: 213).

D. Generation of Viruses Encoding Tyrp1 (GLV-1h328, 1h329)

The insertion within the TK locus of GLV-1h311 was replaced with an expression cassette containing cDNA encoding Tyrosinase Related Protein-1 (Tyrp1) under the regulation of a vaccinia virus SE (weaker) or SEL(stronger) promoter to generate GLV-1h328 and GLV-1h329, respectively.

To generate the Tyrp expression cassette, Tyrp1 encoding cDNA was PCR amplified using plasmid pLENTI6N5-Tyrp1 as the template with primers Tyrp-5 (5′-GTCGAC(SalI) CACCATGAGTGCTCCTAAACTCCTCTCT-3′; SEQ ID NO: 21) and Tyrp-3 (5′-TTAATTAA(PacI) TTAGACCACAGACTGATTAGGATTCTGG-3′; SEQ ID NO: 22). Following gel purification, the PCR product was cloned into the pCR-Blunt II-TOPO vector (Invitrogen; SEQ ID NO: 52) using Zero Blunt TOPO PCR Cloning Kit (Invitrogen). The resulting construct pCRII-Tyrp1-1 was sequence confirmed. The Tyrp1 cDNA was then released from pCRII-Tyrp1-1 by SalI/PacI digestion, and subcloned into likewise digested TK-(P_{SE})mLIGHT2 (SEQ ID NO: 76) and TK-(P_{SEL})mLIGHT1 (SEQ ID NO: 75) replacing the mLIGHT-encoding cDNA. The resulting constructs TK-(P_{SE})Tyrp1 (SEQ ID NO: 25) and TK-(P_{SEL})Tyrp1 (SEQ ID NO: 26) were sequence confirmed and then recombined with parental virus GLV-1h311 (SEQ ID NO: 65), exchanging the expression cassettes in the TK locus, to create recombinant viruses GLV-1h328 and GLV-1h329, respectively.

E. Generation of Viruses Encoding Melanin-Producing Enzymes, mTyr and Tyrp1

Recombinant viruses encoding human Tyrosinase-related protein 1 (Tyrp 1) and mTyr were created by ligating an expression cassette containing mTyr encoding cDNA, under the regulation of an SE or SEL vaccinia virus promoter, with an expression cassette containing Tyrp1 encoding cDNA, also under the regulation of an SE or SEL vaccinia virus promoter, and inserting the generated double constructs into the TK locus of GLV-1h311 to generate the recombinant viruses below.

i. Generation of GLV-1h322 ((P_{SE})Tyrp1/(P_{SE})-mTyr)

(P_{SE})mTyr and (P_{SE})Tyrp1 expression cassettes were released from TK-(P_{SE}) mTyr-1 (SEQ ID NO: 12; described in part D) and TK-(P_{SE})Tyrp1 (SEQ ID NO: 25; described above), respectively, by SacI digestion. The (P_{SE})mTyr and (P_{SE})Tyrp1 fragments, flanked by cohesive SacI restriction sites, were gel purified and ligated together with Quick Ligation Kit (New England BioLabs). The ligation products were then used as the template for PCR with primers (P_{SE})Sal-Tyrp1 (5′-TTTTTTTGCTCGAAGTCGAC(SalI) CACCATGAGTGCTCC-3′; SEQ ID NO: 27) and Ter-PacI-mTyr (5′-GATCGATAAAAATTAATTAA(PacI) TCACAGATGGCTCTG-3′; SEQ ID NO: 14). The PCR product was then cloned into the gel-purified TK-SE fragment of TK-(P_{SE})mTyr-1, resulting from SalI/PacI digestion, using the In-Fusion PCR Cloning System (Clontech). The resulting construct TK-(P_{SE})Tyrp1-(P_{SE})mTyr (SEQ ID NO: 15) was sequence confirmed, and was recombined with the TK locus of GLV-1h311 (SEQ ID NO: 65) to generate GLV-1h322 (SEQ ID NO: 67).

ii. Generation of GLV-1h310 and GLV-1h323 ((P_{SEL})Tyrp1/(P_{SEL})mTyr)

Expression cassettes (P_{SEL})mTyr and (P_{SEL})Tyrp1 were released from TK-(P_{SEL})mTyr-1 (SEQ ID NO: 13) and TK-(P_{SEL})Tyrp1 (SEQ ID NO: 26), respectively by SalI digestion. The (P_{SEL})mTyr and (P_{SEL})Tyrp1 fragments, flanked by cohesive SalI restriction sites were gel-purified, and ligated together with Quick Ligation Kit (New England BioLabs). The ligation products were then used as the template for PCR with primers (P_{SEL})Sal-Tyrp1 (5′-TAAATAAGCTCGAAGTCGAC(SalI) CACCATGAGTGCTCC-3′; SEQ ID NO: 28) and Ter-PacI-mTyr (5′-GATCGATAAAAATTAATTAA(PacI) TCACAGATGGCTCTG-3′; SEQ ID NO: 14). The PCR product was cloned, using the In-Fusion PCR Cloning System (Clontech), into the gel-purified TK-SEL fragment of SalI/PacI digested TK-(P_{SEL})mTyr-1. The resulting construct, TK-(P_{SEL})Tyrp1-(P_{SEL})mTyr (SEQ ID NO: 17) was sequence confirmed, and was recombined with the TK locus of GLV-1h254 (SEQ ID NO: 3) to generate GLV-1h310 (SEQ ID NO: 64). The TK-(P_{SEL})Tyrp1-(P_{SEL})mTyr construct (SEQ ID NO: 17) also was recombined with GLV-1h311 (SEQ ID NO: 65) to generate GLV-1h323 (SEQ ID NO: 206).

iii. Generation of GLV-1h324 and GLV-1h458((P_{SE})Tyrp1/(P_{SEL})mTYR)

Expression cassettes (P_{SEL})mTyr and (P_{SE})Tyrp1 were released from TK-(P_{SEL})mTyr-1 and TK-(P_{SE})Tyrp1, respectively, by SacI digestion. The gel-purified (P_{SEL})mTyr and (P_{SE})Tyrp1 fragments were then ligated together using Quick Ligation Kit (New England BioLabs), and the ligation products were used as the template for PCR with primers (P_{SE})Sal-Tyrp1 (5′-TTTTTTTGCTCGAAGTCGAC(SalI) CACCATGAGTGCTCC-3′; SEQ ID NO: 27) and Ter-PacI-mTyr (5′-GATCGATAAAAATTAATTAA(PacI) TCACAGATGGCTCTG-3′; SEQ ID NO: 14). The (P_{SE})Tyrp1-SEL PCR product was then cloned, using the In-Fusion PCR Cloning System (Clontech), into the TK-SE fragment SalI/Pad-digested TK-(P_{SE})mTyr-1. The resulting construct, TK-(P_{SE})Tyrp1-(P_{SEL})mTyr (SEQ ID NO: 18), was sequence confirmed, and used for homologous recombination at the TK locus of GLV-1h311 (SEQ ID NO: 65) to generate GLV-1h324 (SEQ ID NO: 68).

The F-SE-hNIS (SEQ ID NO: 224) construct, containing the transgene for human sodium iodide symporter under the regulation of the PSE promoter, was generated as described in Example 2C(ii) and recombined with GLV-1h324 (SEQ ID NO: 68) to insert the (P_{SE})hNIS transgene into the F14.5L locus to thereby generate GLV-1h458 (SEQ ID NO: 212).

iv. Generation of GLV-1h325 ((P_{SEL})Tyrp1/(P_{SE})mTYR)

(P_{SE}) mTyr and (P_{SEL})Tyrp1 expression cassette were released from TK-(P_{SE})mTyr-1 (SEQ ID NO: 12; described in part D) and TK-(P_{SEL})Tyrp1 (SEQ ID NO: 26; described above), respectively, by SalI digestion. The (P_{SE})mTyr and (P_{SEL})TyrP1 fragments, flanked by cohesive SalI restriction sites, were gel purified and ligated together with Quick Ligation Kit (New England BioLabs). The ligation products were then used as the template for PCR with primers (P_{SEL})Sal-Tyrp1 (5′-TAAATAAGCTCGAAGTCGAC(SalI) CACCATGAGTGCTCC-3′; SEQ ID NO: 28) and Ter-PacI-mTyr (5′-GATCGATAAAAATTAATTAA(PacI) TCACAGATGGCTCTG-3′; SEQ ID NO: 14). The PCR product was cloned into the gel-purified TK-SEL fragment of SalI/PacI digested TK-(P_{SEL})mTyr-1, using the In-Fusion PCR Cloning System (Clontech). The resulting construct TK-(P_{SEL})Tyrp1-(P_{SE})mTyr was sequence confirmed and recombined with the TK locus of GLV-1h311 (SEQ ID NO: 65) to generate GLV-1h325.

v. Tetracycline-Inducible mTyr and Tyrp1 from Single Promoter

PCR fragments mTyr-2A and E2A-Tyrp1 were amplified using Phusion High-fidelity DNA polymerase (Finnzymes). For mTyr-2A, the primers attB1-mTyr-for (SEQ ID NO: 33) and mTyr-E2A rev (SEQ ID NO: 228) were used with pENTR221-mTyr (SEQ ID NO: 8) serving as template. For E2A-Tyrp1 the template was pENTR221-TYRP1 (SEQ ID NO: 23) using the primers E2A-Tyrp1 for (SEQ ID NO: 229) and attB2-Tyrp1-rev (SEQ ID NO: 36).

The PCR fragments were digested with AatII (New England Biolabs) and subsequently ligated using T4-Ligase (New England Biolabs). The ligated fragment was gel purified and used in a BP-Clonase reaction (Invitrogen, Multisite Gateway® system) with pDONR221 (Invitrogen; SEQ ID NO: 5) to generate pENTR221-MTT2 (SEQ ID NO: 232). pENTR221-MTT2 served as template for another PCR using the primers SalI-tetO-mTyr (SEQ ID NO: 230) and Tyrp1-3 (SEQ ID NO: 22) and Accu Prime Pfx Super Mix.

The resulting PCR fragment was cloned into pCR-BluntII-TOPO (Invitrogen; SEQ ID NO: 52) resulting in pCR-BluntII-TOPO-SalI-tetO-mTyr-E2A-hTyrp1-inverse (SEQ ID NO: 233). The pCR-BluntII-TOPO-SalI-tetO-mTyr-E2A-hTyrp1-inverse vector was sequence confirmed and was digested with SalI and PacI and ligated with the TK-SL fragment of the SalI and PacI-digested TK-(P_{SL})mLIGHT1 transfer vector (SEQ ID NO: 207), resulting in TK-SL-tetO-mTyr-E2A-hTyrp1 (SEQ ID NO: 231).

### Example 3

### Construction of Modified Clonal Isolates of LIVP

LIVP clonal isolates were isolated and sequenced, as described below. The clonal isolate, LIVP 1.1.1 was then modified by homologous recombination, using the methods described in Example 2 above.

### A. Isolation of Clonal Isolates of LIVP

Isolation of clonal isolates of LIVP were generated as described in related U.S. Patent Publication No. 2012/0308484. Briefly, African green monkey kidney fibroblast CV-1 cells (ATCC No. CCL-70; American Type Culture Collection (Manassas, Va.)) were plated in a 6-well plate at 5×10⁵ cells per well and grown in Dulbecco's modified Eagle's medium (DMEM, Mediatech, Inc., Herndon, Va.) supplemented with 1% antibiotic-antimycotic solution (Mediatech, Inc., Herndon, Va.) and 10% fetal bovine serum (FBS, Mediatech, Inc., Herndon, Va.) overnight at 37° C. in a humidified incubator supplied with 5% CO₂ until cells reached 90% confluency. CV-1 cells were then infected with 10-fold serial dilutions of the vaccinia virus LIVP (a vaccinia virus strain, originally derived by adapting the Lister strain (ATCC Catalog No. VR-1549) to calf skin (Institute of Viral Preparations, Moscow, Russia, Al′tshtein et al., (1983) Dokl. Akad. Nauk USSR 285:696-699)) in duplicate. Serial dilutions were employed for infection to ensure isolation of well-separated plaques. Two days post infection, eight well-isolated plaques exhibiting a large plaque phenotype relative to the other plaques on the plate were picked. These plaques were subjected to two more rounds of plaque purification in CV-1 cells and designated as LIVP clonal isolates 1.1.1, 2.1.1, 3.1.1, 4.1.1, 5.1.1, 6.1.1, 7.1.1, and 8.1.1, respectively. LIVP 1.1.1 and 4.1.1 formed larger plaques in CV-1 cells than other isolates. The LIVP clonal isolates were then sequenced and analyzed, and the results were compared to known sequences for Vaccinia virus strains GLV-1h68, Western Reserve and Copenhagen, as described in Section B below.

### B. Sequencing Clonal Isolates of LIVP

LIVP clonal isolates 1.1.1, 2.1.1, 3.1.1, 4.1.1, 5.1.1, 6.1.1, 7.1.1, and 8.1.1 were propagated and titrated in CV-1 cell monolayers. CV-1 cells infected with LIVP clonal isolates 1.1.1, 2.1.1, 3.1.1, 4.1.1, 5.1.1, 6.1.1, 7.1.1 or 8.1.1 were harvested by centrifugation and disrupted by three cycles of freeze and thaw. Cell debris and nucleic were removed from cell lysates by low-speed centrifugation. The recovered viral particles were purified by centrifugation through sucrose cushions and sucrose gradients (20-40%) using standard protocols. Genomic viral DNA was extracted from purified virions after treatment with proteinase K and followed by phenol-chloroform extraction (see, Earl et al., in Ausubel et al., (eds) Current protocols in molecular biology, vol. 3, pages 16.17.1-16.19-7 (1998)).

The genomic DNA was sequenced by shotgun approach and assembled by AGOWA GmbH. Gap closure was performed by additional sequencing runs on the shotgun clones or by PCR using viral genomic DNA. Inverted terminal repeats were reconstructed by comparing the reverse complement sequence to the sequence, identifying identical sequences and extending the flanking sequence. The ORFs were compared to those of known Vaccinia strains GLV-1h68 (SEQ ID NO:2, GenBank Accession No. EU410304), Western Reserve (SEQ ID NO:62, GenBank Accession No. AY243312) and Copenhagen (SEQ ID NO:63, GenBank Accession No. M35027). Tables comparing the major and minor ORFs in these strains can be found in Zhang et al., Mol Genet Genomics 282:417-438 (2009). The sequence of each individual LIVP clonal isolate was aligned to the parental LIVP isolate of GLV-1h68 without the insertions (SEQ ID NO:188) using a Pairwise Sequence Alignment program available from Iowa State University (deepc2.psi.iastate.edu/aat/align/align.html; Huang, X. (1994) Computer Applications in the Biosciences 10:227-235). The alignment was computed as a “Global Alignment with GAP.” The GAP program computes an optimal global alignment of two sequences without penalizing terminal gaps. A long gap in the shorter sequence is given a constant penalty. The two sequences must be of the same type, that is, both are DNA sequences or both are protein sequences. GAP delivers the alignment in linear space, so long sequences can be aligned. The default parameters were set at “Max Match” score 10, “Min Mismatch,” score −15, “Gap-Open penalty” score 30, and “Gap-Extension penalty” Score 3.

Table 15 below sets forth the clonal isolates, including the SEQ ID NO, genome size, nucleotides corresponding to the inverted terminal repeats and percent (%) identity to the parental LIVP isolate set forth in SEQ ID NO:188. Sequencing revealed LIVP 1 isolate 3.1.1 was a mixture of at least 2 sequences.

TABLE 15
LIVP clonal isolates

| LIVP | SEQ | | | | % Identity |
|---|---|---|---|---|---|
| iso- | ID | Genome | | | to SEQ |
| late | NO | size | Left ITR | Right ITR | ID NO: 10 |
| LIVP | 188 | 190,217 | 1 . . . 10,072 | 180,096 . . . 190,167 | 100 |
| 1.1.1 | 55 | 183,369 | 1 . . . 2255 | 181,115 . . . 183,369 | 93 |
| 2.1.1 | 56 | 193,964 | 1 . . . 11,242 | 182,722 . . . 193,964 | 97 |
| 4.1.1 | 57 | 187,653 | 1 . . . 6,263 | 181,391 . . . 187,653 | 93 |
| 5.1.1 | 58 | 188,863 | 1 . . . 7,043 | 181,821 . . . 188,863 | 94 |
| 6.1.1 | 59 | 188,082 | 1 . . . 6,673 | 181,410 . . . 188,082 | 94 |
| 7.1.1 | 60 | 188,082 | 1 . . . 6,715 | 181,368 . . . 188,082 | 94 |
| 8.1.1 | 61 | 188,768 | 1 . . . 6,898 | 181,871 . . . 188,768 | 94 |

### C. Modified LIVP 1.1.1 Viruses

Exemplary recombinant LIVP 1.1.1 vaccinia viruses, set forth in Table 16 below, were generated by insertion of one or more transgenes into the thymidine kinase (TK) locus via homologous recombination, using the methods described above in Example 2. Details of the experimental procedures to generate clonal isolates and modified viruses are provided in the sections that follow. These methods can be adapted to modify any of the clonal isolates described herein to express tyrosinase and/or tyrosinase related protein-1 for production of melanin in infected cells. Also, the tyrosinase and/or tyrosinase related protein-1 encoding nucleic acid(s) can be inserted into loci other than those exemplified, particularly into any non-essential locus.

TABLE 16
Summary of modified LIVP 1.1.1 viruses

| virus | SEQ ID | parent | F14.5L | TK | HA |
|---|---|---|---|---|---|
| name | NO: | strain | locus | locus | locus |
| GLV- | 194 | LIVP | wt | (P_{SEL})FUKW | wt |
| 2b372 | | 1.1.1 | | | |
| GLV- | 198 | GLV- | wt | (P_{SEL})Tyrp1- | wt |
| 2b452 | | 2b372 | | (P_{SEL})mTyr | |
| GLV- | 199 | GLV- | wt | (P_{SE})Tyrp1- | wt |
| 2b453 | | 2b372 | | (P_{SEL})mTyr | |
| GLV- | 200 | GLV- | wt | (P_{SEL})mTyr | wt |
| 2b482 | | 2b372 | | | |

1. Generation of GLV-2b372

GLV-2b372 was generated by modifying LIVP 1.1.1 using homologous recombination, whereby an expression cassette containing TurboFP635 (SEQ ID NO:49), encoding far-red fluorescent protein “katushka” (SEQ ID NO: 50), under the control of the vaccinia synthetic early/late (SEL) promoter was inserted at the TK locus of LIVP 1.1.1 using the methods described in Example 2B above. The genome of GLV-2b372 has the sequence of nucleotides set forth in SEQ ID NO:194. GLV-2b372 was used as the starting strain for the generation of GLV-2b452, GLV-2b453, and GLV-2b482 as described below.

2. Generation of GLV-2b452 (SEL-Tyrp1/SEL-mTyr)

The TK-SEL-Tyrp1-SEL-mTyr (SEQ ID NO: 17) construct, generated as described in Example 2E(ii), was recombined with the TK locus of GLV-2b372 to create GLV-2b452 (SEQ ID NO: 198). GLV-2b452 expresses Tyrp1 and mTyr, each under the control of an SEL promoter.

3. Generation of GLV-2b453 (SE-Tyrp1/SEL-mTyr)

The TK-SE-Tyrp1-SEL-mTyr (SEQ ID NO: 18) construct, generated as described in Example 2E(iii), was recombined with the TK locus of GLV-2b372 to create GLV-2b453 (SEQ ID NO: 199). Thus, GLV-2b453 expresses Tyrp 1 under the control of an SE promoter and mTyr under the control of an SEL promoter.

4. Generation of GLV-2b482 (SEL-mTyr)

The TK-SEL-mTyr (SEQ ID NO: 13) construct, generated as described in Example 2C(ii), was recombined GLV-2b372 to exchange expression cassettes in the TK locus and create GLV-2b482 (SEQ ID NO: 200). Thus the mTyr transgene contained within GLV-2b482 is regulated by the SEL promoter.

### Example 4

### Construction of Modified Western Reserve Vaccinia Viruses

The Western Reserve (WR) vaccinia virus (ATCC Catalog No. VR-1354; GenBank Accession No.: AY243312.1; SEQ ID NO: 62) also was modified to generate virus strains that contain one or more chromophore producing enzymes. Modified WR vaccinia viruses described herein were generated by replacing nucleic acid or inserting nucleic acid in the thymidine kinase (TIC), and/or hemagglutinin (HA) gene loci in the WR genome via homologous recombination as described in Example 2. The heterologous DNA inserted was an expression cassette containing one or two genes of interest, or a reporter protein-encoding DNA, operably linked to a promoter, in the correct or reverse orientation to a vaccinia virus promoter.

Exemplary modified WR viruses are set forth in Table 17 below. Details of the experimental procedures to generate the modified viruses are provided in the sections that follow. These methods can be adapted to insert the tyrosinase and/or tyrosinase related protein-1 encoding nucleic acid into loci other than those exemplified, particularly any non-essential locus.

TABLE 17
Summary of modified WR viruses

| virus | SEQ ID | parent | F14.5L | TK | HA |
|---|---|---|---|---|---|
| name | NO: | strain | locus | locus | locus |
| GLV- | 201 | WR | wt | (P_{SL})-tetO- | wt |
| 0b348 | | | | CBG99- | |
| | | | | mRFP1 | |
| GLV- | 202 | GLV- | wt | (P_{SE})EFNA1 | wt |
| 0b358 | | 0b348 | | | |
| GLV- | 203 | GLV- | wt | (P_{SE})EFNA1 | (P_{SEL})FUKW |
| 0e365 | | 0b358 | | | |
| GLV- | 204 | GLV- | wt | (P_{SE})Tyrp1- | (P_{SEL})FUKW |
| 0e406 | | 0e365 | | (P_{SEL})mTyr | |
| GLV- | 205 | GLV- | wt | (P_{SEL})mTyr | (P_{SEL})FUKW |
| 0e407 | | 0e365 | | | |

A. Generation of the GLV-0b348 Parental Strain

GLV-0b348 was generated by replacing the TK locus of WR strain (SEQ ID NO: 62) with an expression cassette containing CBG99-2A-mRFP1 (SEQ ID NO: 44), encoding a fusion protein of a green-emitting Chroma-Luc luciferase (CBG99) linked with monomeric red fluorescent protein (mRFP) (SEQ ID NO: 43) by a “self-cleaving” 2A peptide, under the regulation of a tet operator (tetO) sequence and the vaccinia virus SL promoter. The TK-SL-tetO-CBG99-mRFP construct was generated as described in Example 2B(iv)(a). SL-tetO-CBG99-mRFP was then inserted into the TK locus of WR (SEQ ID NO: 62) from the construct by homologous recombination to generate GLV-0b348 (SEQ ID NO: 201).

B. Generation of GLV-0b358 (SE-EFNA1)

The cDNA encoding EFNA1 (Homo sapiens ephrin-A1, transcript variant 1) was PCR amplified using Human Universal QUICK-Clone II (Clontech) as the template with primers EFNA1-5 (5′-GTCGAC(Sal I) CACCATGGAGTTCCTCTGGGCCC-3′; SEQ ID NO: 217) and EFNA1-3 (5′-TTAATTAA(Pac I) TCACGGGGTTTGCAGCAGC-3′; SEQ ID NO: 218). The PCR product was gel-purified, and cloned into the pCR-Blunt H-TOPO vector using Zero Blunt TOPO PCR Cloning Kit (Invitrogen). The resulting construct pCRII-EFNA1-1 was sequence confirmed. The EFNA1 cDNA was then released from pCRII-EFNA1-1 with Sal I and Pac I, and subcloned into SacI and PacI-digested TK-SE-mLIGHT2 (SEQ ID NO: 76), placing the expression of EFNA1 under the control of vaccinia synthetic early promoter (P_{SE}). The resulting construct, TK-SE-EFNA1 (SEQ ID NO: 219) was sequence confirmed and used for homologous recombination at the TK locus of GLV-0b348 (SEQ ID NO: 201) to generate GLV-0b358 (SEQ ID NO: 202).

C. Generation of GLV-0b365 (SEL-FUKW)

The cDNA encoding TurboFP635 (Far-red fluorescent protein “katushka”) was PCR amplified using pFUKW (Dr. Marco J. Herold, University of Wurzburg) as the template with primers FUKW-5 (5′-GTCGAC(Sal I) CACCATGGTGGGTGAGGATAGCGTGC-3′; SEQ ID NO: 220) and FUKW-3 (5′-TTAATTAA(Pac I) TCAGCTGTGCCCCAGTTTGC-3′; SEQ ID NO: 221). The PCR product was gel-purified, and cloned into the pCR-Blunt II-TOPO vector using Zero Blunt TOPO PCR Cloning Kit (Invitrogen). The resulting construct pCRH-FUKW was sequence confirmed. The TurboFP635 cDNA was then released from pCRH-FUKW with Sal I and Pac I, and subcloned into HA-SL-hNIS-1 (SEQ ID NO: 225) with the same cuts, replacing the hNIS coding sequence. The resulting construct HA-SEL-FUKW4 (SEQ ID NO: 222), was sequence confirmed, and used for homologous recombination at the HA locus of GLV-0b358 (SEQ ID NO: 202) to generate GLV-0e365 (SEQ ID NO: 203).

D. Generation of GLV-0e406 (SE-Tyrp1/SEL-mTyr)

The TK-SE-Tyrp1-SEL-mTyr (SEQ ID NO: 18) construct, generated as described in Example 2E(iii), was recombined with the TK locus of GLV-0e365 (SEQ ID NO: 203) to create GLV-0e406 (SEQ ID NO: 204). GLV-0e406 expresses Tyrp1 under the control of an SE promoter and mTyr under the control of an SEL promoter.

E. Generation of GLV-0e407 (SEL-mTyr)

The TK-SEL-mTyr (SEQ ID NO: 13) construct, generated as described in Example 2C(ii), was recombined GLV-0e365 (SEQ ID NO: 203) to exchange the expression cassettes in the TK locus and create GLV-0e407 (SEQ ID NO: 205). Thus GLV-0e407 expresses mTyr under the control of the SEL promoter.

### Example 5

### Generation of PC-3 Cells Constitutively Expressing Red Fluorescent Protein (RFP)

PC-3 cells, constitutively expressing RFP were generated by stably inserting cDNA encoding monomeric red fluorescent protein (mRFP) (SEQ ID NO: 42 (cDNA); SEQ ID NO: 43 (protein)) into the PC-3 cell genome by lentiviral transduction using the ViraPower™ Lentiviral Expression System Kit (Invitrogen GmbH, Germany) in accordance with the manufacturer's instructions. The RFP gene for cloning into the lentiviral vector was obtained by PCR from the mRFP-encoding plasmid pCR-TK-(P_{SEL})mRFP (SEQ ID NO: 46) using the following primers, which contain attB recombination sites for gateway cloning: forward-attB 1-mRFP (5′-GGGGACAAGTTTGTACAAAAAAGCAGGCTGCCACCATGGCCTCCTCCG AGG-3′; SEQ ID NO: 39) and reverse-attB2-mRFP (5′-GGGGACCACTTTGTACAAGAAAGCTGGGTCAGAATTCGCCCTTTCATT AGG-3; SEQ ID NO: 40).

The PCR product was cloned into a Gateway entry vector (Invitrogen). Site-specific recombination was then carried out between the Gateway vector and the pLENTI6/V5-DEST retroviral vector (Invitrogen Cat. No. V496-10; SEQ ID NO: 4) according to the manufacturer's instructions to produce pLENTI6N5-DEST-mRFP, which contains the mRFP gene under the control of the human CMV immediate early promoter for constitutive expression. Replication-incompetent mRFP-encoding lentiviruses were produced in 293FT cells via a co-transfection of the Vira Power™ Packaging Mix and the pLENTI6/V5-DEST-mRFP expression plasmid using Lipofectamine™ 2000. After transduction of PC-3 cells with mRFP-coding Lentiviruses, stable RFP-expressing PC-3 clones were selected using 10 μg/mL blasticidin. Approximately 3 months were required for the selection of a stable PC-3-RFP cell line. Expression of RFP in the PC-3 cell line was observed in 100% of the cells as 90 days post-transduction as confirmed by observation using a fluorescent microscope equipped with the appropriate filter. PC-3 RFP cells were cultured at 37° C., 5% CO₂ in DMEM, supplemented with 10% FBS, 1% antibiotic/antimycotic solution an blasticidin (10 μg/mL).

### Example 6

### Visual and Absorbance Detection of Melanin Production in Mammalian Cells Following rVACV Infection

Tyrosinase (Tyr)- and/or tyrosinase related protein 1 (Tyrp-1)-expressing vaccinia viruses (tyr-rVACV) were used to infect mammalian cells in culture to examine production of melanin by visual inspection and measurement of optical density/absorbance of infected tumor cells in vitro.

### A. Melanin Production in A549 Lung Tumor Cells

i. In Vitro Melanin Production in A549 Cells

A549 tumor cells were seeded in T75 flasks at 5.4×10⁶ A549 cells per flask. The following day, recombinant vaccinia viruses (GLV-1h68 (control), GLV-1h322, GLV-1h324, GLV-1h326, and GLV-1h327; see Example 2) were diluted in 10 mL DMEM (without phenol red), supplemented with 2% fetal bovine serum (FBS), and the tumor cells were infected with the diluted virus, using an MOI of 1.0. After 1 h at 37° C., an additional 10 mL DMEM (without phenol red), supplemented with 20% FBS, were added to the cells followed by incubation at 37° C. for 3 days prior to harvesting.

To harvest the cells, the cell culture medium, containing any floating cells was collected in a 50 mL tube. The cells remaining in the flask were washed with 10 mL PBS which were collected and added to 50 mL tube containing the culture medium. The still remaining cells were trypsinized (using 4 mL of Trypsin/EDTA solution), collected, and then also transferred to the same 50 mL tube. 200 μL of the cell suspension were used for absorption spectra measurements, while the rest was centrifuged (1800×g, 7 min). The resultant cell pellet was resuspended in 100 μL PBS before freezing at −80° C. After thawing, 50 μL Zfix (Anatech Ltd.), a zinc formaldehyde fixative, were added to the sample. Once settled, the cells were photographed with a Panasonic DMC-FH27, or iPhone, camera for visual inspection and comparison.

The darkest pigmentation was observed in cells infected with the (P_{SE})Tyrp1/(P_{SEL})mTYR (GLV-1h324), and (P_{SEL})mTyr (GLV-1h327) vaccinia viruses. The (P_{SE})Tyrp1/(P_{SE})mTyr (GLV-1h322) virus infected cells exhibited less pigmentation than the GLV-1h324 and GLV-1h327 virus infected cells. This is likely due to the decreased expression of mTyr from the weaker SE promoter. The GLV-1h322 infected cells, however, were darker in pigmentation than the (P_{SE})mTyr alone (GLV-1h326), indicating the additional expression of (P_{SE})Tyrp1 in GLV-1h322 infected cells increased melanin production, or specifically, increased eumelanin production. No pigmentation was observed in the control GLV-1h68 infected cells.

Absorption spectra measurements were performed as described in Example 1C, in 5 nm increments from 350 to 1000 nm, using a SpectraMax M5 plate reader (Molecular Devices). Relative optical density, compared to GLV-1h68 infected cells, was calculated by dividing the absorbance data from at each wavelength by the absorbance data of GLV-1h68 at the same wavelength.

Consistent with visual observation, the results from the absorbance measurements demonstrated that infection with (P_{SE})Tyrp1/(P_{SEL})mTYR (GLV-1h324) results in the highest optical density and absorption rates over the whole measured spectrum, compared to the other virus infected samples. The relative optical density compared to GLV-1h68 infected samples also showed a strong increase in absorption for GLV-1h324 infected cells. (P_{SEL})mTyr (GLV-1h327) virus infected cells also exhibited high optical density and absorption rates, corresponding to observations made following visual inspection of the cell pellets.

ii. Melanin Production in A549 Xenografts

In a further study, melanin production was tested in A549 tumors (xenografts in live mice) infected with Western Reserve (WR)-derived vaccinia virus strains. A549 tumor xenograft tumors were developed in 6-wk-old male nude mice by implanting 5×10⁶ A549 cells in 100 μL subcutaneously in the posterior thigh of the right and left hind legs (2 tumors per mouse). 21 days after tumor cell implantation, groups of mice were injected with a single retro-orbital injection dose of 5×10⁶ plaque forming units (pfu) GLV-0e406 or GLV-0e407. At 57 days post virus injection, the mice were anesthetized and perfused with 20 or 25 mL (PBS-injected mice) PBS to wash out the blood from all organs and the tumors followed by perfusion with 10 mL Z-fix (Anatech Ltd.) fixative. Visual inspection of the A549 tumors confirmed the production of melanin by WR-derived viruses.

### B. Melanin Production in Other Cell Lines

Melanin production upon mTyr- and/or Tyrp1-expressing rVACV (i.e., GLV-1h310, GLV-1h322, GLV-1h323, GLV-1h324, GLV-1h326, GLV-1h327, GLV-1h458, GLV-1h459, GLV-2b452, GLV-2b453, GLV-2b482, GLV-0e406, GLV-0e407, GLV-1h461, GLV-1h462 and GLV-1h460) infection of CV-1 cells was assessed. Melanin production was assessed by visual inspection and comparison. Melanin production was detected from all tyrosinase encoding virus. In the case of inducible viruses (GLV-1h461, GLV-1h462 and GLV-1h460), melanin production was detected in the presence of doxycycline. In the absence of doxycycline, melanin could not be detected by the naked eye.

Melanin production also was assessed upon mTyr- and/or Tyrp1-expressing rVACV infection in sixteen (16) additional tumor cell lines, including cell lines 1858Mel (Dr. Francesco Marincola, NIH), 1936Mel (Dr. Francesco Marincola, NIH), CHAS, DU-145, FaDu, HeLa, HCT-116, HCT-15, HT-29, MCB3901 (ATCC), PC-3-RFP (see Example 5), and Ripley cells. All cells lines infected with an rVACV strain expressing mTyr with or without co-expression of Tyrp 1 produced melanin, even in melanoma cells lines that exhibit reduced, or no, melanin production on their own.

C. Melanin Production in Cells Co-Infected with Viruses that Express mTyr and Tyrp1

HeLa cells were seeded and infected with GLV-1h311 or GLV-1h354 as previously described. Following infection, the cells were then transfected with pCR-BluntII-TOPO-SalI-tetO-mTyr-E2A-hTyrp1-inverse (SEQ ID NO: 233). The following day, the transfection medium was replaced in each flask with fresh medium that was either untreated or supplemented with 1 μg/mL doxycycline. Following transfection, the cells were harvested and visually inspected for melanin production. Cells transfected with the double construct (pCR-BluntII-TOPO-SalI-tetO-mTyr-E2A-hTyrp1-inverse; SEQ ID NO: 233) expressed melanin in the presence of doxycycline.

### D. Fluorescence and Melanin Production in Cell Culture

Because several of the recombinant viruses contain the expression cassette, (P_{SEL})Ruc-GFP, within the F14.5L locus (see Example 2), in addition to the melanin producing enzyme expression cassette, GFP fluorescence also was assessed by imaging and analyzing the A549 and PC-3-RFP cell pellets in a Carestream small animal imager (Carestream; excitation 490 nm, emission 530 nm), using Carestream Molecular Imaging Second Edition software (version 5.0.6.20). The background fluorescence was subtracted and data were presented as relative fluorescent units (RFU). Melanin production significantly attenuated the fluorescent signal of GFP in the melanin-rVACV-infected cells.

In a further experiment, the RFP signal from PC-3-RFP cells, which constitutively express RFP independent of the virus, was assessed and compared to PC-3 cells, which do not express RFP. PC-3 cells were cultured in DMEM with 10% FBS, 1% antibiotic/antimycotic solution, and blasticidin (10 μg/mL). The cells were seeded the day prior to infection so that the day of infection, the cells were about 80-90% confluent. The cells were infected with GLV-1h68 (control), GLV-1h324 and GLV-1h327 viruses (MOI 0.1) and harvested by trypsinization and centrifugation as described above. The cell pellets were then imaged in a small animal imager (Carestream), using an excitation wavelength of 530 nm and an emission wavelength of 600 nm. Fluorescence signal was measured using the Carestream Molecular Imaging software. Data were presented as RFU following subtraction of background fluorescence. The fluorescence signal measured in melanin-expressing infected PC-3-RFP cells was reduced by approximately 30 fold, compared to PC-3-RFP cells infected with the GLV-1h68 control virus. The reduced signal was comparable to the autofluorescence signal of GLV-1h68-infected PC-3 control cells not expressing RFP. Melanin expression also reduced the autofluorescence signal in PC-3 cells by approximately 25 fold.

### Example 7

### Photoacoustic Imaging of Melanin-rVACV Infected Tumor Cells

Melanin-producing vaccinia viruses (melanin-rVACV), encoding mTyr, or mTyr and Tyrp1, were used to infect tumor cells, and detection of infected tumor cells was assessed, in vitro, ex vivo and in vivo, by photoacoustic imaging.

### A. In Vitro Photoacoustic Imaging of Melanin-rVACV Infected A549 Cells

A549 cells were seeded, infected with GLV-1h68 (control), GLV-1h322 ((P_{SE})Tyrp¹/(P_{SE})mTyr), GLV-1h324 ((P_{SE})TYrP1/(P_{SEL})mTyr), GLV-1h32 ((P_{SE})mTyr) or GLV-1h327 ((P_{SEL})mTyr) using an MOI of 0.1, and harvested 3 days post-infection as described in Example 6. Cell pellets were resuspended to achieve a final concentration of 1×10⁷ cells/100 μL in polyethylene PCR tubes cross-sections for the samples were imaged at multiple wavelengths, including Spectro mode, using a Vevo 2100/LAZR System (Visualsonics). Spectro mode analysis characterized the photoacoustic signal throughout the 680-970 nm range.

Spectral analysis of A549 cells infected with GLV-1h322 ((P_{SE})Tyrp1/(P_{SE})mTyr), GLV-1h324 ((P_{SE})TYrP1/(P_{SEL})mTyr), and GLV-1h327 ((P_{SEL})mTyr) showed measurable absorbance across all wavelengths. When the absorbance spectra for the melanin-rVACV infected cells were normalized based on the spectrum of control cells infected with GLV-1h68, the spectral curves of the melanin-rVACV infected cells were almost identical.

### B. Ex Vivo Photoacoustic Imaging of Melanin-rVACV Infected A549 Xenografts

A549 tumor xenograft tumors were developed in 6-wk-old male nude mice by implanting 5×10⁶ A549 cells in 100 μL subcutaneously in the posterior thigh of the right and left hind legs (2 tumors per mouse). 21 days after tumor cell implantation, groups of mice were injected with a single retro-orbital injection dose of 5×10⁶ plaque forming units (pfu) GLV-1h324 (n=5) in 100 μL PBS, or 100 μL PBS only (n=5). At 30 days post injection (PBS group) or 57 days post virus injection, the mice were anesthetized and perfused with 20 (GLV-1h324 injected mice) or 25 mL (PBS-injected mice) PBS to wash out the blood from all organs and the tumors followed by perfusion with 10 mL Z-fix (Anatech Ltd.) fixative. The tumor tissue samples were dissected and analyzed by multispectral photoacoustic imaging, using a Vevo 2100/LAZR System (Visualsonics). 2-dimensional (2D) and 3-dimensional (3D) images of the tumor tissue samples were generated at all wavelengths between 680-970 nm.

Intense absorbance was observed in the GLV-1h324-infected tumor tissue, compared with the PBS-injected control tumor at all wavelengths. Multiplexing the images for the GLV-1h324-infected tumor revealed an area with differential absorption to the dominant signal. Spectral analysis revealed that the spectral curve of the dominant signal was indistinguishable from that of GLV-1h324 infected cells from part A, above. In contrast, the region exhibiting differential absorption generated a very different spectral curve than the dominant signal.

### C. Whole Animal Photoacoustic Imaging of Melanin-rVACV Infected PC-3 RFP Xenografts

5×10⁶ PC-3-RFP cells were subcutaneously implanted in the posterior thigh of the right hind leg of a 6-wk old male nude mouse. 29 days after tumor cell implantation, the mouse was injected with a single injection of 5×10⁶ pfu GLV-1h327 in 100 μL PBS into the retro-orbital sinus vein. At 58 days post implantation (29 days post injection), the mice were anesthetized and perfused with 20 mL PBS to wash out the blood from all organs and the tumors followed by perfusion with 10 mL Z-fix (Anatech Ltd.) fixative. The internal organs of the mouse were removed exposing the tumor and ventral cavity. While the tumor was the same color as the surrounding tissue, a black lymph node metastasis at the midline, ventral to the spine was apparent within the ventral cavity. 2D and 3D images of the tumor and the black melanin-producing metastasis were analyzed as described above in part B.

The tumor lacked photoacoustical signal, but the melanin-containing metastasis was clearly visible at all wavelengths measured. Spectral analysis of the black metastasis revealed a spectral curve that had similar absorbance characteristics as the region of the tumor tissue exhibiting differential absorption in part B above.

### Example 8

### Magnetic Resonance Imaging (MRI) of Melanin-rVACV Infected Tumor Cells

Melanin-producing vaccinia viruses (melanin-rVACV), encoding mTyr, or mTyr and Tyrp1, were used to infect tumor cells, and detection of infected tumor cells was assessed, in vitro, in vivo and ex vivo, by magnetic resonance imaging (MRI).

### A. In Vitro MRI Detection of Melanin-rVACV Infected PC-3-RFP Cells

PC-3-RFP cells were seeded in T75 flasks. The following day, the cells in two flasks per strain were infected with recombinant vaccinia viruses GLV-1h68 (control), GLV-1h322, GLV-1h324, GLV-1h326 and GLV-1h327 using an MOI of 0.1 and the procedure described in previous examples. Mock-infected cells also were used as a control. For each viral strain, one flask of cells was cultured as described above, and the other flask was supplemented with 150 μM ferric citrate.

Three (3) days after infection, the cells were harvested as described in part A above, and the concentration of cells was adjusted to 1×10⁷ cells/100 μL in PCR tubes. T1 and T2 relaxation times were measured in the small animal 7 Tesla MRI scanner (Bruker BioSpec; RARE-T1+T2-map saturation recovery TR/TE5000-200/11-99 ms). Images were acquired on a single plane, going through the center of the cell pellet.

No major difference in T1 or T2 relaxation times could be discerned between any of the unsupplemented cell samples. In marked contrast, iron supplementation significantly decreased the T1-relaxation times for melanin-expressing cells and also decreased T2-relaxation times. Further, in cells supplemented with iron, increased pigment (melanin) was strongly inversely correlated with T1 relaxation times. For example cells transfected with GLV-1h324, which produced the darkest pigment and most absorbance, exhibited the shortest T1 relaxation time. Infection with the control virus, GLV-1h68 had no effect on T1 or T2 relaxation times, compared to uninfected (mock infected) cells. The numerical results are set forth in Table 18 below.

TABLE 18
Effect of melanin production and Fe(III)-citrate supplementation on T1
and T2 relaxation times.

| | T1 tubes | | | | T2 tubes | | | |
|---|---|---|---|---|---|---|---|---|
| | | | 150 μM | | | | 150 μM | |
| | no extra Fe | | Fe(III)-citrate | | no extra Fe | | Fe(III)-citrate | |
| | average | stdev | average | stdev | average | stdev | average | stdev |
| mock | 2356.1 | 79.5 | 2204.9 | 74 | 290.5 | 20.11 | 250.33 | 11.56 |
| GLV-1h68 | 2339.7 | 121.8 | 2202.15 | 78.3 | 349.96 | 33.37 | 259.12 | 11.14 |
| GLV-1h322 | 2240.4 | 129.1 | 1750.11 | 40.7 | 375.74 | 38.99 | 223.28 | 9.63 |
| GLV-1h324 | 2166.5 | 119.5 | 1315.9 | 9.8 | 312.8 | 26.07 | 160.03 | 1.68 |
| GLV-1h326 | 2206 | 106.7 | 1540.18 | 29.5 | 351.8 | 33.43 | 198.51 | 6.29 |
| GLV-1h327 | 2189.3 | 81.6 | 1450.1 | 8.4 | 276.66 | 14.09 | 154.57 | 1.82 |

### B. Sensitivity of MRI Detection of Melanin-rVACV Infected A549 Cells

The sensitivity of MRI detection of melanin-rVACV (GLV-1h324) infected A549 cells was tested by detection of melanin-rVACV infected A549 cells, diluted among uninfected cells and also, as a function of melanin production.

i. T1 Relaxation Based on Concentration of GLV-1h324 Infected Cells

A549 cells were seeded, infected with GLV-1h324, supplemented with ferric citrate, and harvested three days after infection as described above. Serial 2-fold dilutions of infected cells in uninfected A549 cells were made up to a 1:128 dilution. The cell mixtures, containing 1×10⁷ cells in 100 μL supernatant, were placed in PCR tubes for measurement of the T1-relaxation times in a small animal 7 Tesla MRI scanner (Bruker BioSpec).

No change in T1-relaxation, compared to non-infected cells (T1=1940±11 ms), was observed when cells producing melanin were 1 in 128 (T1=1954±15 ms). However, a significant reduction in T1 relaxation could be discerned in mixtures containing melanin-producing cells diluted 1:64 (T1=1860±16 ms). The T1 relaxation times continued to decrease (correlating with increased contrast) as the proportions of GLV-1h324 infected cells increased.

ii. MRI Detection Based on Melanin Production

A549 cells were seeded, infected with GLV-1h68 (control), GLV-1h324 or GLV-1h327 using an MOI of 1.0, supplemented with ferric citrate, harvested at 24 and 48 hpi, and analyzed for T1 relaxation. T1 relaxation of the cell suspension was conducted as described above. A significant reduction in T1 relaxation was evident for all of the melanin-producing cells 24 h post infection, which was further reduced after 48 hpi.

Samples of GLV-1h68 (control) and GLV-1h324 infected A549 cells were also collected at 12 and 18 hpi. Melanin production, in harvested GLV-1h324-infected cell suspensions, was detected by visual inspection at 18 h, but not 12 h, post-infection. Correlating with visual detection of melanin production, T1 relaxation of the GLV-1h324 infected cells was significantly reduced after 18 hpi, but not at 12 hpi.

### C. In Vivo MRI Detection of Melanin-rVACV Infected A549 Tumor Xenografts

A549 tumor xenograft tumors were developed in 6-wk-old male nude mice by implanting 5×10⁶ A549 cells in 100 μL subcutaneously in the posterior thigh of the right and left hind legs (2 tumors per mouse). 21 days after tumor cell implantation, groups of mice each were injected with a single retro-orbital injection dose of 5×10⁶ pfu GLV-1h324 (n=3) or control-rVACV (n=12) (GLV-1h311 (n=4) or GLV-1h312 (n=8)) in 100 μL PBS. 38 days after virus injection, 2 live mice per group were imaged on a 7 T small animal imaging system (Bruker BioSpec). For each mouse, T1-weighted (T1W) imaging through the tumor was performed using the MSME sequence: TR=927 ms, TE=10.6 ms, NEX4, matrix 256×256 FOV 2.8 cm, slice thickness=0.5 mm, 35 slices, to obtain 3 dimensional data sets. T1 and T2 relaxation time measurements (RARE-T1+T2-map saturation recovery TR/TE5000-200/11-99 ms) were acquired on a single plane, going through the center of both tumors, with the exception of one animal in which only one tumor was imaged.

After imaging, the mice were perfused with 20 mL PBS, followed by 10 mL Zfix (AnaTech) and then frozen at −80° C. The samples were then cryosectioned, using a CM 1950 cryotome (Leica Microsystems, Wetzlar, Germany), and each section was photographed, and a z-stack of images was made to generate a 3D-reconstruction of the mouse tissue (Sarantopoulos et al., (2010) Mol Imaging Biol. 13(5):874-885). The volume viewer plug-in function in the open source ImageJ software (NIH) was used to find the slices obtained from the MR imaging results.

The T1 map results revealed significant (p<0.014, Student's-t-test) T1-relaxation time shortening for the GLV-1h324 infected tumors (1248.3±122.6 ms; n=3 tumors/2 mice) compared to the control-rVACV infected tumors (1828.4±18.5 ms; n=4 tumors/2 mice). T1-relaxation time of muscle tissue in the same mice was indistinguishable between the two groups (1706.1±6.3 ms for the GLV-1h324 group and 1709.0±23.9 ms for the control-rVACV group; n=2 for each group).

Reconstructed T1-weighted images of a GLV-1h68-infected mouse, and a GLV-1h324-infected mouse, were generated and compared. The 2 tumors were clearly visible in both images. But while tumors of the control-rVACV injected mice appeared evenly grey, the melanin-rVACV colonized A549 tumors were characterized by regions of very high contrast (almost white when using the same contrast settings as for the control mice). Color images of cryosections of the same mice, in roughly the same section imaged by MRI, revealed tumors with regions that were nearly all black in the GLV-1h324 mice, due to melanin deposition. No dark (melanin-containing) regions were observed in the control tumor. The bright regions in the MR image of the GLV-1h324 injected mouse corresponded very well with the black (melanin-rich) regions observed in the cryosections. Tumor regions of the GLV-1h324 injected mouse that appeared to not yet produce melanin at the time of the study, according to the cryosection images, showed a similar grey-level by MRI as the tumors of the control mice. Thus, infection of tumor bearing mice with GLV-1h324 resulted in melanin production, that decreased T1 relaxation time and generated melanin-specific contrast in MRI, resulting in readily visible tumors in live mice.

### D. Ex Vivo MRI Detection of Melanin-rVACV Infected A549 Tumor Xenografts

A549 tumor xenograft tumors were developed in 6-wk-old male nude mice by implanting 5×10⁶ A549 cells in 100 μL subcutaneously in the posterior thigh of the right and left hind legs (2 tumors per mouse). 21 days after tumor cell implantation, groups of mice each were injected with a single retro-orbital injection dose of 5×10⁶ pfu GLV-1h324 (n=5), GLV-1h327 (n=5), or control-GLV-1h68 (n=5) in 100 μL PBS or 100 μL PBS only (n=5). At 30 days post injection (PBS group) or 57 days post virus injection, the mice were anesthetized and perfused with 20 mL PBS to wash out the blood from all organs and the tumors followed by perfusion with 10 mL Z-fix (Anatech Ltd.) fixative. The tumors were located and analyzed by visual inspection. Melanin production was clearly visible in tumors of GLV-1h324-, and GLV-1h327-infected cells. No pigment was detected in tumors of GLV-1h68-infected cells compared to the PBS injected control tumor.

One tumor from each group was extracted and embedded into low melting agarose (2% in PBS), and the tumors were imaged together on a 7 Tesla small animal imaging system (Bruker BioSpec), using the T1W Sequence described above, and on a 3 Tesla clinical MRI-scanner (Discovery MR 750, General Electric, Milwaukee, USA) using a T1W Sequence: FRFSE, slice thickness of 1 mm, TE: 14 ms, TR: 120 ms, 3 averages, Matrix 256×256. Relaxation time measurements (RARE-T1+T2-map) were conducted in a single plane going through all tumors as described in section D above. T1 (and T2)-relaxation times were determined in the center of the tumor as well as a region in the rim of tumors. The numerical results are set forth in Table 19.

TABLE 19
T1 and T2 relaxation times for melanin-expressing tumors

| | | | | | T2 Tumors | | | |
|---|---|---|---|---|---|---|---|---|
| | T1 Tumors | | | | center | | | |
| | center | | rim | | av- | | rim | |
| | average | stdev | average | stdev | erage | stdev | average | stdev |
| mock | 1416.6 | 9.04 | 1677.7 | 12.24 | 79.01 | 0.49 | 132.97 | 1.59 |
| GLV- | 1202 | 12.77 | 1403.2 | 11.17 | 53.65 | 2.7 | 53.33 | 0.41 |
| 1h68 | | | | | | | | |
| GLV- | 1017.08 | 11.53 | 1051.36 | 23.83 | 50.8 | 0.49 | 50.97 | 1.24 |
| 1h324 | | | | | | | | |
| GLV- | 1007.6 | 10.39 | 1447.91 | 5.85 | 50.64 | 0.49 | 97.21 | 2.23 |
| 1h327 | | | | | | | | |

Expression of melanin in the melanin-rVACV-infected tumors resulted in a significant decrease in the T1-relaxation times in the center of the tumors imaged and T1- and T2-relaxation times on the rim, with the exception of the GLV-1h327-infected tumor. Notably, the chosen tumor rim region of the GLV-1h327-infected tumor did not contain visible amounts of melanin which explains the higher T1- and T2-relaxation times.

The same tumors were also imaged on a 3 Tesla clinical MRI-scanner (3 Tesla, Discovery MR 750—General Electric. Sequence: FRFSE-sequence with a slice thickness of 1 mm, TE: 14 ms, TR: 120 ms, 3 averages, Matrix 256-256; 16 bit data). Again, melanin expression greatly increased the contrast of the image.

### Example 9

### Magnetic Resonance Imaging (MRI) of Melanin-rVACV Infected Metastases

5×10⁵ PC-3-RFP cells (described in Example 5) were implanted subcutaneously into the hind legs of athymic nude mice. 29 days after cell implantation, 5×10⁶ pfu GLV-1h68, GLV-1h324, or GLV-1h327 in 100 μL phosphate buffered saline (PBS) or 100 μL (PBS) were administered into the retro-orbital sinus vein. 14 or 20 days after virus injection, live tumor-bearing mice were imaged on a small animal 7 Tesla MRI scanner (Bruker BioSpec) as described above.

After MR imaging, the mice were perfused with 20 mL PBS, followed by 10 mL Zfix (AnaTech). After perfusion, the internal organs were removed, and pictures were taken of the ventral cavities. The mice were then decalcified (5 days in Z-fix with 10% formic acid at 4° C. with daily changes of the solution), dehydrated, embedded in paraffin and sectioned in 10 μm slices. Paraffin sections, within 100 μm, were transferred to glass slides and stained with a) hematoxylin (Vector Laboratories) and eosin (Carl-Roth GmbH+Co KG), b) hematoxylin and an anti-rVACV staining (rabbit anti-vaccinia A27L primary antibody detected by ImmPRESS™ Reagent (HRP) Anti-Rabbit stained with ImmPACT™ AMEC Red Peroxidase staining solution by Vector Laboratories), c) the anti-rVACV staining only, or d) no staining with the brown melanin providing the only contrast. The specimens were then examined with a stereo-fluorescence microscope (MZ16 FA; Leica) equipped with a digital CCD camera (DC500, Leica) and the Leica IM1000 4.0 software (1300×1030 pixel RGB-color images). Higher magnification images were obtained using an Axiovert 200M microscope equipped with a digital AxioCam MRc5 (Zeiss) and Axiovision 4.5 software (1293×968 fast bin 2×2 color, RGB-color imaged). All stainings were visualized using white light. Digital images were processed with Photoshop 7.0 (Adobe Systems).

The MR images revealed large tumors and lymph node (LN) metastases. The metastases were preferentially infected by the rVACV. The lymph node metastases of PBS or control-rVACV injected mice had similar grey levels as the primary tumor. The mouse injected with melanin-rVACV, GLV-1h324, had one LN metastasis (LN1) that was similar in color to the primary tumor and another LN metastasis (LN2) in same region that was much brighter than the tumor (and LN1). Following perfusion and dissection of the melanin-rVACV injected mouse, melanin production in LN2 was clearly visible. Lymph vessels, filled with melanin-containing cancer cells, were also apparent. An overlay of images from paraffin-embedded sections and MR images demonstrated that the bright regions in the lymph node metastases co-localized with melanin expression. Results from the animals injected with the other melanin-rVACVs were similar to those observed for GLV-1h324. Immunohistochemical analysis confirmed the presence of rVACV in the lymph node metastases, demonstrating that melanin was actually produced intracellularly in the metastasized lymph nodes and vessels and not transported into these structures from the primary tumor. Stained images also revealed a higher amount of necrotic and dead cells in the virus-infected LN metastases compared to those of the PBS control LNs.

### Example 10

### Multispectral Optoacoustic Tomography (MSOT) of Melanin-rVACV Infected Tumor Cells

Given the optical absorption of melanin in the near infrared, optoacoustic imaging, was used to assess detection and visualization of melanin-producing cells. Multispectral optoacoustic tomographic (MSOT) imaging was used to detect melanin-containing tumors and metastases in live mice, using a melanin-specific algorithm developed for this purpose.

### A. Analysis of Optoacoustic Signal

Optoacoustic images of melanin-rVACV infected cells were analyzed in cell culture to evaluate the relative optoacoustic signal of the different melanin-rVACV strains, the time course of signal detection following melanin-rVACV infection, and the sensitivity of the optoacoustic signal.

i. Quantitative Optoacoustic Signals for Melanin-rVACV Infected cells

PC-3-RFP and A549 cells were mock infected or infected with GLV-1h68 (control), GLV-1h322, GLV-1h324, GLV-1h326, or GLV-1h327 as described in previous Examples. Three days after infection, harvested cell suspensions containing approximately 10⁷ cells in 100 μL PBS were added to 0.2 mL PCR-tubes and pelleted by centrifugation. The cell pellet-containing vials were placed in the center of the field of view of the MSOT imaging system in a Vision128 small animal imaging system (iThera Medical GmbH, Neuherberg, Germany) with a resolution of 150 μm in the imaging plane. Short laser pulses in the nanosecond range with variable wavelengths in the near infrared optical window were generated by a wavelength-tunable Nd:YAG pumped optical parametric oscillator (OPO) laser for illumination, while an arc shape tomographic ultrasound detector array with 128 elements (coverage: 270°, central frequency: 5 MHz, bandwidth: 55%) acquired acoustic signals in the μV range that were converted to digital values with 12 bit accuracy. The transducers were focused cylindrically to collect signals only from a selected imaging plane. The subject was translated perpendicular to the imaging plane to acquire images from multiple positions.

A vial containing black India ink solution was placed adjacent to the experimental samples within the MSOT imaging system. The known optical properties (μₐ=0.5 and 1 cm⁻¹, μ′ₛ=10 cm⁻¹) of the black India ink solution were used for normalization and absolute quantification of the absorption by cells. The vials were imaged in the 690-900 nm range averaging 30 acquisitions and the absorption of the cells was determined by normalizing the pixel intensities of the reconstructed images to the ink solution and subtracting background signals.

Mock and GLV-1h68 (control)-infected cells registered no optoacoustic signal. GLV-1h324 and GLV-1h327 exhibited the highest optoacoustic signal and GLV-1h322 and GLV-1h326 exhibited intermediate signal levels. The optoacoustic signals produced by Melanin-rVACV infected cells were in the near infrared range and were equivalent to the optical absorption of up to 2 cm⁻¹, which were an order of magnitude above the 0.3-0.5 cm⁻¹ tissue optical absorption in near infrared and are comparable with the 2 cm⁻¹ absorption of whole blood. These resulted confirmed there was a sufficient signal to background ratio for detection of melanin-producing cells.

ii. Time Course of Optoacoustic Signal Following Infection

A549 cells were seeded, infected with GLV-1h68 (control) or GLV-1h324 using an MOI of 1.0 as described above. The infected cells were harvested at 7.5, 12, 18, and 24 hours post infection (hpi) and analyzed by MSOT, as described in part A(i) above. Almost no optoacoustic signal was detected for GLV-1h68 (control)-infected cells at any time point. Minimal melanin-specific optoacoustic signal was detected in GLV-1h324-infected cells until 18 hpi, when a significant increase in signal was observed. By 24 hpi, the optoacoustic signal for GLV-1h324-infected cells increased, relative to the signal at 18 hpi, by approximately 3-fold.

iii. Sensitivity of Optoacoustic Signal

A549 cells were seeded, infected with GLV-1h324, and harvested three days after infection as described above. Serial 2-fold dilutions of infected cells in uninfected A549 cells were made up to a 1:128 dilution. The cell mixtures, containing 1×10⁷ cells in 100 μL supernatant, were placed in PCR tubes for MSOT analysis, as described in part A(i) above. Even at the lowest concentration of infected cells (1 infected cell in 128 total cells), an optoacoustic signal was detected that was significantly higher than that of control cells. The melanin signal increased asymptotically with increasing percentages of infected cells.

### B. Generation of Melanin-Specific Algorithm

PC-3-RFP tumors and lymph node metastases were generated in live mice and infected with GLV-1h68 (control) or melanin-producing GLV-1h324 as described in Example 9 above. 14 days after rVACV injection, images of the animals were acquired using the Vision 128 small animal imaging system (iThera Medical GmbH, Neuherberg, Germany) described in part A, above. Inside the imaging system, the animal was mounted in a holder and submerged in a water bath to facilitate acoustic coupling between the tissue and the detector. To prevent drowning, the mouse was wrapped with a thin polyethylene membrane while gas anesthesia using isoflurane was supplied through a breathing mask. Images were acquired at 11 wavelengths in 15 nm intervals from 700 nm to 850 nm. For each position and wavelength, 25 acquisitions were averaged, resulting in a total acquisition time of 2.5 seconds per individual image. Tomographic reconstruction algorithms that modeled detector geometry (see Rosenthal et al., (2010) IEEE Trans Med Imaging. 29(6), 1275-1285) were used to produce an accurate image of the acoustic pressure distribution map inside the animal.

After image reconstruction, linear spectral unmixing was applied to resolve melanin-specific signals. The melanin-specific signal was determined by multispectral processing by way of independent component analysis (Glatz et al., (2011) Opt Express 19(4): 3175-3184). For each pixel in the image, the total measured optoacoustic spectrum was fit to the known absorption spectra profiles of melanin and oxy- and deoxyhemoglobin, which were assumed to be the greatest absorbers in the tissue. The melanin-specific signal distribution was determined by subtracting the background signals of deoxygenated and oxygenated hemoglobin from the melanin signal. In order to retrieve reproducible results, the method was calibrated on a positive cross-section image using literature values for the absorption profiles of melanin and hemoglobin. The resulting optimized unmixing matrix was then saved and re-applied for all other datasets to create reproducible results. Cryosections, as described above, were obtained from approximately the same plane as was imaged to validate the results.

### C. Detection of Melanin Production in Tumors by Multispectral Optoacoustic Imaging In Vivo

The algorithm developed in part B above was used to specifically visualize the presence and distribution of melanin, produced from melanin-rVACV, in live mice using MSOT.

i. Imaging Superficial A549 Tumors

A549 cells were cultured and injected into the hind legs of 6-wk old male nude mice as described in Example 7B. 21 days after tumor cell implantation, groups of mice each were injected with a single retro-orbital injection dose of 5×10⁶ pfu GLV-1h324 (n=2) or control-rVACV, GLV-1h68 (n=1), in 100 μL PBS. Approximately 4 weeks post virus injection, live mice from control and melanin-rVACV groups were imaged by MSOT, using the melanin-specific algorithm and parameters described in part B.

As expected, control-rVACV injected mice did not produce a melanin signal. In contrast, the melanin-expressing tumor was easily detected above the background signal. Although melanin was produced throughout the tumor in the melanin-rVACV injected mouse, the optoacoustic melanin signal was only detected at the rim of the tumor mass. This is probably due to the strong absorption of the excitation and emission light, preventing deeper penetration into the tumor. Cryosections (procedure described above) taken from approximately the same plane as were imaged by MSOT were inspected to confirm the localization of the melanin.

ii. Imaging PC-3-RFP Lymph Node Metastases

PC-3-RFP tumors and metastases were generated in athymic nude mice, and infected with control rVACV (GLV-1h68; n=2) or melanin-rVACV (GLV-1h324; n=3) as described in Example 9. The live mice were subjected to MSOT, as described in part C(i) above, 2 or 3 weeks after virus infection, and the images were processed using the melanin-specific algorithm, described in part B.

The average signal intensity was calculated for each tumor or lymph node metastasis (LN) emitting a melanin-specific signal >1. The mean melanin signal intensities were significantly greater in the melanin-rVACV injected animals than in controls. In addition to increased mean intensities, the area containing signals with melanin signature was much greater in the melanin-rVACV injected mice than in controls, and regions of high intensity were only detected in the melanin-rVACV injected mice.

Cryosections of the regions corresponding to the images taken were inspected and compared to the MSOT images. Large tumors and lymph node metastases were observed in the cryosections. Consistent with previous observations, the cryosections of the melanin-rVACV injected animals revealed strong melanin expression throughout the lymph node metastases generated by the PC-3-RFP cells and puncta of melanin expression within the tumors, indicating preferential viral colonization of the metastases over the primary tumor. The regions of high intensity in the MSOT images correlated with the distribution of melanin in the lymph node metastases and the tumor in corresponding cryosections. The melanin-specific signals detected in tumors and lymph node metastases (LN1 and LN2) in the melanin-rVACV injected mice indicated that MSOT was capable of detecting the presence and distribution of deeply internal melanin-expressing entities, in addition to superficial tumors containing melanin, as observed in part C(i) above.

### Example 11

### Effect of Melanin Production on Vaccinia Virus Replication and Cell Toxicity

The influence of melanin production on viral replication and virus-mediated cell lysis was determined by measuring the activity of the virus-encoded beta glucuronidase activity in cell lysates and the cell culture supernatant, respectively (Hess et al., J. Transl Med. 2011; 9:172). Cell-associated glucuronidase reflects virus-mediated protein expression which correlates with virus replication. Glucuronidase is not secreted by the cells, and therefore, glucuronidase in the cell culture supernatant is reflective of cell toxicity as a result of virus mediated lysis of the tumor cells. Cell toxicity was also measured by MTT assay.

1×10⁵ PC-3-RFP cells were seeded in 24-well plates and infected the following day with recombinant vaccinia viruses (GLV-1h68 (control), GLV-1h322, GLV-1h324, GLV-1h326, and GLV-1h327), as described in Example 6, using an MOI of 0.005. Media and infected cells were harvested from the wells at 10, 24, 48 and 72 hours post virus infection (hpi). The samples of cells and medium were frozen at −80° C. The cell samples were thawed and resuspended in 1 mL PBS with 1% FBS and 0.1% Triton X-100: 5 μL of each sample were analyzed for glucuronidase activity by detecting cleavage of the 4-MUG substrate to generate the fluorescent compound 4-MU, as described in Hess et al., J. Transl Med. 2011; 9:172.

Up to 24 hpi, all cell samples exhibited similar levels of glucuronidase activity. At 48 and 72 hpi, a decrease in glucuronidase activity was observed in cells infected with strong melanin producing rVACV (i.e., (P_{SE})Tyrp1/(P_{SEL})mTYR (GLV-1h324), and (P_{SEL})mTyr (GLV-1h327)), indicating decreased viral replication. The viral replication was not affected in PC-3-RFP infected with rVACV which expressed no or low levels of melanin. In contrast, the amount of released glucuronidase in the supernatant of rVACV-infected cells increased at each time point measured and the toxicities observed were similar at all time points among all viruses tested.

Similar results were observed when cell viability was measured by an MTT assay (Sigma Aldrich), using 2.5 mg MTT (3-(4,5-Dimethylthiazol-2-yl)-2,5-diphenyltetrazolium bromide)/mL DMEM, without phenol red, according to the manufacturer's instructions. Virus-infected cells exhibited decreased cell viability over time, but the rate of cell toxicity was independent of melanin expression levels.

### Example 12

### Oncolytic Activity of Melanin-rVACV

Tumor regression was measured to assess the oncolytic activity of melanin-rVACV. 5×10⁶ A549 cells were implanted subcutaneously into athymic nude mice (n=5). When the tumors reached approximately 300 mm³, 5×10⁶ pfu GLV-1h68 (control), GLV-1h324, or GLV-1h327 resuspended in 100 μL PBS, or 100 μL PBS only, were injected into the retro-orbital sinus vein. Tumor growth was monitored weekly. At the end of the experiment (5 weeks for PBS control mice, 8 weeks for rVACV-injected mice), the tumors were isolated form perfused mice and cut open to confirm the production of melanin in the melanin-rVACV-colonized tumors. Net body weight (i.e., tumor weight subtracted from the whole body weight) at the end of the study also was measured as a marker for “well being.”

Melanin-rVACV injected mice exhibited oncolytic activity, but the tumor regression was decelerated compared to the control rVACV strain, GLV-1h68. Net body weight of the melanin-rVACV injected animals was somewhat higher than those injected with the control strain, although the variability was high. Analyses of isolated tumor tissues from perfused mice at the end of the experiments clearly demonstrated that melanin-rVACV colonized tumors, in contrast to controls, produced so much melanin that they appeared coal-black.

### Example 13

### Laser-Induced Heat Therapy of Melanin-rVACV Infected Cells

A549 cells, approximately 50% confluent in T225-flasks, were infected with 5×10⁶ pfu (MOI 0.1) of GLV-1h68, GLV-1h324, or GLV-1h327. Three days later, cells were harvested by trypsinization and centrifugation at 1800×g for 5 min. The cell pellets were resuspended in 350 μL PBS and frozen at −80° C. before further use. After thawing, 250 μL of the cell suspension were transferred to a 1.5 mL reagent tube and placed in front of a 808 nm laser (MDL-III-808 with PSU-III-LED power supply; 2.00 Watt; Opto Engine LLC). The temperature of the cell suspension was measured by placing an AllTemp™ Infrared digital thermometer (Franmara) at the top of the tube, approximately 26 mm from the cell suspension surface before exposure (0 s), every 10 seconds (from 10 to 180 s), and at 300 s after placing the tube in front of the laser. The temperature was measured in ° F., which was converted to ° C. The results are set forth in Table 20.

TABLE 20
Temperature of rVACV infected cell suspensions were exposed to 808 nm
laser light

Temperature

| | | | | | |
|---|---|---|---|---|---|
| ° F. | ° C. | ° F. | ° C. | ° F. | ° C. |

| | | | |
|---|---|---|---|
| time (s) | GLV-1h68 | GLV-1h324 | GLV-1h327 |

| | | | | | | |
|---|---|---|---|---|---|---|
| 0 | 80.4 | 26.9 | 77.6 | 25.3 | 78.7 | 25.9 |
| 10 | 87.9 | 31.1 | 79.3 | 26.3 | 81.6 | 27.6 |
| 20 | 87.3 | 30.7 | 81 | 27.2 | 83.4 | 28.6 |
| 30 | 87.2 | 30.7 | 83.3 | 28.5 | 85.3 | 29.6 |
| 40 | 87.6 | 30.9 | 86.2 | 30.1 | 87.6 | 30.9 |
| 50 | 87.7 | 30.9 | 89.4 | 31.9 | 90.3 | 32.4 |
| 60 | 88 | 31.1 | 92.5 | 33.6 | 92.8 | 33.8 |
| 70 | 88 | 31.1 | 96 | 35.6 | 95.6 | 35.3 |
| 80 | 88 | 31.1 | 99.6 | 37.6 | 98.3 | 36.8 |
| 90 | 88.3 | 31.3 | 103.2 | 39.6 | 100.7 | 38.2 |
| 100 | 88.3 | 31.3 | 106.4 | 41.3 | 103.5 | 39.7 |
| 110 | 88.3 | 31.3 | 111.3 | 44.1 | 106.3 | 41.3 |
| 120 | 88.8 | 31.6 | 116.1 | 46.7 | 109.3 | 42.9 |
| 130 | 88.8 | 31.6 | 118.8 | 48.2 | 111.9 | 44.4 |
| 140 | 89.1 | 31.7 | 121.9 | 49.9 | 114.6 | 45.9 |
| 150 | 89 | 31.7 | 126.5 | 52.5 | 116.8 | 47.1 |
| 160 | 89.1 | 31.7 | 129.4 | 54.1 | 122.4 | 50.2 |
| 170 | 89.3 | 31.8 | 130.6 | 54.8 | 122.4 | 50.2 |
| 180 | 89.4 | 31.9 | 132.7 | 55.9 | 128.9 | 53.8 |
| 300 | 89 | 31.7 | 150.2 | 65.7 | 143.1 | 61.7 |

The temperature of the GLV-1h68 infected cells increased by a few degrees, which may have in part been due to handling of the tube with warm fingers. In contrast, the temperature of the melanin producing cell suspension increased dramatically. For example, the GLV-1h324 injected cells increased by 40.3° C. by 300 s of exposure, resulting in a temperature higher than 65° C.

### Example 14

### Toxicity Caused by Laser-Induced Heat Therapy of Melanin-rVACV Infected Cells

Frozen cell pellets of mock, GLV-1h68, GLV-1h324, or GLV-1h327 infected A549 cells were generated as described in Example 13. After thawing, the cell pellets were resuspended in 350 μL DMEM medium, without phenol red. From each cell suspension, three aliquots of 100 μL were transferred to 1.5 mL reaction tubes. The tubes were exposed to the 808 nm laser light (MDL-III-808 with PSU-III-LED power supply; 2.00 Watt; Opto Engine LLC) for 60 s, 120 s, or not exposed, but otherwise handled exactly as the other tubes. The temperature of the cell suspensions was measured immediately after treatment by touching the thermometer to the tube next to the cell suspension. Following the temperature measurement 100 μL DMEM medium (without phenol red) and 2-fold serial dilutions in DMEM, in final volumes of 100 μL, were assayed for cell viability using an XTT Cell Proliferation Kit II (Roche; Cat. #11 465 015 001). 50 μL of the pre-mixed cell proliferation kit solution (98% solution 1, 2% solution 2) were added to each dilution, and mixed. After 4 hours incubation at 37° C. in a humidified 5% CO₂ atmosphere, the samples were centrifuged at 13,000 rpm for 1 min, and 100 μL supernatant were transferred to a 96-well plate to determine the optical density at 450 nm and 700 nm. A blank, containing 100 L from a mix of 100 μL DMEM and 50 μL pre-mixed cell proliferation kit solution, that was co-incubated with the other samples, was subtracted from all measurements. The relative survival was calculated as the average of the first 4 dilutions having an optical density (O.D. at 450 nm minus O.D. at 700 nm) >0.4, divided by the average optical density of the non-laser exposed samples.

Exposure to the 808 nm laser light had little effect on the temperature of mock or GLV-1h68 infected cells, raising the temperature about 3° C. after 2 minutes of exposure. In contrast, suspensions of cells infected with melanin-rVACVs exhibited an increase in temperature that correlated exposure time. The temperature of GLV-1h327 infected cells increased by 35° C., and GLV-1h324 infected cells increased by 41° C., following 2 minutes of laser treatment, resulting in a final temperature that was greater than 65° C.

Elevated temperatures correlated with reduced viability in the melanin-rVACV infected cells. After 120 s exposure to the 808 nm laser, survival of GLV-1h327 infected cells was reduced to approximately 60% (p<0.05 by statistical T-test) and survival of GLV-1h324-infected cells was approximately 5% (p<0.0001). Thus exposure to a near infrared laser results in specific hyperthermia, resulting in the death, of melanin-producing cells.

### Example 15

### Laser-Induced Heat Therapy of Melanin-rVACV Infected Tumors

Laser induced hyperthermia treatment was tested in mice that had GLV-1h68 or GLV-1h324 colonized A549 tumors. GLV-1h68 or GLV-1h324 colonized A549 tumors were generated in 6 wk old male nude mice as described in Example 7C. 50 days post tumor cell implantation and 36 days post virus injection, the tumors were exposed to external laser light (MDL-III-808 with PSU-III-LED power supply; 2.00 Watt; Opto Engine LLC; n=4 animals for each virus strain group) for 120 s or not exposed to laser light (n=5 for each virus strain group). The tumor volume was measured immediately before laser treatment and on days 1 and 5 following laser treatment. Observations were also recorded immediately following laser treatment and on days 1 and 5 post laser treatment.

The control group of mice, containing GLV-1h68-colonized tumors, were unaffected by laser treatment, with the exception of 1 mouse which experienced some minor skin damage 1 day after laser treatment. Immediately after treatment, the skin above the laser-exposed section of the tumor became discolored in all mice within the group containing GLV-1h324-colonized tumors. The day after the laser treatment, the discolored skin became necrotic. There was no difference in tumor volume between the laser-treated and untreated animals of the GLV-1h68 control group at any time point. A highly variable, but slightly reduced tumor volume, compared to the tumor volume immediately before GLV-1h68 infection was measured in both laser-treated and untreated animals.

The tumor volumes of the GLV-1h324-infected animals, measured 1 and 5 days after laser treatment, demonstrated that laser treatment of melanin-producing tumors significantly reduced tumor size. The tumor volume of laser-treated GLV-1h324-infected animals decreased by approximately 20% by day 1 which was slightly further reduced by day 5, compared to the tumor volume measured immediately prior to infection. In the absence of laser treatment, the tumor volume of the GLV-1h324-infected animals increased by slightly less than 25% by day 1 and by about 25% by day 5. The difference between laser-treated and untreated GLV-1h324-infected animals was significant on day 1 (p<0.05) and even more so by day 5 (p<0.005).

In a further study, 1×10⁵ B16F10 melanoma cells, which express melanin, were implanted subcutaneously into athymic nude mice. Three days post tumor cell implantation, the tumor diameter was approximately 2-3 mm, and the implanted tumors were subjected to laser treatment (MDL-III-808 with PSU-III-LED power supply; 2.00 Watt; Opto Engine LLC). Five days later, the volume of the laser treated and untreated B16F10 tumors was determined (n=5 for each group). Results from this study were consistent with the previous study in that the laser treated melanin-producing tumors decreased in size following laser treatment. These results demonstrate that near-infrared lasers can be used for hyperthermia therapy to reduce the size of tumors expressing melanin “naturally” or by way of melanin-rVACV infection.

Since modifications will be apparent to those of skill in this art, it is intended that this invention be limited only by the scope of the appended claims.

1. A method of killing or inhibiting growth or proliferation of cells, which are involved in a disease, by an energy-absorbing therapy, comprising:
a) administering a nucleic acid molecule encoding a chromophore-producing enzyme(s) to a subject, wherein:
the nucleic acid molecule is directed to or localized to cells in the subject that are involved in the disease process, whereby the chromophore is expressed in the cells in the subject; and
expression of the encoded chromophore-producing enzyme produces a chromophore product in the cell in the subject into which cell the nucleic acid molecule is delivered; and
b) exposing the subject or cells in the subject to an energy source that is absorbed by the chromophore product to effect local production of heat and/or toxic chemicals in the cells that express the chromophore, thereby killing the cells or inhibiting the proliferation of the cells, thereby effecting treatment of the disease.

2. The method of claim 1, wherein the nucleic acid molecule is heterologous to the cell to which the nucleic acid is directed or localized.

3. The method of claim 1, wherein the cell involved in the disease to which the nucleic acid is directed or localized is a not a melanoma cell.

4. The method of claim 1, wherein the disease is one or more of a proliferative disease or disorder, an inflammatory disease or an immune-mediated disease.

5. The method of claim 1, wherein the disease is a myeloproliferative disease, a lymphoproliferative disease, or a solid tumor disease.

6. The method of claim 4, wherein the disease is a proliferative disorder that is a tumor or a metastasis.

7. The method of claim 1, wherein the cell is a tumor cell that is a solid tumor cell, a circulating tumor cell or a metastatic cell.

8. The method of claim 1, wherein the nucleic acid encoding the chromophore-producing enzyme(s) is operatively inserted into a vector for expression in the cell.

9. The method of claim 8, wherein the vector is a viral vector or a non-viral vector.

10. The method of claim 9 wherein the vector is a viral vector that is an oncolytic virus that accumulates in tumor cells.

11. The method of claim 10, wherein the oncolytic virus is selected from among a Newcastle Disease virus, parvovirus, vaccinia virus, measles virus, reovirus, vesicular stomatitis virus (VSV), oncolytic adenoviruses, poliovirus and herpes viruses, or a derivative thereof that is modified to contain nucleic acid encoding a heterologous gene product.

12. The method of claim 11, wherein the virus is a vaccinia virus selected from among Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC16m8, LC16M0, LIVP, ACAM2000, WR 65-16, Connaught, New York City Board of Health (NYCBH), EM-63 and NYVAC strains.

13. The method of claim 12, wherein the vaccinia virus is a Lister strain virus.

14. The method of any of claim 13, wherein the vaccinia virus is an LIVP virus or derivative thereof.

15. The method of claim 14, wherein the LIVP virus or derivative thereof comprises a sequence of nucleotides set forth in SEQ ID NO:1 or 188, or a sequence of nucleotides that has at least 85% sequence identity to SEQ ID NO:1 or 188.

16. The method of claim 10, wherein the oncolytic virus comprises nucleic acid encoding an additional heterologous gene product.

17. The method of claim 16, wherein the heterologous gene product is a therapeutic and/or a reporter gene product.

18. The method of claim 1, wherein the nucleic acid molecule is operatively inserted for expression in a cell into a non-viral vector.

19. The method of claim 18, wherein the non-viral vector is selected from among a plasmid, cosmid, minicircle and artificial chromosome.

20. The method of claim 1, wherein expression of the encoded chromophore-producing enzyme produces melanin and/or precursors of melanin.

21. The method of claim 20, wherein the melanin comprises eumelanin and/or pheomelanin.

22. The method of claim 1, wherein the nucleic acid molecule encodes a chromophore-producing enzyme that is a tyrosinase, enzymatically active portion thereof or an enzymatically active variant thereof that catalyzes production of a melanin.

23. The method of claim 22, wherein the nucleic acid molecule encodes a tyrosinase enzyme having the sequence of amino acids set forth in any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence or a sequence of amino acids that exhibits at least 75% sequence identity to any of SEQ ID NOS: 7, 81, 82, 84, 85, 87, 89, 91, 93, 95, 97, 99, 101, 103, 105, 107, 109, 111, 113, 115, or 117, or a mature form thereof lacking the signal sequence.

24. The method of claim 1, wherein:
the nucleic acid molecule encodes a tyrosinase having the sequence of amino acids set forth in SEQ ID NO: 7, 81, 82 or 84, or a sequence of amino acids that exhibits at least 75% sequence identity to any of SEQ ID NO: 7, 81, 82 or 84; or
the nucleic acid molecule comprises the sequence of nucleotides set forth in SEQ ID NO: 6, 80 or 83, or a sequence of nucleotides that exhibits at least 75% sequence identity to any of SEQ ID NOS: 6, 80 or 83.

25. The method of claim 22, wherein the nucleic acid molecule encoding the tyrosinase also encodes a chromophore-producing enzyme that is a tyrosinase-related protein 1 (TRP-1) and/or a dopachrome tautomerase (DCT), or an enzymatically active portion thereof or an enzymatically active variant thereof.

26. The method of claim 1, wherein the nucleic acid encodes a chromophore-producing enzyme that is a tyrosinase or enzymatically active portion thereof or enzymatically active variant thereof and a tyrosinase-related protein 1 or enzymatically active portion thereof or enzymatically active variant thereof.

27. The method of claim 26, wherein the nucleic acid molecule encodes a TRP-1 enzyme having the sequence of amino acids set forth in any of SEQ ID NOS: 20, 120, 121, 123, 124, 126, 127, 129, 131, 133, 135, 137, 139, 141, 142 or 144, or a mature form thereof lacking the signal sequence, or a sequence of amino acids that exhibits at least 75% or more sequence identity to any of SEQ ID NOS: 20, 120, 121, 123, 124, 126, 127, 129, 131, 133, 135, 137, 139, 141, 142 or 144 or the mature form thereof lacking the signal sequence

28. The method of claim 1, wherein:
the subject is exposed to the energy source a predetermined time for expression of the nucleic acid in the cell after administration of the nucleic acid molecule encoding the chromophore-producing enzyme(s); and
the predetermined time is sufficient for the nucleic acid molecule to express the chromophore-producing enzyme(s) in a cell in the subject and produce the chromophore product in the cell.

29. The method of claim 1, wherein the energy source is selected from among chemical energy, electric energy, radiant energy, electromagnetic energy, microwave energy, nuclear energy, magnetic energy, elastic energy, sound energy, mechanical energy and luminous energy.

30. The method of claim 1, wherein the energy is applied externally to an area of the subject to be treated.

31. The method of claim 1, wherein the energy source is applied internally directly to a tissue or cell or organ in the subject.

32. The method of claim 31, wherein the energy source is applied directly to an internal tumor or metastasis.

33. The method of claim 1, wherein the energy source is electromagnetic energy applied to effect photothermal therapy or photodynamic therapy.

34. The method of claim 1, wherein the nucleic acid molecule is administered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, topically, ocularly, vaginally, or rectally.

35. The method of claim 1, wherein the nucleic acid molecule is administered intravenously.

36. The method of claim 1, wherein the nucleic acid molecule is administered locally inside a body cavity.

37. The method of claim 1, wherein the subject is a human or is a non-human animal.

38. The method of claim 1, that comprises a method of diagnosis, wherein the nucleic acid molecule encoding a chromophore-producing enzyme is administered to the subject and the method of diagnosing the subject comprises detecting the chromophore-product in the subject to diagnose a tumor and/or a metastasis.

39. A vaccina virus, comprising a sequence of nucleotides encoding a tyrosinase or an enzymatically active portion, or an enzymatically active variant, of the enzyme operatively linked to a strong promoter.

40. The vaccinia virus of claim 39 that is selected from among Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC16m8, LC16M0, LIVP, ACAM2000, WR 65-16, Connaught, New York City Board of Health (NYCBH), EM-63 and NYVAC strain.

41. The vaccinia virus of claim 39, wherein the vaccinia virus is a Lister strain virus.

42. The vaccinia virus of claim 39 that is an LIVP virus or a clonal strain thereof.

43. The vaccinia virus of claim 39, wherein the promoter is selected from among adenoviral major late promoter, vaccinia synthetic early-late promoter (P_{SEL}), vaccinia synthetic late promoter (P_{SL}), simian virus 40 (SV40) promoter, cytomegalovirus (CMV) promoter, respiratory syncytial virus (RSV) promoter, human elongation factor 1α-subunit (EF1-1α) promoter, a ubiquitin C promoter (Ubc), a phosphoglycerate kinase-1 (PGK) promoter, small nuclear RNA U1b promoter and glucose 6-phosphate dehydrogenase promoter.

44. A vaccina virus, comprising a sequence of nucleotides encoding a tyrosinase or an enzymatically active portion, or an enzymatically active variant, comprising an inducible expression system for expression of the tyrosinase enzyme.

45. The Lister strain virus or vaccinia virus of claim 44, wherein the inducible expression system is a tetracycline-inducible gene expression system.

46. A Lister strain virus, comprising:
a first sequence of nucleotides encoding a tyrosinase or an enzymatically active portion or an enzymatically active variant of the enzyme, that catalyzes the production of melanin, operatively linked to a promoter; and
a second sequence of nucleotides encoding an accessory melanin-producing enzyme operatively linked to a promoter, wherein the accessory melanin-producing enzyme is a tyrosinase-related protein 1 (TRP-1) and/or a dopachrome tautomerase (DCT), or an enzymatically active portion thereof or an enzymatically active variant thereof.

47. The Lister strain virus or vaccinia virus of claim 46, wherein the accessory melanin-producing enzyme is a tyrosinase-related protein 1 (TRP-1) or an enzymatically active portion thereof or an enzymatically active variant thereof.

48. A Lister strain virus, comprising a sequence of nucleotides encoding a tyrosinase or an enzymatically active portion, or an enzymatically active variant, of the enzyme that catalyzes production of a melanin, that is selected from among viruses designated GLV-1h326, GLV-1h327, GLV-1h459, GLV-1h460, GLV-1h461, GLV-2b482, GLV-0e407, GLV-1h310, GLV-1h322, GLV-1h323, GLV-1h324, GLV-1h325, GLV-1h458, GLV-2b452, GLV-2b453 and GLV-0e406.

49. A pharmaceutical composition, comprising the vaccinia virus of claim 39 in a pharmaceutically acceptable carrier.

50. The pharmaceutical composition of claim 49 that is formulated for local or systemic administration.

51. A pharmaceutical composition, comprising the Lister strain virus of claim 46 in a pharmaceutically acceptable carrier.

52. The pharmaceutical composition of claim 51 that is formulated for local or systemic administration.

53. A pharmaceutical composition, comprising the Lister strain virus of claim 48 in a pharmaceutically acceptable carrier.

54. The pharmaceutical composition of claim 53 that is formulated for local or systemic administration.
